(19) United States
(12) Patent Application Publication
(10) Pub. No.: US 2022/0265655 A1
(43) Pub. Date: Aug. 25, 2022

(54) METHODS OF TREATING INFLAMMATORY DISEASES

(71) Applicant: Ribon Therapeutics, Inc., Cambridge, MA (US)

(72) Inventors: Mario Niepel, Cambridge, MA (US); Kevin Wayne Kuntz, Cambridge, MA (US); Laurie B. Schenkel, Cambridge, MA (US); Kerren Kalai Swinger, Cambridge, MA (US); Melissa Marie Vasbinder, Cambridge, MA (US)

(21) Appl. No.: 17/586,962

(22) Filed: Jan. 28, 2022

Related U.S. Application Data
(60) Provisional application No. 63/143,317, filed on Jan. 29, 2021.
(60) Provisional application No. 63/166,646, filed on Mar. 26, 2021.

(51) Int. Cl. A61K 31/517; A61K 45/06; A61P 1/00

(52) U.S. Cl. CPC A61K 31/517; A61P 1/00; A61K 45/06

(57) ABSTRACT

The present invention relates to use of quinazolinones and related compounds which are inhibitors of PARP14 for the treatment and prevention of an inflammatory disease.

### CLAIM OF PRIORITY

This application claims priority to U.S. Provisional Patent Application Ser. No. 63/143,317, filed on Jan. 29, 2021 and U.S. Provisional Patent Application Ser. No. 63/166,646, filed on Mar. 26, 2021, the entire contents of which are hereby incorporated by reference.

### FIELD OF THE INVENTION

The present invention relates to use of quinazolinones and related compounds which are inhibitors of PARP14 for the treatment and prevention of inflammatory diseases.

### BACKGROUND OF THE INVENTION

Inflammatory responses triggered by the detection of danger- and pathogen associated molecular patterns are the primary mechanism of the innate immune system to cope with insults. However, uncontrolled, chronic inflammation is the driver of numerous diseases such as allergic asthma, atopic dermatitis, atherosclerosis, pulmonary fibrosis, irritable bowel syndrome, and multiple sclerosis. Despite advances in the treatment of many of these diseases, incidence in the US and globally is increasing with a massive impact on people's health, quality of life, economic productivity, and healthcare expenditure. For example, asthma alone affects approximately 25 million patients in the US, with about 20% of patients being children (https://www.cdc.gov/nchs/fastats/asthma.htm). In most patients, the disease is well-controlled with current medication, but between 5-10% of patients suffer from severe forms of asthma that are not controlled with inhaled corticosteroids or long-acting beta-agonists. Further, atopic dermatitis and psoriasis are chronic inflammatory skin disorders that affect 7-10% and about 3% of adults in the United States, respectively (He et al., J. Allergy Clin. Immunol. 2021, 147(1), pp. 199-212).

Existing treatments of inflammatory diseases are often limited. For example, while multiple intravenously-injected biologic treatments are approved for the treatment of severe asthma, they only provide partial relief and do not control the disease in all patients. In addition, thus far no oral small-molecule therapeutic has been shown to effectively treat any asthma endotype. Further, keloids show high recurrence rates and current therapies have limited efficacies (Diaz, A. et al., “Keloid lesions show increased IL-4/IL-13 signaling and respond to TH2-targeting dupilumab therapy,” JEADV 2019, 34, e159-e209). Accordingly, new means for treatment of inflammatory diseases (e.g., asthma, atopic dermatitis, psoriasis, eosinophilic disorders, and keloids) are continually being sought.

### SUMMARY OF THE INVENTION

The present invention is directed to a method of treating or preventing inflammatory diseases in a patient comprising administering to the patient a therapeutically effective amount of a PARP14 inhibitor, for example, a compound of Formula I:

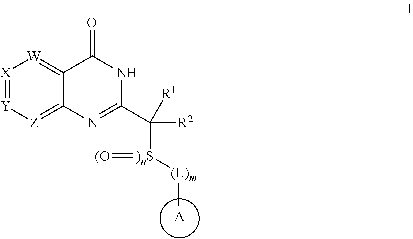

or a pharmaceutically acceptable salt thereof, wherein constituent members are defined below. Compounds of Formula I are described in U.S. Pat. Publication No: US 2019/0194174, the entirety of which is incorporated herein by reference.

The present invention is further directed to a method of treating or preventing asthma in a patient comprising administering to the patient a therapeutically effective amount of a PARP14 inhibitor, for example, a compound of Formula I:

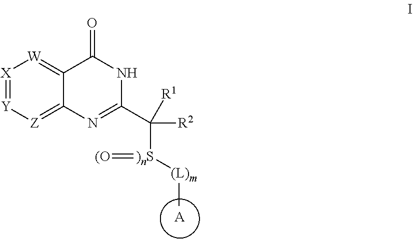

or a pharmaceutically acceptable salt thereof, wherein constituent members are defined below.

The present invention further provides a method of suppression of the immune cell infiltration and activation caused by an inflammatory disease in a patient comprising administering to said patient a therapeutically effective amount of a PARP14 inhibitor, for example, a compound of Formula I, or a pharmaceutically acceptable salt thereof.

The present invention further provides a method of suppression of the immune cell infiltration and activation caused by asthma in a patient comprising administering to said patient a therapeutically effective amount of a PARP14 inhibitor, for example, a compound of Formula I, or a pharmaceutically acceptable salt thereof.

The present invention further provides a method of suppression of the inflammatory cytokines caused by an inflammatory disease in a patient comprising administering to said patient a therapeutically effective amount of a PARP14 inhibitor, for example, a compound of Formula I, or a pharmaceutically acceptable salt thereof.

The present invention further provides a method of suppression of the inflammatory cytokines caused by asthma in a patient comprising administering to said patient a therapeutically effective amount of a PARP14 inhibitor, for example, a compound of Formula I, or a pharmaceutically acceptable salt thereof.

The present invention further provides a PARP14 inhibitor, for example, a compound of Formula I, or a pharmaceutically acceptable salt thereof, for use in the treatment or prevention of an inflammatory disease in a patient.

The present invention further provides a PARP14 inhibitor, for example, a compound of Formula I, or a pharmaceutically acceptable salt thereof, for use in the treatment or prevention of asthma in a patient.

The present invention further provides use of a PARP14 inhibitor, for example, a compound of Formula I, or a pharmaceutically acceptable salt thereof, for the preparation of a medicament for use in the treatment and prevention of an inflammatory disease in a patient.

The present invention further provides use of a PARP14 inhibitor, for example, a compound of Formula I, or a pharmaceutically acceptable salt thereof, for the preparation of a medicament for use in the treatment and prevention of asthma in a patient.

### BRIEF DESCRIPTION OF THE DRAWINGS

FIG. 1. shows percentage of mucin in airway mucosa of Alternaria-challenged mice given Compound 1.

FIG. 2. shows IgE levels (ng/mL) in the serum of Alternaria-challenged mice given varying doses of Compound 1.

FIG. 3. shows relative levels of eosinophils, neutrophils, lymphocytes, and macrophages in bronchoalveolar lavage fluid of Alternaria-challenged mice given varying doses of Compound 1.

FIG. 4. shows IL-4, IL-5, IL-13, IL-33, and TSLP levels in (pg/mL) in bronchoalveolar lavage fluid of Alternaria-challenged mice given varying doses of Compound 1.

FIG. 5. shows IL-4, IL-5, IL-13, IL-33, and TSLP levels in (pg/mg) lung tissue of Alternaria-challenged mice given varying doses of Compound 1.

FIG. 6. shows percentage of mucin in airway mucosa of Alternaria-challenged mice given Compound 1 and Compound 2.

FIG. 7. shows IgE levels (ng/mL) in the serum of Alternaria-challenged mice given Compound 1 and Compound 2.

FIG. 8. shows the number of eosinophils, neutrophils, lymphocytes, and macrophages per mL of bronchoalveolar lavage fluid of Alternaria-challenged mice given Compound 1 and Compound 2.

FIG. 9. shows IL-4, IL-5, IL-13, and IL-33 levels in (pg/mL) in bronchoalveolar lavage fluid of Alternaria-challenged mice given Compound 1 and Compound 2 FIG. 10. shows IL-4, IL-5, IL-13, and IL-33 levels in (pg/mL) in lung tissue of Alternaria-challenged mice given varying doses of Compound 1 and Compound 2.

FIG. 11A is an immunohistochemistry image, at 20× magnification, of normal human skin.

FIG. 11B is an immunohistochemistry image, at 20× magnification, of skin from a patient with systemic sclerosis.

FIG. 12 shows a plot of the body temperature for Groups 1 and 4, up to 60 hours after dosing in the DNCB-induced atopic dermatitis model.

FIG. 13A shows a plot of the clinical score of each group 9 to 24 days after the first DNCB challenge in the DNCB-induced atopic dermatitis model.

FIG. 13B shows the area under the curve of the clinical score of each group after the first DNCB challenge in the DNCB-induced atopic dermatitis model.

FIG. 14 shows a plot of the trans-epidermal water loss over time in the DNCB-induced atopic dermatitis model.

FIG. 15 shows a plot of bodyweight over time in the DNCB-induced atopic dermatitis model.

FIG. 16 shows a plot of the body temperature for the vehicle and Compound 1-treated groups in the IMQ-induced psoriasis model.

FIG. 17 shows a plot of ear thickness for the days preceding and following dosing with Compound 1, DEX, and vehicle in the IMQ-induced psoriasis model.

FIG. 18A shows a plot of the total clinical score in the days after first IMQ challenge.

FIG. 18B shows the area under the curve of the total clinical score for each group in the IMQ-induced psoriasis model.

FIG. 19 shows a plot of the body weight of the vehicle, DEX, Compound 1, and naïve groups over time in the IMQ-induced psoriasis model.

FIG. 20 shows a plot of the body temperature of the vehicle and Compound 1-treated animals in the hIL-23 induced psoriasis model.

FIG. 21A shows a plot of ear thickness for the naïve, vehicle, and Compound 1 groups in the hIL-23 induced psoriasis model.

FIG. 21B shows a graph of the normalized ear thickness AUC for the naïve, vehicle, and Compound 1 groups in the hIL-23 induced psoriasis model.

FIG. 22A shows a graph of ear thickness of the naïve, vehicle, and Compound 1 groups in the hIL-23 induced psoriasis model.

FIG. 22B shows a graph of the increased ear weight for the naïve, vehicle, and Compound 1 groups in the hIL-23 induced psoriasis model.

FIG. 23 shows a plot of the bodyweight of the naïve, vehicle, and Compound 1 groups in the hIL-23 induced psoriasis model.

FIG. 24A shows the body weight and lung weight of mice in each treatment group of the bleomycin (BLM)-induced pulmonary fibrosis model study.

FIG. 24B shows the left lung weight of mice of each treatment group of the BLM-induced pulmonary fibrosis model study.

FIG. 24C shows the post-caval lobe weight of mice in each treatment group of the BLM-induced pulmonary fibrosis model study.

FIG. 24D shows the right inferior lobe weight of mice in each treatment group of the BLM-induced pulmonary fibrosis model study.

FIG. 25A shows the total number of immune cells of the bronchoalveolar lavage fluid of mice in each treatment group of the BLM-induced pulmonary fibrosis model study.

FIG. 25B shows the BALF monocyte ratio of mice in each treatment group of the BLM-induced pulmonary fibrosis model study.

FIG. 25C shows the BALF lymphocyte ratio of mice in each treatment group of the BLM-induced pulmonary fibrosis model study.

FIG. 25D shows the BALF neutrophil ratio of mice in each treatment group of the BLM-induced pulmonary fibrosis model study.

FIG. 26A shows the BALF IL-6 concentrations in mice for each treatment group of the BLM-induced pulmonary fibrosis model study.

FIG. 26B shows the BALF IL-10 concentrations in mice for each treatment group of the BLM-induced pulmonary fibrosis model study.

FIG. 27 shows the lung hydroxyproline content of each treatment group of the BLM-induced pulmonary fibrosis model study.

FIG. 28A shows photomicrographs of Masson's trichrome-stained lung section in the Sham treatment group of the BLM-induced pulmonary fibrosis model study.

FIG. 28B shows photomicrographs of Masson's trichrome-stained lung section in the Vehicle treatment group of the BLM-induced pulmonary fibrosis model study.

FIG. 28C shows photomicrographs of Masson's trichrome-stained lung section in the Compound 1 treatment group of the BLM-induced pulmonary fibrosis model study.

FIG. 28D shows photomicrographs of Masson's trichrome-stained lung section in the Tofacitinib free base treatment group of the BLM-induced pulmonary fibrosis model study.

FIG. 29 shows the Ashcroft score of mice in each of the treatment groups of the BLM-induced pulmonary fibrosis model study.

FIG. 30A is an immunohistochemistry image (20×) of normal human skin.

FIG. 30B is an immunohistochemistry image (20×) of skin from a patient with atopic dermatitis. PARP14 was visualized using DAB (brown chromogen) with a hematoxalin counterstain.

FIG. 30C is an immunohistochemistry image (20×) of skin from a patient with psoriasis. PARP14 was visualized using DAB (brown chromogen) with a hematoxalin counterstain.

FIG. 31A is an immunohistochemistry image (20×) of normal human lung tissue.

FIG. 31B is an immunohistochemistry image (20×) of bronchial epithelial cells of lung tissue from a patient with idiopathic pulmonary fibrosis. PARP14 was visualized using DAB (brown chromogen) with a hematoxalin counterstain.

FIG. 31C is an immunohistochemistry image (20×) of inflammatory cells of lung tissue from a patient with idiopathic pulmonary fibrosis. PARP14 was visualized using DAB (brown chromogen) with a hematoxalin counterstain.

FIG. 32A shows the in-life body weight loss scoring of disease severity in the OXZ-induced colitis mouse model.

FIG. 32B shows the in-life DAI scoring of disease severity in the OXZ-induced colitis mouse model.

FIG. 32C shows the in-life AUC of body weight change scoring of disease severity in the OXZ-induced colitis mouse model.

FIG. 32D shows the in-life AUC of DAI scoring of disease severity in the OXZ-induced colitis mouse model.

FIG. 33A shows a graph of the colon density in the OXZ-induced colitis mouse model.

FIG. 33B shows a graph of the colon length in the OXZ-induced colitis mouse model.

FIG. 33C shows a graph of the colon weight in the OXZ-induced colitis mouse model.

FIG. 34A shows the H&E (hematoxylin and eosin) staining photo at 200× magnification of a sample from the sham group in the OXZ-induced colitis mouse model.

FIG. 34B shows the H&E staining photo at 200× magnification of a sample from the vehicle group in the OXZ-induced colitis mouse model.

FIG. 34C shows the H&E staining photo at 200× magnification of a sample from the tofacitinib group in the OXZ-induced colitis mouse model.

FIG. 34D shows the H&E staining photo at 200× magnification of a sample from the Compound 1 group in the OXZ-induced colitis mouse model.

FIG. 35 shows the pathological score of the severity of colitis in the OXZ-induced colitis mouse model.

### DETAILED DESCRIPTION

The present invention is directed to a method of treating or preventing an inflammatory disease in a patient comprising administering to the patient a therapeutically effective amount of a PARP14 inhibitor, such as a compound of Formula I.

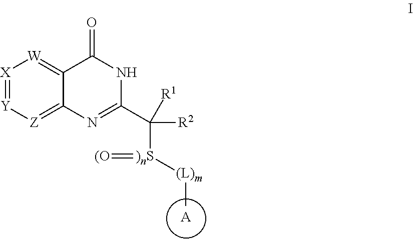

or a pharmaceutically acceptable salt thereof, wherein:

W is CR^{W} or N;

X is CR^{X} or N;

Y is CR^{Y} or N;

Z is CR^{Z} or N;

wherein no more than two of W, X, Y, and Z are simultaneously N;

Ring A is monocyclic or polycyclic C₃₋₁₄ cycloalkyl or Ring A is monocyclic or polycyclic 4-18 membered heterocycloalkyl, wherein Ring A is optionally substituted by 1, 2, 3, or 4 R^{A}, and Ring A is attached to the -(L)ₘ- moiety of Formula I through a non-aromatic ring when Ring A is polycyclic;

L is —(CR⁵R⁶)ₜ—, —(CR⁵R⁶)ₚ—O—(CR⁵R⁶)_{q}—, —(CR⁵R⁶)ₚ—S—(CR⁵R⁶)_{q}—, —(CR⁵R⁶)ₚ—NR³—(CR⁵R⁶)_{q}—, —(CR⁵R⁶)ₚ—CO—(CR⁵R⁶)_{q}—, —(CR⁵R⁶)ᵣ—C(O)O—(CR⁵R⁶)ₛ—, —(CR⁵R⁶)ᵣ—CONR³—(CR⁵R⁶)ₛ—, —(CR⁵R⁶)ₚ—SO—(CR⁵R⁶)_{q}—, —(CR⁵R⁶)ₚ—SO₂—(CR⁵R⁶)_{q}—, —(CR⁵R⁶)ᵣ—SONR³—(CR⁵R⁶)ₛ—, or —NR³CONR⁴—;

R¹ and R² are each, independently, selected from H and methyl;

R³ and R⁴ are each, independently, selected from H and C₁₋₄ alkyl;

R⁵ and R⁶ are each, independently, selected from H, halo, C₁₋₄ alkyl, C₁₋₄ alkoxy, C₁₋₄ haloalkyl, amino, C₁₋₄ alkylamino, and C₂₋₈ dialkylamino;

each R^{A} is independently selected from halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{A} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

R^{W}, R^{X}, R^{Y}, and R^{Z} are each, independently, selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{W}, R^{X}, R^{Y}, or R^{Z} are each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

wherein when W is CR^{W}, X is CR^{X}, Y is CR^{Y}, and Z is CR^{Z}, then at least one of R^{W}, R^{X}, R^{Y}, and R^{Z} is other than H;

each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

each R^{a1}, R^{b1}, R^{c1}, R^{d1}, R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a1}, R^{b1}, R^{c1}, R^{d1}, R^{a2}, R^{b2}, R^{c2}, or R^{d2} is optionally substituted with 1, 2, 3, 4, or 5 substituents independently selected from Cy³, Cy³-C₁₋₄ alkyl, halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, C(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

each Cy³ is C₆₋₁₀ aryl, C₃₋₇ cycloalkyl, 5-10 membered heteroaryl, or 4-10 membered heterocycloalkyl, each optionally substituted by 1, 2, 3, or 4 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

R^{a3}, R^{b3}, R^{c3}, and R^{d3} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;

or R^{c1} and R^{d1} together with the N atom to which they are attached form a 4-7 membered heterocycloalkyl group optionally substituted with 1, 2, or 3 substituents independently selected from halo, C₁₋₄ alkyl, C₁₋₄haloalkyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

or R^{c2} and R^{d2} together with the N atom to which they are attached form a 4-7 membered heterocycloalkyl group optionally substituted with 1, 2, or 3 substituents independently selected from halo, C₁₋₄ alkyl, C₁₋₄haloalkyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

each R^{e1}, R^{e2}, and R^{e3} is independently selected from H, C₁₋₄ alkyl, and CN;

m is 0 or 1,

n is 0, 1, or 2;

p is 0, 1, or 2;

q is 0, 1, or 2, wherein p+q is 0, 1, or 2;

r is 0 or 1;

s is 0 or 1, where r+s is 0 or 1; and

t is 1, 2, or 3;

wherein any aforementioned heteroaryl or heterocycloalkyl group comprises 1, 2, 3, or 4 ring-forming heteroatoms independently selected from O, N, and S;

wherein one or more ring-forming C or N atoms of any aforementioned heterocycloalkyl group is optionally substituted by an oxo (═O) group; and

wherein one or more ring-forming S atoms of any aforementioned heterocycloalkyl group is optionally substituted by one or two oxo (═O) groups.

The present invention is directed to a method of treating or preventing asthma in a patient comprising administering to the patient a therapeutically effective amount of a PARP14 inhibitor, such as a compound of Formula I:

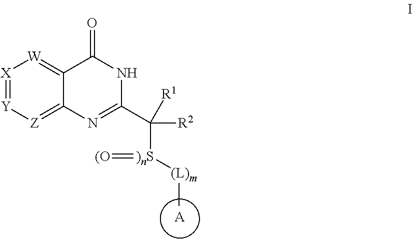

or a pharmaceutically acceptable salt thereof, wherein:

W is CR^{W} or N;

X is CR^{X} or N;

Y is CR^{Y} or N;

Z is CR^{Z} or N;

wherein no more than two of W, X, Y, and Z are simultaneously N;

Ring A is monocyclic or polycyclic C₃₋₁₄ cycloalkyl or Ring A is monocyclic or polycyclic 4-18 membered heterocycloalkyl, wherein Ring A is optionally substituted by 1, 2, 3, or 4 R^{A}, and Ring A is attached to the -(L)ₘ- moiety of Formula I through a non-aromatic ring when Ring A is polycyclic;

L is —(CR⁵R⁶)ₜ—, —(CR⁵R⁶)ₚ—O—(CR⁵R⁶)_{q}—, —(CR⁵R⁶)ₚ—S—(CR⁵R⁶)_{q}—, —(CR⁵R⁶)ₚ—NR³—(CR⁵R⁶)_{q}—, —(CR⁵R⁶)ₚ—CO—(CR⁵R⁶)_{q}—, —(CR⁵R⁶)ᵣ—C(O)O—(CR⁵R⁶)ₛ—, —(CR⁵R⁶)ᵣ—CONR³—(CR⁵R⁶)ₛ—, —(CR⁵R⁶)ₚ—SO—(CR⁵R⁶)_{q}—, —(CR⁵R⁶)ₚ—SO₂—(CR⁵R⁶)_{q}—, —(CR⁵R⁶)ᵣ—SONR³—(CR⁵R⁶)ₛ—, or —NR³CONR⁴—;

R¹ and R² are each, independently, selected from H and methyl;

R³ and R⁴ are each, independently, selected from H and C₁₋₄ alkyl;

R⁵ and R⁶ are each, independently, selected from H, halo, C₁₋₄ alkyl, C₁₋₄ alkoxy, C₁₋₄ haloalkyl, amino, C₁₋₄ alkylamino, and C₂₋₈ dialkylamino;

each R^{A} is independently selected from halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{A} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

R^{W}, R^{X}, R^{Y}, and R^{Z} are each, independently, selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{W}, R^{X}, R^{Y}, or R^{Z} are each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR², C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

wherein when W is CR^{W}, X is CR^{X}, Y is CR^{Y}, and Z is CR^{Z}, then at least one of R^{W}, R^{X}, R^{Y}, and R^{Z} is other than H;

each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

each R^{a1}, R^{b1}, R^{c1}, R^{d1}, R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a1}, R^{b1}, R^{c1}, R^{d1}, R^{a2}, R^{b2}, R^{c2}, or R^{d2} is optionally substituted with 1, 2, 3, 4, or 5 substituents independently selected from Cy³, Cy³-C₁₋₄ alkyl, halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

each Cy³ is C₆₋₁₀ aryl, C₃₋₇ cycloalkyl, 5-10 membered heteroaryl, or 4-10 membered heterocycloalkyl, each optionally substituted by 1, 2, 3, or 4 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

R^{a3}, R^{b3}, R^{c3}, and R^{d3} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;

or R^{c1} and R^{d1} together with the N atom to which they are attached form a 4-7 membered heterocycloalkyl group optionally substituted with 1, 2, or 3 substituents independently selected from halo, C₁₋₄ alkyl, C₁₋₄haloalkyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

or R^{c2} and R^{d2} together with the N atom to which they are attached form a 4-7 membered heterocycloalkyl group optionally substituted with 1, 2, or 3 substituents independently selected from halo, C₁₋₄ alkyl, C₁₋₄haloalkyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

each R^{e1}, R^{e2}, and R^{e3} is independently selected from H, C₁₋₄ alkyl, and CN;

m is 0 or 1,

n is 0, 1, or 2;

p is 0, 1, or 2;

q is 0, 1, or 2, wherein p+q is 0, 1, or 2;

r is 0 or 1;

s is 0 or 1, where r+s is 0 or 1; and

t is 1, 2, or 3;

wherein any aforementioned heteroaryl or heterocycloalkyl group comprises 1, 2, 3, or 4 ring-forming heteroatoms independently selected from O, N, and S;

wherein one or more ring-forming C or N atoms of any aforementioned heterocycloalkyl group is optionally substituted by an oxo (═O) group; and

wherein one or more ring-forming S atoms of any aforementioned heterocycloalkyl group is optionally substituted by one or two oxo (═O) groups.

In some embodiments, when W is CR^{W}, X is CR^{X}, Y is CR^{Y}, and Z is CR^{Z} and when m is 1 or 2, then R^{X} and R^{Y} are not both methoxy;

In some embodiments, the compound is other than:

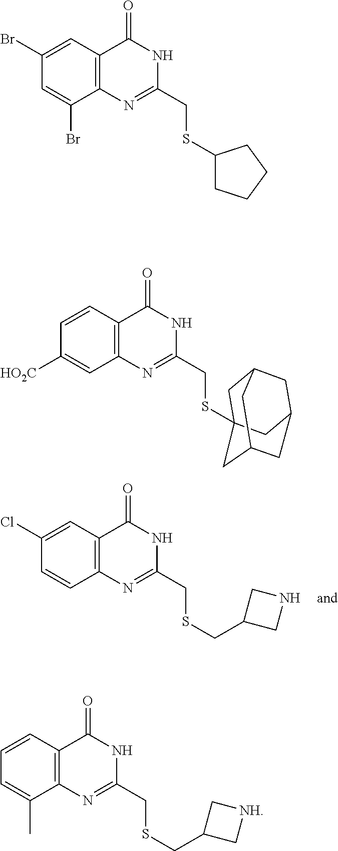 [SMILES: O=c1[nH]c(CSC2CCCC2)nc2c(Br)cc(Br)cc12 ; O=C(O)c1ccc2c(=O)[nH]c(CSC34CC5CC(CC(C5)C3)C4)nc2c1 ; O=c1[nH]c(CSCC2CNC2)nc2ccc(Cl)cc12 ; Cc1cccc2c(=O)[nH]c(CSCC3CNC3)nc12]

In some embodiments, W is CR^{W}; X is CR; Y is CR^{Y}; and Z is CR^{Z}.

In some embodiments, W is N; X is CR^{X}; Y is CR^{Y}; and Z is CR^{Z}.

In some embodiments, W is CR^{W}; X is N; Y is CR^{Y}; and Z is CR^{Z}.

In some embodiments, W is CR^{W}; X is CR^{X}; Y is N; and Z is CR.

In some embodiments, W is CR^{W}; X is CR^{X}; Y is CR^{Y}; and Z is N.

In some embodiments, Ring A is monocyclic or polycyclic C₃₋₁₄ cycloalkyl optionally substituted by 1, 2, 3, or 4 R^{A}, wherein Ring A is attached to the -(L)ₘ- moiety of Formula I through a non-aromatic ring when Ring A is polycyclic.

In some embodiments, Ring A is monocyclic C₃₋₇ cycloalkyl optionally substituted by 1, 2, 3, or 4 R^{A}.

In some embodiments, Ring A is cyclobutyl, cyclopentyl, cyclohexyl, or cycloheptyl optionally substituted by 1, 2, 3, or 4 R^{A}.

In some embodiments, Ring A is cyclobutyl, cyclopentyl, cyclohexyl, or cycloheptyl.

In some embodiments, Ring A is cyclohexyl or cycloheptyl optionally substituted by 1, 2, 3, or 4 R^{A}.

In some embodiments, Ring A is cyclohexyl or cycloheptyl.

In some embodiments, Ring A is cyclohexyl optionally substituted by 1, 2, 3, or 4 R^{A}.

In some embodiments, Ring A is cyclohexyl.

In some embodiments, Ring A is monocyclic or polycyclic 4-18 membered heterocycloalkyl optionally substituted by 1, 2, 3, or 4 R^{A}, and wherein Ring A is attached to the -(L)ₘ- moiety of Formula I through a non-aromatic ring when Ring A is polycyclic.

In some embodiments, Ring A is monocyclic 4-7 membered heterocycloalkyl optionally substituted by 1, 2, 3, or 4 R^{A}.

In some embodiments, Ring A is monocyclic 4-7 membered heterocycloalkyl.

In some embodiments, Ring A is oxetanyl, tetrahydropyranyl, oxepanyl, azetidinyl, pyrrolidinyl, piperidinyl, or azepanyl, optionally substituted by 1, 2, 3, or 4 R^{A}.

In some embodiments, Ring A is oxetanyl, tetrahydropyranyl, oxepanyl, azetidinyl, pyrrolidinyl, piperidinyl, or azepanyl.

In some embodiments, Ring A is oxetanyl, tetrahydropyranyl, oxepanyl, azetidinyl, pyrrolidinyl, piperidinyl, azepanyl, or tetrahydrothiopyranyl optionally substituted by 1, 2, 3, or 4 R^{A}.

In some embodiments, Ring A is oxetanyl, tetrahydropyranyl, oxepanyl, azetidinyl, pyrrolidinyl, piperidinyl, azepanyl, or tetrahydrothiopyranyl.

In some embodiments, Ring A is piperidinyl optionally substituted by 1, 2, 3, or 4 R^{A}.

In some embodiments, Ring A is piperidinyl.

In some embodiments, Ring A is piperidin-4-yl optionally substituted by 1, 2, 3, or 4 R^{A}.

In some embodiments, Ring A is piperidin-4-yl.

In some embodiments, Ring A is tetrahydropyranyl optionally substituted by 1, 2, 3, or 4 R^{A}.

In some embodiments, Ring A is tetrahydropyranyl.

In some embodiments, Ring A is tetrahydropyran-4-yl optionally substituted by 1, 2, 3, or 4 R^{A}.

In some embodiments, Ring A is tetrahydropyran-4-yl.

In some embodiments, L is —(CR⁵R⁶)ₜ—.

In some embodiments, L is —(CR⁵R⁶)ₜ— and t is 1.

In some embodiments, L is —(CR⁵R⁶)ₜ— and t is 2.

In some embodiments, L is —(CR⁵R⁶)ₜ— and t is 3.

In some embodiments, L is —CH₂—.

In some embodiments, m is 0.

In some embodiments, m is 1.

In some embodiments, n is 0.

In some embodiments, n is 1.

In some embodiments, n is 2.

In some embodiments, R¹ and R² are both H.

In some embodiments, one of R¹ and R² is H and the other is methyl.

In some embodiments, each R^{A} is independently selected from C₁₋₆ alkyl, OR^{a1}, C(O)R^{b1}, NR^{c1}R^{d1}, and S(O)₂R^{b1}; wherein said C₁₋₆ alkyl is optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, each R^{A} is independently selected from C₁₋₆ alkyl, halo, C₁₋₆ haloalkyl, OR^{a1}, C(O)R^{b1}, C(O)NR^{c1}R^{d1}, C(O)OR^{a1}, NR^{c1}R^{d1}, S(O)₂R^{b1}, 4-10 membered heterocycloalkyl, 5-10 membered heteroaryl, 4-10 membered heterocycloalkyl-C₁₋₄ alkyl and 5-10 membered heteroaryl-C₁₋₄ alkyl; wherein said C₁₋₆ alkyl, C₁₋₆ haloalkyl, 4-10 membered heterocycloalkyl, 5-10 membered heteroaryl, 4-10 membered heterocycloalkyl-C₁₋₄ alkyl and 5-10 membered heteroaryl-C₁₋₄ alkyl are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, each R^{A} is independently selected from halo, C₁₋₆ haloalkyl, OR^{a1}, C(O)NR^{c1}R^{d1}, and C(O)OR^{a1}.

In some embodiments, R^{A} is OR^{a1}.

In some embodiments, each R^{A} is independently selected from halo, C₁₋₆ alkyl, C₁₋₆ haloalkyl, C₆₋₁₀ aryl-C₁₋₄ alkyl, 5-10 membered heteroaryl-C₁₋₄ alkyl, 4-10 membered heterocycloalkyl-C₁₋₄ alkyl, CN, OR^{a1}, NR^{c1}R^{d1}, C(O)NR^{c1}R^{d1}, NR^{c1}C(O)R^{b1}, C(O)R^{b1}, C(O)OR^{a1}, and S(O)₂R^{b1}, wherein said C₁₋₆ alkyl, C₁₋₆ haloalkyl, C₆₋₁₀ aryl-C₁₋₄ alkyl, 5-10 membered heteroaryl-C₁₋₄ alkyl, and 4-10 membered heterocycloalkyl-C₁₋₄ alkyl are each optionally substituted with 1, 2, 3, 4, or 5 substituents independently selected from halo, CN, OR^{a1}, NR^{c1}R^{d1}, C(O)R^{b1}, and NR^{c1}C(O)R^{b1}.

In some embodiments, each R^{W}, R^{X}, R^{Y}, and R^{Z} is independently selected from H, halo, C₁₋₆ alkyl, C₁₋₆ haloalkyl, 5-10 membered heteroaryl, 4-10 membered heterocycloalkyl, C₆₋₁₀ aryl-C₁₋₄ alkyl, CN, OR^{a2}, C(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, and NR^{c2}S(O)₂NR^{c2}R^{d2}; wherein said C₁₋₆ alkyl, C₁₋₆ haloalkyl, 5-10 membered heteroaryl, 4-10 membered heterocycloalkyl, and C₆₋₁₀ aryl-C₁₋₄ alkyl of R^{W}, R^{X}, R^{Y}, and R^{Z} are each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}.

In some embodiments, each R^{W}, R^{X}, R^{Y}, and R^{Z} is independently selected from H, halo, C₁₋₆ alkyl, C₁₋₆ haloalkyl, 5-10 membered heteroaryl, 4-10 membered heterocycloalkyl, C₆₋₁₀ aryl-C₁₋₄ alkyl, CN, OR^{a2}, C(O)NR^{c2}R^{d2}, NR^{c2}R^{d2}, and NR^{c2}C(O)R^{b2}; wherein said C₁₋₆ alkyl, C₁₋₆ haloalkyl, 5-10 membered heteroaryl, 4-10 membered heterocycloalkyl, and C₆₋₁₀ aryl-C₁₋₄ alkyl of R^{W}, R^{X}, R^{Y}, and R^{Z} are each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}.

In some embodiments, W is CR^{W} and R^{W} is other than H.

In some embodiments, W is CR^{W} and R^{W} is H.

In some embodiments, R^{W} is halo.

In some embodiments, R^{W} is F.

In some embodiments, R^{W} is selected from C₁₋₆ alkyl, C₁₋₆ haloalkyl, halo, and OR^{a2}, wherein said C₁₋₆ alkyl and C₁₋₆ haloalkyl are each optionally substituted with OR^{a2}.

In some embodiments, R^{W} is selected from C₁₋₆ alkyl, C₁₋₆ haloalkyl, CN, halo, and OR^{a2}, wherein said C₁₋₆ alkyl and C₁₋₆ haloalkyl are each optionally substituted with OR^{a2}.

In some embodiments, R^{X} and R^{Z} are not both halogen.

In some embodiments, R^{Z} is H.

In some embodiments, when W is CR^{W}, X is CR^{X}, Y is CR^{Y}, and Z is CR^{Z} and when m is 1 or 2, then R^{X} and R^{Y} are not both C₁₋₆ alkoxy.

In some embodiments, when W is CR^{W}, X is CR^{X}, Y is CR^{Y}, and Z is CR^{Z} and when m is 1 or 2, then R^{X} and R^{Y} are not the same.

In some embodiments, X is CR^{X} and R^{X} is other than H.

In some embodiments, X is CR^{X} and R^{X} is H.

In some embodiments, R^{X} is selected from C₁₋₆ alkyl, halo, and OR^{a2}.

In some embodiments, Y is CR^{Y} and R^{Y} is other than H.

In some embodiments, Y is CR^{Y} and R^{Y} is H.

In some embodiments, Y is CR^{Y} and R^{Y} is independently selected from NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, and NR^{c2}S(O)₂NR^{c2}R^{d2}.

In some embodiments, Y is CR^{Y} and R^{Y} is independently selected from C₁₋₆ alkyl, OR^{a2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, and NR^{c2}S(O)₂NR^{c2}R^{d2}.

In some embodiments, Y is CR^{Y} and R^{Y} is independently selected from NR^{c2}R^{d2} and NR^{c2}C(O)R^{b2}.

In some embodiments, R^{Y} is independently selected from C₁₋₆ alkyl, C₃₋₇ cycloalkyl-C₁₋₄ alkyl, 5-10 membered heteroaryl, 4-10 membered heterocycloalkyl, halo, CN, OR^{a2}, SR^{a2}, C(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, and NR^{c2}S(O)₂NR^{c2}R^{d2}, wherein said C₁₋₆ alkyl, C₃₋₇ cycloalkyl-C₁₋₄ alkyl, 5-10 membered heteroaryl, and 4-10 membered heterocycloalkyl of R are each optionally substituted with 1, 2, 3, 4, or 5 substituents independently selected from halo, C₁₋₆ alkyl, C₁₋₆ haloalkyl, CN, NO₂, OR^{a2}, NR^{c2}R^{d2}, and S(O)₂R^{b2}.

In some embodiments, Y is CR^{Y} and R^{Y} is independently selected from C₁₋₆ alkyl and OR^{a2}.

In some embodiments, Y is CR^{Y} and R^{Y} is OR^{a2}.

In some embodiments, Z is CR^{Z} and R^{Z} is other than H.

In some embodiments, Z is CR^{Z} and R^{Z} is H.

In some embodiments, Z is CR^{Z} and R^{Z} is C₁₋₆ alkyl.

In some embodiments, Z is CR^{Z} and R^{Z} is C₁₋₆ alkyl, halo, or CN.

In some embodiments, each R^{a1}, R^{b1}, R^{c1}, R^{d1}, R^{a2}, R^{b2}, R^{c2}, and R^{d2} is independently selected from H, C₁₋₆ alkyl, and C₁₋₆ haloalkyl, wherein the C₁₋₆ alkyl is optionally substituted with 1, 2, 3, 4, or 5 substituents independently selected from Cy³, Cy³-C₁₋₄ alkyl, halo, C₁₋₄ alkyl, C₁₋₄ haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3}.

In some embodiments, each R^{a1}, R^{b1}, R^{c1}, R^{d1}, R^{a2}, R^{b2}, R^{c2}, and R^{d2} is independently selected from H, C₁₋₆ alkyl, and C₁₋₆ haloalkyl.

In some embodiments, each R^{a1}, R^{b1}, R^{c1}, R^{d1}, R^{a2}, R^{b2}, R^{c2}, and R^{d2} is independently selected from H, C₁₋₆ alkyl, C₁₋₆ haloalkyl, C₆₋₁₀ aryl, C₃₋₇ cycloalkyl, 4-10 membered heterocycloalkyl, C₆₋₁₀ aryl-C₁₋₄ alkyl, C₃₋₇ cycloalkyl-C₁₋₄ alkyl, and 4-10 membered heterocycloalkyl-C₁₋₄ alkyl, wherein said C₁₋₆ alkyl, C₁₋₆ haloalkyl, C₆₋₁₀ aryl, C₃₋₇ cycloalkyl, 4-10 membered heterocycloalkyl, C₆₋₁₀ aryl-C₁₋₄ alkyl, C₃₋₇ cycloalkyl-C₁₋₄ alkyl, and 4-10 membered heterocycloalkyl-C₁₋₄ alkyl are each optionally substituted with 1, 2, 3, 4, or 5 substituents independently selected from C₁₋₄ alkyl, C₁₋₄ haloalkyl, halo, CN, OR^{a3}, C(O)R^{b3}, C(O)OR^{a3} and S(O)₂R^{b3}.

In some embodiments, R^{a2} is selected from H, C₁₋₆ alkyl, C₁₋₆ haloalkyl, C₆₋₁₀ aryl, C₃₋₇ cycloalkyl, 4-10 membered heterocycloalkyl, C₆₋₁₀ aryl-C₁₋₄ alkyl, C₃₋₇ cycloalkyl-C₁₋₄ alkyl, and 4-10 membered heterocycloalkyl-C₁₋₄ alkyl, wherein said C₁₋₆ alkyl, C₁₋₆ haloalkyl, C₆₋₁₀ aryl, C₃₋₇ cycloalkyl, 4-10 membered heterocycloalkyl, C₆₋₁₀ aryl-C₁₋₄ alkyl, C₃₋₇ cycloalkyl-C₁₋₄ alkyl, and 4-10 membered heterocycloalkyl-C₁₋₄ alkyl are each optionally substituted with 1, 2, 3, 4, or 5 substituents independently selected from C₁₋₄ alkyl, C₁₋₄ haloalkyl, halo, CN, OR^{a3}, C(O)R^{b3}, C(O)OR^{a3} and S(O)₂R^{b3}.

In some embodiments, R^{c2} and R^{d2} are each independently selected from H, C₁₋₆ alkyl, C₁₋₆ haloalkyl, C₆₋₁₀ aryl, C₃₋₇ cycloalkyl, 4-10 membered heterocycloalkyl, C₆₋₁₀ aryl-C₁₋₄ alkyl, C₃₋₇ cycloalkyl-C₁₋₄ alkyl, and 4-10 membered heterocycloalkyl-C₁₋₄ alkyl, wherein said C₁₋₆ alkyl, C₁₋₆ haloalkyl, C₆₋₁₀ aryl, C₃₋₇ cycloalkyl, 4-10 membered heterocycloalkyl, C₆₋₁₀ aryl-C₁₋₄ alkyl, C₃₋₇ cycloalkyl-C₁₋₄ alkyl, and 4-10 membered heterocycloalkyl-C₁₋₄ alkyl are each optionally substituted with 1, 2, 3, 4, or 5 substituents independently selected from C₁₋₄ alkyl, C₁₋₄haloalkyl, halo, CN, OR^{a3}, C(O)R^{b3}, C(O)OR^{a3} and S(O)₂R^{b3}.

In some embodiments, Cy³ is 4-10 membered heterocycloalkyl optionally substituted with 1, 2, 3, 4, or 5 substituents independently selected from C₁₋₄ alkyl, C₁₋₄ haloalkyl, halo, CN, OR^{a3}, C(O)R^{b3}, C(O)OR^{a3} and S(O)₂R^{b3}.

In some embodiments, Cy³ is 4-10 membered heterocycloalkyl optionally substituted with 1, 2, 3, 4, or 5 substituents independently selected from C(O)R^{b3}.

In some embodiments, Cy³ is piperidinyl optionally substituted with 1, 2, 3, 4, or 5 substituents independently selected from halo and C(O)CH₃.

In some embodiments, compounds described herein have Formula II:

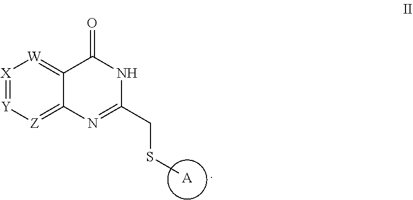

In some embodiments, compounds described herein have Formula IIIA, IIIB, IIIC, IIID, or IIIE:

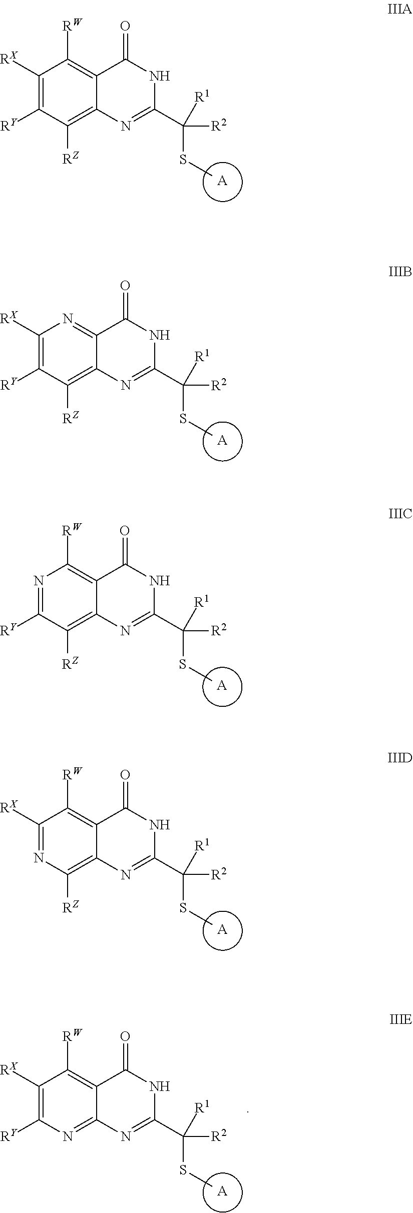

In some embodiments, compounds described herein have Formula IVA or IVB:

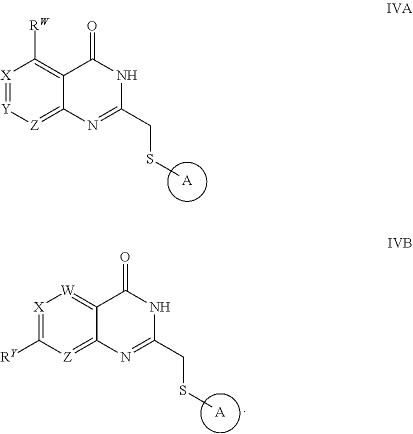

In some embodiments, the PARP14 inhibitor is selected from:
4-Oxo-2-(((tetrahydro-2H-pyran-4-yl)thio)methyl)-3,4-dihydroquinazoline-7-carbonitrile;
8-Methyl-2-(((tetrahydro-2H-pyran-4-yl)thio)methyl)quinazolin-4(3H)-one;
6-Methyl-2-(((tetrahydro-2H-pyran-4-yl)thio)methyl)quinazolin-4(3H)-one;
6-Methoxy-2-(((tetrahydro-2H-pyran-4-yl)thio)methyl)quinazolin-4(3H)-one;
8-Chloro-2-(((tetrahydro-2H-pyran-4-yl)thio)methyl)quinazolin-4(3H)-one;
8-Methoxy-2-(((tetrahydro-2H-pyran-4-yl)thio)methyl)quinazolin-4(3H)-one;
8-Methyl-2-(1-((tetrahydro-2H-pyran-4-yl)thio)ethyl)quinazolin-4(3H)-one;
5-Fluoro-8-methyl-2-(((tetrahydro-2H-pyran-4-yl)thio)methyl)quinazolin-4(3H)-one;
5-Methyl-2-(((tetrahydro-2H-pyran-4-yl)thio)methyl)quinazolin-4(3H)-one;
8-Benzyl-2-(((tetrahydro-2H-pyran-4-yl)thio)methyl)quinazolin-4(3H)-one;
7-Benzyl-2-(((tetrahydro-2H-pyran-4-yl)thio)methyl)quinazolin-4(3H)-one;
8-Methyl-2-((((tetrahydro-2H-pyran-4-yl)methyl)thio)methyl)quinazolin-4(3H)-one;
8-Methyl-2-((piperidin-4-ylthio)methyl)quinazolin-4(3H)-one trifluoroacetate;
8-Methyl-2-(((1-methylpiperidin-4-yl)thio)methyl)quinazolin-4(3H)-one;
8-Methyl-2-((pyrrolidin-3-ylthio)methyl)quinazolin-4(3H)-one;
8-Methyl-2-(((1-methylpyrrolidin-3-yl)thio)methyl)quinazolin-4(3H)-one;
2-(((1-Acetylpiperidin-4-yl)thio)methyl)-8-methylquinazolin-4(3H)-one;
8-Methyl-2-(((1-(pyridin-2-ylmethyl)piperidin-4-yl)thio)methyl)quinazolin-4(3H)-one;
8-Methyl-2-(((tetrahydro-2H-pyran-4-yl)sulfonyl)methyl)quinazolin-4(3H)-one;
2-((Azepan-4-ylthio)methyl)-8-methylquinazolin-4(3H)-one;
2-(((4-(Dimethylamino)cyclohexyl)thio)methyl)-8-methylquinazolin-4(3H)-one;
2-(((4-Hydroxycyclohexyl)thio)methyl)-8-methylquinazolin-4(3H)-one;
2-((((trans)-4-Hydroxycyclohexyl)thio)methyl)-8-methylquinazolin-4(3H)-one;
2-((((cis)-4-Hydroxycyclohexyl)thio)methyl)-8-methylquinazolin-4(3H)-one;
2-((Azetidin-3-ylthio)methyl)-8-methylquinazolin-4(3H)-one;
2-((((trans)-4-Methoxycyclohexyl)thio)methyl)-8-methylquinazolin-4(3H)-one;
2-((((cis)-4-Methoxycyclohexyl)thio)methyl)-8-methylquinazolin-4(3H)-one;
4-Oxo-2-(((tetrahydro-2H-pyran-4-yl)thio)methyl)-3,4-dihydroquinazoline-8-carbonitrile;
7-Phenoxy-2-(((tetrahydro-2H-pyran-4-yl)thio)methyl)quinazolin-4(3H)-one;
7-Fluoro-2-(((tetrahydro-2H-pyran-4-yl)thio)methyl)quinazolin-4(3H)-one;
7-Methoxy-2-(((tetrahydro-2H-pyran-4-yl)thio)methyl)quinazolin-4(3H)-one;
8-Methyl-2-(((1-methylpiperidin-3-yl)thio)methyl)quinazolin-4(3H)-one;
7-Fluoro-8-methyl-2-(((tetrahydro-2H-pyran-4-yl)thio)methyl)quinazolin-4(3H)-one;
5-Chloro-8-methyl-2-(((tetrahydro-2H-pyran-4-yl)thio)methyl)quinazolin-4(3H)-one;
8-Methyl-2-(((tetrahydro-2H-pyran-4-yl)thio)methyl)-5-(trifluoromethyl)quinazolin-4(3H)-one;
2-(((Tetrahydro-2H-pyran-4-yl)thio)methyl)pyrido[3,2-d]pyrimidin-4(3H)-one;
2-(((Tetrahydro-2H-pyran-4-yl)thio)methyl)pyrido[3,4-d]pyrimidin-4(3H)-one;
2-((((trans)-3-(Benzyloxy)cyclobutyl)thio)methyl)-8-methylquinazolin-4(3H)-one;
8-Methyl-2-((oxetan-3-ylthio)methyl)quinazolin-4(3H)-one;
2-(((Tetrahydro-2H-pyran-4-yl)thio)methyl)pyrido[4,3-d]pyrimidin-4(3H)-one;
8-Methyl-2-(((tetrahydro-2H-pyran-4-yl)thio)methyl)pyrido[3,2-d]pyrimidin-4(3H)-one;
8-Methyl-2-(((tetrahydro-2H-pyran-4-yl)thio)methyl)pyrido[3,4-d]pyrimidin-4(3H)-one;
2-(((Tetrahydro-2H-pyran-4-yl)thio)methyl)pyrido[2,3-d]pyrimidin-4(3H)-one;
6-Chloro-8-methyl-2-(((tetrahydro-2H-pyran-4-yl)thio)methyl)quinazolin-4(3H)-one;
7,8-Difluoro-2-(((tetrahydro-2H-pyran-4-yl)thio)methyl)quinazolin-4(3H)-one;
7-Fluoro-2-((((trans)-4-hydroxycyclohexyl)thio)methyl)quinazolin-4(3H)-one;
2-(((trans-3-Hydroxycyclobutyl)thio)methyl)-8-methylquinazolin-4(3H)-one;
8-Methyl-2-((piperidin-3-ylthio)methyl)quinazolin-4(3H)-one;
2-(((trans-4-Aminocyclohexyl)thio)methyl)-8-methylquinazolin-4(3H)-one;
2-(((cis-4-Aminocyclohexyl)thio)methyl)-8-methylquinazolin-4(3H)-one;
5-Fluoro-8-methyl-2-((piperidin-4-ylthio)methyl)quinazolin-4(3H)-one;
2-(((trans-3-Aminocyclobutyl)thio)methyl)-8-methylquinazolin-4(3H)-one;
2-(((4-Aminocycloheptyl)thio)methyl)-8-methylquinazolin-4(3H)-one;
2-(((trans-4-Aminocycloheptyl)thio)methyl)-8-methylquinazolin-4(3H)-one;
2-(((cis-4-Aminocycloheptyl)thio)methyl)-8-methylquinazolin-4(3H)-one;
5-Fluoro-2-(((4-hydroxycyclohexyl)thio)methyl)-8-methylquinazolin-4(3H)-one;
5-Fluoro-2-(((trans-4-hydroxycyclohexyl)thio)methyl)-8-methylquinazolin-4(3H)-one;
5-Fluoro-2-(((cis-4-hydroxycyclohexyl)thio)methyl)-8-methylquinazolin-4(3H)-one;
2-(((4-Hydroxycyclohexyl)thio)methyl)-8-methyl-5-(trifluoromethyl)quinazolin-4(3H)-one;
2-(((trans-4-Hydroxycyclohexyl)thio)methyl)-8-methyl-5-(trifluoromethyl) quinazolin-4(3H)-one;
2-(((cis-4-Hydroxycyclohexyl)thio)methyl)-8-methyl-5-(trifluoromethyl) quinazolin-4(3H)-one;
2-(((trans-4-(Hydroxymethyl)cyclohexyl)thio)methyl)-8-methylquinazolin-4(3H)-one;
2-(((cis-4-(Hydroxymethyl)cyclohexyl)thio)methyl)-8-methylquinazolin-4(3H)-one;
2-(((4-(Aminomethyl)cyclohexyl)thio)methyl)-8-methylquinazolin-4(3H)-one;
2-(((cis-4-(Aminomethyl)cyclohexyl)thio)methyl)-8-methylquinazolin-4(3H)-one;
2-(((trans-4-(Aminomethyl)cyclohexyl)thio)methyl)-8-methylquinazolin-4(3H)-one;
2-(((4-((Dimethylamino)methyl)cyclohexyl)thio)methyl)-8-methylquinazolin-4(3H)-one;
2-(((cis-4-((Dimethylamino)methyl)cyclohexyl)thio)methyl)-8-methyl quinazolin-4(3H)-one;
2-(((trans-4-((Dimethylamino)methyl)cyclohexyl)thio)methyl)-8-methyl quinazolin-4(3H)-one;
2-(((trans-3-(Hydroxymethyl)cyclohexyl)thio)methyl)-8-methylquinazolin-4(3H)-one;
2-(((cis-3-(Hydroxymethyl)cyclohexyl)thio)methyl)-8-methylquinazolin-4(3H)-one;
2-((((cis)-3-((Dimethylamino)methyl)cyclohexyl)thio)methyl)-8-methyl quinazolin-4(3H)-one;
8-Methyl-2-(((trans-4-((methylamino)methyl)cyclohexyl)thio)methyl) quinazolin-4(3H)-one;
7-Amino-2-(((tetrahydro-2H-pyran-4-yl)thio)methyl)quinazolin-4(3H)-one;
N-(4-Oxo-2-(((tetrahydro-2H-pyran-4-yl)thio)methyl)-3,4-dihydroquinazolin-7-yl)acetamide;
N-(4-Oxo-2-(((tetrahydro-2H-pyran-4-yl)thio)methyl)-3,4-dihydroquinazolin-7-yl)benzamide;
N-Methyl-4-oxo-2-(((tetrahydro-2H-pyran-4-yl)thio)methyl)-3,4-dihydro quinazoline-7-carboxamide;
4-Oxo-N-phenyl-2-(((tetrahydro-2H-pyran-4-yl)thio)methyl)-3,4-dihydro quinazoline-7-carboxamide;
7-(Phenylamino)-2-(((tetrahydro-2H-pyran-4-yl)thio)methyl)quinazolin-4(3H)-one;
7-(Pyridin-3-ylamino)-2-(((tetrahydro-2H-pyran-4-yl)thio)methyl)quinazolin-4(3H)-one;
7-(Pyridin-2-ylamino)-2-(((tetrahydro-2H-pyran-4-yl)thio)methyl)quinazolin-4(3H)-one;
7-((4-Methoxyphenyl)amino)-2-(((tetrahydro-2H-pyran-4-yl)thio)methyl)quinazolin-4(3H)-one;
7-((3-Methoxyphenyl)amino)-2-(((tetrahydro-2H-pyran-4-yl)thio)methyl)quinazolin-4(3H)-one;
7-((2-Methoxyphenyl)amino)-2-(((tetrahydro-2H-pyran-4-yl)thio)methyl)quinazolin-4(3H)-one;
7-(Pyrazin-2-ylamino)-2-(((tetrahydro-2H-pyran-4-yl)thio)methyl)quinazolin-4(3H)-one;
7-(Pyridin-4-ylamino)-2-(((tetrahydro-2H-pyran-4-yl)thio)methyl)quinazolin-4(3H)-one;
7-(Pyrimidin-5-ylamino)-2-(((tetrahydro-2H-pyran-4-yl)thio)methyl)quinazolin-4(3H)-one;
7-((1-Methyl-1H-imidazol-2-yl)amino)-2-(((tetrahydro-2H-pyran-4-yl)thio)methyl)quinazolin-4(3H)-one;
2-(((Tetrahydro-2H-pyran-4-yl)thio)methyl)-7-(thiazol-2-ylamino)quinazolin-4(3H)-one;
7-((2-Methylpyridin-3-yl)amino)-2-(((tetrahydro-2H-pyran-4-yl)thio)methyl)quinazolin-4(3H)-one;
7-((4-Methylpyridin-3-yl)amino)-2-(((tetrahydro-2H-pyran-4-yl)thio)methyl)quinazolin-4(3H)-one;
7-((5-Methylpyridin-3-yl)amino)-2-(((tetrahydro-2H-pyran-4-yl)thio)methyl)quinazolin-4(3H)-one;
7-(4-Amino-1H-pyrazol-1-yl)-2-(((tetrahydro-2H-pyran-4-yl)thio)methyl) quinazolin-4(3H)-one;
7-(Isoxazol-3-ylamino)-2-(((tetrahydro-2H-pyran-4-yl)thio)methyl)quinazolin-4(3H)-one;
8-Methyl-7-(phenylamino)-2-(((tetrahydro-2H-pyran-4-yl)thio)methyl) quinazolin-4(3H)-one;
7-(Benzyloxy)-2-(((tetrahydro-2H-pyran-4-yl)thio)methyl)quinazolin-4(3H)-one;
2-(((4-Hydroxycyclohexyl)thio)methyl)-7-(phenylamino)quinazolin-4(3H)-one;
2-(((trans-4-Hydroxycyclohexyl)thio)methyl)-7-(phenylamino)quinazolin-4(3H)-one;
2-(((cis-4-Hydroxycyclohexyl)thio)methyl)-7-(phenylamino)quinazolin-4(3H)-one;
2-(((cis-4-Hydroxycyclohexyl)thio)methyl)-7-(pyridin-3-ylamino)quinazolin-4(3H)-one;
2-(((trans-4-Hydroxycyclohexyl)thio)methyl)-7-(pyridin-3-ylamino)quinazolin-4(3H)-one;
7-(Cyclopentylamino)-2-(((trans-4-hydroxycyclohexyl)thio)methyl) quinazolin-4(3H)-one;
7-(Cyclopentylamino)-2-(((cis-4-hydroxycyclohexyl)thio)methyl) quinazolin-4(3H)-one;
2-(((trans-4-(Hydroxymethyl)cyclohexyl)thio)methyl)-7-(phenylamino) quinazolin-4(3H)-one;
2-(((cis-4-(Hydroxymethyl)cyclohexyl)thio)methyl)-7-(phenylamino) quinazolin-4(3H)-one;
2-(((cis-4-(Hydroxymethyl)cyclohexyl)thio)methyl)-7-(pyridin-3-ylamino) quinazolin-4(3H)-one;
2-(((trans-4-(Hydroxymethyl)cyclohexyl)thio)methyl)-7-(pyridin-3-ylamino) quinazolin-4(3H)-one;
7-(Cyclohexylamino)-2-(((tetrahydro-2H-pyran-4-yl)thio)methyl)quinazolin-4(3H)-one;
7-(Dimethylamino)-2-(((tetrahydro-2H-pyran-4-yl)thio)methyl)quinazolin-4(3H)-one;
7-(Methylamino)-2-(((tetrahydro-2H-pyran-4-yl)thio)methyl)quinazolin-4(3H)-one;
7-Morpholino-2-(((tetrahydro-2H-pyran-4-yl)thio)methyl)quinazolin-4(3H)-one;
7-(4-Methylpiperazin-1-yl)-2-(((tetrahydro-2H-pyran-4-yl)thio)methyl)quinazolin-4(3H)-one;
7-((1-Methylpiperidin-4-yl)amino)-2-(((tetrahydro-2H-pyran-4-yl)thio)methyl)quinazolin-4(3H)-one;
7-((Tetrahydro-2H-pyran-4-yl)amino)-2-(((tetrahydro-2H-pyran-4-yl)thio)methyl)quinazolin-4(3H)-one;
7-(Cyclopentylamino)-2-(((tetrahydro-2H-pyran-4-yl)thio)methyl)quinazolin-4(3H)-one;
7-(Isopropylamino)-2-(((tetrahydro-2H-pyran-4-yl)thio)methyl)quinazolin-4(3H)-one;
7-((Pyridin-4-ylmethyl)amino)-2-(((tetrahydro-2H-pyran-4-yl)thio)methyl)quinazolin-4(3H)-one;
7-((Pyridin-2-ylmethyl)amino)-2-(((tetrahydro-2H-pyran-4-yl)thio)methyl)quinazolin-4(3H)-one;
7-(Benzylamino)-2-(((tetrahydro-2H-pyran-4-yl)thio)methyl)quinazolin-4(3H)-one;
7-((1-Phenylethyl)amino)-2-(((tetrahydro-2H-pyran-4-yl)thio)methyl)quinazolin-4(3H)-one;
2-(((Tetrahydro-2H-pyran-4-yl)thio)methyl)-7-((tetrahydrofuran-3-yl)amino)quinazolin-4(3H)-one;
7-(Cyclobutylamino)-2-(((tetrahydro-2H-pyran-4-yl)thio)methyl)quinazolin-4(3H)-one;
7-((Pyridin-3-ylmethyl)amino)-2-(((tetrahydro-2H-pyran-4-yl)thio)methyl)quinazolin-4(3H)-one;
7-(Cyclopropylamino)-2-(((tetrahydro-2H-pyran-4-yl)thio)methyl)quinazolin-4(3H)-one;
7-(Cyclohexyl(methyl)amino)-2-(((tetrahydro-2H-pyran-4-yl)thio)methyl)quinazolin-4(3H)-one;
7-[(1-Benzyl-3-piperidyl)amino]-2-(tetrahydropyran-4-ylsulfanylmethyl)-3H-quinazolin-4-one;
7-(3-Piperidylamino)-2-(tetrahydropyran-4-ylsulfanylmethyl)-3H-quinazolin-4-one;
7-((1-Benzylpiperidin-4-yl)amino)-2-(((tetrahydro-2H-pyran-4-yl)thio)methyl) quinazolin-4(3H)-one;
7-(Piperidin-4-ylamino)-2-(((tetrahydro-2H-pyran-4-yl)thio)methyl)quinazolin-4(3H)-one;
7-(Pyrrolidin-3-ylamino)-2-(((tetrahydro-2H-pyran-4-yl)thio)methyl)quinazolin-4(3H)-one;
7-((1-Acetylpiperidin-4-yl)amino)-2-(((tetrahydro-2H-pyran-4-yl)thio)methyl) quinazolin-4(3H)-one;
7-((1-Acetylpiperidin-3-yl)amino)-2-(((tetrahydro-2H-pyran-4-yl)thio)
methyl)quinazolin-4(3H)-one;
7-((1-Methylpiperidin-3-yl)amino)-2-(((tetrahydro-2H-pyran-4-yl)thio)
methyl)quinazolin-4(3H)-one
7-((1-Acetylpyrrolidin-3-yl)amino)-2-(((tetrahydro-2H-pyran-4-yl)thio)
methyl)quinazolin-4(3H)-one;
8-Methyl-7-phenoxy-2-(((tetrahydro-2H-pyran-4-yl)thio)methyl)quinazolin-4(3H)-one;
7-(Cyclohexylamino)-2-(((trans-4-(hydroxymethyl)cyclohexyl)thio)methyl)quinazolin-4(3H)-one;
7-(Cyclohexylamino)-2-(((cis-4-(hydroxymethyl)cyclohexyl)thio)methyl)quinazolin-4(3H)-one;
8-Methyl-2-(((1-((1-methyl-1H-imidazol-2-yl)methyl)piperidin-4-yl)thio)
methyl)quinazolin-4(3H)-one;
N-(4-((4-(((8-Methyl-4-oxo-3,4-dihydroquinazolin-2-yl)methyl)thio)piperidin-1-yl)methyl)phenyl)acetamide;
2-(((1-(4-(Dimethylamino)benzyl)piperidin-4-yl)thio)methyl)-8-methylquinazolin-4(3H)-one;
4-((4-(((8-Methyl-4-oxo-3,4-dihydroquinazolin-2-yl)methyl)thio)piperidin-1-yl)methyl)benzonitrile;
2-(((1-((1H-Pyrazol-3-yl)methyl)piperidin-4-yl)thio)methyl)-8-methylquinazolin-4(3H)-one;
8-Methyl-2-(((1-((1-methyl-1H-indazol-3-yl)methyl)piperidin-4-yl)thio)methyl)-quinazolin-4(3H)-one;
2-(((1-((1,3-Dimethyl-1H-pyrazol-4-yl)methyl)piperidin-4-yl)thio)methyl)-8-methylquinazolin-4(3H)-one;
8-Methyl-2-(((1-((6-methylpyridin-2-yl)methyl)piperidin-4-yl)thio)methyl)quinazolin-4(3H)-one;
8-Methyl-2-(((1-((3-methylpyridin-2-yl)methyl)piperidin-4-yl)thio)methyl)quinazolin-4(3H)-one;
8-Methyl-2-(((1-phenethylpiperidin-4-yl)thio)methyl)quinazolin-4(3H)-one;
8-Methyl-2-(((1-((1-methyl-1H-indazol-6-yl)methyl)piperidin-4-yl)thio)methyl)quinazolin-4(3H)-one;
8-Methyl-2-(((1-((3-methyl-1H-pyrazol-4-yl)methyl)piperidin-4-yl)thio)methyl)quinazolin-4(3H)-one;
N-(3-((4-(((8-Methyl-4-oxo-3,4-dihydroquinazolin-2-yl)methyl)thio)piperidin-1-yl)methyl)phenyl)acetamide;
2-(((1-((1H-Pyrrolo[3,2-c]pyridin-3-yl)methyl)piperidin-4-yl)thio)methyl)-8-methylquinazolin-4(3H)-one;
2-(((1-(Imidazo[1,2-a]pyridin-3-ylmethyl)piperidin-4-yl)thio)methyl)-8-methylquinazolin-4(3H)-one;
2-(((1-((1-Benzyl-1H-imidazol-5-yl)methyl)piperidin-4-yl)thio)methyl)-8-methylquinazolin-4(3H)-one;
2-(((1-((1-Benzyl-1H-pyrazol-4-yl)methyl)piperidin-4-yl)thio)methyl)-8-methylquinazolin-4(3H)-one;
2-(2-((4-(((8-Methyl-4-oxo-3,4-dihydroquinazolin-2-yl)methyl)thio)piperidin-1-yl)methyl)phenoxy)acetonitrile;
8-Methyl-2-(((1-((2-oxoindolin-6-yl)methyl)piperidin-4-yl)thio)methyl)quinazolin-4(3H)-one;
2-(((1-((5-Methoxypyridin-2-yl)methyl)piperidin-4-yl)thio)methyl)-8-methylquinazolin-4(3H)-one;
8-Methyl-2-(((1-((4-methyl-3,4-dihydro-2H-benzo[b][1,4]oxazin-7-yl)methyl)piperidin-4-yl)thio)methyl)quinazolin-4(3H)-one;
(S)-2-(((1-(2,3-Dihydroxypropyl)piperidin-4-yl)thio)methyl)-8-methylquinazolin-4(3H)-one;
(R)-2-(((1-(2,3-Dihydroxypropyl)piperidin-4-yl)thio)methyl)-8-methylquinazolin-4(3H)-one;
(S)-8-Methyl-2-(((1-(pyrrolidin-2-ylmethyl)piperidin-4-yl)thio)methyl)quinazolin-4(3H)-one;
2-(((1-(2-Hydroxyethyl)piperidin-4-yl)thio)methyl)-8-methylquinazolin-4(3H)-one;
2-(((1-(2-Aminoethyl)piperidin-4-yl)thio)methyl)-8-methylquinazolin-4(3H)-one;
N-(2-(4-(((8-Methyl-4-oxo-3,4-dihydroquinazolin-2-yl)methyl)thio)piperidin-1-yl)ethyl)picolinamide;
2-(((1-(3-Aminopropyl)piperidin-4-yl)thio)methyl)-8-methylquinazolin-4(3H)-one;
2-(((1-Glycylpiperidin-4-yl)thio)methyl)-8-methylquinazolin-4(3H)-one;
2-(((1-(3-Aminopropanoyl)piperidin-4-yl)thio)methyl)-8-methylquinazolin-4(3H)-one;
2-(((1-(3-(Dimethylamino)propanoyl)piperidin-4-yl)thio)methyl)-8-methylquinazolin-4(3H)-one;
(R)-1-(4-Amino-5-(4-(((8-methyl-4-oxo-3,4-dihydroquinazolin-2-yl) methyl)thio)piperidin-1-yl)-5-oxopentyl)guanidine;
(S)-1-(4-Amino-5-(4-(((8-methyl-4-oxo-3,4-dihydroquinazolin-2-yl)methyl) thio)piperidin-1-yl)-5-oxopentyl)guanidine;
2-(((1-(L-Lysyl)piperidin-4-yl)thio)methyl)-8-methylquinazolin-4(3H)-one;
2-(((1-(D-Lysyl)piperidin-4-yl)thio)methyl)-8-methylquinazolin-4(3H)-one;
8-Methyl-2-(((1-(3-(pyridin-2-yl)propanoyl)piperidin-4-yl)thio)methyl)quinazolin-4(3H)-one;
8-Methyl-2-(((1-(methylsulfonyl)piperidin-4-yl)thio)methyl)quinazolin-4(3H)-one;
8-Methyl-2-(((1-(pyridin-2-ylsulfonyl)piperidin-4-yl)thio)methyl)quinazolin-4(3H)-one;
7-(Cyclopentylamino)-5-fluoro-2-((piperidin-4-ylthio)methyl)quinazolin-4(3H)-one;
7-(Cyclobutylamino)-5-fluoro-2-((piperidin-4-ylthio)methyl)quinazolin-4(3H)-one;
N-(((trans)-4-(((8-Methyl-4-oxo-3,4-dihydroquinazolin-2-yl)methyl)thio)cyclohexyl)methyl)acetamide;
7-(Cyclopentylamino)-2-((piperidin-4-ylthio)methyl)quinazolin-4(3H)-one;
7-(Cyclopentylamino)-2-((((1R,4R)-4-(hydroxymethyl)cyclohexyl)thio)-methyl)quinazolin-4(3H)-one;
2-((((trans)-4-(2-Aminoethyl)cyclohexyl)thio)methyl)-8-methylquinazolin-4(3H)-one;
2-(((3-(Aminomethyl)cyclobutyl)thio)methyl)-8-methylquinazolin-4(3H)-one;
2-((((trans)-3-(2-Aminoethyl)cyclopentyl)thio)methyl)-8-methylquinazolin-4(3H)-one
7-(Cyclopentylamino)-5-fluoro-2-(((tetrahydro-2H-pyran-4-yl)thio)methyl)quinazolin-4(3H)-one;
7-(Cyclopentylamino)-5-fluoro-2-((((1R,4R)-4-hydroxycyclohexyl)thio)methyl)-quinazolin-4(3H)-one;
7-(Cyclopentylamino)-2-(((tetrahydro-2H-pyran-4-yl)thio)methyl)pyrido[2,3-d]pyrimidin-4(3H)-one;
(S)-7-((Tetrahydro-2H-pyran-3-yl)amino)-2-(((tetrahydro-2H-pyran-4-yl)thio)methyl)quinazolin-4(3H)-one;
7-(Cyclopentylamino)-2-(((tetrahydro-2H-pyran-4-yl)thio)methyl)pyrido[3,2-d]pyrimidin-4(3H)-one;
7-(Cyclopentylamino)-2-(((tetrahydro-2H-pyran-4-yl)thio)methyl)pyrido[4,3-d]pyrimidin-4(3H)-one;
2-((Azepan-4-ylthio)methyl)-7-(cyclopentylamino)quinazolin-4(3H)-one;
2-(((3-(Aminomethyl)cyclopentyl)thio)methyl)-8-methylquinazolin-4(3H)-one;
7-((3-Methylisoxazol-5-yl)amino)-2-(((tetrahydro-2H-pyran-4-yl)thio)methyl)quinazolin-4(3H)-one;
(R)-7-((1-(Methylsulfonyl)piperidin-3-yl)amino)-2-(((tetrahydro-2H-pyran-4-yl)thio)methyl)quinazolin-4(3H)-one;
7-(Cyclobutylamino)-2-((((1R,4R)-4-hydroxycyclohexyl)thio)methyl)quinazolin-4(3H)-one;
7-((1-(Methylsulfonyl)azetidin-3-yl)amino)-2-(((tetrahydro-2H-pyran-4-yl)thio)methyl)quinazolin-4(3H)-one;
(R)-7-((1-(Methylsulfonyl)piperidin-3-yl)amino)-2-((piperidin-4-ylthio)methyl)quinazolin-4(3H)-one;
7-(Cyclopentyloxy)-2-(((tetrahydro-2H-pyran-4-yl)thio)methyl)quinazolin-4(3H)-one;
8-Methyl-2-((oxepan-4-ylthio)methyl)quinazolin-4(3H)-one;
7-(Cyclopentylamino)-2-((((1R,4R)-4-hydroxycyclohexyl)thio)methyl)-5-(trifluoromethyl)quinazolin-4(3H)-one;
7-(Cyclobutylamino)-2-((piperidin-4-ylthio)methyl)quinazolin-4(3H)-one;
(R)-7-((1-(Methylsulfonyl)piperidin-3-yl)amino)-2-(((tetrahydro-2H-pyran-4-yl)thio)methyl)pyrido[2,3-d]pyrimidin-4(3H)-one;
7-Isobutyl-2-(((tetrahydro-2H-pyran-4-yl)thio)methyl)quinazolin-4(3H)-one;
7-(Cyclopentylamino)-5-methyl-2-(((tetrahydro-2H-pyran-4-yl)thio)methyl) quinazolin-4(3H)-one;
cis-4-(((8-Methyl-4-oxo-3,4-dihydroquinazolin-2-yl)methyl)thio)cyclohexane-1-carboxamide;
trans-4-(((8-Methyl-4-oxo-3,4-dihydroquinazolin-2-yl)methyl)thio)cyclohexane-1-carboxamide;
5-Chloro-7-(cyclopentylamino)-2-((piperidin-4-ylthio)methyl)quinazolin-4(3H)-one;
7-(Cyclopentylamino)-5-methoxy-2-(((tetrahydro-2H-pyran-4-yl)thio)methyl)quinazolin-4(3H)-one;
Methyl 4-(((7-(cyclopentylamino)-4-oxo-3,4-dihydroquinazolin-2-yl)methyl)thio)piperidine-1-carboxylate;
2-((trans)-4-(((8-Methyl-4-oxo-3,4-dihydroquinazolin-2-yl)methyl)thio)cyclohexyl) acetamide;
7-(Cyclopentylamino)-5-fluoro-2-(((trans-3-fluoropiperidin-4-yl)thio)methyl)quinazolin-4(3H)-one;
7-(Cyclopentylamino)-5-fluoro-2-((((3S,4S)-3-fluoropiperidin-4-yl)thio)methyl)quinazolin-4(3H)-one;
7-(Cyclopentylamino)-5-fluoro-2-((((3R,4R)-3-fluoropiperidin-4-yl)thio)methyl)quinazolin-4(3H)-one;
7-(Cyclopentylamino)-5-fluoro-2-((((cis)-3-fluoropiperidin-4-yl)thio)methyl)quinazolin-4(3H)-one;
7-(Cyclopentylamino)-5-fluoro-2-((((3R,4S)-3-fluoropiperidin-4-yl)thio)methyl)quinazolin-4(3H)-one;
7-(Cyclopentylamino)-5-fluoro-2-((((3S,4R)-3-fluoropiperidin-4-yl)thio)methyl)quinazolin-4(3H)-one;
7-(Cyclopentylamino)-5-fluoro-2-(((1-(2-hydroxyacetyl)piperidin-4-yl)thio)methyl)quinazolin-4(3H)-one;
2-((Cyclohexylthio)methyl)-7-(cyclopentylamino)-5-fluoroquinazolin-4(3H)-one;
cis-4-(((7-(Cyclopentylamino)-5-fluoro-4-oxo-3,4-dihydroquinazolin-2-yl)methyl)thio)cyclohexane-1-carboxylic acid;
trans-4-(((7-(Cyclopentylamino)-5-fluoro-4-oxo-3,4-dihydroquinazolin-2-yl)methyl)thio)cyclohexane-1-carboxylic acid;
trans-4-(((7-(Cyclopentylamino)-5-fluoro-4-oxo-3,4-dihydroquinazolin-2-yl)methyl)thio)cyclohexane-1-carboxamide;
7-(Cyclopropylmethoxy)-2-(((tetrahydro-2H-pyran-4-yl)thio)methyl)quinazolin-4(3H)-one;
4-(((7-(Cyclopentylamino)-5-fluoro-4-oxo-3,4-dihydroquinazolin-2-yl)methyl)thio)-N,N-dimethylpiperidine-1-carboxamide;
2-(((cis-6-(Hydroxymethyl)tetrahydro-2H-pyran-3-yl)thio)methyl)-8-methylquinazolin-4(3H)-one;
7-(Cyclopentylamino)-5-fluoro-2-(((trans-3-(trifluoromethyl)piperidin-4-yl)thio)methyl)quinazolin-4(3H)-one;
7-(Cyclopentylamino)-5-fluoro-2-(((cis-4-fluoropyrrolidin-3-yl)thio)methyl)quinazolin-4(3H)-one;
7-(Cyclopentylamino)-5-(hydroxymethyl)-2-(((tetrahydro-2H-pyran-4-yl)thio)methyl)quinazolin-4(3H)-one;
7-(Cyclopentylamino)-5-(fluoromethyl)-2-(((tetrahydro-2H-pyran-4-yl)thio)methyl)quinazolin-4(3H)-one;
7-(Cyclopentylamino)-6-fluoro-2-((piperidin-4-ylthio)methyl)quinazolin-4(3H)-one;
7-(Cyclopentylamino)-5-fluoro-2-(((trans-2-(trifluoromethyl)piperidin-4-yl)thio)methyl)quinazolin-4(3H)-one;
7-(Cyclopentylamino)-5-fluoro-2-(((cis-2-(trifluoromethyl)piperidin-4-yl)thio)methyl)quinazolin-4(3H)-one;
7-(Cyclopropylmethoxy)-2-((piperidin-4-ylthio)methyl)pyrido[2,3-d]pyrimidin-4(3H)-one;
7-((Cyclobutylmethyl)amino)-6-methoxy-2-((piperidin-4-ylthio)methyl)quinazolin-4(3H)-one;
7-((2,2-Difluorocyclopentyl)amino)-5-fluoro-2-(((tetrahydro-2H-pyran-4-yl)thio)methyl)quinazolin-4(3H)-one;
7-(Cyclopentylamino)-5,6-difluoro-2-((piperidin-4-ylthio)methyl)quinazolin-4(3H)-one;
5-Fluoro-7-((trans-4-morpholinocyclohexyl)amino)-2-(((tetrahydro-2H-pyran-4-yl)thio)methyl)quinazolin-4(3H)-one;
5-Fluoro-7-((cis-4-morpholinocyclohexyl)amino)-2-(((tetrahydro-2H-pyran-4-yl)thio)methyl)quinazolin-4(3H)-one;
7-(Cyclopropylmethoxy)-5-fluoro-2-(((trans-4-hydroxycyclohexyl)thio)methyl)quinazolin-4(3H)-one;
5-Fluoro-7-((tetrahydro-2H-pyran-4-yl)methoxy)-2-(((tetrahydro-2H-pyran-4-yl)thio)methyl)quinazolin-4(3H)-one;
7-(Cyclobutylmethoxy)-5-methyl-2-(((tetrahydro-2H-pyran-4-yl)thio)methyl)quinazolin-4(3H)-one;
5-Fluoro-2-(((tetrahydro-2H-pyran-4-yl)thio)methyl)-7-((tetrahydrofuran-3-yl)methoxy)quinazolin-4(3H)-one;
(R)-5-Fluoro-2-(((tetrahydro-2H-pyran-4-yl)thio)methyl)-7-((tetrahydrofuran-3-yl)methoxy)quinazolin-4(3H)-one
(S)-5-Fluoro-2-(((tetrahydro-2H-pyran-4-yl)thio)methyl)-7-((tetrahydrofuran-3-yl)methoxy)quinazolin-4(3H)-one;
7-(Cyclopentylamino)-5-fluoro-2-(((trans-6-fluoroazepan-4-yl)thio)methyl)quinazolin-4(3H)-one;
7-(Cyclopentylamino)-5-fluoro-2-(((cis-6-fluoroazepan-4-yl)thio)methyl)quinazolin-4(3H)-one;
2-((((cis)-6-(Aminomethyl)tetrahydro-2H-pyran-3-yl)thio)methyl)-7-(cyclopentylamino)-5-fluoroquinazolin-4(3H)-one;
2-(((trans-4-(Aminomethyl)-4-fluorocyclohexyl)thio)methyl)-7-(cyclopentylamino)-5-fluoroquinazolin-4(3H)-one;
2-(((cis-4-(Aminomethyl)-4-fluorocyclohexyl)thio)methyl)-7-(cyclopentylamino)-5-fluoroquinazolin-4(3H)-one;
6-Fluoro-7-((tetrahydro-2H-pyran-4-yl)amino)-2-(((tetrahydro-2H-pyran-4-yl)thio)methyl)quinazolin-4(3H)-one;
7-(Cyclopentylamino)-5-fluoro-2-(((1-methylpiperidin-4-yl)thio)methyl)quinazolin-4(3H)-one;
7-(Cyclohexylamino)-5-fluoro-2-(((tetrahydro-2H-pyran-4-yl)thio)methyl)quinazolin-4(3H)-one;
7-(Cyclohexylamino)-5-fluoro-2-((piperidin-4-ylthio)methyl)quinazolin-4(3H)-one;
7-(Cyclohexylamino)-5-fluoro-2-((((1r,4r)-4-hydroxycyclohexyl)thio)methyl)quinazolin-4(3H)-one;
(R)-5-Fluoro-7-((1-(methylsulfonyl)piperidin-3-yl)amino)-2-(((tetrahydro-2H-pyran-4-yl)thio)methyl)quinazolin-4(3H)-one;
7-(Cyclobutylamino)-5-fluoro-2-(((tetrahydro-2H-pyran-4-yl)thio)methyl)quinazolin-4(3H)-one;
7-((2-Cyclopentylethyl)amino)-2-(((tetrahydro-2H-pyran-4-yl)thio)methyl)quinazolin-4(3H)-one;
5-Chloro-7-(cyclopentylamino)-2-(((tetrahydro-2H-pyran-4-yl)thio)methyl)quinazolin-4(3H)-one;
7-(Cyclopentylamino)-2-(((1-(2,2,2-trifluoroethyl)piperidin-4-yl)thio)methyl)quinazolin-4(3H)-one;
7-(Cyclopentylamino)-5-fluoro-2-(((1-(oxetan-3-yl)piperidin-4-yl)thio)methyl)quinazolin-4(3H)-one;
7-((2-(Tetrahydro-2H-pyran-4-yl)ethyl)amino)-2-(((tetrahydro-2H-pyran-4-yl)thio)methyl)quinazolin-4(3H)-one;
7-(Cyclopentylamino)-5-methyl-2-((piperidin-4-ylthio)methyl)quinazolin-4(3H)-one;
7-(Cyclopentylamino)-2-(((1-(2,2-difluoroethyl)piperidin-4-yl)thio)methyl)quinazolin-4(3H)-one;
7-(Cyclopentylamino)-2-(((1-(3,3,3-trifluoropropyl)piperidin-4-yl)thio)methyl)quinazolin-4(3H)-one;
2-(((cis-6-(Hydroxymethyl)tetrahydro-2H-pyran-2-yl)thio)methyl)-8-methylquinazolin-4(3H)-one;
7-((Cyclobutylmethyl)amino)-5-fluoro-2-((piperidin-4-ylthio)methyl)quinazolin-4(3H)-one;
7-(((2,2-Difluorocyclopropyl)methyl)amino)-5-fluoro-2-((piperidin-4-ylthio)methyl)quinazolin-4(3H)-one;
7-(Cyclopentylamino)-5-fluoro-2-(((1-(2,2,2-trifluoroethyl)piperidin-4-yl)thio)methyl)quinazolin-4(3H)-one;
7-(Cyclopentylamino)-2-(((1-(2,2-difluoropropyl)piperidin-4-yl)thio)methyl)quinazolin-4(3H)-one;
7-((Cyclopropylmethyl)amino)-5-fluoro-2-((piperidin-4-ylthio)methyl)quinazolin-4(3H)-one;
7-((3,3-Difluorocyclopentyl)amino)-5-fluoro-2-((piperidin-4-ylthio)methyl)quinazolin-4(3H)-one;
2-(((trans-4-Hydroxycyclohexyl)thio)methyl)-7-(((R)-1-(methylsulfonyl)piperidin-3-yl)amino)quinazolin-4(3H)-one;
(R)-2-(((1-Acetylpiperidin-4-yl)thio)methyl)-7-((1-(methylsulfonyl)piperidin-3-yl)amino)quinazolin-4(3H)-one;
5-Fluoro-2-(((trans-4-hydroxycyclohexyl)thio)methyl)-7-(((R)-1-(methylsulfonyl)piperidin-3-yl)amino)quinazolin-4(3H)-one;
7-(Cyclopentylamino)-2-(((1,1-dioxidotetrahydro-2H-thiopyran-4-yl)thio)methyl)-5-fluoroquinazolin-4(3H)-one;
7-((Cyclopropylmethyl)amino)-5-fluoro-2-(((trans-4-hydroxycyclohexyl)thio)methyl)quinazolin-4(3H)-one;
5-Fluoro-2-((piperidin-4-ylthio)methyl)-7-(((tetrahydro-2H-pyran-4-yl)methyl)amino)quinazolin-4(3H)-one;
7-(Cyclopentylamino)-2-(((1-(1,1-dioxidothietan-3-yl)piperidin-4-yl)thio)methyl)-5-fluoroquinazolin-4(3H)-one;
7-((Cyclopropylmethyl)amino)-5-fluoro-2-(((1-(oxetan-3-yl)piperidin-4-yl)thio)methyl)quinazolin-4(3H)-one;
7-(Cyclopropylmethoxy)-2-((piperidin-4-ylthio)methyl)quinazolin-4(3H)-one;
7-(Cyclopentylamino)-5-fluoro-2-(((1-(2-(methylsulfonyl)ethyl)piperidin-4-yl)thio)methyl)quinazolin-4(3H)-one;
5-Fluoro-7-((2-morpholinoethyl)amino)-2-((piperidin-4-ylthio)methyl)quinazolin-4(3H)-one;
7-(Cyclopropylmethoxy)-5-fluoro-2-((piperidin-4-ylthio)methyl)quinazolin-4(3H)-one;
7-(Cyclopentylamino)-5-fluoro-2-(((1-(2-hydroxy-2-methylpropanoyl)piperidin-4-yl)thio)methyl)quinazolin-4(3H)-one;
7-(Cyclobutylmethoxy)-5-fluoro-2-((piperidin-4-ylthio)methyl)quinazolin-4(3H)-one;
7-(Cyclopentylamino)-5-fluoro-2-(((1-(pyridin-2-ylmethyl)piperidin-4-yl)thio)methyl)quinazolin-4(3H)-one;
7-(Cyclopentylmethoxy)-5-fluoro-2-((piperidin-4-ylthio)methyl)quinazolin-4(3H)-one;
2-(4-(((7-(Cyclopentylamino)-5-fluoro-4-oxo-3,4-dihydroquinazolin-2-yl)methyl)thio)piperidin-1-yl)-N-methylacetamide;
7-(((2,2-Difluorocyclopropyl)methyl)amino)-5-methyl-2-((piperidin-4-ylthio)methyl)quinazolin-4(3H)-one;
2-(4-(((7-(Cyclopentylamino)-5-fluoro-4-oxo-3,4-dihydroquinazolin-2-yl)methyl)thio)piperidin-1-yl)acetonitrile;
2-(trans-4-(((7-(Cyclopentylamino)-5-fluoro-4-oxo-3,4-dihydroquinazolin-2-yl)methyl)thio)cyclohexyl)acetamide;
5-Fluoro-7-((2-morpholinoethyl)amino)-2-(((tetrahydro-2H-pyran-4-yl)thio)methyl)quinazolin-4(3H)-one;
5-Fluoro-2-(((tetrahydro-2H-pyran-4-yl)thio)methyl)-7-((1-(2,2,2-trifluoroethyl)piperidin-4-yl)amino)quinazolin-4(3H)-one;
7-((Cyclobutylmethyl)amino)-6-fluoro-2-((piperidin-4-ylthio)methyl)quinazolin-4(3H)-one;
7-(Cyclohexylamino)-6-fluoro-2-(((tetrahydro-2H-pyran-4-yl)thio)methyl)quinazolin-4(3H)-one;
7-(Cyclopropylmethoxy)-5-fluoro-2-(((tetrahydro-2H-pyran-4-yl)thio)methyl)quinazolin-4(3H)-one;
7-((Cyclopropylmethyl)amino)-6-fluoro-2-(((tetrahydro-2H-pyran-4-yl)thio)methyl)quinazolin-4(3H)-one;
7-(Cyclopentylamino)-6-fluoro-2-(((trans-4-hydroxycyclohexyl)thio)methyl)quinazolin-4(3H)-one;
7-(Cyclopentylamino)-5,6-difluoro-2-(((tetrahydro-2H-pyran-4-yl)thio)methyl)quinazolin-4(3H)-one;
7-(Cyclopropylmethoxy)-5-fluoro-2-(((cis-3-fluoropiperidin-4-yl)thio)methyl)quinazolin-4(3H)-one;
7-((Cyclobutylmethyl)amino)-2-(((1,1-dioxidotetrahydro-2H-thiopyran-4-yl)thio)methyl)-6-fluoroquinazolin-4(3H)-one;
7-(Cyclopropylmethoxy)-5-fluoro-2-(((trans-3-fluoropiperidin-4-yl)thio)methyl)quinazolin-4(3H)-one;
5-Fluoro-2-(((tetrahydro-2H-pyran-4-yl)thio)methyl)-7-((1-(3,3,3-trifluoropropyl)piperidin-4-yl)methoxy)quinazolin-4(3H)-one;
7-((1-(2,2-Difluoropropyl)piperidin-4-yl)methoxy)-5-fluoro-2-(((tetrahydro-2H-pyran-4-yl)thio)methyl)quinazolin-4(3H)-one;
7-((1-(2,2-Difluoroethyl)piperidin-4-yl)methoxy)-5-fluoro-2-(((tetrahydro-2H-pyran-4-yl)thio)methyl)quinazolin-4(3H)-one;
5-Fluoro-7-((1-(oxetan-3-yl)piperidin-4-yl)methoxy)-2-(((tetrahydro-2H-pyran-4-yl)thio)methyl)quinazolin-4(3H)-one;
5-Fluoro-7-((1-(oxetan-3-yl)piperidin-4-yl)amino)-2-(((tetrahydro-2H-pyran-4-yl)thio)methyl)quinazolin-4(3H)-one;
7-((Cyclobutylmethyl)amino)-6-fluoro-2-(((cis-3-fluoropiperidin-4-yl)thio)methyl)quinazolin-4(3H)-one;
7-(Cyclobutylmethoxy)-2-(((1,1-dioxidotetrahydro-2H-thiopyran-4-yl)thio)methyl)-5-fluoroquinazolin-4(3H)-one;
5-Fluoro-7-((trans-2-fluorocyclopentyl)amino)-2-(((tetrahydro-2H-pyran-4-yl)thio)methyl)quinazolin-4(3H)-one;
5-Fluoro-7-isobutoxy-2-(((tetrahydro-2H-pyran-4-yl)thio)methyl)quinazolin-4(3H)-one;
7-(Cyclobutylmethoxy)-5-fluoro-2-(((1-(2-hydroxyacetyl)piperidin-4-yl)thio)methyl)quinazolin-4(3H)-one;
7-(Cyclobutylmethoxy)-2-(((2,2-dimethyltetrahydro-2H-pyran-4-yl)thio)methyl)-5-fluoroquinazolin-4(3H)-one;
7-(Cyclobutylmethoxy)-2-((cyclohexylthio)methyl)-5-fluoroquinazolin-4(3H)-one;
2-((Cyclohexylthio)methyl)-7-(cyclopentylamino)-5,6-difluoroquinazolin-4(3H)-one;
trans-4-(((7-(Cyclobutylmethoxy)-5-fluoro-4-oxo-3,4-dihydroquinazolin-2-yl)methyl)thio)cyclohexane-1-carboxamide;
7-((1-(2,2-Difluoroethyl)piperidin-3-yl)methoxy)-5-fluoro-2-(((tetrahydro-2H-pyran-4-yl)thio)methyl)quinazolin-4(3H)-one;
7-(Cyclopentylamino)-5,6-difluoro-2-(((trans-4-hydroxycyclohexyl)thio)methyl)quinazolin-4(3H)-one;
7-(Cyclopentylmethoxy)-5-fluoro-2-(((trans-4-hydroxycyclohexyl)thio)methyl)quinazolin-4(3H)-one;
7-((2,2-Difluorocyclopropyl)methoxy)-5-fluoro-2-(((tetrahydro-2H-pyran-4-yl)thio)methyl)quinazolin-4(3H)-one;
7-(Cyclopentylamino)-2-(((1,1-dioxidotetrahydro-2H-thiopyran-4-yl)thio)methyl)-5,6-difluoroquinazolin-4(3H)-one;
7-((3,3-Difluorocyclobutyl)methoxy)-5-fluoro-2-(((tetrahydro-2H-pyran-4-yl)thio)methyl)quinazolin-4(3H)-one;
5-Fluoro-2-(((trans-4-hydroxycyclohexyl)thio)methyl)-7-((tetrahydro-2H-pyran-3-yl)methoxy)quinazolin-4(3H)-one;
5-Fluoro-7-((tetrahydro-2H-pyran-3-yl)methoxy)-2-(((tetrahydro-2H-pyran-4-yl)thio)methyl)quinazolin-4(3H)-one;
5-Fluoro-2-(((tetrahydro-2H-pyran-4-yl)thio)methyl)-7-((tetrahydrofuran-2-yl)methoxy)quinazolin-4(3H)-one;
(R)-5-Fluoro-2-(((tetrahydro-2H-pyran-4-yl)thio)methyl)-7-((tetrahydrofuran-2-yl)methoxy)quinazolin-4(3H)-one;
(S)-5-Fluoro-2-(((tetrahydro-2H-pyran-4-yl)thio)methyl)-7-((tetrahydrofuran-2-yl)methoxy)quinazolin-4(3H)-one;
5,6-Difluoro-2-(((tetrahydro-2H-pyran-4-yl)thio)methyl)-7-(((tetrahydrofuran-3-yl)methyl)amino)quinazolin-4(3H)-one;
(S)-5,6-Difluoro-2-(((tetrahydro-2H-pyran-4-yl)thio)methyl)-7-(((tetrahydrofuran-3-yl)methyl)amino)quinazolin-4(3H)-one;
(R)-5,6-Difluoro-2-(((tetrahydro-2H-pyran-4-yl)thio)methyl)-7-(((tetrahydrofuran-3-yl)methyl)amino)quinazolin-4(3H)-one;
5-Fluoro-2-((((trans)-4-hydroxycyclohexyl)thio)methyl)-7-((tetrahydrofuran-3-yl)methoxy)quinazolin-4(3H)-one;
5-Fluoro-2-((((trans)-4-hydroxycyclohexyl)thio)methyl)-7-(((R)-tetrahydrofuran-3-yl)methoxy)quinazolin-4(3H)-one;
5-Fluoro-2-((((trans)-4-hydroxycyclohexyl)thio)methyl)-7-(((S)-tetrahydrofuran-3-yl)methoxy)quinazolin-4(3H)-one;
5-Fluoro-7-(((trans)-3-fluoro-1-methylpiperidin-4-yl)methoxy)-2-(((tetrahydro-2H-pyran-4-yl)thio)methyl)quinazolin-4(3H)-one;
5-Fluoro-7-(((3S,4S)-3-fluoro-1-methylpiperidin-4-yl)methoxy)-2-(((tetrahydro-2H-pyran-4-yl)thio)methyl)quinazolin-4(3H)-one;
5-Fluoro-7-(((3R,4R)-3-fluoro-1-methylpiperidin-4-yl)methoxy)-2-(((tetrahydro-2H-pyran-4-yl)thio)methyl)quinazolin-4(3H)-one;
5,6-Difluoro-7-(((cis)-3-methoxycyclobutyl)amino)-2-(((tetrahydro-2H-pyran-4-yl)thio)methyl)quinazolin-4(3H)-one;
N-((cis)-4-(((7-(Cyclopropylmethoxy)-5-fluoro-4-oxo-3,4-dihydroquinazolin-2-yl)methyl)thio)cyclohexyl)acetamide;
N-((trans)-4-(((7-(Cyclopropylmethoxy)-5-fluoro-4-oxo-3,4-dihydroquinazolin-2-yl)methyl)thio)cyclohexyl)acetamide;
7-(((cis)-3-Ethoxycyclobutyl)amino)-5,6-difluoro-2-(((tetrahydro-2H-pyran-4-yl)thio)methyl)quinazolin-4(3H)-one;
5-Fluoro-2-((((cis)-4-hydroxy-4-methylcyclohexyl)thio)methyl)-7-((tetrahydro-2H-pyran-4-yl)methoxy)quinazolin-4(3H)-one;
7-((1-Acetylpiperidin-4-yl)methoxy)-5-fluoro-2-(((tetrahydro-2H-pyran-4-yl)thio)methyl)quinazolin-4(3H)-one;
2-((((trans)-4-(Aminomethyl)-4-fluorocyclohexyl)thio)methyl)-7-(cyclobutylmethoxy)-5-fluoroquinazolin-4(3H)-one;
5-Fluoro-7-(((3S,4S)-3-fluoro-1-methylpiperidin-4-yl)methoxy)-2-((((trans)-4-hydroxycyclohexyl)thio)methyl)quinazolin-4(3H)-one;
5-Fluoro-7-(((3R,4R)-3-fluoro-1-methylpiperidin-4-yl)methoxy)-2-((((trans)-4-hydroxycyclohexyl)thio)methyl)quinazolin-4(3H)-one;
7-((Cyclopropylmethyl)amino)-5,6-difluoro-2-(((tetrahydro-2H-pyran-4-yl)thio)methyl)quinazolin-4(3H)-one;
5,6-Difluoro-7-(((tetrahydro-2H-pyran-4-yl)methyl)amino)-2-(((tetrahydro-2H-pyran-4-yl)thio)methyl)quinazolin-4(3H)-one;
7-((Cyclobutylmethyl)amino)-2-(((1,1-dioxidotetrahydro-2H-thiopyran-4-yl)thio)methyl)-5,6-difluoroquinazolin-4(3H)-one;
5-Fluoro-7-(((trans)-2-fluorocyclopentyl)amino)-2-((((trans)-4-hydroxycyclohexyl)thio)methyl)quinazolin-4(3H)-one;
5-Fluoro-7-(((cis)-2-fluorocyclopentyl)amino)-2-((((trans)-4-hydroxycyclohexyl)thio)methyl)quinazolin-4(3H)-one;
5-Fluoro-2-((((trans)-4-hydroxycyclohexyl)thio)methyl)-7-((tetrahydro-2H-pyran-4-yl)methoxy)quinazolin-4(3H)-one;
5-Fluoro-7-(oxetan-3-ylmethoxy)-2-(((tetrahydro-2H-pyran-4-yl)thio)methyl)quinazolin-4(3H)-one;
7-((1,4-Dioxan-2-yl)methoxy)-5-fluoro-2-(((tetrahydro-2H-pyran-4-yl)thio)methyl)quinazolin-4(3H)-one;
7-((2,2-Difluorocyclohexyl)amino)-5-fluoro-2-(((tetrahydro-2H-pyran-4-yl)thio)methyl)quinazolin-4(3H)-one;
5,6-Difluoro-7-(((trans)-4-(4-methylpiperazin-1-yl)cyclohexyl)amino)-2-(((tetrahydro-2H-pyran-4-yl)thio)methyl)quinazolin-4(3H)-one;
5,6-Difluoro-7-(((cis)-4-(4-methylpiperazin-1-yl)cyclohexyl)amino)-2-(((tetrahydro-2H-pyran-4-yl)thio)methyl)quinazolin-4(3H)-one;
(R)-5,6-Difluoro-7-((tetrahydro-2H-pyran-3-yl)amino)-2-(((tetrahydro-2H-pyran-4-yl)thio)methyl)quinazolin-4(3H)-one;
7-(((R)-1-Acetylpyrrolidin-3-yl)amino)-5,6-difluoro-2-((((trans)-4-hydroxycyclohexyl)thio)methyl)quinazolin-4(3H)-one;
7-((2,2-Difluorocyclopentyl)amino)-5-fluoro-2-((((trans)-4-hydroxycyclohexyl)thio)methyl)quinazolin-4(3H)-one;
7-((1,1-Dioxidotetrahydro-2H-thiopyran-4-yl)methoxy)-5-fluoro-2-(((tetrahydro-2H-pyran-4-yl)thio)methyl)quinazolin-4(3H)-one;
5-Fluoro-7-(((trans)-3-fluoropiperidin-4-yl)methoxy)-2-(((tetrahydro-2H-pyran-4-yl)thio)methyl)quinazolin-4(3H)-one;
5-Chloro-7-((tetrahydro-2H-pyran-4-yl)methoxy)-2-(((tetrahydro-2H-pyran-4-yl)thio)methyl)quinazolin-4(3H)-one;
5,6-Difluoro-2-((((trans)-4-hydroxycyclohexyl)thio)methyl)-7-((1-(3,3,3-trifluoropropyl)piperidin-4-yl)amino)quinazolin-4(3H)-one;
7-((5,5-Dimethyltetrahydrofuran-3-yl)methoxy)-5-fluoro-2-((((trans)-4-hydroxycyclohexyl)thio)methyl)quinazolin-4(3H)-one;
5-Fluoro-2-((((trans)-4-methoxycyclohexyl)thio)methyl)-7-((tetrahydro-2H-pyran-4-yl)methoxy)quinazolin-4(3H)-one;
5-Fluoro-2-((((cis)-4-methoxycyclohexyl)thio)methyl)-7-((tetrahydro-2H-pyran-4-yl)methoxy)quinazolin-4(3H)-one;
5-Fluoro-2-(((4-methyltetrahydro-2H-pyran-4-yl)thio)methyl)-7-((tetrahydro-2H-pyran-4-yl)methoxy)quinazolin-4(3H)-one;
5-Fluoro-7-(((cis)-2-hydroxycyclopentyl)methoxy)-2-(((tetrahydro-2H-pyran-4-yl)thio)methyl)quinazolin-4(3H)-one;
(trans)-4-((5,6-Difluoro-4-oxo-2-(((tetrahydro-2H-pyran-4-yl)thio)methyl)-3,4-dihydroquinazolin-7-yl)amino)cyclohexane-1-carbonitrile;
(cis)-4-((5,6-Difluoro-4-oxo-2-(((tetrahydro-2H-pyran-4-yl)thio)methyl)-3,4-dihydroquinazolin-7-yl)amino)cyclohexane-1-carbonitrile;
5,6-Difluoro-7-(((trans)-3-methoxycyclobutyl)amino)-2-(((tetrahydro-2H-pyran-4-yl)thio)methyl)quinazolin-4(3H)-one;
5,6-Difluoro-2-((((trans)-4-hydroxycyclohexyl)thio)methyl)-7-(((cis)-3-methoxycyclobutyl)amino)quinazolin-4(3H)-one;
5-Methyl-7-((tetrahydro-2H-pyran-4-yl)methoxy)-2-(((tetrahydro-2H-pyran-4-yl)thio)methyl)quinazolin-4(3H)-one;
5-Fluoro-2-((((cis)-4-hydroxycyclohexyl)thio)methyl)-7-((tetrahydro-2H-pyran-4-yl)methoxy)quinazolin-4(3H)-one;
2-(((4,4-Difluorocyclohexyl)thio)methyl)-5-fluoro-7-((tetrahydro-2H-pyran-4-yl)methoxy)quinazolin-4(3H)-one;
7-((1-Acetylpyrrolidin-3-yl)methoxy)-5-fluoro-2-(((tetrahydro-2H-pyran-4-yl)thio)methyl)quinazolin-4(3H)-one;
7-(2-Cyclohexylethyl)-5-fluoro-2-(((tetrahydro-2H-pyran-4-yl)thio)methyl)quinazolin-4(3H)-one;
7-(((1-Acetylpiperidin-4-yl)methyl)amino)-5,6-difluoro-2-(((tetrahydro-2H-pyran-4-yl)thio)methyl)quinazolin-4(3H)-one;
5-Fluoro-7-(((tetrahydro-2H-pyran-4-yl)methyl)thio)-2-(((tetrahydro-2H-pyran-4-yl)thio)methyl)quinazolin-4(3H)-one;
5-Fluoro-7-(((cis)-4-fluoropyrrolidin-3-yl)methoxy)-2-(((tetrahydro-2H-pyran-4-yl)thio)methyl)quinazolin-4(3H)-one;
5-Fluoro-7-(((cis)-4-fluoro-1-methylpyrrolidin-3-yl)methoxy)-2-(((tetrahydro-2H-pyran-4-yl)thio)methyl)quinazolin-4(3H)-one;
5-Fluoro-2-((((cis)-4-hydroxy-4-methylcyclohexyl)thio)methyl)-7-((tetrahydrofuran-3-yl)methoxy)quinazolin-4(3H)-one;
5,6-Difluoro-2-((((cis)-4-hydroxy-4-methylcyclohexyl)thio)methyl)-7-(((cis)-3-methoxycyclobutyl)amino)quinazolin-4(3H)-one;
5-Fluoro-7-((tetrahydro-2H-pyran-4-yl)methoxy)-2-((((trans)-4-(trifluoromethoxy)cyclohexyl)thio)methyl)quinazolin-4(3H)-one;
5-Bromo-2-(((tetrahydro-2H-pyran-4-yl)thio)methyl)-7-((tetrahydrofuran-3-yl)methoxy)quinazolin-4(3H)-one;
5,6-Difluoro-2-((((trans)-4-hydroxycyclohexyl)thio)methyl)-7-(((trans)-4-methoxycyclohexyl)amino)quinazolin-4(3H)-one;
N-((trans)-4-(((7-(Cyclopropylmethoxy)-5-fluoro-4-oxo-3,4-dihydroquinazolin-2-yl)methyl)thio)cyclohexyl)propionamide;
5,6-Difluoro-2-((((trans)-4-hydroxycyclohexyl)thio)methyl)-7-(((cis)-4-methoxycyclohexyl)amino)quinazolin-4(3H)-one;
N-(4-(((7-(Cyclopropylmethoxy)-5-fluoro-4-oxo-3,4-dihydroquinazolin-2-yl)methyl)thio)-1-methylcyclohexyl)acetamide;
5,6-Difluoro-2-((((trans)-4-hydroxycyclohexyl)thio)methyl)-7-(((R)-tetrahydro-2H-pyran-3-yl)amino)quinazolin-4(3H)-one;
5-Fluoro-2-((((trans)-3-hydroxycyclobutyl)thio)methyl)-7-((tetrahydro-2H-pyran-4-yl)methoxy)quinazolin-4(3H)-one;
4-Oxo-2-(((tetrahydro-2H-pyran-4-yl)thio)methyl)-7-((tetrahydrofuran-3-yl)methoxy)-3,4-dihydroquinazoline-5-carbonitrile;
5,6-Difluoro-7-(neopentylamino)-2-(((tetrahydro-2H-pyran-4-yl)thio)methyl)quinazolin-4(3H)-one;
5-Fluoro-7-(((cis)-3-hydroxy-3-methylcyclobutyl)methoxy)-2-(((tetrahydro-2H-pyran-4-yl)thio)methyl)quinazolin-4(3H)-one;
5-Fluoro-7-(((trans)-3-hydroxy-3-methylcyclobutyl)methoxy)-2-(((tetrahydro-2H-pyran-4-yl)thio)methyl)quinazolin-4(3H)-one;
N-((cis)-3-(((5-Fluoro-4-oxo-7-((tetrahydro-2H-pyran-4-yl)methoxy)-3,4-dihydroquinazolin-2-yl)methyl)thio)cyclobutyl)acetamide;
5-Fluoro-7-(((cis)-3-fluoro-1-methylpiperidin-4-yl)methoxy)-2-(((tetrahydro-2H-pyran-4-yl)thio)methyl)quinazolin-4(3H)-one;
N-((trans)-4-(((5,6-Difluoro-7-(((cis)-3-methoxycyclobutyl)amino)-4-oxo-3,4-dihydroquinazolin-2-yl)methyl)thio)cyclohexyl)acetamide;
7-((1-(Cyclopropanecarbonyl)piperidin-4-yl)methoxy)-5-fluoro-2-(((tetrahydro-2H-pyran-4-yl)thio)methyl)quinazolin-4(3H)-one;
N-((trans)-4-(((5-Fluoro-4-oxo-7-((tetrahydrofuran-3-yl)methoxy)-3,4-dihydroquinazolin-2-yl)methyl)thio)cyclohexyl)acetamide;
N-((trans)-4-(((7-(Cyclobutylamino)-5,6-difluoro-4-oxo-3,4-dihydroquinazolin-2-yl)methyl)thio)cyclohexyl)acetamide;
N-((trans)-3-(((5-Fluoro-4-oxo-7-((tetrahydro-2H-pyran-4-yl)methoxy)-3,4-dihydroquinazolin-2-yl)methyl)thio)cyclobutyl)acetamide;
7-(1-Cyclopentylethoxy)-5-fluoro-2-(((tetrahydro-2H-pyran-4-yl)thio)methyl)-5,6,7,8-tetrahydroquinazolin-4(3H)-one;
N-((trans)-4-(((7-(Cyclopropylmethoxy)-5-fluoro-4-oxo-3,4-dihydroquinazolin-2-yl)methyl)thio)cyclohexyl)cyclopropanecarboxamide;
7-((1-Acetylpiperidin-4-yl)methoxy)-5-fluoro-2-((((trans)-4-hydroxycyclohexyl)thio)methyl)quinazolin-4(3H)-one;
5-Fluoro-7-((1-isobutyrylpiperidin-4-yl)methoxy)-2-(((tetrahydro-2H-pyran-4-yl)thio)methyl)quinazolin-4(3H)-one;
5-Fluoro-7-((1-propionylpiperidin-4-yl)methoxy)-2-(((tetrahydro-2H-pyran-4-yl)thio)methyl)quinazolin-4(3H)-one;
5-Fluoro-7-(piperidin-4-ylmethoxy)-2-(((tetrahydro-2H-pyran-4-yl)thio)methyl)quinazolin-4(3H)-one;
5,6-Difluoro-7-((1-(tetrahydro-2H-pyran-4-yl)ethyl)amino)-2-(((tetrahydro-2H-pyran-4-yl)thio)methyl)quinazolin-4(3H)-one;
7-((1-Acetylpiperidin-3-yl)methoxy)-5-fluoro-2-(((tetrahydro-2H-pyran-4-yl)thio)methyl)quinazolin-4(3H)-one;
5,6-Difluoro-2-(((tetrahydro-2H-pyran-4-yl)thio)methyl)-7-(((cis)-3-(trifluoromethoxy)cyclobutyl)amino)quinazolin-4(3H)-one;
7-Amino-5,6-difluoro-2-(((tetrahydro-2H-pyran-4-yl)thio)methyl)quinazolin-4(3H)-one;
7-(Cyclopropylmethoxy)-2-((((trans)-4-(dimethylamino)cyclohexyl)thio)methyl)-5-fluoro-7,8-dihydroquinazolin-4(3H)-one;
5-Fluoro-2-((((cis)-3-hydroxycyclobutyl)thio)methyl)-7-((tetrahydro-2H-pyran-4-yl)methoxy)quinazolin-4(3H)-one;
5,6-Difluoro-7-((tetrahydro-2H-pyran-4-yl)methoxy)-2-(((tetrahydro-2H-pyran-4-yl)thio)methyl)quinazolin-4(3H)-one;
5,6-Difluoro-7-((2-methoxy-2-methylpropyl)amino)-2-(((tetrahydro-2H-pyran-4-yl)thio)methyl)quinazolin-4(3H)-one;
5,6-Difluoro-7-((((cis)-3-fluoro-1-methylpiperidin-4-yl)methyl)amino)-2-(((tetrahydro-2H-pyran-4-yl)thio)methyl)quinazolin-4(3H)-one;
7-((1-Acetylpiperidin-4-yl)methoxy)-5-fluoro-2-((((cis)-4-hydroxy-4-methylcyclohexyl)thio)methyl)quinazolin-4(3H)-one;
Methyl 4-(((5-fluoro-4-oxo-2-(((tetrahydro-2H-pyran-4-yl)thio)methyl)-3,4-dihydroquinazolin-7-yl)oxy)methyl)piperidine-1-carboxylate;
5-Fluoro-2-((((trans)-4-hydroxy-4-methylcyclohexyl)thio)methyl)-7-((tetrahydro-2H-pyran-4-yl)methoxy)quinazolin-4(3H)-one;
7-(Cyclopentylamino)-5-fluoro-2-((((trans)-3-fluoro-1-methylpiperidin-4-yl)thio)methyl)quinazolin-4(3H)-one;
7-(Cyclopentylamino)-5-fluoro-2-((((cis)-3-fluoro-1-methylpiperidin-4-yl)thio)methyl)quinazolin-4(3H)-one;
5-Fluoro-7-((4-methylmorpholin-2-yl)methoxy)-2-(((tetrahydro-2H-pyran-4-yl)thio)methyl)quinazolin-4(3H)-one;
5-Fluoro-7-((1-methylpiperidin-4-yl)methoxy)-2-(((tetrahydro-2H-pyran-4-yl)thio)methyl)quinazolin-4(3H)-one;
5-Fluoro-7-(neopentyloxy)-2-(((tetrahydro-2H-pyran-4-yl)thio)methyl)quinazolin-4(3H)-one;
7-((1-Acetylpiperidin-4-yl)methoxy)-5-fluoro-2-((((trans)-4-hydroxy-4-methylcyclohexyl)thio)methyl)quinazolin-4(3H)-one;
5-Fluoro-7-((tetrahydro-2H-pyran-4-yl)methoxy)-2-((((cis)-4-(trifluoromethoxy)cyclohexyl)thio)methyl)quinazolin-4(3H)-one;
7-(((1-Acetylpiperidin-4-yl)methyl)amino)-5,6-difluoro-2-((((trans)-4-hydroxycyclohexyl)thio)methyl)quinazolin-4(3H)-one;
5,6-Difluoro-7-(methylamino)-2-(((tetrahydro-2H-pyran-4-yl)thio)methyl)quinazolin-4(3H)-one;
5-Fluoro-2-(((tetrahydro-2H-pyran-4-yl)thio)methyl)-7-(3,3,3-trifluoro-2,2-dimethylpropoxy)quinazolin-4(3H)-one;
7-((1-Acetylpiperidin-4-yl)methoxy)-5-fluoro-2-((((cis)-4-hydroxycyclohexyl)thio)methyl)quinazolin-4(3H)-one;
7-((1-Acetylpiperidin-4-yl)methoxy)-5-chloro-2-(((tetrahydro-2H-pyran-4-yl)thio)methyl)quinazolin-4(3H)-one;
5-Fluoro-7-((1-(2-methoxyacetyl)piperidin-4-yl)methoxy)-2-(((tetrahydro-2H-pyran-4-yl)thio)methyl)quinazolin-4(3H)-one;
5,6-Difluoro-7-((((trans)-3-fluoro-1-methylpiperidin-4-yl)methyl)amino)-2-(((tetrahydro-2H-pyran-4-yl)thio)methyl)quinazolin-4(3H)-one;
N-((trans)-4-(((5-Fluoro-4-oxo-7-((tetrahydro-2H-pyran-4-yl)methoxy)-3,4-dihydroquinazolin-2-yl)methyl)thio)cyclohexyl)acetamide; and
7-((3,3-Difluoro-1-methylpiperidin-4-yl)methoxy)-5-fluoro-2-(((tetrahydro-2H-pyran-4-yl)thio)methyl)quinazolin-4(3H)-one, or a pharmaceutically acceptable salt thereof.

The PARP14 inhibitors referred to herein are described in U.S. Pat. Publication No: US2019/0194174, the entirety of which is incorporated herein by reference.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, substituents of compounds of the invention are disclosed in groups or in ranges. It is specifically intended that the invention include each and every individual subcombination of the members of such groups and ranges.

For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “pyridinyl,” “pyridyl,” or “a pyridine ring” may refer to a pyridin-2-yl, pyridin-3-yl, or pyridin-4-yl ring.

The term “n-membered,” where “n” is an integer, typically describes the number of ring-forming atoms in a moiety where the number of ring-forming atoms is “n”. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

For compounds of the invention in which a variable appears more than once, each variable can be a different moiety independently selected from the group defining the variable. For example, where a structure is described having two R groups that are simultaneously present on the same compound, the two R groups can represent different moieties independently selected from the group defined for R.

As used herein, the phrase “optionally substituted” means unsubstituted or substituted.

As used herein, the term “substituted” means that a hydrogen atom is replaced by a non-hydrogen group. It is to be understood that substitution at a given atom is limited by valency.

As used herein, the term “Cᵢ₋ⱼ” where i and j are integers, employed in combination with a chemical group, designates a range of the number of carbon atoms in the chemical group with i-j defining the range. For example, C₁₋₆ alkyl refers to an alkyl group having 1, 2, 3, 4, 5, or 6 carbon atoms.

As used herein, the term “alkyl,” employed alone or in combination with other terms, refers to a saturated hydrocarbon group that may be straight-chain or branched. In some embodiments, the alkyl group contains 1 to 7, 1 to 6, 1 to 4, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, n-heptyl, and the like. In some embodiments, the alkyl group is methyl, ethyl, or propyl.

As used herein, “alkenyl,” employed alone or in combination with other terms, refers to an alkyl group having one or more carbon-carbon double bonds. In some embodiments, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some embodiments, the alkynyl moiety contains 2 to 6 or 2 to 4 carbon atoms.

As used herein, “halo” or “halogen”, employed alone or in combination with other terms, includes fluoro, chloro, bromo, and iodo. In some embodiments, halo is F or Cl.

As used herein, the term “haloalkyl,” employed alone or in combination with other terms, refers to an alkyl group having up to the full valency of halogen atom substituents, which may either be the same or different. In some embodiments, the halogen atoms are fluoro atoms. In some embodiments, the alkyl group has 1 to 6 or 1 to 4 carbon atoms. Example haloalkyl groups include CF₃, C₂F₅, CHF₂, CCl₃, CHCl₂, C₂Cl₅, and the like.

As used herein, the term “alkoxy,” employed alone or in combination with other terms, refers to a group of formula —O-alkyl. Example alkoxy groups include methoxy, ethoxy, propoxy (e.g., n-propoxy and isopropoxy), t-butoxy, and the like. In some embodiments, the alkyl group has 1 to 6 or 1 to 4 carbon atoms.

As used herein, “haloalkoxy,” employed alone or in combination with other terms, refers to a group of formula —O-(haloalkyl). In some embodiments, the alkyl group has 1 to 6 or 1 to 4 carbon atoms. An example haloalkoxy group is —OCF₃.

As used herein, “amino,” employed alone or in combination with other terms, refers to NH₂.

As used herein, the term “alkylamino,” employed alone or in combination with other terms, refers to a group of formula —NH(alkyl). In some embodiments, the alkylamino group has 1 to 6 or 1 to 4 carbon atoms. Example alkylamino groups include methylamino, ethylamino, propylamino (e.g., n-propylamino and isopropylamino), and the like.

As used herein, the term “dialkylamino,” employed alone or in combination with other terms, refers to a group of formula —N(alkyl)₂. Example dialkylamino groups include dimethylamino, diethylamino, dipropylamino (e.g., di(n-propyl)amino and di(isopropyl)amino), and the like. In some embodiments, each alkyl group independently has 1 to 6 or 1 to 4 carbon atoms.

As used herein, the term “cycloalkyl,” employed alone or in combination with other terms, refers to a non-aromatic cyclic hydrocarbon including cyclized alkyl and alkenyl groups. Cycloalkyl groups can include mono- or polycyclic (e.g., having 2, 3, or 4 fused, bridged, or spiro rings) ring systems. Also included in the definition of cycloalkyl are moieties that have one or more aromatic rings (e.g., aryl or heteroaryl rings) fused (i.e., having a bond in common with) to the cycloalkyl ring, for example, benzo derivatives of cyclopentane, cyclohexene, cyclohexane, and the like, or pyrido derivatives of cyclopentane or cyclohexane. Ring-forming carbon atoms of a cycloalkyl group can be optionally substituted by oxo. Cycloalkyl groups also include cycloalkylidenes. The term “cycloalkyl” also includes bridgehead cycloalkyl groups (e.g., non-aromatic cyclic hydrocarbon moieties containing at least one bridgehead carbon, such as admantan-1-yl) and spirocycloalkyl groups (e.g., non-aromatic hydrocarbon moieties containing at least two rings fused at a single carbon atom, such as spiro[2.5]octane and the like). In some embodiments, the cycloalkyl group has 3 to 10 ring members, or 3 to 7 ring members. In some embodiments, the cycloalkyl group is monocyclic or bicyclic. In some embodiments, the cycloalkyl group is monocyclic. In some embodiments, the cycloalkyl group is a C₃₋₇ monocyclic cycloalkyl group. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbomyl, norpinyl, norcamyl, tetrahydronaphthalenyl, octahydronaphthalenyl, indanyl, and the like. In some embodiments, the cycloalkyl group is cyclopropyl, cyclobutyl, cyclopentyl, or cyclohexyl.

As used herein, the term “cycloalkylalkyl,” employed alone or in combination with other terms, refers to a group of formula cycloalkyl-alkyl-. In some embodiments, the alkyl portion has 1 to 4, 1 to 3, 1 to 2, or 1 carbon atom(s). In some embodiments, the alkyl portion is methylene. In some embodiments, the cycloalkyl portion has 3 to 10 ring members or 3 to 7 ring members. In some embodiments, the cycloalkyl group is monocyclic or bicyclic. In some embodiments, the cycloalkyl portion is monocyclic. In some embodiments, the cycloalkyl portion is a C₃₋₇ monocyclic cycloalkyl group.

As used herein, the term “heterocycloalkyl,” employed alone or in combination with other terms, refers to a non-aromatic ring or ring system, which may optionally contain one or more alkenylene or alkynylene groups as part of the ring structure, which has at least one heteroatom ring member independently selected from nitrogen, sulfur, oxygen, and phosphorus. Heterocycloalkyl groups can include mono- or polycyclic (e.g., having 2, 3 or 4 fused, bridged, or spiro rings) ring systems. In some embodiments, the heterocycloalkyl group is a monocyclic or bicyclic group having 1, 2, 3, or 4 heteroatoms independently selected from nitrogen, sulfur and oxygen. Also included in the definition of heterocycloalkyl are moieties that have one or more aromatic rings (e.g., aryl or heteroaryl rings) fused (i.e., having a bond in common with) to the non-aromatic heterocycloalkyl ring, for example, 1,2,3,4-tetrahydro-quinoline and the like. Heterocycloalkyl groups can also include bridgehead heterocycloalkyl groups (e.g., a heterocycloalkyl moiety containing at least one bridgehead atom, such as azaadmantan-1-yl and the like) and spiroheterocycloalkyl groups (e.g., a heterocycloalkyl moiety containing at least two rings fused at a single atom, such as [1,4-dioxa-8-aza-spiro[4.5]decan-N-yl] and the like). In some embodiments, the heterocycloalkyl group has 3 to 10 ring-forming atoms, 4 to 10 ring-forming atoms, or about 3 to 8 ring forming atoms. In some embodiments, the heterocycloalkyl group has 2 to 20 carbon atoms, 2 to 15 carbon atoms, 2 to 10 carbon atoms, or about 2 to 8 carbon atoms. In some embodiments, the heterocycloalkyl group has 1 to 5 heteroatoms, 1 to 4 heteroatoms, 1 to 3 heteroatoms, or 1 to 2 heteroatoms. The carbon atoms or heteroatoms in the ring(s) of the heterocycloalkyl group can be oxidized to form a carbonyl, an N-oxide, or a sulfonyl group (or other oxidized linkage) or a nitrogen atom can be quaternized. In some embodiments, the heterocycloalkyl portion is a C₂₋₇ monocyclic heterocycloalkyl group. In some embodiments, the heterocycloalkyl group is a morpholine ring, pyrrolidine ring, piperazine ring, piperidine ring, tetrahydropyran ring, tetrahydropyridine, azetidine ring, or tetrahydrofuran ring.

As used herein, the term “heterocycloalkylalkyl,” employed alone or in combination with other terms, refers to a group of formula heterocycloalkyl-alkyl-. In some embodiments, the alkyl portion has 1 to 4, 1 to 3, 1 to 2, or 1 carbon atom(s). In some embodiments, the alkyl portion is methylene. In some embodiments, the heterocycloalkyl portion has 3 to 10 ring members, 4 to 10 ring members, or 3 to 7 ring members. In some embodiments, the heterocycloalkyl group is monocyclic or bicyclic. In some embodiments, the heterocycloalkyl portion is monocyclic. In some embodiments, the heterocycloalkyl portion is a C₂₋₇ monocyclic heterocycloalkyl group.

As used herein, the term “aryl,” employed alone or in combination with other terms, refers to a monocyclic or polycyclic (e.g., a fused ring system) aromatic hydrocarbon moiety, such as, but not limited to, phenyl, 1-naphthyl, 2-naphthyl, and the like. In some embodiments, aryl groups have from 6 to 10 carbon atoms or 6 carbon atoms. In some embodiments, the aryl group is a monocyclic or bicyclic group. In some embodiments, the aryl group is phenyl or naphthyl.

As used herein, the term “arylalkyl,” employed alone or in combination with other terms, refers to a group of formula aryl-alkyl-. In some embodiments, the alkyl portion has 1 to 4, 1 to 3, 1 to 2, or 1 carbon atom(s). In some embodiments, the alkyl portion is methylene. In some embodiments, the aryl portion is phenyl. In some embodiments, the aryl group is a monocyclic or bicyclic group. In some embodiments, the arylalkyl group is benzyl.

As used herein, the term “heteroaryl,” employed alone or in combination with other terms, refers to a monocyclic or polycyclic (e.g., a fused ring system) aromatic hydrocarbon moiety, having one or more heteroatom ring members independently selected from nitrogen, sulfur and oxygen. In some embodiments, the heteroaryl group is a monocyclic or a bicyclic group having 1, 2, 3, or 4 heteroatoms independently selected from nitrogen, sulfur and oxygen. Example heteroaryl groups include, but are not limited to, pyridyl, pyrimidinyl, pyrazinyl, pyridazinyl, triazinyl, furyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, pyrrolyl, azolyl, quinolinyl, isoquinolinyl, benzisoxazolyl, imidazo[1,2-b]thiazolyl or the like. The carbon atoms or heteroatoms in the ring(s) of the heteroaryl group can be oxidized to form a carbonyl, an N-oxide, or a sulfonyl group (or other oxidized linkage) or a nitrogen atom can be quaternized, provided the aromatic nature of the ring is preserved. In some embodiments, the heteroaryl group has from 3 to 10 carbon atoms, from 3 to 8 carbon atoms, from 3 to 5 carbon atoms, from 1 to 5 carbon atoms, or from 5 to 10 carbon atoms. In some embodiments, the heteroaryl group contains 3 to 14, 4 to 12, 4 to 8, 9 to 10, or 5 to 6 ring-forming atoms. In some embodiments, the heteroaryl group has 1 to 4, 1 to 3, or 1 to 2 heteroatoms.

As used herein, the term “heteroarylalkyl,” employed alone or in combination with other terms, refers to a group of formula heteroaryl-alkyl-. In some embodiments, the alkyl portion has 1 to 4, 1 to 3, 1 to 2, or 1 carbon atom(s). In some embodiments, the alkyl portion is methylene. In some embodiments, the heteroaryl portion is a monocyclic or bicyclic group having 1, 2, 3, or 4 heteroatoms independently selected from nitrogen, sulfur and oxygen. In some embodiments, the heteroaryl portion has 5 to 10 carbon atoms.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Geometric isomers of olefins, C═N double bonds, and the like can also be present in the compounds described herein, and all such stable isomers are contemplated in the present invention. Cis and trans geometric isomers of the compounds of the present invention may be isolated as a mixture of isomers or as separated isomeric forms.

The compounds described herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

The compounds described herein also include all isotopes of atoms occurring in the intermediates or final compounds. Isotopes include those atoms having the same atomic number but different mass numbers. For example, isotopes of hydrogen include tritium and deuterium. In some embodiments, the compounds of the invention include at least one deuterium atom.

The term “compound,” as used herein, is meant to include all stereoisomers, geometric isomers, tautomers, and isotopes of the structures depicted, unless otherwise specified.

All compounds, and pharmaceutically acceptable salts thereof, can be found together with other substances such as water and solvents (e.g., in the form of hydrates and solvates) or can be isolated.

In some embodiments, the compounds described herein, or salts thereof, are substantially isolated. By “substantially isolated” is meant that the compound is at least partially or substantially separated from the environment in which it was formed or detected. Partial separation can include, for example, a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Methods of Use

It was found that genetic inactivation of Poly(ADP-Ribose) Polymerase Family Member 14 (PARP14), also referred to as ADP-Ribosyltransferase Diphtheria Toxin-Like 8 (ARTD8) or B Aggressive Lymphoma Protein (BAL2), protected mice against allergen-induced airway disease (Mehrothra et al. U.S. Pat. No. 22,841,009, Cho et al. 23956424), suppressed the infiltration of immune cells such as eosinophils and neutrophils into the lung, and reduced the release of inflammatory Th2 cytokines. In addition, treatment with a PARP14 inhibitor protected mice in a severe asthma model induced by a sensitization and recall challenge with inhaled Alternaria alternata extract. PARP14 inhibitor-treated animals showed a reduced level of airway mucus, blood serum IgE, infiltration of immune cells (eosinophils, neutrophils, and lymphocytes), Th2 cytokines (IL-4, IL-5, and IL13) and alarmins (IL-33 and TSLP) (Ribon internal data).

While not being bound by theory, PARP14 has been shown to affect STAT6 signaling and STAT3 signaling, signaling induced by Th2 cytokines and Th17 cytokines, M1/M2 macrophage polarization, and signaling by lymphocytes. PARP14 has also been shown to be a regulator of Th2/Th17/THF T cell development, involved in B cell development, and involved in eosinophils/neutrophils recruitment/activation. It is believed that the lymphocytes are likely the ILCs (e.g., ILC2 and ILC3) that get activated by the alarmins (e.g., TSLP and IL-33) and are the main producers of the downstream cytokines (e.g., IL-4, IL-5, and IL-13).

It is suggested that PARP14 inhibition affects the asthma phenotype not only at the level of the second order cytokines (e.g., IL-4, IL-5, and IL-13) and the signaling to the myeloid cells, but that PARP14 inhibition also suppresses the alarmins TSLP and IL-33, which are the key upstream drivers of asthma that get released in response to the allergens.

The present invention is directed, inter alia, to a method of treating or preventing an inflammatory disease in a patient comprising administering to the patient a therapeutically effective amount of a PARP14 inhibitor such as any of the compounds described herein, or their pharmaceutically acceptable salts.

Exemplary inflammatory diseases that are treatable by the disclosed methods include, e.g., asthma, atopic dermatitis, psoriasis, rhinitis, systemic sclerosis, keloids, eosinophilic disorders, pulmonary fibrosis, and other type 2 cytokine pathologies. In some embodiments, the pulmonary fibrosis is idiopathic pulmonary fibrosis.

Additional exemplary inflammatory diseases that are treatable by the disclosed methods include inflammatory bowel diseases (“IBD”), which include ulcerative colitis (“UC” or “colitis”) and Crohn's disease. In some embodiments, the inflammatory disease is inflammatory bowel disease. In some embodiments, the inflammatory disease is ulcerative colitis. In some embodiments, the inflammatory disease is Crohn's disease.

In some embodiments, the inflammatory disease is irritable bowel syndrome.

Eosinophilic disorders that are treatable by the disclosed methods include, e.g., eosinophilic esophagitis (esophagus—EoE), eosinophilic gastritis (stomach—EG), eosinophilic gastroenteritis (stomach and small intestine—EGE), eosinophilic enteritis (small intestine—EE), eosinophilic colitis (large intestine—EC), and eosinophilic chronic rhinosinusitis.

The present invention is further directed, inter alia, to a method of treating or preventing asthma in a patient comprising administering to the patient a therapeutically effective amount of a PARP14 inhibitor such as any of the compounds described herein, or their pharmaceutically acceptable salts.

In some embodiments, the asthma is steroid-insensitive asthma, steroid-refractory asthma, steroid-resistant asthma, atopic asthma, nonatopic asthma, persistent asthma, severe asthma, or steroid-refractory severe asthma. In some embodiments, the severe asthma is T2 high endotype, T2 low endotype, or non-T2 endotype. In some embodiments, the severe asthma is T2 high endotype. In some embodiments, the severe asthma is T2 low endotype or non-T2 endotype. In some embodiments, the severe asthma is T2 low endotype. In some embodiments, the severe asthma is non-T2 endotype.

The present invention is further directed, inter alia, to a method of treating or preventing fibrotic diseases such as, but not limited to, pulmonary fibrosis, renal fibrosis, hepatic fibrosis (e.g., NASH and NAFLD), systemic fibrosis, and idiopathic pulmonary fibrosis (IPF). In some embodiments, the fibrotic disease is systemic fibrosis.

The present invention is further directed, inter alia, to a method of treating or preventing chronic obstructive pulmonary disease (COPD), emphysema, and chronic bronchitis.

The present invention is further directed, inter alia, to a method of treating or preventing a skin inflammatory disease such as atopic dermatitis or rosacea.

The present invention further provides a method of:

(a) reducing the level of airway mucus in lung tissue,

(b) reducing blood serum IgE,

(c) reducing immune cell infiltration and activation in bronchoalveolar fluid,

(d) reducing the level of one or more inflammatory cytokines in bronchoalveolar fluid or in lung tissue, or

(e) reducing the level of one or more alarmins in bronchoalveolar fluid or lung tissue,

in a patient, where the method comprises administering to the patient a therapeutically effective amount of a PARP14 inhibitor, such as a compound disclosed herein, or a pharmaceutically acceptable salt thereof.

In some embodiments, the present invention provides a method of reducing the level of airway mucus in lung tissue in a patient.

In some embodiments, the present invention provides a method of reducing immune cell infiltration and activation in bronchoalveolar fluid in a patient. In some embodiments, the immune cells are eosinophils, neutrophils, or lymphocytes.

In some embodiments, the present invention provides a method of reducing one or more inflammatory cytokines in bronchoalveolar fluid or in lung tissue in a patient. In some embodiments, the inflammatory cytokine is a Th2 cytokine or Th17 cytokine. In some embodiments, the inflammatory cytokine is a Th2 cytokine. In some embodiments, the inflammatory cytokine is IL-4, IL-5, IL13, or IL-17A. In some embodiments, the inflammatory cytokine is IL-4, IL-5, or IL 13.

In some embodiments, the present invention provides a method of reducing an alarmin in bronchoalveolar fluid or in lung tissue in a patient. In some embodiments, the alarmin is IL-25, IL-33 or TSLP.

As used herein, the term “reducing” is with respect to the level in the patient prior to administration. More specifically, when a biomarker or symptom is reduced in a patient, the reduction is with respect to the level of or severity of the biomarker or symptom in the patient prior to administration of the compound of Formula (I), or a pharmaceutically acceptable salt thereof.

As used herein, the term “individual” or “patient,” used interchangeably, refers to mammals, and particularly humans. The patient can be in need of treatment.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease in an individual who is experiencing or displaying the pathology or symptomatology of the disease (i.e., arresting further development of the pathology and/or symptomatology), or 2) ameliorating the disease in an individual who is experiencing or displaying the pathology or symptomatology of the disease (i.e., reversing the pathology and/or symptomatology).

As used herein the term “preventing” or “prevention” refers to preventing the disease in an individual who may be predisposed to the disease but does not yet experience or display the pathology or symptomatology of the disease.

### Combination Therapy

The compounds of the present invention can be administered in combination with one or more additional pharmaceutical agent or treatment method which can be, for example, an inhaled coriticosteroid, an inhaled long-acting beta antagonist, or intravenously-injected monoclonal antibodies. The compound of the present invention can also be administered in combination with other treatments or other targeted therapies. The agents can be combined with the present compounds in a single dosage form, or the agents can be administered simultaneously or sequentially as separate dosage forms.

Suitable agents for use in combination with the compounds of the present invention for the treatment of inflammatory diseases include but are not limited to corticosteroids (e.g., prednisone, prednisolone, methylprednisolone, and hydrocortisone); disease-modifying antirheumatic drugs (“DMARDs”, e g, immunosuppressive or anti-inflammatory agents); anti-malarial agents (e.g. hydroxychloroquine and chloroquine); immunosuppressive agents (e.g., cyclophosphamide, azathioprine, mycophenolate mofetil, methotrexate); anti-inflammatory agents (e.g., aspirin, NSAIDs (e.g., ibuprofen, naproxen, indomethacin, nabumetone, celecoxib)); anti-hypertensive agents (e.g., calcium channel blockers (e.g., amlodipine, nifedipine) and diuretics (e.g., furosemide)); statins (e.g., atorvastatin, fluvastatin, lovastatin, pitavastatin, pravastatin, rosuvastatin and simvastatin); anti-B-cell agents (e.g., anti-CD20 (e.g., rituximab), anti-CD22); anti-B-lymphocyte stimulator agents (“anti-BLyS”, e.g., belimumab, blisibimod); type-1 interferon receptor antagonist (e.g., anifrolumab); T-cell modulators (e.g., rigerimod); abatacept; anticoagulants (e.g., heparin, warfarin); and vitamin D supplements.

Additional suitable agents for use in combination of the present invention for the treatment of inflammatory diseases include but not are not limited to sulfonylureas, meglitinides, biguanides, alpha-glucosidase inhibitors, peroxisome proliferators-activated receptor-gamma (i.e., PPAR-gamma) agonists, insulin, insulin analogues, HMG-CoA reductase inhibitors, cholesterol-lowering drugs (for example, fibrates that include: fenofibrate, bezafibrate, gemfibrozil, clofibrate and the like; bile acid sequestrants which include: cholestyramine, colestipol and the like; and niacin), anti-platelet agents (for example, aspirin and adenosine diphosphate receptor antagonists that include: clopidogrel, ticlopidine and the like), angiotensin-converting enzyme inhibitors, angiotensin II receptor antagonists and adiponectin.

Suitable agents for use in combination with the compounds of the present invention for the treatment of asthma include but are not limited to beclomethasone (Qvar™), budesonide (Pulmicort Flexhaler™), budesonide/formoterol (Symbicort™), ciclesonide (Alvesco™), flunisolide (Aerospan™), fluticasone (Flovent Diskus™, flovent HFA™ Amuity Ellipta™), fluticasone/salmeterol (Advair™), mometasone (Asmanex™), mometasone/formoterol (Dulera™), albuterol sulfate (VoSpireER™), formoterol fumarate (Aerolizer™), salmeterol xinafoate (Serevent™), arformoterol tartrate (Brovana™), olodaterol (Striverdi™), fluticasone furoate/vilanterol (Breo Ellipta™), fluticasone furoate/umeclidinium/vilanterol (Trelegy Ellipta™), fluticasone propionate/salmeterol (AirDuo™), glycopyrrolate/formoterol fumarate (Bevespi Aerosphere™), indacaterol/glycopyrrolate (Utibron Neohaler™), tiotropium/olodaterol (Stiolto Respimat™), umeclidinium/vilanterol (Anoro Ellipta™), omalizumab (Xolair™), mepolizumab (NUCALA™), benralizumab (Fasenra™), reslizumab (CinqaiT™), dupilumab, tralokinumab, lebrikizumab, etanercept, golimumab brodalumab, and tezepelumab.

### Pharmaceutical Formulations and Dosage Forms

When employed as pharmaceuticals, the compounds of the invention can be administered in the form of pharmaceutical compositions. A pharmaceutical composition refers to a combination of a compound of the invention, or its pharmaceutically acceptable salt, and at least one pharmaceutically acceptable carrier. These compositions can be prepared in a manner well known in the pharmaceutical art, and can be administered by a variety of routes, depending upon whether local or systemic treatment is desired and upon the area to be treated. Administration may be oral, topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, or parenteral.

This invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

The compositions can be formulated in a unit dosage form. The term “unit dosage form” refers to a physically discrete unit suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid pre-formulation composition containing a homogeneous mixture of a compound of the present invention. When referring to these pre-formulation compositions as homogeneous, the active ingredient is typically dispersed evenly throughout the composition so that the composition can be readily subdivided into equally effective unit dosage forms such as tablets, pills and capsules. This solid pre-formulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques or may be sterile filtered. Aqueous solutions can be packaged for use as is, or lyophilized, the lyophilized preparation being combined with a sterile aqueous carrier prior to administration.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds of the invention can also be formulated in combination with one or more additional active ingredients which can include any pharmaceutical agent such as anti-viral agents, anti-cancer agents, vaccines, antibodies, immune enhancers, immune suppressants, anti-inflammatory agents, and the like.

### EXAMPLES

Inter-group deviations were statistically analyzed by a one-way analysis of variance (ANOVA). In the case of a significant difference in the mean values among the different levels of treatment, comparisons versus the vehicle group were carried out using the Dunnett's test. p<0.05 will be considered statistically significant.

### Example A. Dose and Efficacy Study of Compound 1 in Alternaria-Induced Asthma Model

This is a dose response evaluation of the effect of the PARP14 inhibitor, Compound 1:

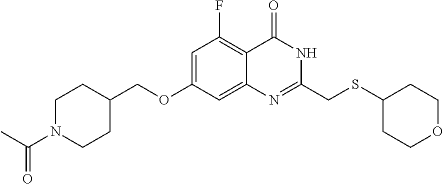 [SMILES: CC(=O)N1CCC(COc2cc(F)c3c(=O)[nH]c(CSC4CCOCC4)nc3c2)CC1]

### 7-((1-Acetylpiperidin-4-yl)methoxy)-5-fluoro-2-(((tetrahydro-2H-pyran-4-yl)thio)methyl)quinazolin-4(3H)-one

on pulmonary inflammation by assessing bronchoalveolar lavage (BAL) fluid cell numbers, BAL fluid supernatant concentrations and lung tissue homogenate concentrations of TSLP, IL-4, IL-5, IL13, and IL-33, total IgE concentrations in serum and production of airway mucus in mice exposed to Alternaria. This Alternaria-induce asthma model in mice is an accepted animal model for assessing potential efficacy in the treatment of asthma, including severe asthma. See U.S. Pat. Publication No: US 2019/0194174 for a description of Compound 1.

### Animal Welfare

All animals throughout the study were held in the biological support unit at Pharmidex's facilities at European Knowledge Centre, Mosquito Way, Hatfield, AL10 9WN, United Kingdom. The facility operates under the United Kingdom home office animal scientific procedures act and as such has a valid establishment license (X07D13023) allowing regulated animal procedures to be carried out in the facility.

On arrival from the supplier, the animals (55 male BalbC mice, 20-30 g on arrival, Charles Rivers, UK) were placed into cages of 5 as outlined below, and the welfare of all animals was checked on a daily basis. Emergency care if required was provided by the named veterinary surgeon, although this was not necessary during the studies.

Throughout the study, the following guidelines (provided by the Home Office) were used to assess non-specific or unexpected adverse effects in animals undergoing regulated activities, relating to either the procedure or test compound dosing. Animals showing two or more of any of the limiting clinical signs in the category equivalent to the protocol severity limit were removed from the study and were killed by a schedule 1 method (cervical dislocation) at the establishment. Where an animal reached the limit of either or both of the first two signs with or without any other signs it was removed from the study and killed by a schedule 1 method at the establishment.

Body weight loss greater than 20% of the highest measured individual body weight.

Marked piloerection with other signs of dehydration such as skin tenting.

Unresponsive to activity and provocation.

Hunched persistently (frozen).

Distressed—persistent vocalization.

Oculo-nasal discharge persistent and copious.

Labored respiration.

Persistent tremors.

Persistent convulsions.

Animals also exposed to severe fight injuries were removed from the study and killed by a schedule 1 method at the establishment.

An acclimatization period of 7 days was allowed, before start of experimental procedures. Mice were housed in cages of 5 on arrival based on weight (equal distribution of animal weights amongst each of the cages by the animal technician) with a 12 hour light dark cycle. Room temperature and humidity was maintained within home office guidelines (17-24° C. and 40-70% respectively). Environmental enrichment was provided in all cages. Mice had access to standard chow ad libitum, and water was available from bottles ad libitum.

### Allergen Exposure

Alternaria alternata was diluted in sterile phosphate-buffered saline (PBS) to 5 μg of Alternaria per 40 μl.

On days 1-5 Balb/c mice were challenged (intranasal) under isoflurane anesthesia. On days 23-25 Balb/c mice were challenged (intranasal) under isoflurane anesthesia one hour post the first dosing of the day. PBS (Group 1) or Alternaria (Groups 2-6) were instilled into each nostril in a drop wise fashion alternating between the two until a volume of 40 μl had been delivered.

### Formulation and Treatment Regime

1-Aminobenzotriazole (1-ABT 25 mg/kg) and Compound 1 (150, 500, and 1500 mg/kg) were each independently formulated in vehicles of 0.5% methylcellulose (MC) and 0.2% Tween in distilled water, and 1-aminobenzotriazole (1-ABT 25 mg/kg) and Compound 1 (150, 500, and 1500 mg/kg) were co-formulated in a vehicle of 0.5% methylcellulose (MC) and 0.2% Tween in distilled water.

Group 1: On days 19-25, the animals were dosed with the vehicle (10 mL/kg, p.o., b.i.d.). On day 26, the animals were dosed with the vehicle 23 hours post the day 25 PBS challenge. All mice were taken down 2 hours later.

Group 2: On days 19-25, the animals were dosed with the vehicle (10 mL/kg, p.o., b.i.d.). On day 26, the animals were dosed with the vehicle 23 hours post the day 25 Alternaria challenge. All mice were taken down 2 hours later.

Groups 3, 4, and 5: On days 19-25, the animals were dosed with the Compound 1 vehicles (10 mL/kg, p.o., b.i.d.). On day 26, the animals were dosed with the Compound 1 vehicles 23 hours post the day 25 Alternaria challenge. All mice were taken down 2 hours later.

Group 6: On day 19, the animals were dosed with the 1-ABT vehicle (10 mL/kg) followed by the Compound 1 vehicle (10 mL/kg) 2 hours later. Twelve hours after the dosing of the Compound 1 vehicle, the animals were dosed with the 1-ABT and Compound 1 vehicle. On days 20-25, the 1-ABT and Compound 1 vehicle was administered (p.o., b.i.d.). On day 26, the 1-ABT and Compound 1 vehicle was dosed 23 hours post the day 25 Alternaria challenge. All mice were taken down 2 hours later.

### Blood Sample Collection

On day 19, Group 3-6 animals 1-5 had blood samples (15-20 μL) collected by venipuncture via the lateral tail vein 2 hr after the first dose of Compound 1, and Group 3-6 animals 6-10 had blood samples (15-20 μL) collected by venipuncture via the lateral tail vein 30 min prior to the second dose of Compound 1 (11.5 hr post 1st dose). On day 26, blood samples were collected from all animals 30 min prior to the last Compound 1 dose and 2 hrs after the last dose as a terminal bleed. Each blood sample (15-20 μL) was placed into EDTA tubes and mixed gently before being centrifuged (2000 g, 15 min at 4° C.) from which the resulting supernatant was extracted, aliquoted, and stored at −80° C.

On day 26, a terminal blood sample was collected from the lateral tail vein and placed into a serum tube. Each serum sample was kept at room temperature for 45 minutes to allow coagulation, before being centrifuged (2000 g, 15 min at 4° C.) from which the resulting supernatant was extracted, aliquoted, and stored at −80° C. until being analyzed for IgE concentrations.

### Bronchoalveolar Lavage (BAL)

Immediately after collecting the terminal blood samples, the animals were culled by an overdose with pentobarbital. The trachea was then isolated by a midline incision in the neck and separation of the muscle layers. A small incision was made into the trachea and a plastic cannula was inserted and secured in place with a suture. The airway was then lavaged by flushing out the lungs using 0.5 mL of phosphate buffered saline. This procedure was repeated until the recovered volume was 1.6 mL. The isolated BAL fluid was then centrifuged at 1500 rpm for 10 mins at 4° C., and the supernatant was aliquoted (400 μL) at −80° C. for cytokine analysis. The cell pellets were then re-suspended in 1.6 mL of phosphate buffered saline, and the BAL cells were then analyzed for total and differential numbers.

### Lung Tissue Collection

Following BAL fluid collection, the thoracic cavity was opened to expose the lungs, which were dissected free of the animal. The left lung lobe was sliced into equal halves along the middle longitudinal. One half was placed in a sterile container filled with RNAlater, while the other half was placed into a separate sterile containers containing 10% neutral buffered formalin for 48 hours before being transferred to 70% ethanol for tissue processing and mucus scoring. Right lung lobes were placed into sterile containers and snap frozen before being stored at −80° C. before being processed and analyzed for cytokine concentrations.

Following fixation of the left lung lobe tissue and processing in paraffin wax, sections (5 μM) were transversely cut, mounted on slides and stained with Period acid-Schiff (PAS) before being analyzed using digital imaging for airway mucus production.

### Airway Mucus Production

Airway mucus production was quantified from the ratio of mucus-positive epithelium to total epithelium as the percentage of the surface covered in mucus using an area quantification algorithm (Halo image analysis software). Compound 1 reduced airway mucus levels.

FIG. 1 shows a bar graph representing the area of PAS positive mucin in airway mucosa of mouse lung tissue following treatment with Compound 1 (150-1500 mg/kg, p.o., b.i.d., days 19-25), Compound 1 (500 mg/kg, p.o., b.i.d., days 19-25) and 1-ABT treatment (25 mg/kg, p.o., b.i.d., days 19-25), and vehicle (10 mL/Kg, p.o., b.i.d., days 19-25) in Alternaria sensitized mice challenged with PBS or Alternaria. Each column represents the mean and each bar represents s.e. mean of n=5-10. Vehicle and Alternaria challenge (Group 2) was compared to all other groups using ANOVA followed by Dunnett's test. *** P<0.001.

Compound 1 dose-dependently reduced airway mucus in lung tissue.

### IgE Assay

Serum supernatant was evaluated for IgE concentrations using ELISA kit (Invitrogen, UK) as per the manufacturer's instructions. Optical density was measured at 450 nM using a microplate reader (SpectraMax 340PC). Concentrations of IgE were determined using SoftMax Pro v. 6.4 (Molecular Devices).

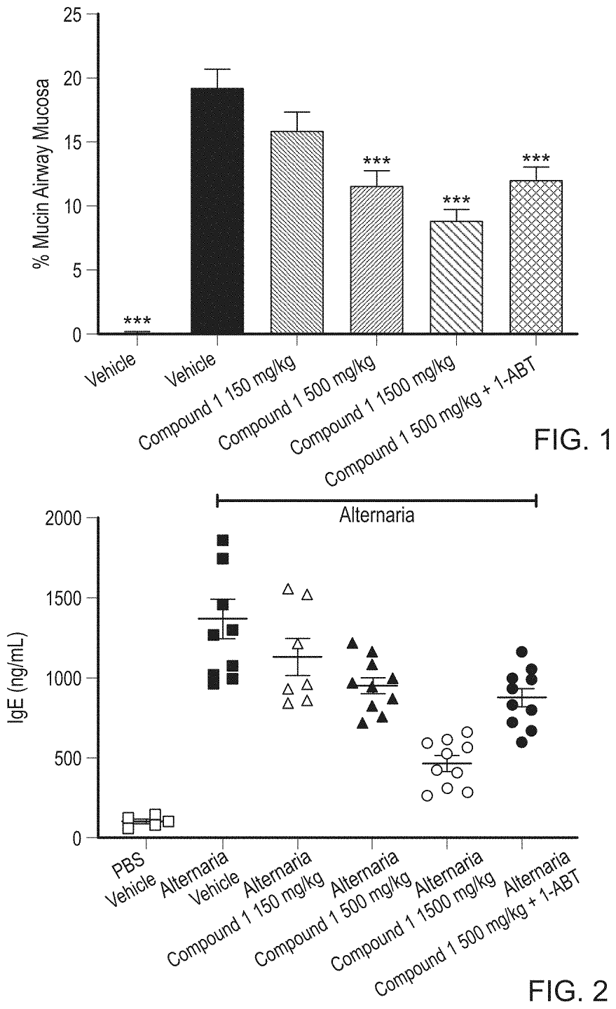

FIG. 2 shows a plot representing IgE levels in blood serum following treatment with vehicle, Compound 1, and Compound 1 and 1-ABT in Alternaria sensitized mice challenged with PBS or Alternaria.

Compound 1 dose-dependently reduced the serum IgE levels.

### Cell Counts

Total and differential cell counts of the BAL fluid samples were measured using a XT-2000iV analyser (Sysmex). Cell types differentially classified were neutrophils, eosinophils, lymphocytes, or macrophages.

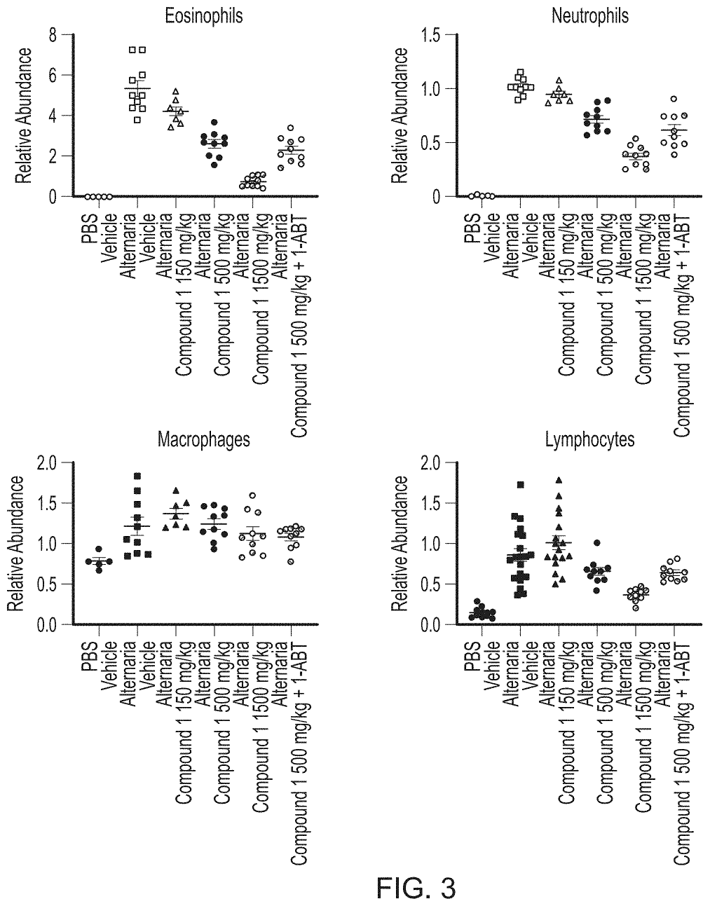

FIG. 3 shows plots representing relative abundance of neutrophils, eosinophils, lymphocytes, and macrophages in the BAL fluid following treatment with treatment with vehicle, Compound 1, and Compound 1 and 1-ABT in Alternaria sensitized mice challenged with PBS or Alternaria.

Compound 1 dose-dependently suppressed eosinophils, neutrophils, and lymphocytes invasion in bronchoalveolar lavage fluid. The total number of both eosinophils and neutrophils were significantly decreased upon Compound 1 treatment, which suggests a suppression of Th2/TH17 signaling. The total number of lymphocytes decreased at highest dose of Compound 1. There was no change in number of macrophages. Compound 1 (500 mg/kg) and 1-ABT showed similar degree of effects compares to Compound 1 (500 mg/kg).

### Cytokine Analysis

Cytokine concentrations (TSLP, IL-4, IL-5, IL-13, and IL-33) of BAL fluid supernatant and lung homogenate (all groups) were measured using ELISA kit (Biotechne, UK) as per the manufacturer's instructions. Optical density was measured at 450 nM using a microplate reader (SpectraMax 340PC). Concentrations of cytokine were determined using SoftMax Pro v. 6.4 (Molecular Devices).

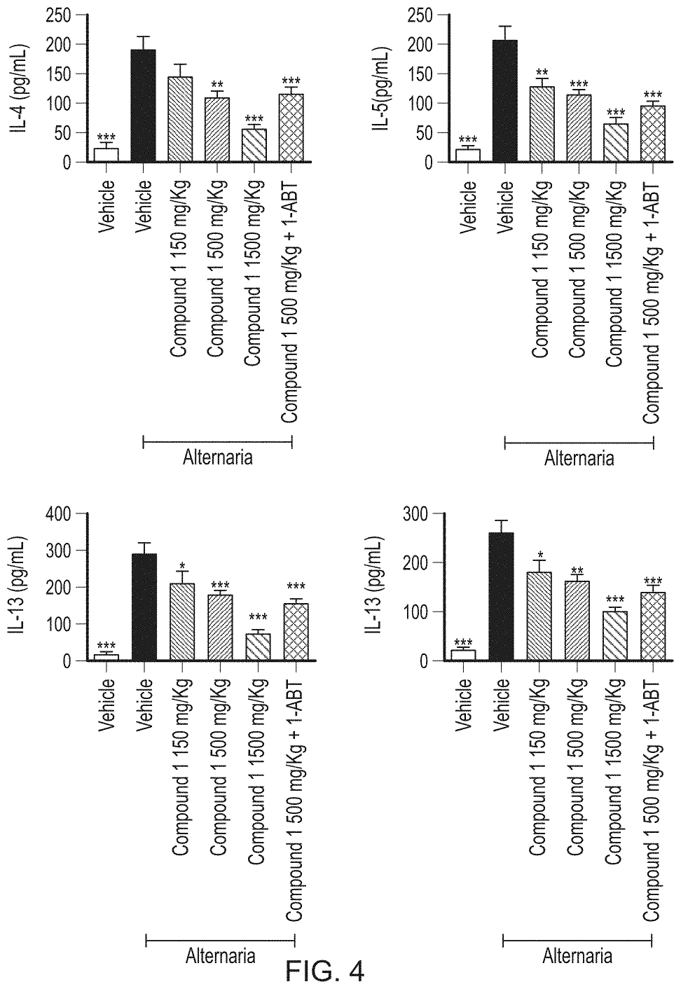

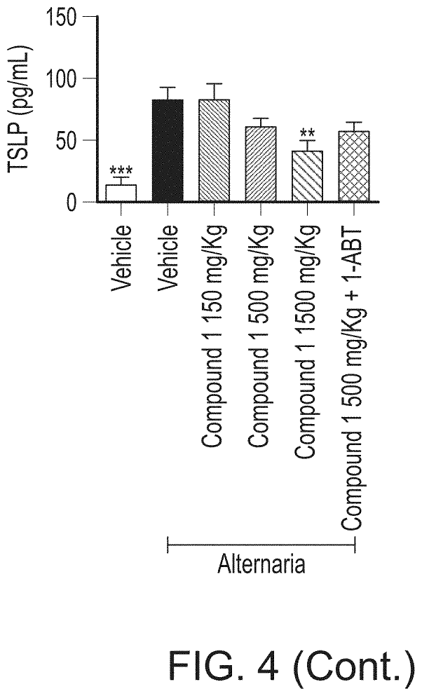

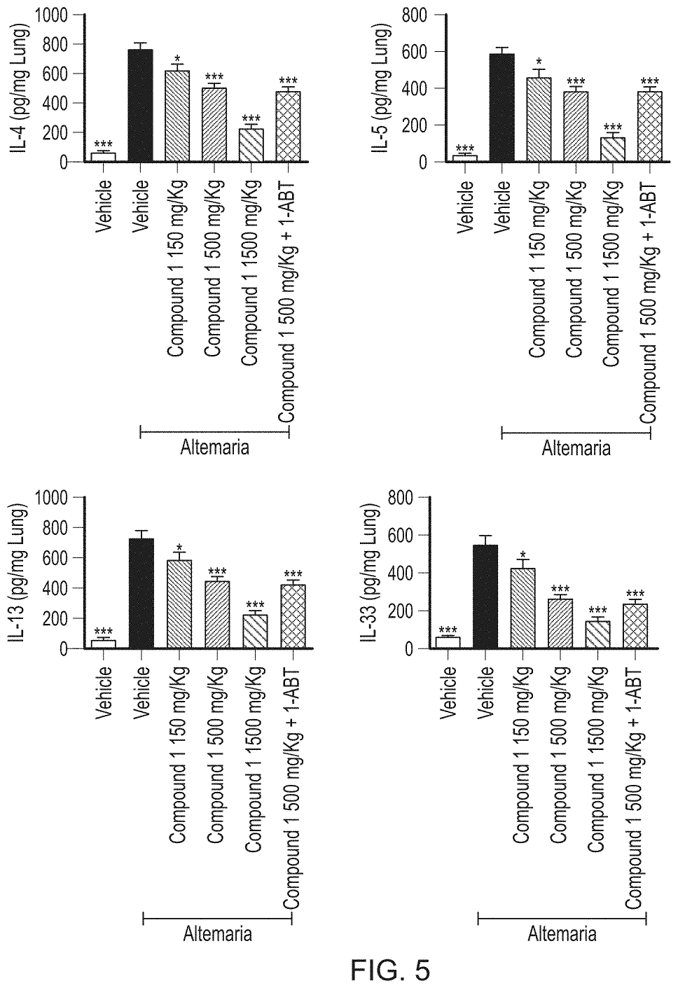

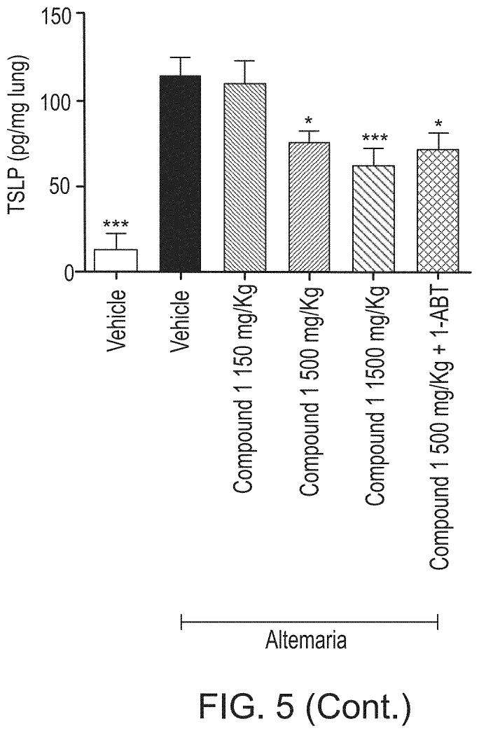

FIG. 4 shows bar graphs representing concentration of TSLP, IL-4, IL-5, IL-13, and IL-33 in BAL fluid supernatant following treatment with vehicle, Compound 1, and Compound 1 and 1-ABT in Alternaria sensitized mice challenged with PBS or Alternaria. Each column represents the mean and each bar represents s.e. mean of n=5-10. Vehicle with Alternaria challenge (Group 2) was compared to all other groups (*) using ANOVA followed by Dunnett's test. *P<0.05, **P<0.01 and *** P<0.001. FIG. 5 shows bar graphs representing concentration of TSLP, IL-4, IL-5, IL-13, and IL-33 in lung homogenate following treatment with vehicle, Compound 1, and Compound 1 and 1-ABT in Alternaria sensitized mice challenged with PBS or Alternaria. Each column represents the mean and each bar represents s.e. mean of n=5-10. Vehicle with Alternaria challenge (Group 2) was compared to all other groups (*) using ANOVA followed by Dunnett's test. *P<0.05, **P<0.01 and *** P<0.001.

Compound 1 dose-dependently reduced levels of Th2 cytokines (IL-4, IL-5, and IL-13) and alarmins (IL-33 and TSLP) in the BAL fluid and lung homogenate.

### Example B. Efficacy Study of Compound 1 and Compound 2 in Alternaria-Induced Asthma Model

This study is a dose response evaluation of the effect of the PARP14 inhibitors, Compound 1:

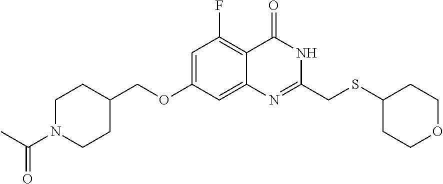 [SMILES: CC(=O)N1CCC(COc2cc(F)c3c(=O)[nH]c(CSC4CCOCC4)nc3c2)CC1]

### 7-((1-Acetylpiperidin-4-yl)methoxy)-5-fluoro-2-(((tetrahydro-2H-pyran-4-yl)thio)methyl)quinazolin-4(3H)-one

### and Compound 2:

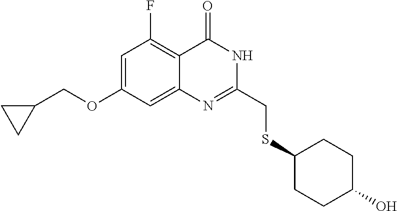 [SMILES: O=c1[nH]c(CS[C@H]2CC[C@H](O)CC2)nc2cc(OCC3CC3)cc(F)c12]

### 7-(Cyclopropylmethoxy)-5-fluoro-2-(((trans-4-hydroxycyclohexyl)thio)methyl)quinazolin-4(3H)-one

on pulmonary inflammation by assessing bronchoalveolar lavage (BAL) fluid cell numbers, BAL fluid supernatant concentrations of IL-4, IL-5, IL13 and IL-33, total IgE concentrations and production of airway mucus in mice exposed to Alternaria. See U.S. Pat. Publication No: US 2019/0194174 for a description of Compounds 1 and 2.

### Animal Welfare

All animals throughout the study were held in the biological support unit at Pharmidex's facilities at European Knowledge Centre, Mosquito Way, Hatfield, AL10 9WN, United Kingdom. The facility operates under the United Kingdom home office animal scientific procedures act and as such has a valid establishment license (X07D13023) allowing regulated animal procedures to be carried out in the facility.

On arrival from the supplier, the animals (35 male BalbC mice, 20-30 g on arrival, Charles Rivers, UK) were placed into cages of 5 as outlined below, and the welfare of all animals was checked on a daily basis. Emergency care if required was provided by the named veterinary surgeon, although this was not necessary during the study.

Throughout the study, the following guidelines (provided by the Home Office) were used to assess non-specific or unexpected adverse effects in animals undergoing regulated activities, relating to either the procedure or test compound dosing. Animals showing two or more of any of the limiting clinical signs in the category equivalent to the protocol severity limit were removed from the study and were killed by a schedule 1 method (cervical dislocation) at the establishment. Where an animal reached the limit of either or both of the first two signs with or without any other signs it was removed from the study and killed by a schedule 1 method at the establishment.

Body weight loss greater than 20% of the highest measured individual body weight.

Marked piloerection with other signs of dehydration such as skin tenting.

Unresponsive to activity and provocation.

Hunched persistently (frozen).

Distressed—persistent vocalization.

Oculo-nasal discharge persistent and copious.

Labored respiration.

Persistent tremors.

Persistent convulsions.

Animals also exposed to severe fight injuries were removed from the study and killed by a schedule 1 method at the establishment.

An acclimatization period of 7 days was allowed, before start of experimental procedures. Mice were housed in cages of 5 on arrival based on weight (equal distribution of animal weights amongst each of the cages by the animal technician) with a 12 hour light dark cycle. Room temperature and humidity was maintained within home office guidelines (17-24° C. and 40-70% respectively). Environmental enrichment was provided in all cages. Mice had access to standard chow ad libitum, and water was available from bottles ad libitum.

### Allergen Exposure

Alternaria alternata was diluted in sterile PBS to 5 μg of Alternaria per 40 μl.

On days 1-5 Balb/c mice were challenged (intranasal) under isoflurane anesthesia. On days 23-25 Balb/c mice were challenged (intranasal) under isoflurane anesthesia one hour post the first dosing of the day. PBS (Group 1) or Alternaria (Groups 2-4) were instilled into each nostril in a drop wise fashion alternating between the two until a volume of 40 μl had been delivered.

### Formulation and Treatment Regime

1-Aminobenzotriazole (1-ABT, 25 mg/kg), Compound 2 (500 mg/kg), and Compound 1 (500 mg/kg) were each individually formulated in vehicles of 0.5% methylcellulose (MC) and 0.2% Tween in distilled water, and 1-aminobenzotriazole (1-ABT, 25 mg/kg) and Compound 1 (500 mg/kg) were co-formulated in a vehicle of 0.5% methylcellulose (MC) and 0.2% Tween in distilled water.

Group 1: On days 19-25, the animals were dosed b.i.d. with the 1-ABT vehicle (10 mL/kg). On day 26, the animals were dosed with the 1-ABT vehicle 23 hours post the day 25 PBS challenge. All mice were taken down 2 hours later.

Group 2: On days 19-25, the animals were dosed b.i.d. with the 1-ABT vehicle (10 mL/kg). On day 26, the animals were dosed with the 1-ABT vehicle 23 hours post the day 25 Alternaria challenge. All mice were taken down 2 hours later.

Group 3: On days 19-25, the animals were dosed b.i.d. with the Compound 2 vehicle (10 mL/kg). On day 26, the animals were dosed with the Compound 2 vehicle 23 hours post the day 25 Alternaria challenge. All mice were taken down 2 hours later.

Group 4: On day 19, the animals were dosed with the 1-ABT vehicle (10 mL/kg) followed by the Compound 1 vehicle (10 mL/kg) 2 hours later. Twelve hours after the dosing of the Compound 1 vehicle, the animals were dosed with the 1-ABT and Compound 1 vehicle. On days 20-25, the 1-ABT and Compound 1 vehicle was administered (p.o., b.i.d.). On day 26, the 1-ABT and Compound 1 vehicle was dosed 23 hours post the day 25 Alternaria challenge. All mice were taken down 2 hours later.

### Blood Collection

On day 19, Group 3 and 4 animals 1-5 had blood samples (15-20 μL) collected by venipuncture via the lateral tail vein 2 hr after the first dose of Compound 2 or Compound 1, and Group 3 and 4 animals 6-10 had blood samples (15-20 μL) collected by venipuncture via the lateral tail vein 30 min prior to the second dose of Compound 2 or Compound 1 (11.5 hr post 1st dose). On day 26, blood samples were collected from all animals 30 min prior to the last Compound 2 or Compound 1 dose and 2 hrs after the last dose as a terminal bleed. Each blood sample (15-20 μL) was placed into EDTA tubes and mixed gently before being centrifuged (2000 g, 15 min at 4° C.) from which the resulting supernatant was extracted, aliquoted, and stored at −80° C.

On day 26, a terminal blood sample was collected from the lateral tail vein and placed into a serum tube. Each serum sample was kept at room temperature for 45 minutes to allow coagulation, before being centrifuged (2000 g, 15 min at 4° C.) from which the resulting supernatant was extracted, aliquoted, and stored at −80° C. until being analyzed for IgE concentrations.

### Bronchoalveolar Lavage (BAL)

Immediately after collecting the terminal blood samples, the animals were culled by an overdose with pentobarbital. The trachea was then isolated by a midline incision in the neck and separation of the muscle layers. A small incision was made into the trachea and a plastic cannula was inserted and secured in place with a suture. The airway was then lavaged by flushing out the lungs using 0.5 mL of phosphate buffered saline. This procedure was repeated until the recovered volume was 1.6 mL. The isolated BAL fluid was then centrifuged at 1500 rpm for 10 mins at 4° C., and the supernatant was aliquoted (400 μL) at −80° C. for cytokine analysis. The cell pellets were then re-suspended in 1.6 mL of phosphate buffered saline, and the BAL cells were analyzed for total and differential numbers.

### Lung and Spleen Tissue Collection

Following BAL fluid collection, the thoracic cavity was opened to expose the lungs. The right and left lung lobes were placed into separate sterile containers containing 10% neutral buffered formalin for 48 hours before being transferred to 70% ethanol for tissue processing and mucus scoring. Lung tissue was analyzed for cytokine concentrations.

Each animal also had their spleen removed following lung extraction. The spleens were dissected into two section and each section was weighed. One section was snap frozen and stored at −80° C. while the second section was placed in RNAlater.

Following fixation of the left lung lobe tissue and processing in paraffin wax, sections (5 μM) were transversely cut, mounted on slides, and stained with Period acid-Schiff (PAS) before being analyzed using digital imaging for airway mucus production.

### Airway Mucus Production

Airway mucus production was quantified from the ratio of mucus-positive epithelium to total epithelium as the percentage of the surface covered in mucus using an area quantification algorithm (Halo image analysis software).

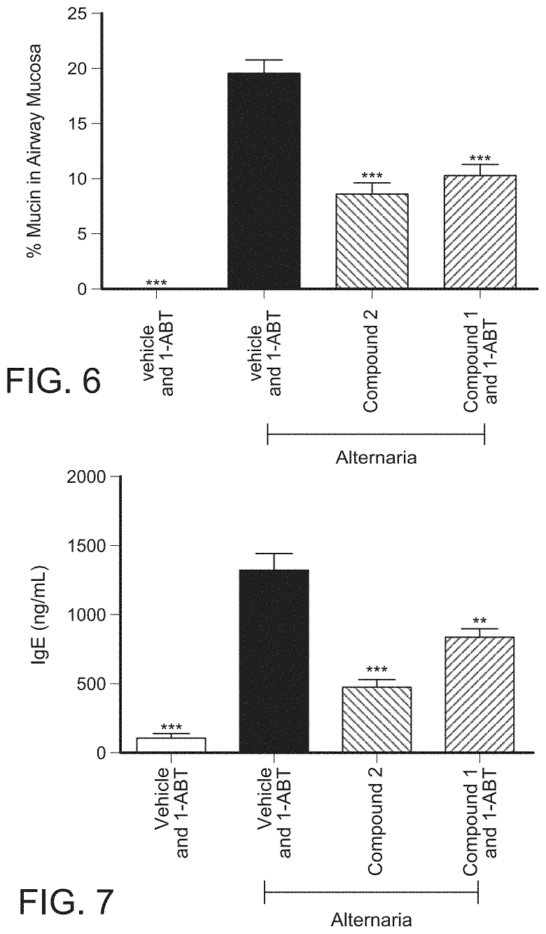

FIG. 6 shows a bar graph representing the area of PAS positive mucin in airway mucosa of mouse lung tissue following treatment with vehicle and 1-ABT, Compound 2, and Compound 1 and 1-ABT in Alternaria sensitized mice challenged with PBS or Alternaria. Each column represents the mean and each bar represents s.e. mean of n=5-10. Vehicle and 1-ABT with Alternaria challenge (Group 2) was compared to all other groups (*) using ANOVA followed by Dunnett's test. *** P<0.001.

Compound 2 and Compound 1 reduced airway mucus in lung tissue.

### IgE Assay

Serum supernatant was evaluated for IgE concentrations using ELISA kit (Invitrogen, UK) as per the manufacturer's instructions. Optical density was measured at 450 nM using a microplate reader (SpectraMax 340PC). Concentrations of IgE were determined using SoftMax Pro v. 6.4 (Molecular Devices). Data were reported as IgE (pg/mL), mean S.E.M. (standard error of the mean).

FIG. 7 shows a bar graph representing concentration of IgE in blood serum following treatment with vehicle and 1-ABT, Compound 2, and Compound 1 and 1-ABT in Alternaria sensitized mice challenged with PBS or Alternaria. Each column represents the mean and each bar represents s.e. mean of n=5-10. Vehicle and 1-ABT with Alternaria challenge (Group 2) was compared to all other groups (*) using ANOVA followed by Dunnett's test. **P<0.01, ***P<0.001.

Compound 2 and Compound 1 reduced IgE blood serum levels.

### Cell Counts

Total and differential cell counts of the BAL fluid samples were measured using a XT-2000iV analyser (Sysmex). Results were expressed as cells/mL (total and differential). Cell types differentially classified were neutrophils, eosinophils, lymphocytes, and macrophages.

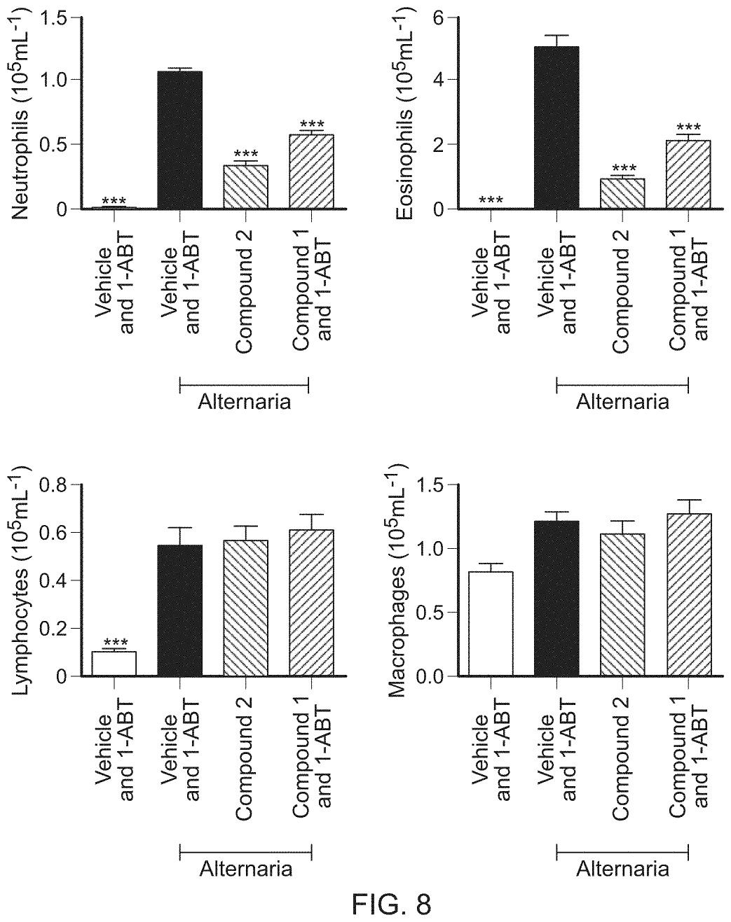

FIG. 8 shows bar graphs representing the number of neutrophils, eosinophils, lymphocytes, and macrophages per mL in the bronchoalveolar lavage fluid following treatment with vehicle and 1-ABT, Compound 2, and Compound 1 and 1-ABT in Alternaria sensitized mice challenged with PBS or Alternaria. Each column represents the mean and each bar represents s.e. mean of n=5-10. Vehicle and 1-ABT with Alternaria challenge (Group 2) was compared to all other groups (*) using ANOVA followed by Dunnett's test. ***P<0.001.

Compound 2 and Compound 1 reduced levels of neutrophils and eosinophils but not lymphocytes or macrophages in BAL fluid.

### Cytokine Analysis

Cytokine concentrations (IL-4, IL-5, IL-13, and IL-33) of BAL fluid supernatant and lung homogenate (all groups) were measured using magnetic multiplex assays (Biotechne, UK) as per the manufacturer's instructions. Levels were measured using a Magpix system (Luminex Corp). Data were reported as cytokine (pg/mL), mean±S.E.M. (standard error of the mean).

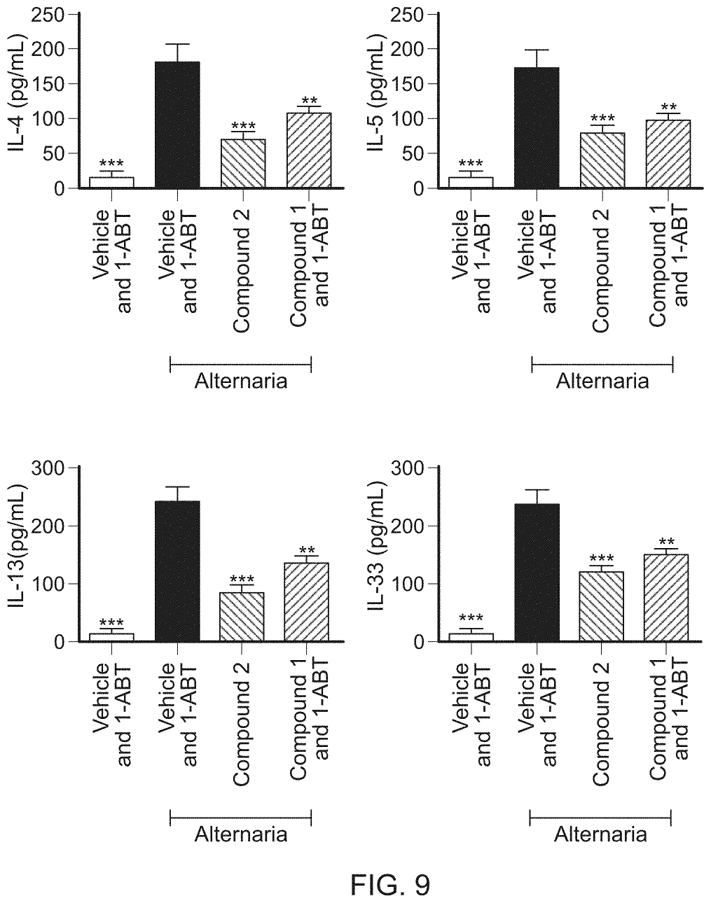

FIG. 9 shows a bar graph representing IL4, IL-5, IL-13, and IL-33 in BAL fluid supernatant following treatment with vehicle and 1-ABT, Compound 2, and Compound 1 and 1-ABT in Alternaria sensitized mice challenged with PBS or Alternaria. Each column represents the mean and each bar represents s.e. mean of n=5-10. Vehicle and 1-ABT with Alternaria challenge (Group 2) was compared to all other groups (*) using ANOVA followed by Dunnett's test. **P<0.01 and ***P<0.001. FIG. 10 shows a bar graph representing IL4, IL-5, IL-13, and IL-33 in lung homogenate following treatment with vehicle and 1-ABT, Compound 2, and Compound 1 and 1-ABT in Alternaria sensitized mice challenged with PBS or Alternaria. Each column represents the mean and each bar represents s.e. mean of n=5-10. Vehicle and 1-ABT with Alternaria challenge (Group 2) was compared to all other groups (*) using ANOVA followed by Dunnett's test. **P<0.01 and ***P<0.001.

Compound 2 and Compound 1 reduced levels of IL4, IL-5, IL-13, and IL-33 in BAL fluid and lung tissue.

### Example C. PARP14 IHC Assay

The staining procedure for immunohistochemistry (“IHC”) analysis of PARP14 (rabbit clone 15B10-1) with DAB chromogen was performed using automated detection on the Leica Bond Rx (Leica Biosystems). All steps occurred at room temperature unless noted otherwise.

### Pre-Processing and Pretreatment

FFPE specimens were sectioned at 4-micron thickness, mounted onto positive-charged glass slides, dried, and baked for 30 minutes at 60° C. in an oven deparaffinized and rehydrated offline in accordance with standard practices. Tissue slides were then placed onto the Leica Bond RX autostainer and underwent pretreatment using Epitope Retrieval Solution 2 (ER2, Catalog #AR9640, Leica) starting with 2 rinses in ER2, then incubation in ER2 for 40 minutes at 100° C. followed by a rinse in ER2. Tissue slides were then rinsed 4 times followed by a 3-minute incubation with Bond Wash Solution (Catalog #AR9590, Leica Biosystems).

### Staining Procedure

The staining protocol for PARP14 (rabbit clone 15B10-1) and the negative control reagent (NCR) was initiated. The tissue slides were incubated with Peroxide Block (Bond Polymer Refine Detection, Catalog #DS9800, Leica Biosystems) for 5 minutes followed by 3 rinses in Bond Wash Solution. The tissue slides were incubated with ISH/IHC Super Blocking (Catalog #PV6122, Leica Biosystems) for 10 minutes. The tissue slides were then incubated with the primary antibody or NCR diluted in ISH/IHC Super Blocking (Leica Biosystems) for 30 minutes followed by 3 rinses in Bond Wash Solution. The tissue slides were incubated with Post Primary (Bond Polymer Refine Detection) for 8 minutes followed by a wash for 3×2 minutes in Bond Wash Solution. The tissue slides were incubated with Polymer (Bond Polymer Refine Detection) for 8 minutes followed by a wash for 3×2 minutes in Bond Wash Solution and then 2 rinses in distilled water. The tissue slides were rinsed with Mixed DAB Refine (Bond Polymer Refine Detection) and then incubated with Mixed DAB Refine for 10 minutes followed by 4 rinses in distilled water. Upon completion of the staining procedure, the tissue slides were counterstained online with hematoxylin (Bond Polymer Refine Detection) for 5 minutes followed by a rinse in distilled water, a rinse in Bond Wash Solution, and a final rinse in distilled water.

### Post-Processing

The tissue slides were removed from the autostainer and dehydrated and cleared offline in accordance with standard practices. Coverslip mounting was performed using an automated tape coverslipper (Sakura Finetek, Torrance, Calif.) or glass coverslipper (Leica).

### Photomicrographs

H&E and PARP14 (rabbit clone 15B10-1) stained slides were scanned using an Aperio AT Turbo or ScanScope CS system (Aperio, Vista, Calif.) to produce whole slide images.

### Human Systemic Sclerosis Testing

PARP14 (rabbit clone 15B10-1) IHC staining was performed on 11 human systemic sclerosis tissues. Tissues were evaluated by pathologist review. One (1) human systemic sclerosis tissue (ML2007935) was unevaluable due to tissue fall off NCR staining was negative for all human systemic sclerosis tissues. Positive staining is rated on a scale of 1+ to 3+, with 3 being the most intense. Endothelial staining was observed as 1+(cytoplasmic) for 3 human systemic sclerosis tissues and 1+(membrane and cytoplasmic) for 1 human systemic sclerosis tissue. Smooth muscle staining was not observed in any of the evaluable human systemic sclerosis tissues. Fibroblast staining was not observed in any of the evaluable human systemic sclerosis tissues. Stroma staining was not observed in any of the evaluable human systemic sclerosis tissues. Inflammatory cell staining was observed as 3+(cytoplasmic, 3 tissues), 2+(cytoplasmic, 3 tissues), or 1+(cytoplasmic, 4 tissues) for all evaluable human systemic sclerosis tissues. Nerve staining was not observed in any of the evaluable human systemic sclerosis tissues.

### PARP14 (Rabbit Clone 15B10-1)—Human Normal Tissue TMA

A specificity analysis was performed using the optimized PARP14 (rabbit clone 15B10-1) IHC assay on a normal TMA slide with 20 unique tissue cores purchased from US Biomax (ML2011751). The normal TMA cores were evaluated by pathologist review.

### Normal Cell Expression

Across the 20 normal tissue cores, expression of PARP14 was observed in prostate tissue (80%; H-Score 80) and adjacent normal endometrium tissue (35%; H-Score 38). All other normal tissue cores were negative for PARP14 expression (0%; H-Score 0).

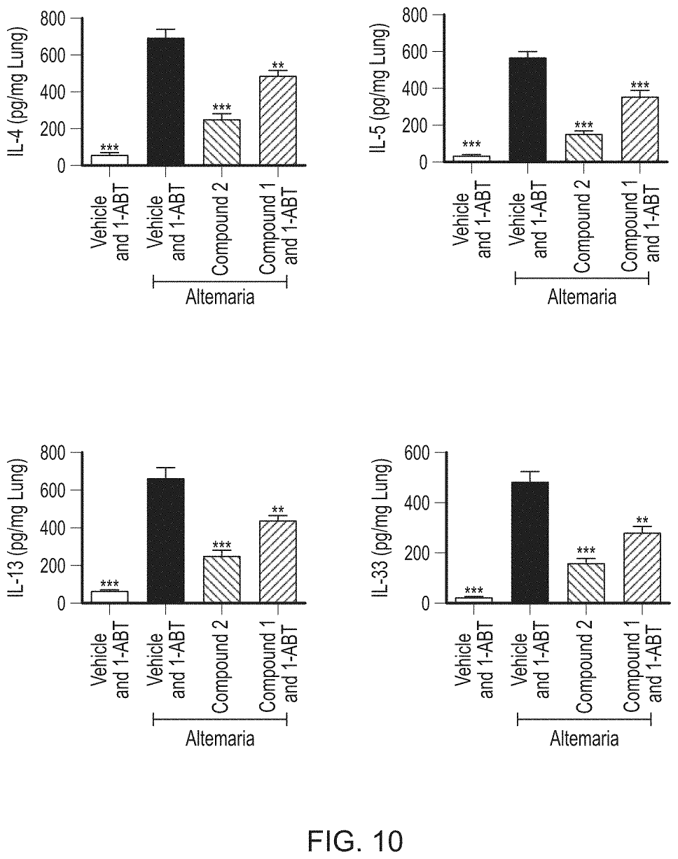

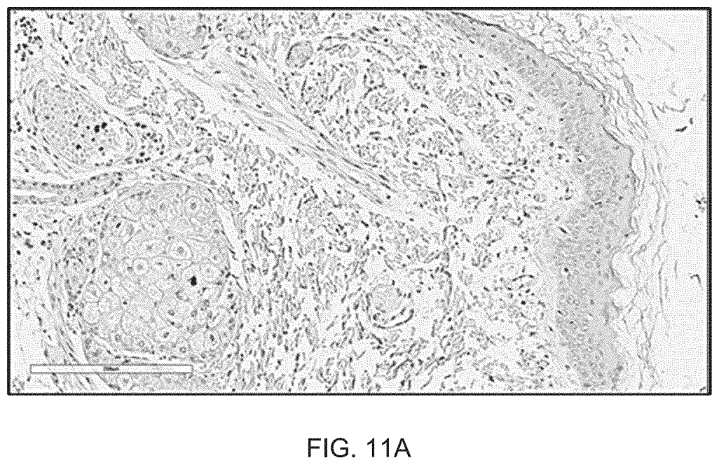

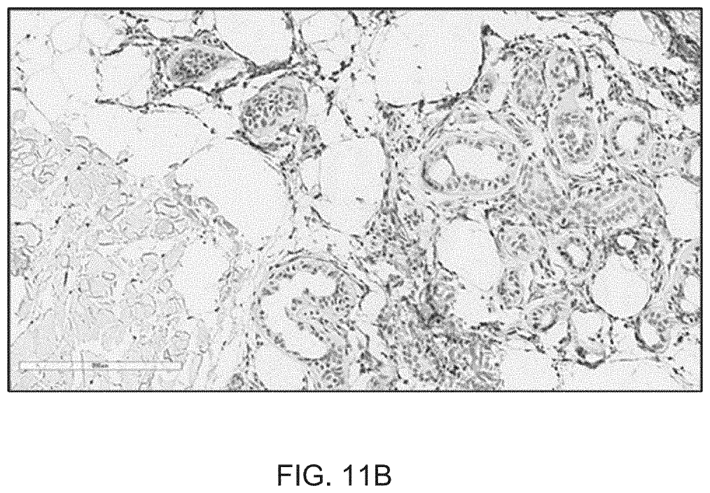

FIG. 11A is an immunohistochemistry image (20×) of normal human skin.

FIG. 11B is an immunohistochemistry image (20×) of skin from a patient with systemic sclerosis. PARP14 was visualized using DAB (brown chromogen) with a hematoxalin counterstain.

Based on the above IHC images, it was determined that PARP14 is highly expressed in chronically inflamed skin from human systemic sclerosis patients but not normal skin tissue.

### Example D. DNCB-Induced Atopic Dermatitis Model

A study was conducted to investigate Compound 1 in a 2-4,-dinitrochlorobenzene (DNCB)-induced atopic dermatitis model. Female BALB/c mice were challenged with DNCB for two cycles to induce atopic dermatitis. The animals were treated with vehicle or Compound 1 twice a day (BID) for two weeks. Dexamethasone (DEX) was included as a comparator. Details of the study are provided below.

### Formulation of Compound 1

Compound 1 was formulated in a vehicle of sesame oil. Each formulation was prepared by adding the required volume of sesame oil to the weighed-out amount of Compound 1 and stirring the solution at room temperature overnight for evenly distributed suspension. Before each animal administration the formulation was turned upside down several times so as to generate a more evenly distributed suspension. The formulation was prepared fresh daily. All vehicle and Compound 1 dosing were performed by oral gavage (PO) at 10 mL/kg. All BID (twice daily dosing) dosing was based on a 12 h cycle with the schedules calculated.

### DNCB Solution

DNCB was weighed (50 mg) and dissolved in 10 ml of 4:1 (v/v) acetone/olive oil solution to make a 0.5% DNCB solution. DNCB was weighed (100 mg) and dissolved in 10 ml of 4:1 (v/v) acetone/olive oil solution to make 1% DNCB solution, freshly prepared before each application.

### Animals

Details of the mice used in the study are provided below.

| | |
|---|---|
| Animal species and strain: | BALB/c mice |
| Breeder/supplier: | Beijing Vital River Laboratory |
| | Animal Co. Ltd |
| Sex, age: | Female, 7-8 weeks |
| Test Facility: | WuXi AppTec Vivarium (Nantong) |
| Adaptation: | 3-7 days |
| Room: | Specific pathogen free room |
| Room temperature: | 20-26° C. |
| Room relative humidity: | 40-70% |
| Light cycle: | Fluorescent light for 12-hour light |
| | and 12-hour dark |
| Animal housing: | 4 mice/cage by treatment group |
| Food and water: | Free access to food and water |

### Equipment

The following equipment was used:

Electronic balance: Changzhou Tianzhiping, YH2000

Semimicro balance: Mettler-Toledo ML203/02

Micrometer: Digimatic QuantuMike Micrometer, 293-185

Thermometer: Omron IR thermometer, MC-872

Anesthesia machine: Raymain, HSIV-μ Quad, four channel

Tewameter: MEDELINK, TM300

### Grouping

Eight-week female BALB/c mice were used (n=28) and divided into 4 groups. One day before the start of study, the backs of all the mice were shaved with a pet hair clipper, the area was 2×3 cm.

On Day 0 to Day 4 and Day 12 to Day 15, DNCB was topically applied on the shaved back (dorsal) of the mice in Groups 2, 3, and 4. The dose regimen and group setting are shown in the below table. The test article was prepared daily. Compound 1 was orally administered from Day 9 to Day 23. Dexamethasone (DEX) was applied from Day 9 to Day 23. Commercial cream was weighed (70 mg for back skin) for each mouse in Group 3.

| Group | N | Test articles | Treatment | | Route | Frequency |
|---|---|---|---|---|---|---|
| 1 | 4 | Naive | — | | — | — |
| 2 | 8 | Vehicle | — | | PO | BID, Day 9 |
| | | | | | | to Day 23 |
| 3 | 8 | DEX ointment | 70 | mg | Topical | QD, Day 9 |
| | | | | | | to Day 23 |
| 4 | 8 | Compound 1 | 500 | mg/kg | PO | BID, Day 9 |
| | | | | | | to Day 23 |

Abbreviations: BID = twice a day; DEX = dexamethasone; PO = oral; QD = once daily.

### DNCB Sensitization and Treatment

For Group 2-Group 4, the back (dorsal) skin of the mice was sensitized with 0.1 ml 0.5% DNCB on Day 1 and Day 2; and sensitized with 0.12 ml 1% DNCB on Day 3 and Day 4 (˜15:30, on every designed day). This cycle was repeated on Day 12-Day 15.

From Day 9 to Day 23, the test compounds and vehicle were given (˜9:30, ˜21:30 for BID dosing, every day) as the regimen indicated in the above table. DEX was given at ˜9:30 every day.

### Results

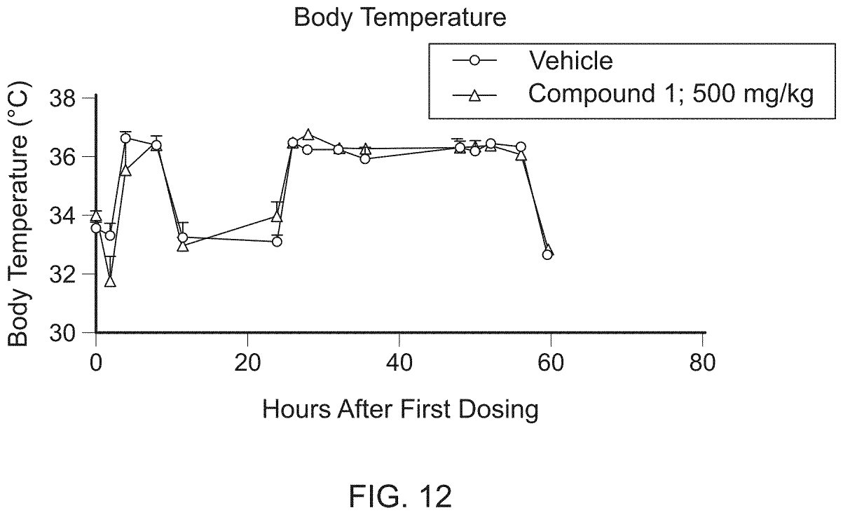

Body temperature of the vehicle and Compound 1 group was measured by Omron IR thermometer at pre-dose, 2 h, 4 h, 8 h and 11.5 h post dose on first 3 days of the treatment with forehead measuring mode at the face. The body temperature ranged from 31° C. to 37° C. in the first 3 days. The body temperature curves of both groups were similar. FIG. 12 shows the body temperature curves for Groups 1 and 4, up to 60 hours after dosing.

The following clinical score parameters were used to assess the severity of inflammation of the back skin:

| Score | Erythema | Thickness | | Scab |
|---|---|---|---|---|
| 0 | Normal | Normal | | Normal |
| 1 | Pink | <0.5 | mm | Sporadic |
| 2 | Mild redness | 0.5-1 | mm | Scab area <⅓ |
| | | | | back skin |
| 3 | Moderate redness | 1-1.5 | mm | Scab area ⅓-⅔ |
| | | | | back skin |
| 4 | Dark redness | >1.5 | mm | Scab area >⅔ |
| | | | | back skin |

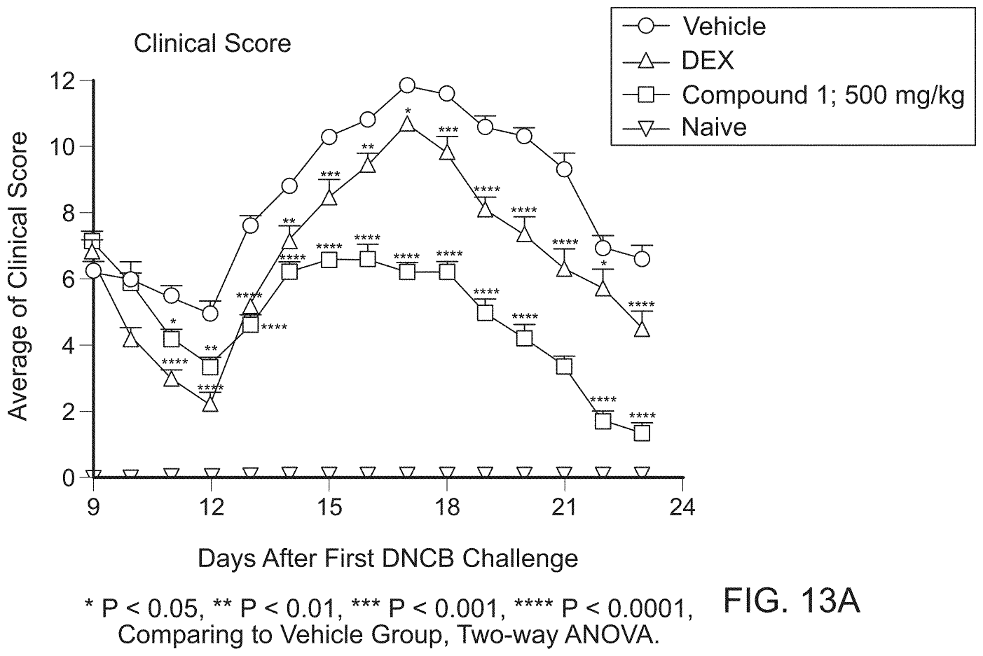

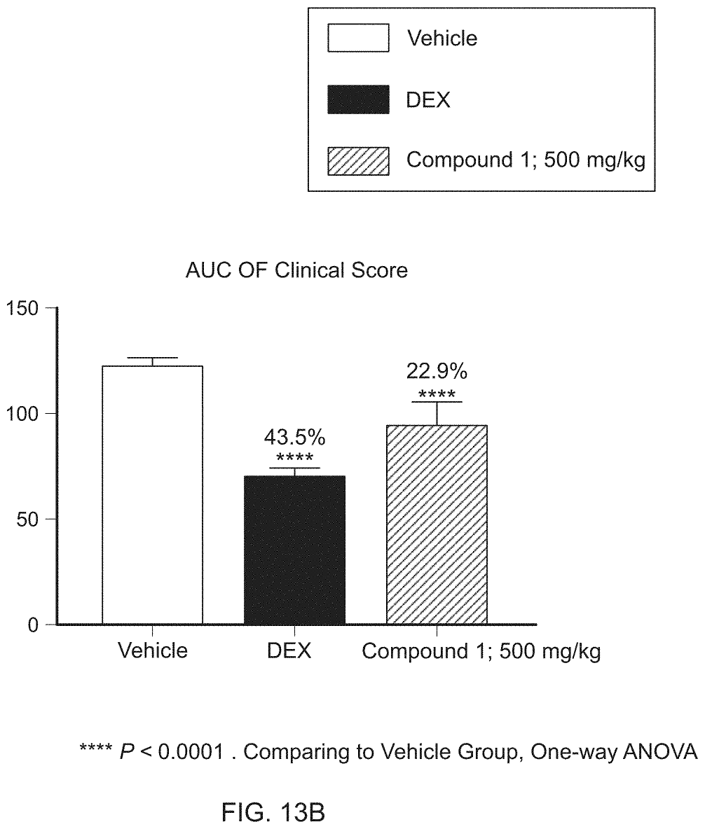

Erythema, scabbing and thickness of the dorsal (back) skin were scored daily. The cumulative scores (Erythema plus Thickness plus Scabbing) are presented as the total score. DNCB challenge induced erythema, thickness and scab in the back skin and the total clinical score peaked at a value of 11.9 on Day 17. The DEX treatment group significantly inhibited inflammation, and high clinical scores ranged from 6-7. Compound 1 treatment showed significant efficacy from Day 11 to Day 23, with a peak clinical score of 10.8 on Day 17. FIG. 13A shows a plot of the clinical score of each group 9 to 24 days after the first DNCB challenge.

Based on the total clinical score curve of each animal in each group, the AUC was calculated, and is shown in FIG. 13B. The inhibition rate was calculated by [AUC (vehicle)-AUC (treatment)]/AUC (vehicle)×100. The DEX group had significantly inhibited clinical scoring by 43.5% (P<0.0001), compared to vehicle. Compound 1 given at an oral dose of 500 mg/kg BID significantly inhibited clinical scoring by 22.9% (P<0.0001), compared to vehicle.

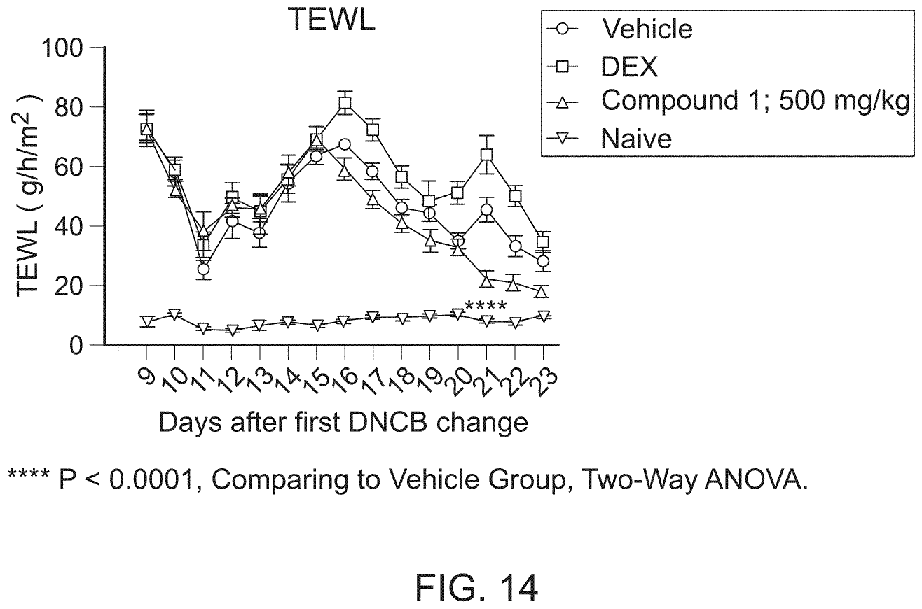

Trans-epidermal water loss (TEWL) of the dorsal (back) skin was monitored by Tewameter daily and expressed as g/h/m². The water loss in back skin induced by DNCB challenge and recovered after the challenge cycle stopped. DEX treatment increased the water loss of the back skin, compared to vehicle. Compound 1 treatment showed a trend towards decreasing water loss and a significant decrease on Day 21 compared to vehicle. FIG. 14 shows a plot of the trans-epidermal water loss over time.

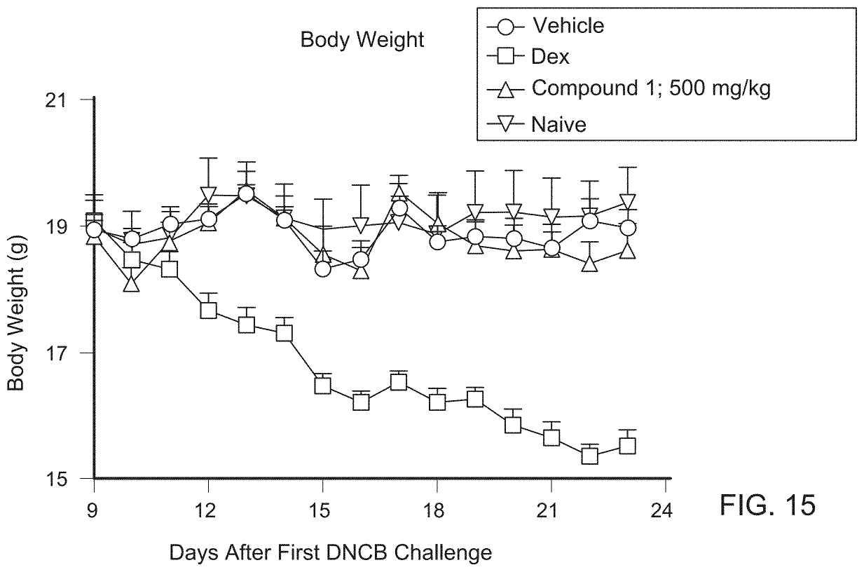

Bodyweight was also monitored daily throughout the study. The bodyweights of vehicle and Compound 1-treated animals was not significantly impacted. DEX treatment caused decreases in bodyweight as expected. There was no statistically significant difference in bodyweights between the Compound 1 group and the vehicle group. A plot of bodyweight over time is provided in FIG. 15.

### Example E. Imiquimod (IMQ)-Induced Psoriasis Model

A study was conducted to investigate the efficacy of Compound 1 in an imiquimod (IMQ)-induced psoriasis model. Female BALB/C mice were given a daily topical dose of 5% IMQ cream on the shaved back and ear for 7 consecutive days. The animals were treated with vehicle or Compound 1 twice a day (BID) for a total of 9 days. Dexamethasone (DEX) was included as a comparator. Details of the study are provided below.

### Formulation of Compound 1

Compound 1 was formulated in a vehicle of sesame oil. Each formulation was prepared by adding the required volume of sesame oil to the weighed-out amount of Compound 1 and stirring the solution at room temperature overnight for evenly distributed suspension. Before each animal administration the formulation was turned upside down several times so as to generate a more evenly distributed suspension. The formulation was prepared fresh daily. All vehicle and Compound 1 dosing were performed by oral gavage (PO) at 10 mL/kg. All BID (twice daily dosing) dosing was based on a 12 h cycle with the schedules calculated.

### Equipment

The following equipment was used:

Electronic balance: Changzhou Tianzhiping, YH2000

Semimicro balance: Sartorius, CPA225D

Micrometer: Digimatic QuantuMike Micrometer, 293-185

Thermometer: Omron IR thermometer, MC-872

### Animals

Details of the mice used in the study are provided below.

| | |
|---|---|
| Animal species and strain: | BALB/C mice |
| Breeder/supplier: | Beijing Vital River Laboratory |
| | Animal Co. Ltd |
| Sex, age: | Female, 7-8 weeks |
| Test Facility: | WuXi AppTec Vivarium (Nantong) |
| Adaptation: | 3-7 days |
| Room: | Specific pathogen free room |
| Room temperature: | 20-26° C. |
| Room relative humidity: | 40-70% |
| Light cycle: | Fluorescent light for 12-hour light |
| | and 12-hour dark |
| Animal housing: | 4 mice/cage by treatment group |
| Food and water: | Free access to food and water |

### Grouping

Eight-week female BALB/c mice were used (n=28) and divided into 4 groups.

On Day 0-Day 6, IMQ was topically applied on one ear and the shaved (dorsal) back of each mouse. The dose regimen and group setting are shown in the below table. The test article was prepared daily. Compound 1 was administered from Day −2 to Day 6. DEX was applied from Day 0 to Day 6.

| Group | N | Test articles | Treatment | | Route | Frequency |
|---|---|---|---|---|---|---|
| 1 | 4 | Naive | — | | — | — |
| 2 | 8 | Vehicle | — | | PO | BID, Day −2 |
| | | | | | | to Day 6 |
| 3 | 8 | DEX | 70 | mg | Topical | QD, Day 0 |
| | | ointment | | | | to Day 6 |
| 4 | 8 | Compound 1 | 500 | mg/kg | PO | BID, Day −2 |
| | | | | | | to Day 6 |

Abbreviations: BID = twice a day; DEX = dexamethasone; PO = oral; QD = once daily.

### IMQ Sensitization and Treatment

From Day 0 to Day 6, the mice from Group 2 to Group 4 received a daily topical application of IMQ cream on the ear and shaved (dorsal) back. (˜13:30 every day).

From Day −2 to Day 6, the test compounds and vehicle were given as the regimen indicated in the above table (˜9:30, ˜21:30 for BID dosing, and every day).

From Day 0 to Day 6, DEX was given as the regimen indicated in the above table (˜9:30, every day).

### Results

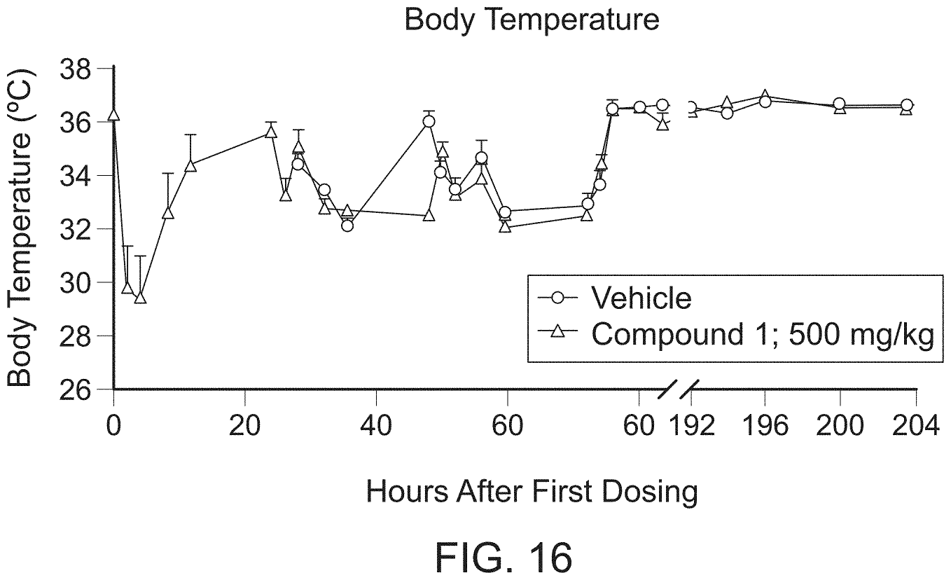

The body temperature of the vehicle and Compound 1 groups were measured by Omron IR thermometer at pre-dose, 2 h, 4 h, 8 h and 11.5 h post dose on first 3 days of the treatment and the last day of the treatment with forehead measuring mode at the face. The body temperature dropped to 30° C. after the first dose on Day −2 and recovered within 24 h. On Day −1 and 0, the administration of Compound 1 and vehicle made the body temperature range from 32 to 36° C., and recovered faster than that on Day −2. On Day 7, the administration of Compound 1 and vehicle had no effect on body temperature. The body temperature curves of both groups were similar. The body temperature curves of both groups is shown in FIG. 16.

Ear thickness was recorded by micrometer every other day. The application of IMQ on the ears increased the ear thickness significantly from Day 3 onwards. DEX treatment potently suppressed the ear thickness (Day 3-Day 7, P<0.0001). Compound 1 also inhibited an increase in ear thickness, starting on Day 5 and especially on Day 7. A plot of ear thickness for the days preceding and following dosing is shown in FIG. 17.

The following clinical score parameters were used to assess erythema, scaling, and thickness of the back.

Clinical scoring parameter

| | |
|---|---|
| Erythema | none (0), mild (1), moderate (2), severe (3) |
| Thickness | none (0), mild (1), moderate (2), severe (3) |
| Scab | none (0), mild (1), moderate (2), severe (3) |

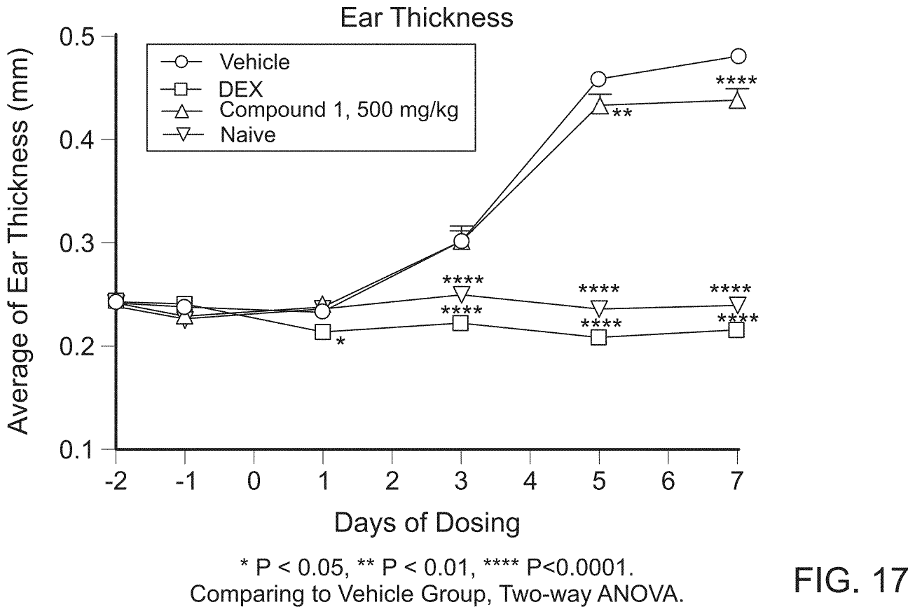

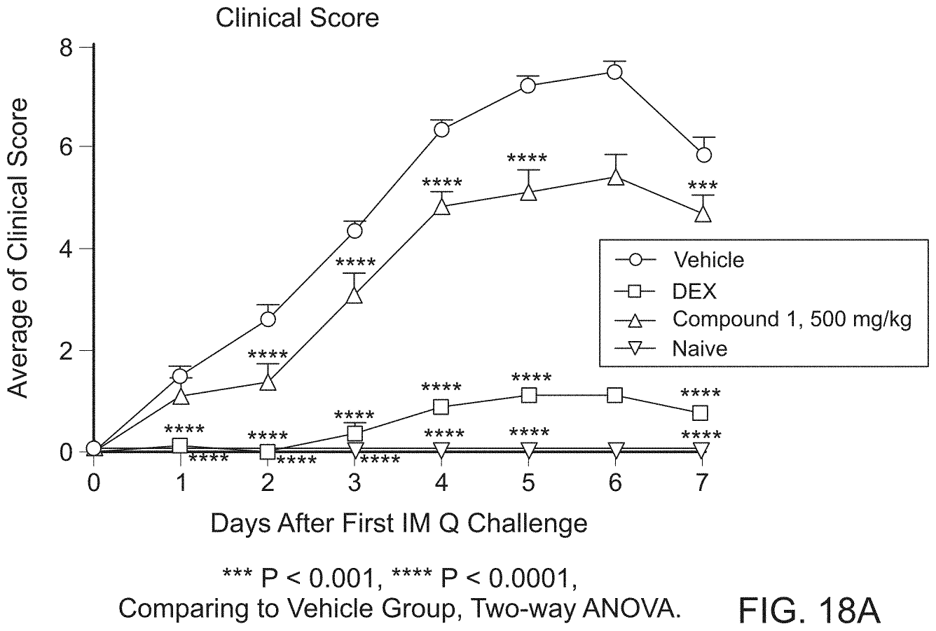

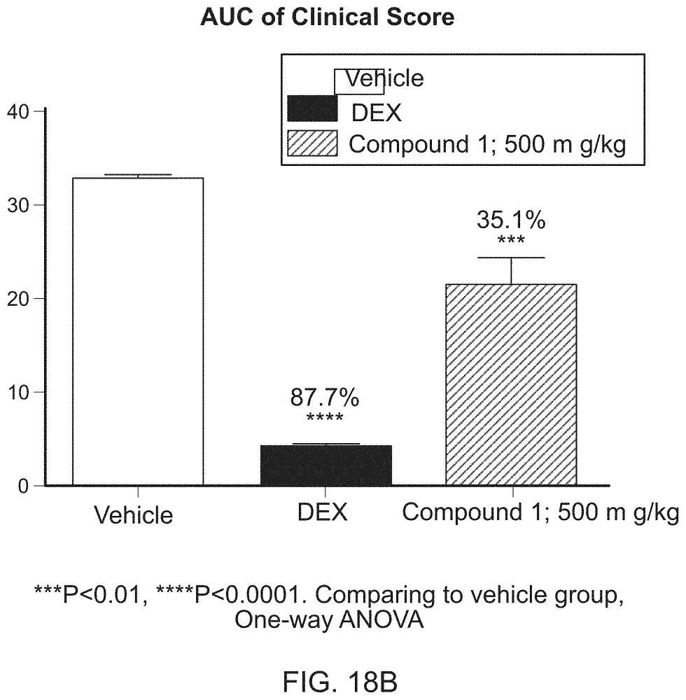

Erythema, scaling and thickness of the dorsal (back) skin were scored daily. The cumulative scores (Erythema plus Thickness plus Scaling) are presented as the total score. The DEX treatment group significantly inhibited inflammation starting on Day 1 (P<0.0001). Compound 1 treatment showed significant efficacy from Day 2 to Day 7 (P<0.001). A plot of the total clinical score in the days after first IMQ challenge is shown in FIG. 18A.

Based on the total clinical score curve of each animal in each group, the AUC was calculated, and is shown in FIG. 18B. The inhibition rate was calculated by [AUC (vehicle)-AUC (treatment)]/AUC (vehicle)×100. The DEX group had significantly inhibited clinical scoring by 87.7% (P<0.0001). Compound 1 at an oral dose of 500 mg/kg BID significantly inhibited clinical scoring by 35.1% (P<0.001).

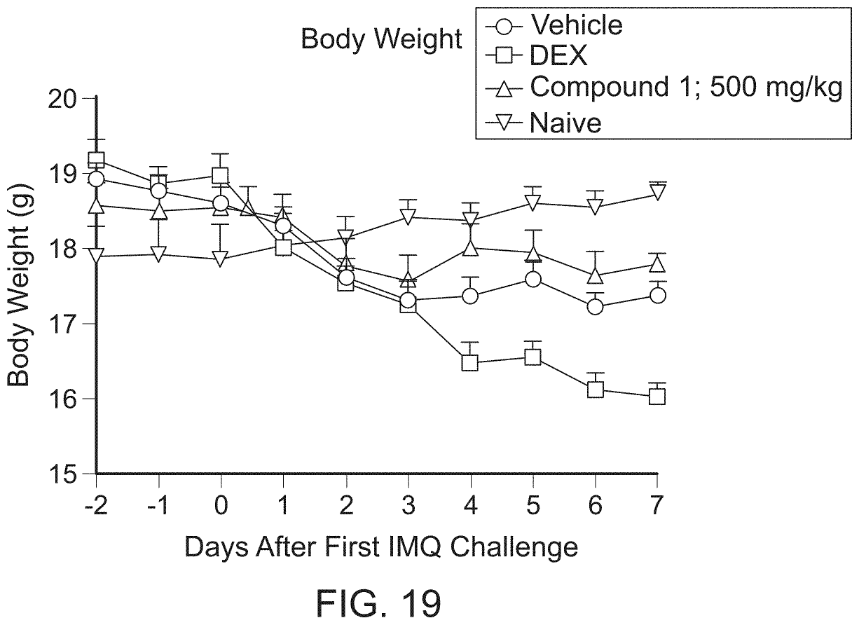

Bodyweight was monitored daily throughout the study. Consecutive treatment of IMQ and dosing caused decreases in mean bodyweights of vehicle and Compound 1 group animals. DEX treatment also caused decreases in bodyweight as expected. There was no statistically significant difference between the Compound 1 group and the vehicle group. A plot of the body weight of the various treatment groups is shown in FIG. 19.

### Example F. Human IL-23-Induced Psoriasis Model

A study was conducted to investigate Compound 1 in the human IL-23-induced psoriasis model. Female C57BL/6J mice were given a daily intradermal injection of recombinant hIL-23 protein for 7 consecutive days to induce ear thickening. The animals were treated with either vehicle or Compound 1 twice a day (BID) for a total of 9 days.

### Formulation of Compound 1

Compound 1 was formulated in a vehicle of sesame oil. Each formulation was prepared by adding the required volume of sesame oil to the weighed-out amount of Compound 1 and stirring the solution at room temperature overnight for evenly distributed suspension. Before each animal administration the formulation was turned upside down several times so as to generate a more evenly distributed suspension. The formulation was prepared fresh daily. All vehicle and Compound 1 dosing were performed by oral gavage (PO) at 10 mL/kg. All BID (twice daily dosing) dosing was based on a 12 h cycle with the schedules calculated.

### Animals

Details of the mice used in the study are provided below.

| | |
|---|---|
| Animal species and strain: | C57BL/6J mice |
| Breeder/supplier: | Beijing Vital River Laboratory |
| | Animal Co. Ltd |
| Sex, age: | Female, 7-8 weeks |
| Test Facility: | WuXi AppTec Vivarium (Nantong) |
| Adaptation: | 3-7 days |
| Room: | Specific pathogen free room |
| Room temperature: | 20-26° C. |
| Room relative humidity: | 40-70% |
| Light cycle: | Fluorescent light for 12-hour light |
| | and 12-hour dark |
| Animal housing: | 4 mice/cage by treatment group |
| Food and water: | Free access to food and water |

### Equipment

The following equipment was used:

Electronic balance: Changzhou Tianzhiping, YH2000

Semimicro balance: Mettler-Toledo ML203/02

Micrometer: Digimatic QuantuMike Micrometer, 293-185

Thermometer: Omron IR thermometer, MC-872

Anesthesia machine, Raymain, HSIV-μ Quad, four channel

### Grouping

Female C57BL/6J mice, 7-8 week of age (n=20), were divided into 3 groups basing on bodyweight and ear thickness on Day −2. Compound 1 and vehicle only dosing started on Day −2. On Days 0-6 the mice were anesthetized with isoflurane and received an intradermal injection 1 μg of hIL-23 in 20 μL PBS or 20 μL PBS (Group 1) to the right ear. The dose regimen and group setting are shown in the below table.

| | | | Test | | | |
|---|---|---|---|---|---|---|
| Group | N | Induction | articles | Treatment | Route | Frequency |
| 1 | 4 | PBS | Naive | — | — | — |
| 2 | 8 | hIL-23 | Vehicle | — | PO | BID, Day |
| | | (1 μg/ | | | | −2 to Day 6 |
| | | mouse) | | | | |
| 3 | 8 | hIL-23 | Com- | 500 | PO | BID, Day |
| | | (1 μg/ | pound 1 | mg/kg | | −2 to Day 6 |
| | | mouse) | | | | |

BID = twice daily.
Exactly 12 h Intervals for BID dosing.

hIL-23 Sensitization and Treatment

hIL-23 protein was diluted with PBS to 0.05 mg/mL immediately before intradermal injection.

From Day 0 to Day 6 (˜13:30), the mice from Group 1 to Group 3 were anesthetized with isoflurane and received a daily intradermal injection of 1 μg hIL-23 in 20 μL PBS to the right ear. The mice from Group 1 received PBS only.

From Day −2 to Day 6, Compound 1 and vehicle were given as the regimen indicated in the above table (˜9:30, ˜21:30 for BID dosing, every day).

### Results

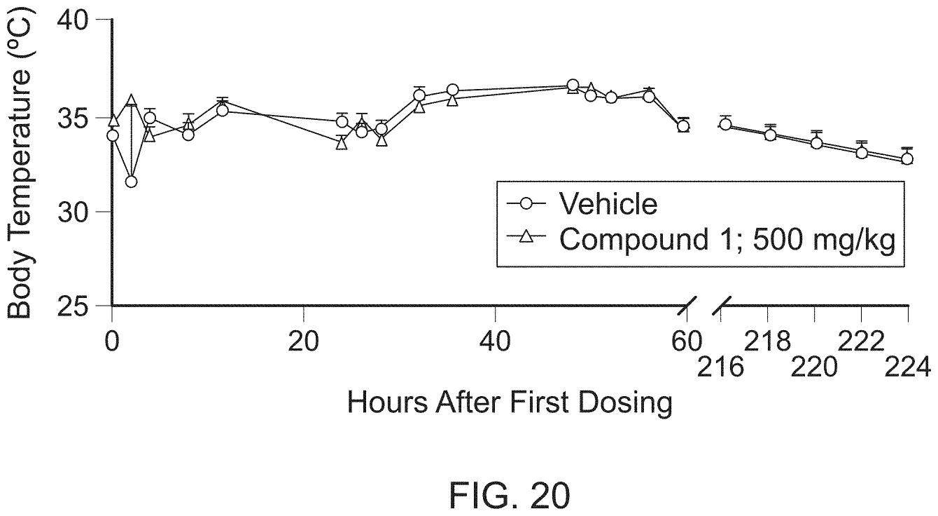

The body temperature of vehicle and Compound 1 group animals was measured by Omron IR thermometer at pre-dose, 2 h, 4 h, 8 h and 11.5 h post-dose on first 3 days of the treatment and the last day of the treatment with forehead measuring mode at the face. The body temperature of the vehicle and Compound 1-treated animals is shown in FIG. 20.

Dosing of sesame oil vehicle led to a sudden drop of the body temperature at 2 h post-dose on Day 0. At the following time points, the body temperature for both vehicle and Compound 1 treated groups remained in a normal range in the first 3 days. The body temperature was relatively lower on Day 7, ranging from 32.6-34.6° C. in 24 h. The body temperature curves of both groups were similar.

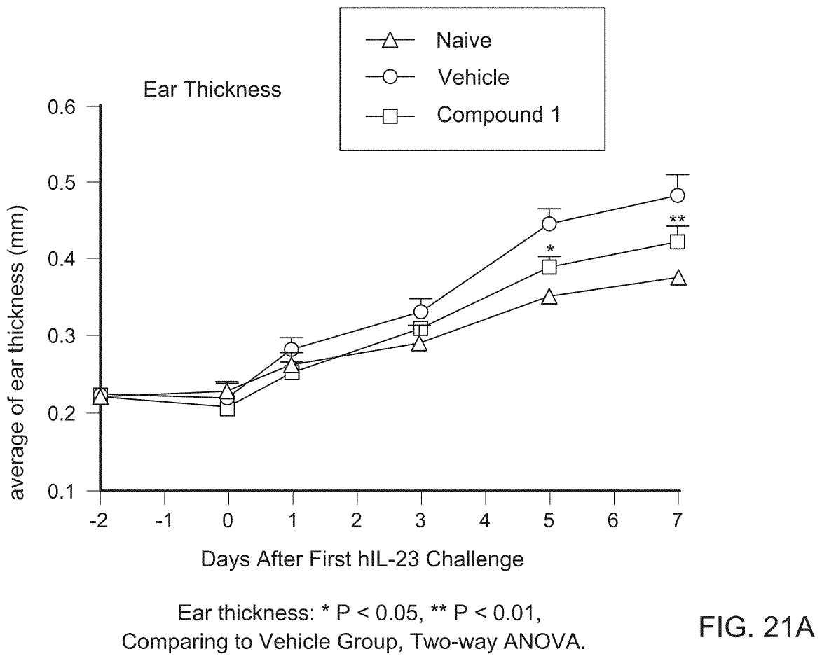

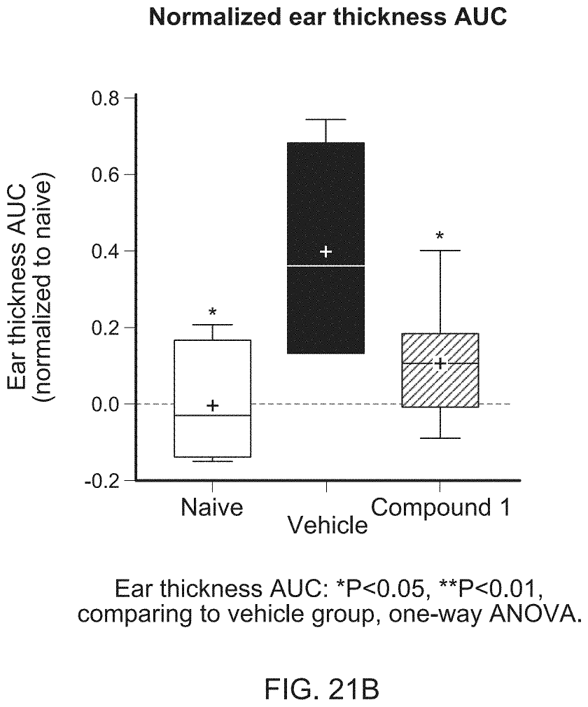

Ear thickness was recorded by micrometer every other day. Intradermal injection of hIL-23 to the right ear increased the ear thickness. A plot of ear thickness for the naïve, vehicle, and Compound 1 groups is shown in FIG. 21A. The inhibition rate for specific days or the area under the curve (AUC) was calculated by [(Thickness vehicle−Thickness Naïve)−(Thickness treatment−Thickness Naïve)]/[Thickness vehicle−Thickness Naive]×100. A graph of the normalized ear thickness AUC for the naïve, vehicle, and Compound 1 groups is shown in FIG. 21B. Compound 1 group exhibited inhibition on ear thickness increases, on Day 5 by 60% (P<0.05) and Day 7 by 60% (P<0.01) and Compound 1 reduced the AUC by 72% (P<0.05).

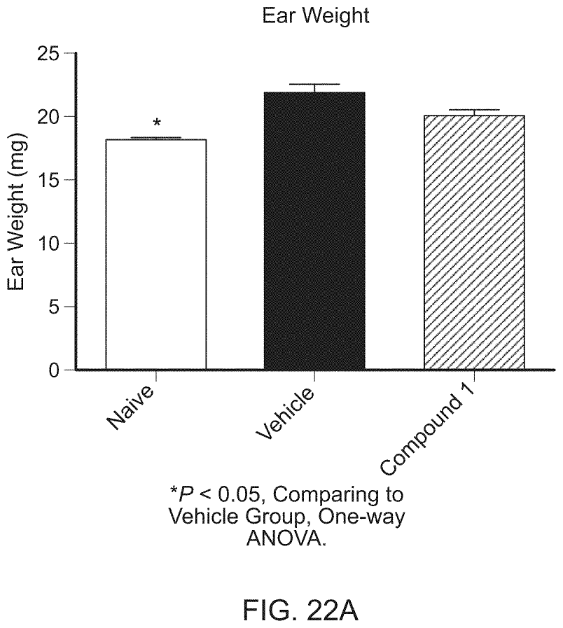

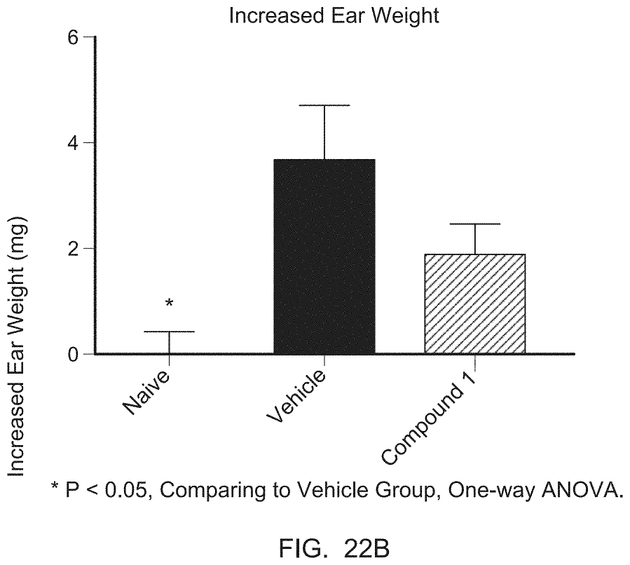

On Day 7, a piece of ear (8 mm in diameter) was taken from each mouse. The weight of the ear was recorded, and the increased weight was calculated by [Weight (treatment) −Weight (naive) mean]. A graph of ear thickness of the naïve, vehicle, and Compound 1 groups is shown in FIG. 22A. A graph of the increased ear weight for the naïve, vehicle, and Compound 1 groups is shown in FIG. 22B. In line with the data of the ear thickness, the ear weight of the vehicle group was significantly higher than that of the naive group. The Compound 1 group showed a trend towards decreased ear weight compared to vehicle control.

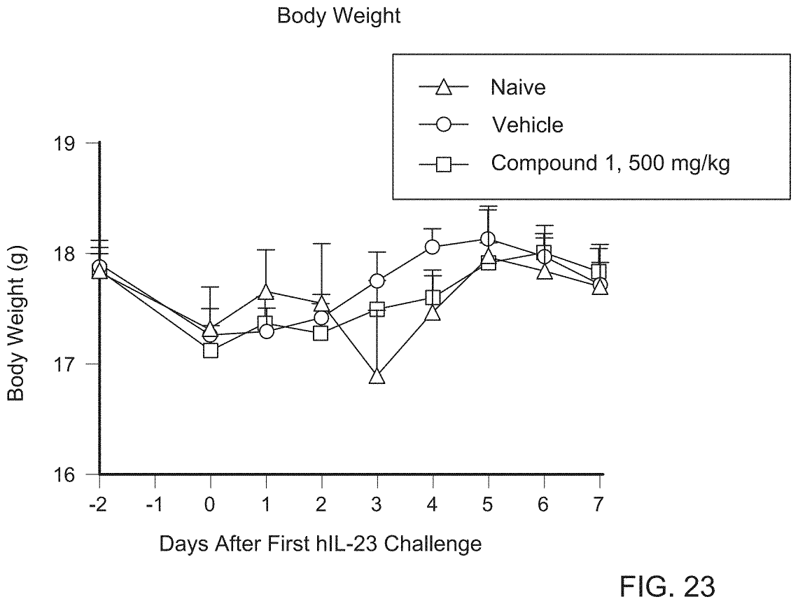

Bodyweights were monitored daily throughout the study. There were no statistically significant differences between the Compound 1 group and the vehicle group. A plot of the bodyweight of the naïve, vehicle, and Compound 1 groups is shown in FIG. 23.

### Example G: Bleomycin (BLM)-Induced Pulmonary Fibrosis Model

A study was conducted to evaluate the efficacy of Compound 1 in a bleomycin (BLM)-induced pulmonary fibrosis model. BLM-induced pulmonary fibrosis is an established disease model for IPF. In the model, alveolar injury and interstitial inflammation/fibrosis are induced by intracheal BLM administration.

### Test Article Preparation

Compound 1: Compound 1 was weighed out and diluted to the required concentrations relevant to the study using sesame oil. For example, Compound 1 (4821 mg) was added into sesame oil (80 mL) with grinding to obtain a suspension with concentration at 60 mg/mL of Compound 1. The formulation was then sonicated using a water bath sonicator for 30 min. Immediately prior to dosing each animal the formulations were vortexed to ensure an equally distributed suspension of Compound 1. Formulations (50 μL/vial) were kept for dose verification on the first and last dosing day (2 vials each, total 4 vials).

Tofacitinib: Tofacitinib (25 mg) was added into 5 mL of 0.5% methylcellulose/0.025% Tween 20 in water with vortexing and sonicating to obtain a suspension with a concentration of about 5 mg/mL of Tofacitinib.

### Animals

Forty-five 6-week-old female C57BL/6 mice were obtained from Japan SLC, Inc. (Japan).

Animals were housed and fed with normal diet (CE-2; CLEA Japan, Japan) under controlled conditions. All animals used in the study were housed and cared for in accordance with the Japanese Pharmacological Society Guidelines for Animal Use.

The animals were maintained in a SPF facility under controlled conditions of temperature (23±3° C.), humidity (50±20%), lighting (12-hour artificial light and dark cycles; light from 8:00 to 20:00) and air exchange. A high pressure was maintained in the experimental room to prevent contamination of the facility.

The animals were housed in TPX cages (CLEA Japan) with a maximum of 5 mice per cage. Sterilized Paper-Clean (Japan SLC) was used for bedding and replaced once a week.

Sterilized normal diet was provided ad libitum, being placed in a metal lid on the top of the cage. RO water was provided ad libitum from a water bottle equipped with a rubber stopper and a sipper tube. Water bottles were replaced once a week, cleaned and sterilized in autoclave and reused. The mice treated with Compound 1 had access to food pellets at the bottom of their cages as well as hydrogel.

Mice were identified by ear punch. Each cage was also given a specific identification code.

Test Groups

Group 1: Sham

Five control mice, following single intratracheal phosphate-buffered saline (PBS) administration, were kept without any treatment until sacrifice.

Group 2: Vehicle

Fifteen BLM-induced pulmonary fibrosis model mice were orally administered vehicle (sesame oil) in a volume of 10 mL/kg twice daily from Day 1 to 14.

Group 3: Compound 1

Fifteen BLM-induced pulmonary fibrosis model mice were orally administered vehicle (sesame oil) supplemented with Compound 1 at a dose of 500 mg/kg in a volume of 10 mL/kg twice daily (total of 1000 mg/kg/day) from Day 1 to 14.

Group 4: Tofacitinib Free Base

Ten BLM-induced pulmonary fibrosis model mice were orally administered vehicle (0.5% methylcellulose/0.025% Tween 20) supplemented with Tofacitinib free base at a dose of 50 mg/kg in a volume of 10 mL/kg twice daily (total of 100 mg/kg/day) from Day 1 to 14.

The study design and treatment schedule is summarized in the below table:

| | No. | | Test | Dose | Volume | | |
|---|---|---|---|---|---|---|---|
| Group | mice | Model | substance | (mg/kg) | (mL/kg) | Regimen | Sacrifice |
| 1 | 5 | Saline | — | — | — | — | Day 14 |
| 2 | 15 | BLM | Vehicle | — | 10 | PO, BID*, | Day 14 |
| | | | | | | Day 1-14 | |
| 3 | 15 | BLM | Compound 1 | 500 | 10 | PO, BID*, | Day 14 |
| | | | | | | Day 1-14 | |
| 4 | 10 | BLM | Tofacitinib | 50 | 10 | PO, BID*, | Day 14 |
| | | | free base | | | Day 1-14 | |

BLM = Bleomycin;
PO = Orally;
BID = Twice daily

Forty-five mice were divided into 1 group of 5 mice, 1 group of 10 mice and 2 groups of 15 mice based on the body weight on the day before the BLM or/saline administration at on Day 0.

### Induction of BLM-Induced Pulmonary Fibrosis Model

At Day 0, 40 mice were induced to develop pulmonary fibrosis by a single intratracheal administration of bleomycin hydrochloride (BLM, Nippon Kayaku, Japan) in PBS at a dose of 3.0 mg/kg, in a volume of 50 μL per animal using Microsprayer® (Penn-Century, USA) under a mixture of medetomidine (0.75 mg/kg), midazolam (4 mg/kg) and butorphanol (5 mg/kg) anesthesia by intraperitoneal administration in a volume of 10 mL/kg. After BLM administration, atipamezole hydrochloride (0.75 mg/kg) was given by intraperitoneal administration in a volume of 10 mL/kg and mice were put on heat pad until anesthetic effect is reversed. Five mice were intratracheally administered PBS, instead of the BLM, which served as the Sham group.

### Route and Dose of Drug Administration

Compound 1 was administered orally at a dose of 500 mg/kg in a volume of 10 mL/kg twice daily (total of 1000 mg/kg/day).

Tofacitinib was administered orally at a dose of 50 mg/kg in a volume of 10 mL/kg twice daily (total of 100 mg/kg/day).

### Animal Monitoring and Sacrifice

The viability, clinical signs and behavior were monitored daily. Body weight was recorded daily from Day 0. Dosing volume was adjusted based on the latest body weight. Mice were observed for significant clinical signs of toxicity, moribundity and mortality approximately 60 min after each administration. The animals were sacrificed on Day 14 by exsanguination through the abdominal vena cava under a mixture of medetomidine, midazolam and butorphanol anesthesia. The time of the dosing and termination was recorded. The left and post-caval lobe weight for all mice were recorded.

Animals showing larger than 40% body weight loss compared to Day 0 were euthanized by cutting the abdominal aorta and vena cava under a mixture of medetomidine (0.75 mg/kg), midazolam (4 mg/kg) and butorphanol (5 mg/kg) anesthesia by intraperitoneally administration in a volume of 10 mL/kg ahead of study termination. The samples were collected from euthanized animals.

### Preparation of PK and PD Plasma Samples (Groups 3 and 4)

Blood (50 μL) was collected from facial vein to prepare K2EDTA (Greiner Bio-One, Austria) plasma at 2 h post 1^{st} dose (in-life, mice 1-7/Group 3, mice 1-5/Group 4) and 30 min prior to 2^{nd} dose (in-life, mice 8-15/Group 3, mice 6-10/Group 4).

Blood (50 μL) was collected from facial vein to prepare K2EDTA plasma at 30 min prior last administration at sacrifice (in-life, mice 1-15/Group 3, mice 1-10/Group 4).

At 2 h post last dose (terminal, mice 1-15/Group 3, mice 1-10/Group 4), non-fasting blood was collected through postcaval vein using pre-cooled syringes at sacrifice. The collected blood was transferred in pre-cooled polypropylene tubes with anticoagulant (K2EDTA, Greiner Bio-One) and stored on ice until centrifugation. The blood samples were centrifuged at 6,000×g for 5 min at 4° C. The supernatant was collected in two separate tubes, with 100 μL for PD analysis and the remaining plasma for PK analysis, and stored at −80° C. until shipping.

### Preparation of PD Plasma Samples

At sacrifice, non-fasting blood was collected through postcaval vein using pre-cooled syringes at sacrifice from animals in Group 1 and 2. The collected blood was transferred in pre-cooled polypropylene tubes with anticoagulant (K2EDTA, Greiner Bio-One) and stored on ice until centrifugation. The blood samples were centrifuged at 6,000×g for 5 min at 4° C. A minimum of 100 μL of the supernatant was collected for PD analysis and stored at −80° C. until shipping.

### Preparation of BALF Samples

For Group 1, BALF samples were collected by flushing the lung via the trachea with sterile PBS (0.8 mL) at sacrifice under a mixture of medetomidine, midazolam and butorphanol anesthesia. The BALF was centrifuged at 3,000×g for 5 min at 4° C. and the supernatant was collected and stored at −80° C.

For Group 2-4, BALF samples were collected by flushing the lung via the trachea with sterile PBS three times (1^{st} BALF, 2^{nd} BALF and 3^{rd} BALF, 0.8 mL each) at sacrifice under a mixture of medetomidine, midazolam and butorphanol anesthesia. The 1^{st} BALF was centrifuged at 3,000×g for 5 min at 4° C. and the supernatant was collected and stored at −80° C. The cell pellet from the first fraction (1^{st} BALF) was resuspended and added to the remaining fractions of lavage fluid (2^{nd} and 3^{rd} BALF). The total BALF was centrifuged at 1,000×g for 5 min at 4° C. and the supernatant was removed. Ammonium-Chloride-Potassium (ACK) lysing buffer was added to the pellet and washed for lysis of erythrocytes. After addition of cold PBS, the fluid was centrifuged at 1,000×g for 3 min at 4° C. and the supernatant was removed. Resuspended in PBS and total cell number of BALF was counted using a hemocytometer (C-Chip disposable hemocytometer, Digital Bio). After counting the total cells, the cell suspension was re-suspended in PBS to 5×10⁵ cells/mL. A smear sample was then prepared by spotting 100 μL of cell suspension onto slides, and air-dried using a dryer on cool setting.

### Preparation of Lung Samples

Left lobe, post-caval lobe and right inferior lobe bronchus were ligated to avoid leakage of the fixative. Indwelling needle was inserted into trachea and connected to instillation route of syringe. The syringe was loaded with 10% neutral buffered formalin and kept at the height of 20 cm. Then, the superior and middle lobes were instilled 10% neutral buffered formalin and ligated after inflation. The two fixed lobes (for histological analyses), the unfixed left lung (for biochemistry) and the unfixed right inferior and post-caval lobes (for shipping) were harvested. The three unfixed lobes were washed with cold saline and measured wet weight.

Two fixed lobes were fixed in 10% neutral buffered formalin for 24 hours. After fixation, these specimens were paraffin-embedded for Masson's trichrome staining.

Right inferior and post-caval lobes were snap frozen in liquid nitrogen and stored at −80° C. until shipping.

For all groups, right inferior lobe (minimum of 30 mg) was snap frozen into RNAlater.

For all groups, remaining right inferior lobe and post-caval lobe (˜50 mg) directly was put into one homogenizer tube with 500-750 μL of MAR lysis buffer and snap frozen in liquid nitrogen.

Left lung was snap frozen in liquid nitrogen and stored at −80° C. for hydroxyproline analysis.

### Measurement of Lung Hydroxyproline Content (all Groups)

To quantify lung hydroxyproline content, frozen left lung samples were processed by an acid hydrolysis method as follows. Lung samples were acid-hydrolyzed with 200 μL of 6N HCl at 121° C. for 20 minutes and neutralized with 200 μL of 4N NaOH containing 10 mg/mL activated carbon. AC buffer (2.2M acetic acid/0.48M citric acid, 200 μL) was added to the samples, followed by centrifugation to collect the supernatant. A standard curve of hydroxyproline was constructed with 16, 8, 4, 2, 1 and 0.5 μg/mL of trans-4-hydroxy-L-proline (Sigma-Aldrich Co. LLC., USA Code: 54409). The prepared samples and standards (each 300 μL) were mixed with 300 μL chloramine T solution (NACALAI TESQUE, INC., Japan, Code: 08005-52) and incubated for 25 minutes at room temperature. The samples were then mixed with Ehrlich's solution (300 μL) and heated at 65° C. for 20 minutes to develop the color. After samples were cooled on ice and centrifuged to remove precipitates, the optical density of each supernatant was measured at 560 nm. The concentrations of hydroxyproline were calculated from the hydroxyproline standard curve. Lung hydroxyproline levels were expressed as pg per left lung.

### Measurement of BALF IL-4, IL-6, IL-10 and IL-17 (Groups 2-4)

For Group 2-4, BALF IL-4, IL-6, IL-10 and IL-17 levels were quantified by Quantikine enzyme linked immunosorbent assay kits (code. M4000B for IL-4, code. M6000B for IL-6, code. M1000B for IL-10, code. M1700 for IL-17, R&D systems) according to manufacturer instructions.

### Blinding

Samples were coded a number for blind evaluation. Each number was generated using the RAND function of Excel software, sorted in ascending order and assigned the slides. The tissue slides were used for the following stains and evaluated by an experimenter.

### Histological Analysis

For all groups, right lung tissues prefixed in 10% neutral buffered formalin were embedded in paraffin and sectioned at 4 μm (2 section/slide).

For Masson's Trichrome staining, the sections were deparaffinized and rehydrated, followed by re-fixation with Bouin's solution for 15 min. The sections were stained in Weigert's iron Hematoxylin working solution (Sigma-Aldrich), Biebrich scarlet-Acid fuchsin solution (Sigma-Aldrich), Phosphotungstic/phosphomolybdic Acid solution, Aniline blue solution and 1% Acetic Acid solution (Sigma-Aldrich). For quantitative analysis of lung fibrosis area, bright field images of Masson's Trichrome-stained sections were randomly captured using a digital camera (DFC295; Leica, Germany) at 100-fold magnification, and the subpleural regions in 20 fields/mouse were evaluated according to the criteria for grading lung fibrosis (Ashcroft, T., et al., J Clin Pathol, 1988; 41:467-70). All sections were blindly analyzed by an experimenter.

Lung fibrosis was graded using the following criteria:

| Grade* | Histological Features |
|---|---|
| 0 | Normal lung |
| 1 | Minimal fibrous thickening of alveolar or bronchiolar walls |
| 2 | |
| 3 | Moderate thickening of walls without obvious damage to |
| | lung architecture |
| 4 | |
| 5 | Increased fibrosis with definite damage to lung structure |
| | and formation of fibrous bands or small fibrous masses |
| 6 | |
| 7 | Severe distortion of structure and large fibrous areas; |
| | “honeycomb lung” is placed in this category. |
| 8 | Total fibrous obliteration of the field |

*If there was any difficulty in deciding between two odd numbered categories, the field would be given the intervening even numbered score.

For Group 2-4, Diff-Quik (Sysmex Corporation) staining was conducted on the prepared smear samples by immersing them in the following order of fixative, staining solution I and staining solution II, as described in the Diff-Quik manual. Using the stained sections, blood cell morphology analysis (lymphocyte, monocyte, neutrophil and eosinophil) was conducted. The slides were viewed at 200× magnification using a bright field microscope (Leica Microsystems). Blood cells were counted until a total of 200 was reached, and the ratio of each blood cell was calculated. The number of each blood cell type was then calculated by applying the ratio to the number of total cells.

### Results

Body weight was expressed as percentage of body weight change from baseline (Day 0). Mean body weight changes of the Sham group were significantly higher than that of the Vehicle group from Day 6 to Day 14. Mean body weight changes of the Compound 1 group were significantly higher than that of the Vehicle group from Day 8 to Day 12. Mean body weight changes of the Tofacitinib free base group were significantly higher than that of the Vehicle group from Day 6 to Day 14.

During the treatment period, mice found dead and euthanized before reaching Day 14 are as follows; two out of 15 mice were euthanized in the Vehicle group. Two out of 15 mice were found dead in the Compound 1 group. There were no dead animals in the Tofacitinib free base group during the treatment period.

Body and lung weight measurements are shown in the below table.

| | | | | Tofacitinib |
|---|---|---|---|---|
| Parameter | Sham | Vehicle | Compound 1 | free base |
| (mean ± SD) | (n = 5) | (n = 13) | (n = 13) | (n = 10) |
| Body weight (g) | 18.8 ± 1.3 | 16.0 ± 3.2 | 17.1 ± 2.0 | 18.1 ± 1.7 |
| Left lung weight | 87 ± 8 | 109 ± 16 | 101 ± 14 | 101 ± 9 |
| (mg) | | | | |
| Post-caval | 21 ± 2 | 34 ± 10 | 28 ± 7 | 27 ± 4 |
| lobe weight | | | | |
| Right inferior | 68 ± 4 | 101 ± 20 | 84 ± 17 | 84 ± 14 |
| lobe weight | | | | |

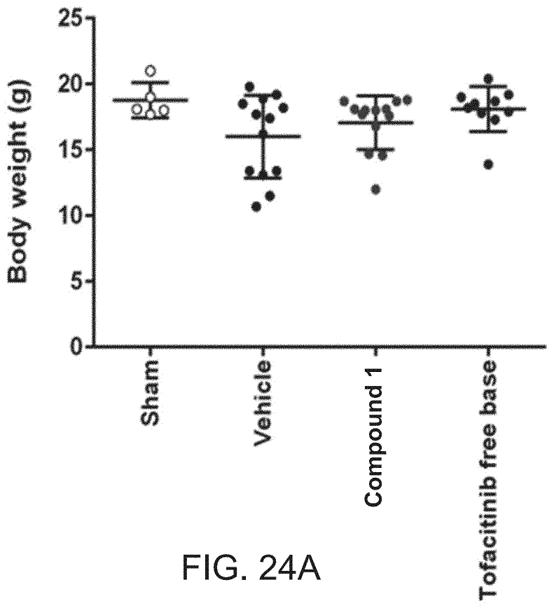

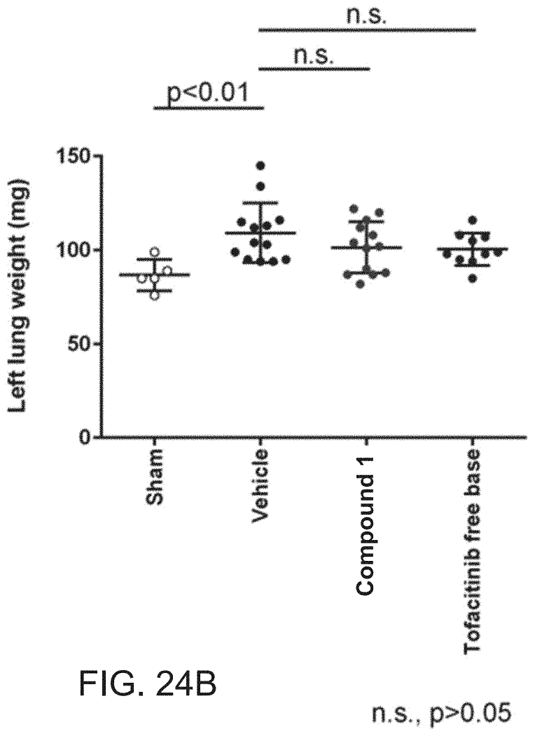

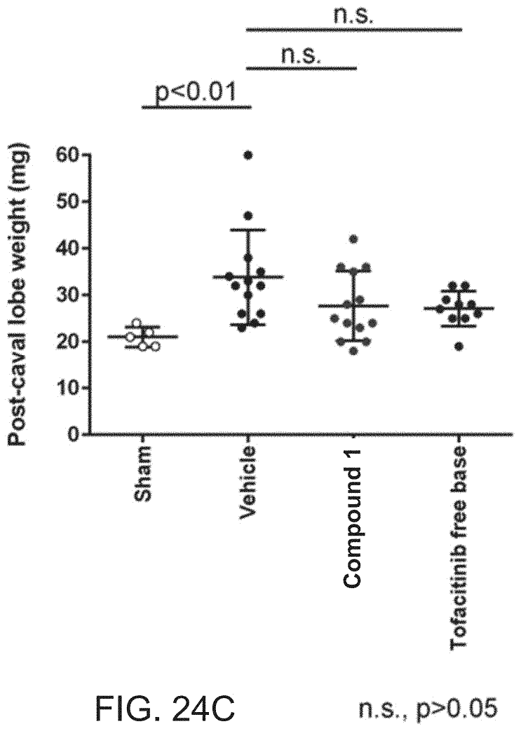

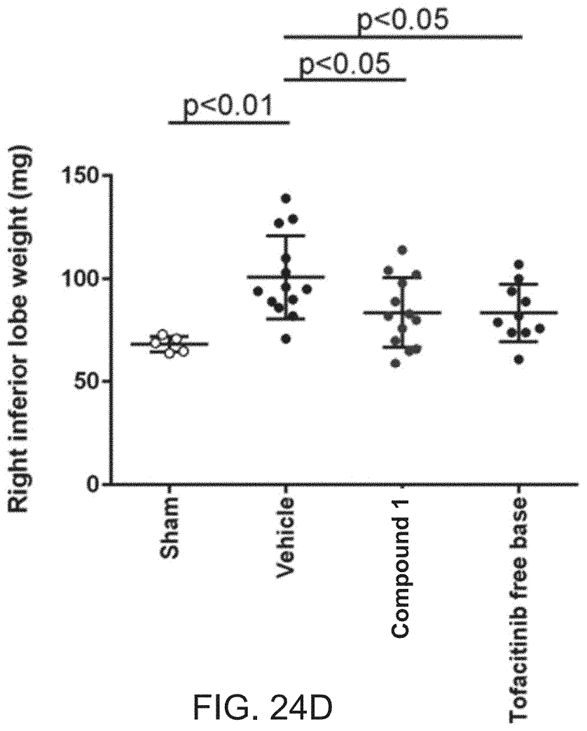

FIG. 24A shows the body weight and lung weight of mice in each treatment group. FIG. 24B shows the left lung weight of mice of each treatment group. FIG. 24C shows the post-caval lobe weight of mice in each treatment group. FIG. 24D shows the right inferior lobe weight of mice in each treatment group.

There were no significant differences in the survival curves between the Vehicle group and the study groups.

There was no significant difference in the body weights on the day of sacrifice. Mean body weight on the day of sacrifice in the Sham group tended to increase compared with the Vehicle group.

The Sham group showed a significant decrease in mean left lung weight compared with the Vehicle group. Mean left lung weight in the Compound 1 and the Tofacitinib free base groups tended to decrease compared with the Vehicle Group.

The Sham group showed a significant decrease in mean post-caval lobe weight compared with the Vehicle group. Mean post-caval lobe weight in the Compound 1 and Tofacitinib free base groups tended to decrease compared with the Vehicle group.

The Sham group showed a significant decrease in mean right inferior lobe weight compared with the Vehicle group. Compound 1 and Tofacitinib free base groups showed significant decreases in mean post-caval lobe weight compared with the Vehicle group.

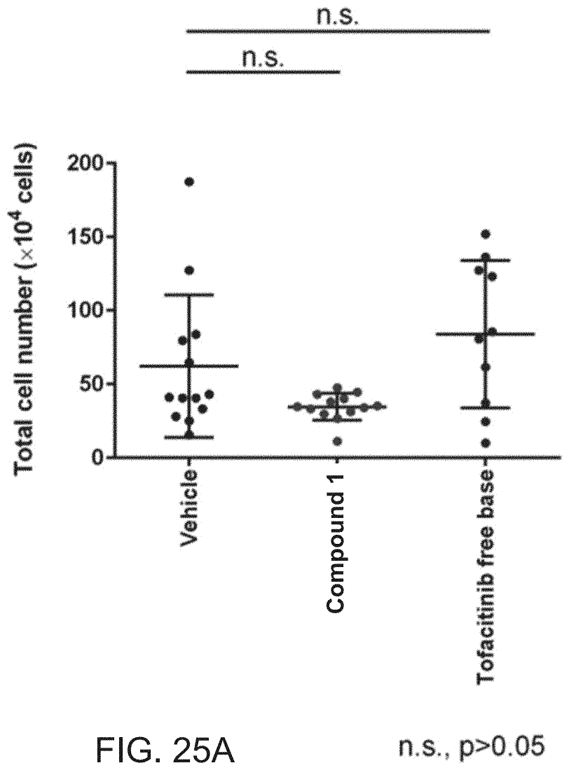

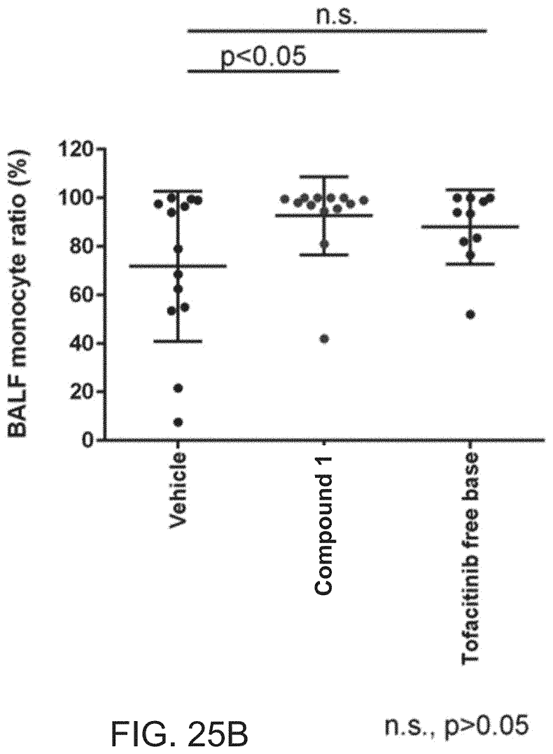

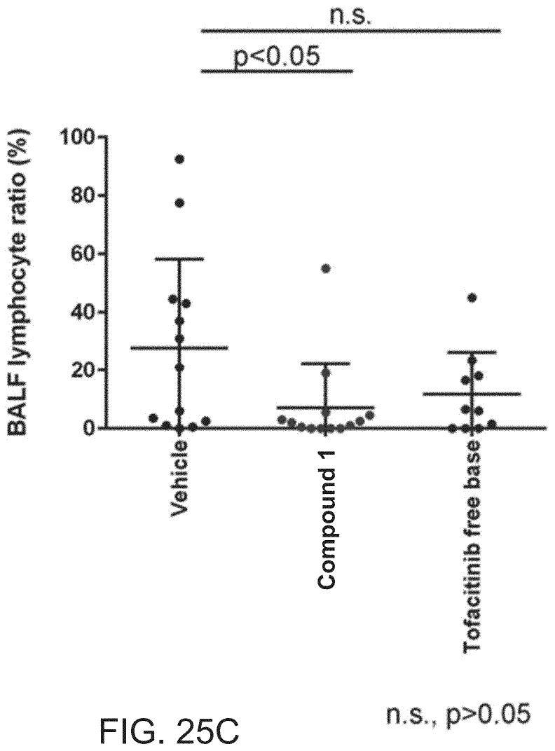

FIG. 25A shows the total number of immune cells of the bronchoalveolar lavage fluid of mice in each treatment group. FIG. 25B shows the BALF monocyte ratio of mice in each treatment group. FIG. 25C shows the BALF lymphocyte ratio of mice in each treatment group. FIG. 25D shows the BALF neutrophil ratio of mice in each treatment group.

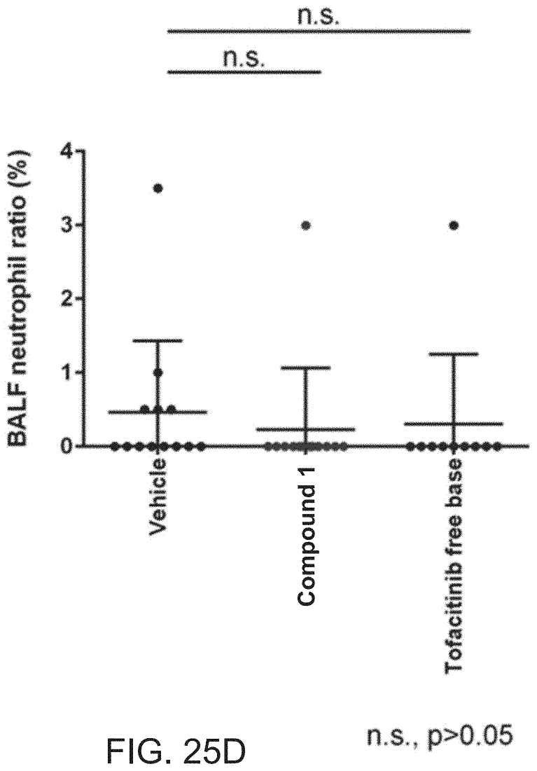

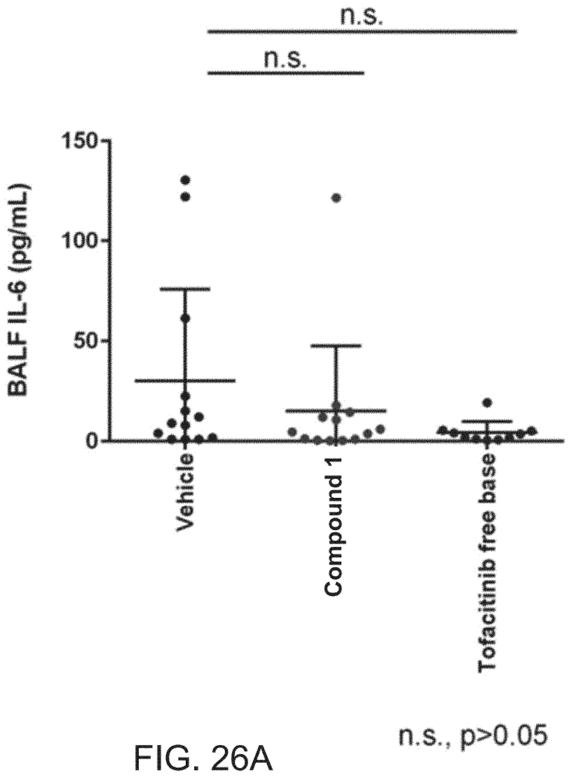

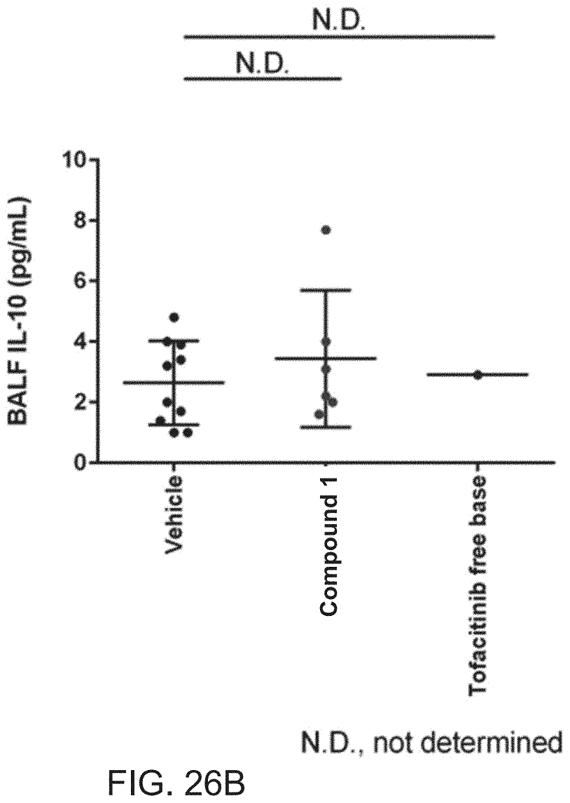

FIG. 26A shows the BALF IL-6 concentrations in mice for each treatment group. FIG. 26B shows the BALF IL-10 concentrations in mice for each treatment group.

Measurements of BALF are shown in the below table.

| | | | | Tofacitinib |
|---|---|---|---|---|
| Parameter | Sham | Vehicle | Compound 1 | free base |
| (mean ± SD) | (n = 5) | (n = 13) | (n = 13) | (n = 10) |
| Total cells | — | 62.3 ± 48.3 | 34.6 ± 9.2 | 83.8 ± 50.0 |
| (×10⁴) | | | | |
| Monocyte | — | 71.8 ± 31.0 | 92.6 ± 16.0 | 88.0 ± 15.2 |
| Lymphocyte | — | 27.7 ± 30.6 | 7.2 ± 15.3 | 11.7 ± 14.5 |
| Neutrophil | — | 0.5 ± 1.0 | 0.2 ± 0.8 | 0.3 ± 0.9 |
| Eosinophil | — | 0.0 | 0.0 | 0.0 |
| BALF IL-4 | — | — | — | — |
| (pg/mL) | | | | |
| BALF IL-6 | — | 30.0 ± 45.7 | 15.0 ± 32.6 | 4.4 ± 5.5 |
| (pg/mL) | | | | |
| BALF IL-10* | — | 2.6 ± 1.4 | 3.4 ± 2.3 | 2.9 |
| (pg/mL) | | | | |
| BALF IL-17 | — | — | — | — |
| (pg/mL) | | | | |

*Vehicle (n = 10), Compound 1 (n = 6), Tofacitinib free base (n = 1)

The total number of cells in BALF in the Compound 1 group tended to decrease compared with the Vehicle group. There was no significant difference in total number of cells in BALF between the Vehicle group and the Tofacitinib free base group.

The Compound 1 group showed a significant increase in the percentage of monocyte in BALF compared with the Vehicle group. The percentage of monocyte in BALF in the Tofacitinib free base group tended to increase compared with the Vehicle group.

The Compound 1 group showed a significant decrease in the percentage of lymphocyte in BALF compared with the Vehicle group. The percentage of lymphocyte in BALF in the Tofacitinib free base group tended to decrease compared with the Vehicle group.

There was no significant difference in percentage of neutrophils.

BALF IL-6 content in the Tofacitinib free base group tended to decrease compared with the Vehicle group.

Lung hydroxyproline content is shown in the below table.

| | | | | Tofacitinib |
|---|---|---|---|---|
| Parameter | Sham | Vehicle | Compound 1 | free base |
| (mean ± SD) | (n = 5) | (n = 13) | (n = 13) | (n = 10) |
| Lung | 40.4 ± 2.9 | 58.1 ± 9.6 | 53.1 ± 10.9 | 49.3 ± 8.3 |
| hydroxyproline | | | | |
| (μg/left lung) | | | | |

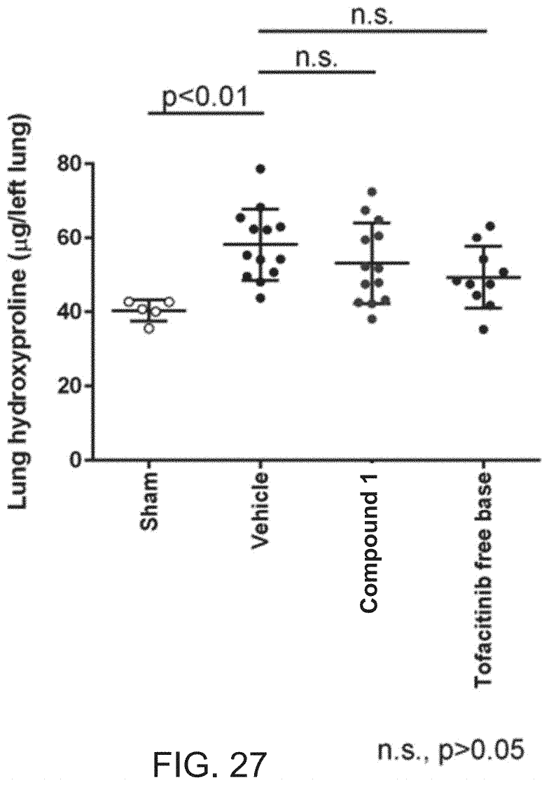

FIG. 27 shows the lung hydroxyproline content of each treatment group.

The Sham group showed a significant decrease in lung hydroxyproline content compared with the Vehicle group. Lung hydroxyproline content in the Tofacitinib free base group tended to decrease with the Vehicle group. There was no significant different in lung hydroxyproline content between the Vehicle group and the Compound 1 group.

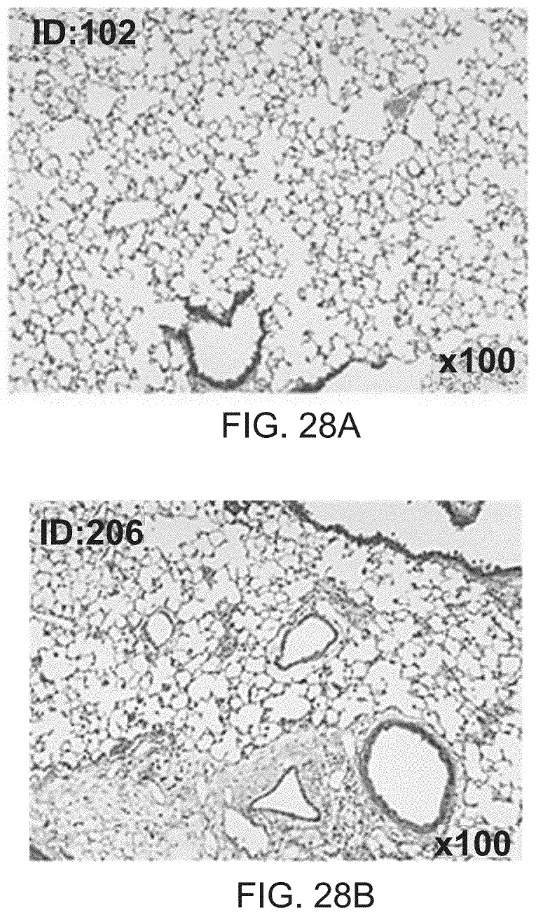

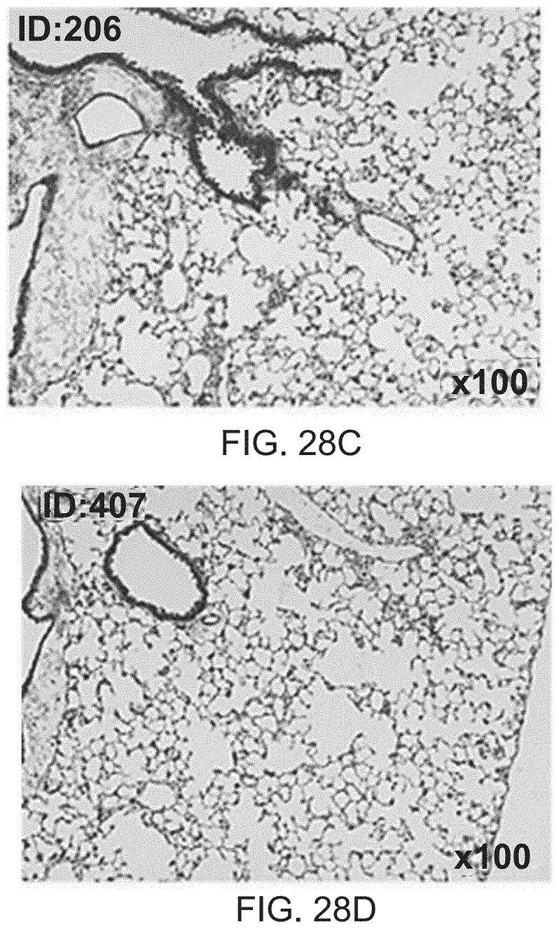

FIG. 28A shows photomicrographs of Masson's trichrome-stained lung section in the Sham treatment group. FIG. 28B shows photomicrographs of Masson's trichrome-stained lung section in the Vehicle treatment group. FIG. 28C shows photomicrographs of Masson's trichrome-stained lung section in the Compound 1 treatment group. FIG. 28D shows photomicrographs of Masson's trichrome-stained lung section in the Tofacitinib free base treatment group.

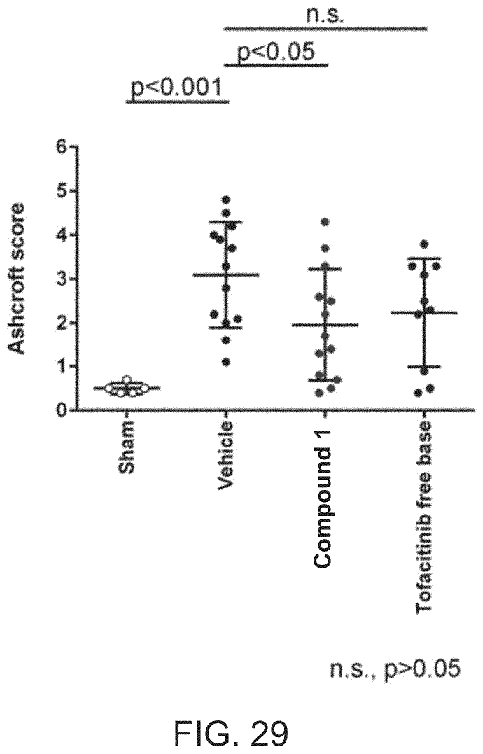

FIG. 29 shows the Ashcroft score of mice in each of the treatment groups.

The Ashcroft score for the various treatment groups is shown in the below table.

| | | | | Tofacitinib |
|---|---|---|---|---|
| Parameter | Sham | Vehicle | Compound 1 | free base |
| (mean ± SD) | (n = 5) | (n = 13) | (n = 13) | (n = 10) |
| Ashcroft score | 0.5 ± 0.1 | 3.1 ± 1.2 | 2.0 ± 1.3 | 2.2 ± 1.2 |

The Sham and Compound 1 groups showed a significant decrease in Ashcroft score compared with the Vehicle group. Ashcroft score in the Tofacitinib free base group tended to decrease compared with the Vehicle group.

### Conclusion

Treatment with Compound 1 showed significant decreases in mean right inferior lobe weight, the percentage of lymphocyte in BALF, and Ashcroft score, and a significant increase in the percentage of monocyte in BALF, and decreasing trends in mean left lung weight, mean post-caval lobe weight, total number of cells in BALF and Ashcroft score compared with the Vehicle group.

Treatment with Tofacitinib free base showed significant decreases in mean right inferior lobe weight, the percentage of lymphocyte in BALF, and a significant increase in the percentage of monocyte in BALF, and an increasing trend in mean body weight on the day of sacrifice, and decreasing trends in mean left lung weight, mean post-caval lobe weight, BALF IL-6 content, lung hydroxyproline content and Ashcroft score compared with the Vehicle group.

### Example H. PARP14 IHC in Inflammation Assay

The staining procedure for immunohistochemistry (“IHC”) analysis of PARP14 (rabbit clone 15B10-1) with DAB chromogen was performed using automated detection on the Leica Bond Rx (Leica Biosystems). All steps occurred at room temperature unless noted otherwise.

### Pre-Processing and Pretreatment

FFPE specimens were sectioned at 4-micron thickness, mounted onto positive-charged glass slides, dried, and baked for 30 minutes at 60° C. in an oven deparaffinized and rehydrated offline in accordance with standard practices. Tissue slides were then placed onto the Leica Bond RX autostainer and underwent pretreatment using Epitope Retrieval Solution 2 (ER2, Catalog #AR9640, Leica) starting with 2 rinses in ER2, then incubation in ER2 for 40 minutes at 100° C. followed by a rinse in ER2. Tissue slides were then rinsed 4 times followed by a 3-minute incubation with Bond Wash Solution (Catalog #AR9590, Leica Biosystems).

### Staining Procedure

The staining protocol for PARP14 (rabbit clone 15B10-1) and the negative control reagent (NCR) was initiated. The tissue slides were incubated with Peroxide Block (Bond Polymer Refine Detection, Catalog #DS9800, Leica Biosystems) for 5 minutes followed by 3 rinses in Bond Wash Solution. The tissue slides were incubated with ISH/IHC Super Blocking (Catalog #PV6122, Leica Biosystems) for 10 minutes. The tissue slides were then incubated with the primary antibody or NCR diluted in ISH/IHC Super Blocking (Leica Biosystems) for 30 minutes followed by 3 rinses in Bond Wash Solution. The tissue slides were incubated with Post Primary (Bond Polymer Refine Detection) for 8 minutes followed by a wash for 3×2 minutes in Bond Wash Solution. The tissue slides were incubated with Polymer (Bond Polymer Refine Detection) for 8 minutes followed by a wash for 3×2 minutes in Bond Wash Solution and then 2 rinses in distilled water. The tissue slides were rinsed with Mixed DAB Refine (Bond Polymer Refine Detection) and then incubated with Mixed DAB Refine for 10 minutes followed by 4 rinses in distilled water. Upon completion of the staining procedure, the tissue slides were counterstained online with hematoxylin (Bond Polymer Refine Detection) for 5 minutes followed by a rinse in distilled water, a rinse in Bond Wash Solution, and a final rinse in distilled water.

### Post-Processing

The tissue slides were removed from the autostainer and dehydrated and cleared offline in accordance with standard practices. Coverslip mounting was performed using an automated tape coverslipper (Sakura Finetek, Torrance, Calif.) or glass coverslipper (Leica).

### Photomicrographs

H&E and PARP14 (rabbit clone 15B10-1) stained slides were scanned using an Aperio AT Turbo or ScanScope CS system (Aperio, Vista, Calif.) to produce whole slide images.

### Atopic Dermatitis Testing

PARP14 (rabbit clone 15B10-1) IHC staining was performed on 12 human atopic dermatitis tissues obtained from a commercial source. Tissues were evaluated by pathologist review. One (1) human atopic dermatitis tissue (M L2103082) was unevaluable due to tissue fall off. Positive staining is rated on a scale of 1+ to 3+, with 3 being the most intense. Endothelial staining was observed as 1+(cytoplasmic) for 3 human atopic dermatitis tissues and 1+(membrane and cytoplasmic) for 3 human atopic dermatitis tissue and 1+(membrane, cytoplasmic, and nuclear) for 1 human atopic dermatitis tissue. Smooth muscle staining was not observed in any of the evaluable human atopic dermatitis tissues. Fibroblast staining was observed the cytoplasm of 2 human atopic dermatitis tissues (1+) and both cytoplasmic and membrane staining was observed in 7 human atopic dermatitis tissues (2+). Stromal staining was not observed in any of the evaluable human atopic dermatitis tissues. Inflammatory cell staining was observed as 3+(cytoplasmic and membranous) for 7 human atopic dermatitis tissue samples or 2+(cytoplasmic and membranous) for 3 human atopic dermatitis tissue samples. Nerve staining was not observed in any of the evaluable human atopic dermatitis tissues.

### Psoriasis Testing

PARP14 (rabbit clone 15B10-1) IHC staining was performed on 12 human psoriasis tissues obtained from a commercial source. Tissues were evaluated by pathologist review. Three (3) human psoriasis tissues were unevaluable for inflammatory regions due to tissue fall off. Positive staining is rated on a scale of 1+ to 3+, with 3 being the most intense. Endothelial staining was observed as 1+(cytoplasmic) for 1 human psoriasis tissue. Smooth muscle staining was not observed in any of the evaluable human psoriasis tissues. Fibroblast staining was observed the cytoplasm of 1 human atopic dermatitis tissues (1+). Stromal staining was not observed in any of the evaluable human atopic dermatitis tissues. Inflammatory cell staining was observed as 1+(cytoplasmic) for 7 human psoriasis tissue samples. Nerve staining was not observed in any of the evaluable human psoriasis tissues.

### Normal Skin Testing

PARP14 (rabbit clone 15B10-1) IHC staining was performed on 10 human normal skin tissues obtained from a commercial source. Tissues were evaluated by pathologist review. One (1) human normal skin tissue (ML2103143) was unevaluable due to tissue fall off Positive staining is rated on a scale of 1+ to 3+, with 3 being the most intense. Endothelial staining was not observed in any of the evaluable human normal skin tissues. Smooth muscle staining was not observed in any of the evaluable human normal skin tissues. Fibroblast staining was not observed in any of the evaluable human normal skin tissues. Stromal staining was not observed in any of the evaluable human normal skin tissues. Inflammatory cell staining was observed as 1+(cytoplasmic) for 1 human normal skin tissue sample. Nerve staining was not observed in any of the evaluable human normal skin tissues.

### Idiopathic Pulmonary Fibrosis Testing

PARP14 (rabbit clone 15B10-1) IHC staining was performed on 12 human idiopathic pulmonary fibrosis (IPF) tissues obtained from a commercial source. Tissues were evaluated by pathologist review. Positive staining is rated on a scale of 1+ to 3+, with 3 being the most intense. Endothelial staining was observed as 1+(membrane and cytoplasmic) for 4 human IPF tissues and 2+(membrane and cytoplasmic) for 8 human IPF tissues. Smooth muscle staining was not observed in any of the evaluable human IPF tissues. Fibroblast staining was not observed in any of the evaluable human IPF tissues. Stromal staining was not observed in any of the evaluable human IPF tissues. Inflammatory cell staining was observed as 1+(cytoplasmic) for 1 human IPF tissue sample or 2+(cytoplasmic and membranous) for 11 human IPF tissue samples. Nerve staining was not observed in any of the evaluable human IPF tissues.

### Normal Lung Tissue Testing

PARP14 (rabbit clone 15B10-1) IHC staining was performed on 10 human normal lung tissues obtained from a commercial source. Tissues were evaluated by pathologist review. Positive staining is rated on a scale of 1+ to 3+, with 3 being the most intense. Endothelial staining was observed as 1+(cytoplasmic and membranous) in 2 human normal lung tissues, 2+(membranous) in 5 human normal lung tissues, 2+(membranous and cytoplasmic) in 1 human normal lung tissue, and 3+(membranous and cytoplasmic) in 1 human normal lung tissue. Smooth muscle staining was not observed in any of the evaluable human normal lung tissues. Fibroblast staining was not observed in any of the evaluable human normal lung tissues. Stromal staining was not observed in any of the evaluable human normal lung tissues. Inflammatory cell staining was observed as 1+(cytoplasmic and membranous) for 1 human normal lung tissue sample and 2+(cytoplasmic and membranous) for 9 human normal lung tissue samples. Nerve staining was not observed in any of the evaluable human normal lung tissues.

### Results

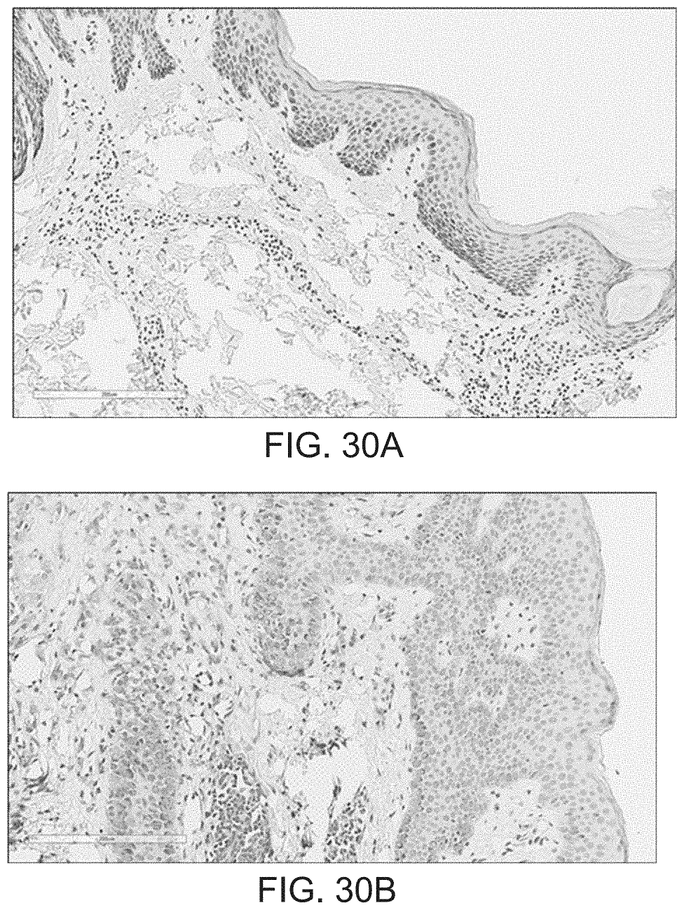

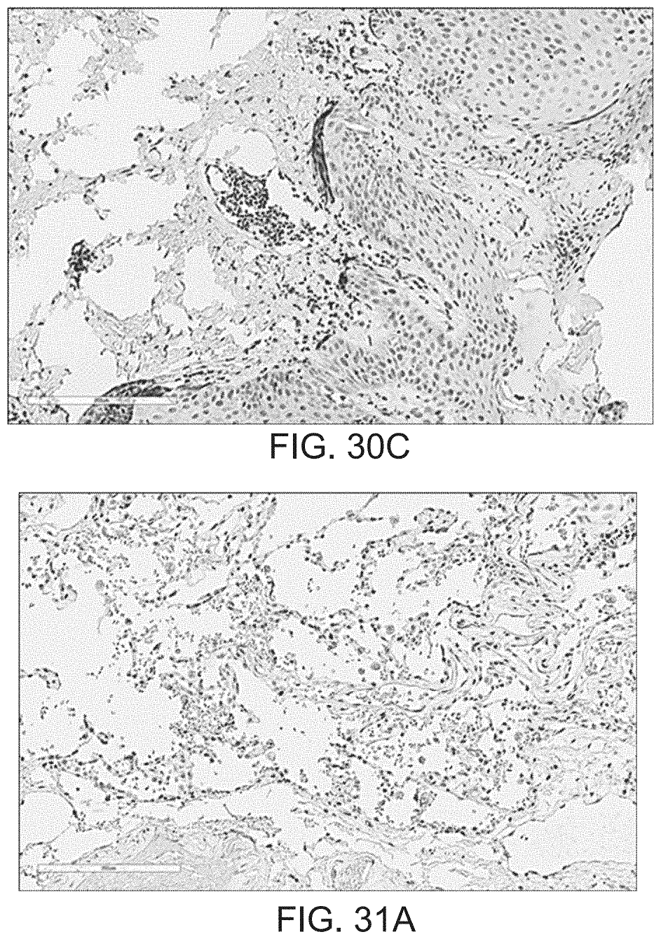

FIG. 30A is an immunohistochemistry image (20×) of normal human skin.

FIG. 30B is an immunohistochemistry image (20×) of skin from a patient with atopic dermatitis. PARP14 was visualized using DAB (brown chromogen) with a hematoxalin counterstain.

FIG. 30C is an immunohistochemistry image (20×) of skin from a patient with psoriasis. PARP14 was visualized using DAB (brown chromogen) with a hematoxalin counterstain.

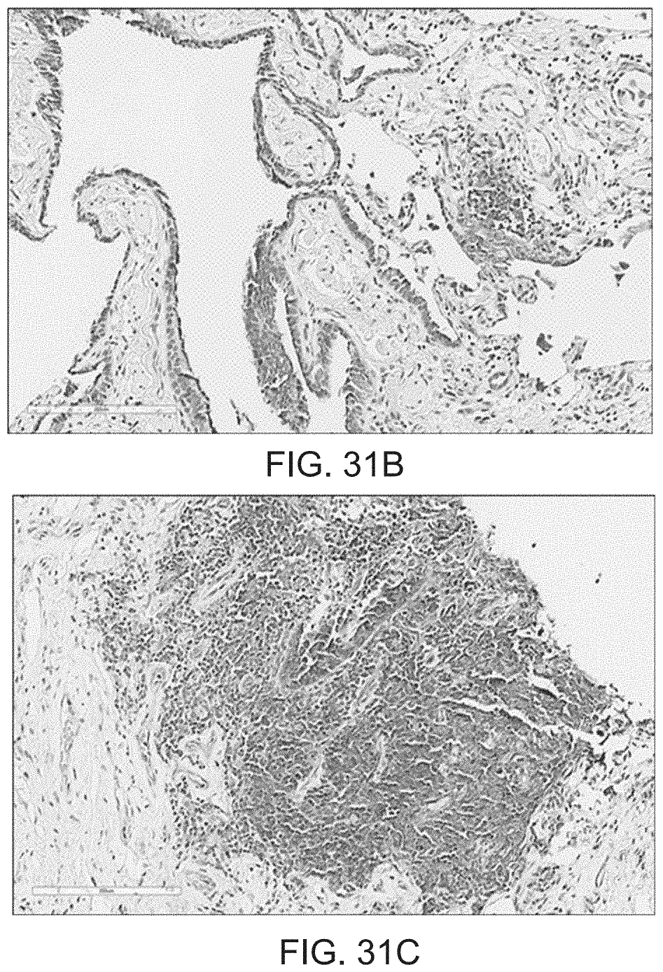

FIG. 31A is an immunohistochemistry image (20×) of normal human lung tissue.

FIG. 31B is an immunohistochemistry image (20×) of bronchial epithelial cells of lung tissue from a patient with idiopathic pulmonary fibrosis. PARP14 was visualized using DAB (brown chromogen) with a hematoxalin counterstain.

FIG. 31C is an immunohistochemistry image (20×) of inflammatory cells of lung tissue from a patient with idiopathic pulmonary fibrosis. PARP14 was visualized using DAB (brown chromogen) with a hematoxalin counterstain.

Based on the above IHC images, it was determined that compared to normal skin, PARP14 was observed to be present in inflammatory cells of atopic dermatitis and psoriasis skin samples. Additionally, compared to normal lung, PARP14 was observed to be more strongly expressed in inflammatory cells as well as bronchial epithelial cells in idiopathic pulmonary fibrosis lung samples.

### Example I: Oxazolone-Induced Colitis Model

A study was conducted to evaluate the efficacy of Compound 1 in the oxazolone (OXZ)-induced colitis model in mice.

Test Article Preparation

Compound 1 was stored at room temperature. Each formulation was prepared daily by adding an appropriate amount of Compound 1 in sesame oil, followed by vortexing and sonicated to dissolve Compound 1.

Animals

Twenty-eight female BALB/c mice (8 weeks old, 18-20 g body weight) were purchased from Beijing Vital River Laboratory Animal Technology Co., Ltd. (Joint Venture of Charles River Laboratories in China) and housed in specific pathogen-free, individually ventilated cages (5 mice in each cage) in a temperature controlled (20±2° C.) room with a 12 h light-dark cycle. Chow pellets and tap water were available ad libitun. All experimental protocols were approved by the Institutional Animal Care and Use Committee of the Wuxi AppTec. The mice were acclimated at the animal facility at Wuxi for at least three days before the experiments.

OXZ Induction for Acute Colitis in Mice

To induce colitis, mice were pre-sensitized with 200 μL of 2% OXZ (4-ethoxymethylene-2-phenyl-2-oxazolin-5-one) dissolved in a 4:1 mixture of acetone and olive oil on a 4 cm² field of the shaved back of mice (Day −5), followed by intracolonic injection of 100 μL of 1.5% oxazolone dissolved in 50% ethanol on the 5th day after pre-sensitization (Day 0). Mice in the sham-operated group were pre-sensitized with 200 μL of 4:1 mixture of acetone and olive oil on the shaved back of mice (Day −5), followed by intracolonic injection of 100 μL of 50% ethanol on Day 0. Before intracolonic perfusion of OXZ, mice were anesthetized with 0.3 mL of avertin (1.25%) and maintained in the head-down position for 2 min following intracolonic administration.

Test Groups

The mice were assigned into four groups. Four mice were assigned to the sham group, and eight mice were assigned to each of other three groups. Animals in the tofacitinib group (Group 3) were dosed twice daily (BID) with 10 mg/kg tofacitinib in 0.5% carboxymethylcellulose sodium, at a dosing volume of 10 mL/kg body weight. Animals in the vehicle group (Group 2) were given sesame oil twice daily (BID) and animals in the Compound 1 treatment group (Group 4) were given 1,000 mg/kg Compound 1 in sesame oil, twice daily (BID). All dosing was performed by oral gavage (PO) on Days −2 to Day 4 (corresponding to Day-2 and Day 4 for PK dosing, and Day-1 to Day 3 for dosing plus evaluation of efficacy endpoints). The dose regimen and groups are shown in the following table.

| | | | Treatment | Systematic dosing | |
|---|---|---|---|---|---|
| Group | N | Test articles | (mg/kg) | Route | Frequency |
| 1 | 4 | Sham | NA | NA | NA |
| 2 | 8 | Vehicle for Compound 1 | NA | PO | BID |
| 3 | 8 | Tofacitinib | 10 | PO | BID |
| 4 | 8 | Compound 1 | 1,000 | PO | BID |

OXZ = Oxazolone;
NA = Not applicable;
PO = Oral gavage;
BID = Twice daily

Evaluation of Colitis Severity

Body weight and disease activity index (DAI) scoring were recorded daily to assess the colitis. The DAI score was calculated as the sum of the weight loss score, stool score and bleeding score. A blinded scoring system was employed to assess the colitis. The DAI scorer was blinded to the group information and animal ID and was responsible for the stool consistency and bleeding scoring (blood in stool) evaluations. DAI scoring standards are described in the below table.

| Score | Weight Loss | Stool Consistency | Blood Score |
|---|---|---|---|
| 0 | no loss | Normal | Occult blood negative |
| 1 | 1~5% | Soft but still formed | Occult blood weak |
| 2 | 6~10% | Very soft | Occult blood positive |
| 3 | 11~20% | Diarrhea | Bleeding |
| 4 | >20% | Watery diarrhea | Bleeding heavily |

Fecal Occult Blood Test

If there was no blood visible with naked eyes, the fecal occult blood (FOB) test was performed with a fecal occult blood test strip using the improved pyrimidin method. The FOB score (0-2) was interpreted as follows: 0, no color appeared after 2 minutes; 1; dim color appeared in 1-2 minutes; 2, a deep color appeared in 1-2 minutes.

Colon Collection and Colon Density Measurement

The colons were harvested and mesentery and adipose tissue were carefully removed. The colons were arranged on the ruled paper for photography. After that, the length of colon was measured, and then colon weight was measured by removing and rinsing out the internal content of colon with cold PBS.

Colon Tissue Harvest for Histological Study

At the end of the study, the whole colon was harvested from mice, Swiss-rolled and fixed in 10% buffered formalin for Hematoxylin and Eosin (H&E) staining and pathological scoring by pathologists.

Blood Collection

Whole blood was collected by cardiac puncture, and left to stand for about 30 minutes at room temperature to allow coagulation. After that, the blood was centrifuged at 8,000 rpm for 10 minutes at 4° C. to separate out the serum, and the serum was frozen at −80° C. and reserved for the subsequent analysis.

Pharmacokinetic Plasma Collection

On Day −2 (two days before OXZ intracolonic injection) and Day 4 (endpoint), the mice were bled for assessment of Compound 1 concentrations in plasma. At 2 h and 11.5 h post dosing, blood was collected to determine the plasma concentration levels of Compound 1.

Histopathological Scoring

The fixed colon was evaluated by pathologists. H&E staining was reviewed and scored blinded to animal grouping and animal ID. The pathology scores are described in the below table.

| Colon Structure | Score |
|---|---|
| crypt architecture | normal, 0 |
| | slight crypt distortion with loss of entire crypts, 1 |
| | crypt distortion with loss of entire crypts, 2 |
| | severe crypt distortion with loss of entire crypts, 3 |
| inflammatory cell | normal, 0 |
| infiltration | slight inflammatory infiltrate, 1 |
| | inflammatory infiltrate, 2 |
| | dense inflammatory infiltrate, 3 |
| muscle thickening | base of crypt sits on the muscularis mucosae, 0 |
| | slight muscle thickening present, 1 |
| | muscle thickening present, 2 |
| | marked muscle thickening present, 3 |
| goblet cell depletion | absent, 0 |
| | present, 1 |
| crypt abscess | absent, 0 |
| | present, 1 |

Statistical Analysis

The data between groups was compared using an ANOVA test. If positive, individual groups were evaluated with a Dunnett's test or t test against the vehicle control group (Group 2) using Graph Pad Prism 6.0 software (San Diego, Calif., USA). P value of P<0.05 was considered as statistically significant difference. Data is shown as mean±standard error of the mean (S.E.M).

Results

A bioanalytical method for the detection of Compound 1 in Balb/c mouse plasma (with a lower limit of quantification [LLOQ] of 2 ng/ml) was used. On Day −2 and Day 4, Compound 1 plasma concentrations were highest at 2 hours post-dose and lowest at 11.5 hours post-dose.

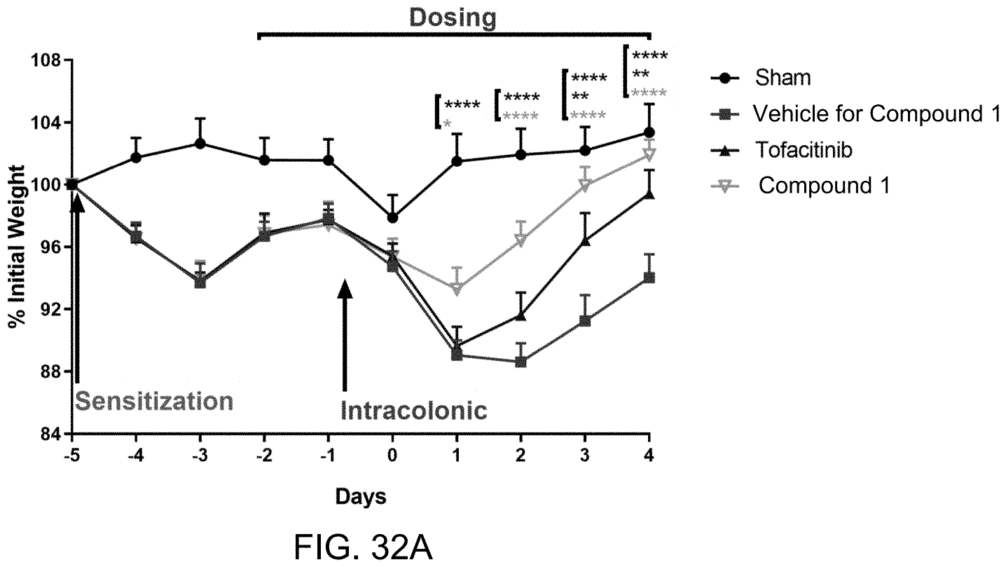

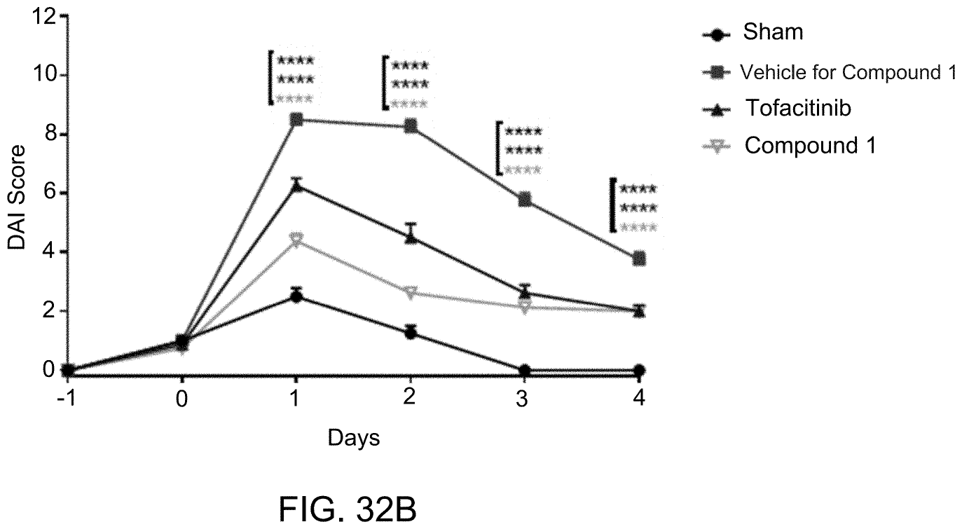

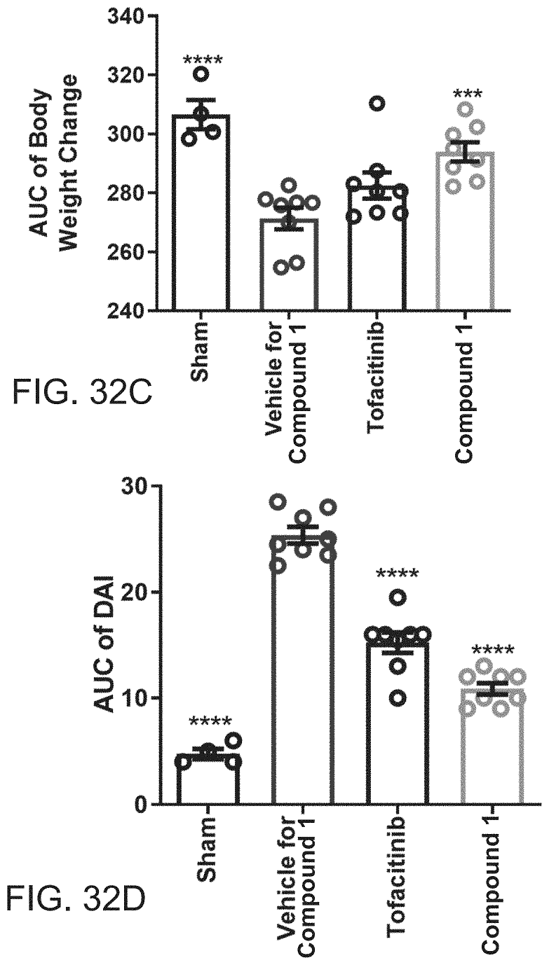

FIG. 32A shows the in-life body weight loss scoring of disease severity in the OXZ-induced colitis mouse model.

FIG. 32B shows the in-life DAI scoring of disease severity in the OXZ-induced colitis mouse model.

FIG. 32C shows the in-life AUC of body weight change scoring of disease severity in the OXZ-induced colitis mouse model.

FIG. 32D shows the in-life AUC of DAI scoring of disease severity in the OXZ-induced colitis mouse model.

Compound 1 was assessed for attenuation of the disease severity in an OXZ-induced model of colitis. In comparison to the sham group (Group 1), animals in the vehicle group (Group 2) showed decreased bodyweight and an increased DAI score following intracolonic administration of OXZ on Day 0 (FIG. 32A-32D). Mice in the Compound 1 treated group (Group 4) showed significantly less loss of body weight when compared with mice in the vehicle group (FIG. 32A). The DAI score of the Compound 1 treated mice was also improved in comparison to the vehicle treated mice (FIG. 32B) as well as the are under curve (AUC) of the DAI score of Compound 1 treated mice (FIG. 32D). As comparator, mice in Group 3 were treated with tofacitinib which also suppressed body weight loss (FIG. 32A), the DAI score (FIG. 32B) and AUC of the DAI score (FIG. 32D) in comparison to the vehicle treated control group. There was significant difference between Compound 1 and the vehicle group in AUC of body weight change, but a statistical difference was not seen between tofacitinib, a comparator, and vehicle group in AUC of body weight change (FIG. 32C). These findings show that Compound 1 can alleviate OXZ-induced colitis severity by reducing the body weight loss and improving the DAI score.

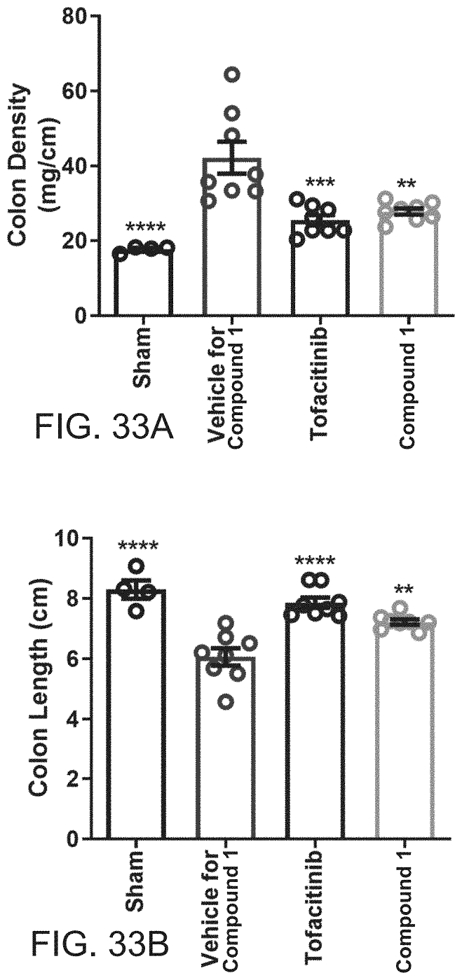

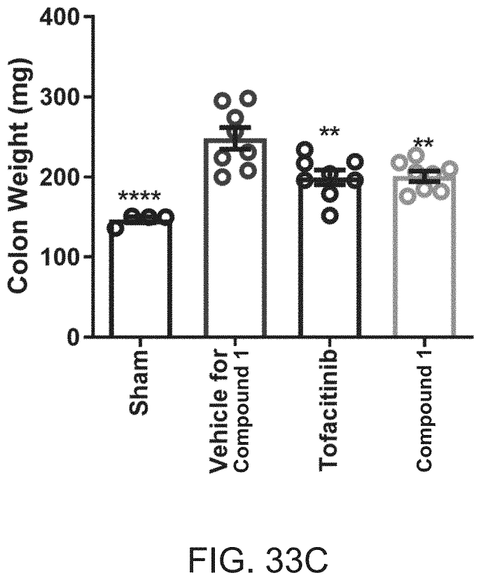

FIG. 33A shows a graph of the colon density in the OXZ-induced colitis mouse model.

FIG. 33B shows a graph of the colon length in the OXZ-induced colitis mouse model.

FIG. 33C shows a graph of the colon weight in the OXZ-induced colitis mouse model.

In comparison to the sham group, the intracolonic administration of OXZ into mice in the vehicle group caused a shortening of the colon, and increased colon weight and density, which can be used to indicate the severity of colitis (FIG. 33A-33C). The animals in the group treated with Compound 1 (Group 4) showed a suppression of the increased colon density induced by OXZ treatment (FIG. 33A). Compound 1 also suppressed the reduced colon length (FIG. 33B) and increased colon weight (FIG. 33C) seen in comparison to the vehicle control group. As a comparator, mice in Group 3 were treated with tofacitinib which also suppressed the increased colon density, decreased colon length, and increased colon weight (FIG. 33A-C). These findings show that Compound 1 can alleviate colitis disease severity by suppressing increases in colon weight and density and decreases in colon length induced by OXZ treatment.

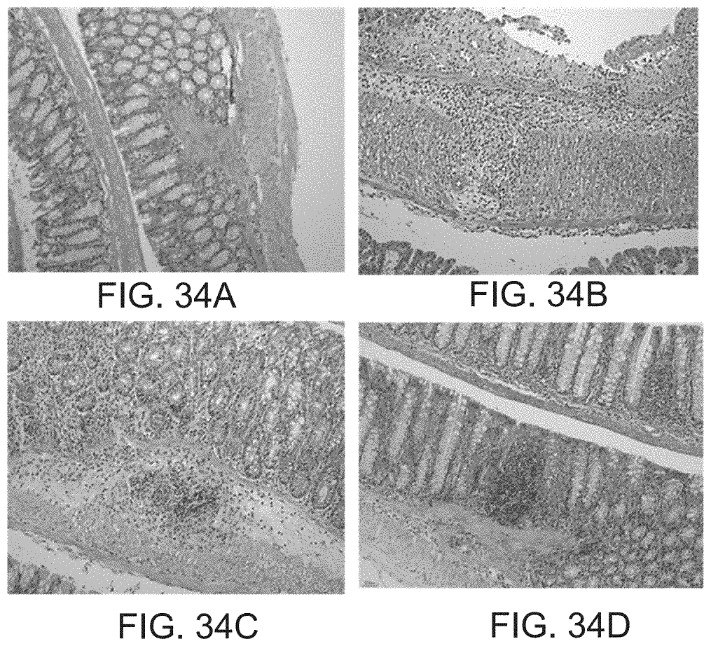

FIG. 34A shows the H&E (hematoxylin and eosin) staining photo at 200× magnification of a sample from the sham group in the OXZ-induced colitis mouse model.

FIG. 34B shows the H&E staining photo at 200× magnification of a sample from the vehicle group in the OXZ-induced colitis mouse model.

FIG. 34C shows the H&E staining photo at 200× magnification of a sample from the tofacitinib group in the OXZ-induced colitis mouse model.

FIG. 34D shows the H&E staining photo at 200× magnification of a sample from the Compound 1 group in the OXZ-induced colitis mouse model.

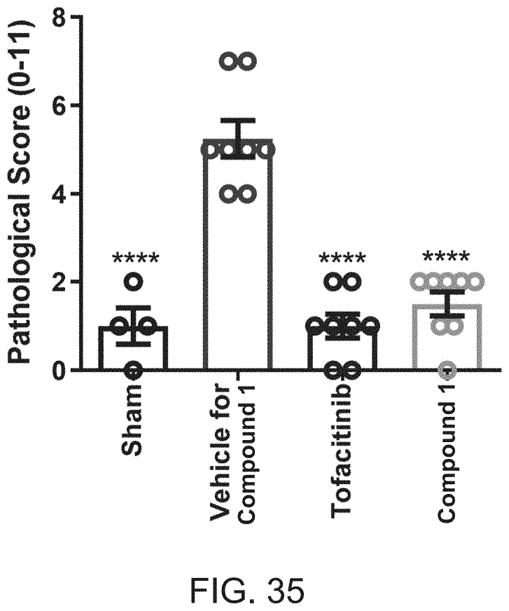

FIG. 35 shows the pathological score of the severity of colitis in the OXZ-induced colitis mouse model.

Treatment with OXZ triggers broad pathological changes in the colon histology (FIG. 34A-34D) of the vehicle treated mice that are quantified by the pathological score (FIG. 35). Compared to the vehicle group, the colon of mice in the groups treated with Compound 1 or with tofacitinib, a comparator, appeared similar to the sham group (FIG. 34A-34D). Both groups demonstrated increased integrity of the crypt structure, less inflammatory cell infiltration, reduced muscle thickening and less crypt abscess in comparison to the vehicle control group (FIG. 35). Thus, Compound 1 suppressed the severity of histological changes in the OXZ-induced colitis model.

Conclusion

Treatment with OXZ induced weight loss, increased colon density (decreased length and increased weight), and led to elevated DAI scores, and grossly altered colon histology (crypt structure, inflammatory cell infiltration, muscle thickening and crypt abscess). Orally administered Compound 1 dosed twice daily at 1,000 mg/kg suppressed the AUC of the DAI score by 70.3% (P<0.0001). Orally administered Compound 1 dosed twice daily at 1,000 mg/kg increased the AUC of the body weight change by 64.7% (P<0.001). Compound 1 also suppressed OXZ-induced changes in the gross morphology of the colon and colon histopathology. Taken together, these data demonstrate efficacy of Compound 1 in this model.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A method of treating or preventing an inflammatory disease in a patient comprising administering to the patient a therapeutically effective amount of a compound of Formula I: 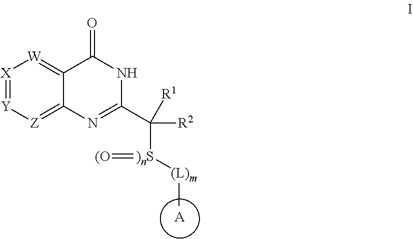
or a pharmaceutically acceptable salt thereof, wherein:
W is CR^{W} or N;
X is CR^{X} or N;
Y is CR^{Y} or N;
Z is CR^{Z} or N;
wherein no more than two of W, X, Y, and Z are simultaneously N;
Ring A is monocyclic or polycyclic C₃₋₁₄ cycloalkyl or Ring A is monocyclic or polycyclic 4-18 membered heterocycloalkyl, wherein Ring A is optionally substituted by 1, 2, 3, or 4 R^{A}, and Ring A is attached to the -(L)ₘ- moiety of Formula I through a non-aromatic ring when Ring A is polycyclic;
L is —(CR⁵R⁶)ₜ—, —(CR⁵R⁶)ₚ—O—(CR⁵R⁶)_{q}—, —(CR⁵R⁶)ₚ—S—(CR⁵R⁶)_{q}—, —(CR⁵R⁶)ₚ—NR³—(CR⁵R⁶)_{q}—, —(CR⁵R⁶)ₚ—CO—(CR⁵R⁶)_{q}—, —(CR⁵R⁶)ᵣ—C(O)O—(CR⁵R⁶)ₛ—, —(CR⁵R⁶)ᵣ—CONR³—(CR⁵R⁶)ₛ—, —(CR⁵R⁶)ₚ—SO—(CR⁵R⁶)_{q}—, —(CR⁵R⁶)ₚ—SO₂—(CR⁵R⁶)_{q}—, —(CR⁵R⁶)ᵣ—SONR³—(CR⁵R⁶)ₛ—, or —NR³CONR⁴—;
R¹ and R² are each, independently, selected from H and methyl;
R³ and R⁴ are each, independently, selected from H and C₁₋₄ alkyl;
R⁵ and R⁶ are each, independently, selected from H, halo, C₁₋₄ alkyl, C₁₋₄ alkoxy, C₁₋₄ haloalkyl, amino, C₁₋₄ alkylamino, and C₂₋₈ dialkylamino;
each R^{A} is independently selected from halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1} NR^{c1}C(O)R^{b1} NR^{c1}C(O)OR^{a1}, NR^{c1}C(O)NR^{c1}R^{d1}, C(═NR^{e1})R^{b1}, C(═NR^{e1})NR^{c1}R^{a1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{A} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
R^{W}, R^{X}, R^{Y}, and R^{Z} are each, independently, selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{e2}R^{d2}, C(O)OR^{a2}, OC(O)R^{b2}, OC(O)NR^{c2}R^{d2}, NR^{c2}R^{d2}, NR^{c2}C(O)R^{b2}, NR²C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{W}, R^{X}, R^{Y}, or R^{Z} are each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
wherein when W is CR^{W}, X is CR^{X}, Y is CR^{Y}, and Z is CR^{Z}, then at least one of R^{W}, R^{X}, R^{Y}, and R^{Z} is other than H;
each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR²C(═NR^{e2})NR^{c2}R^{d2}, NR^{c2}R^{d2}, NR²C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each R^{a1}, R^{b1}, R, R^{d1}, R^{a2}, R^{b2}, R², and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a1}, R^{b1}, R^{c1}, R^{d1}, R^{a2}, R^{b2}, R^{c2}, or R^{d2} is optionally substituted with 1, 2, 3, 4, or 5 substituents independently selected from Cy³, Cy³-C₁₋₄ alkyl, halo, C₁₋₄ alkyl, C₁₋₄ haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
each Cy³ is C₆₋₁₀ aryl, C₃₋₇ cycloalkyl, 5-10 membered heteroaryl, or 4-10 membered heterocycloalkyl, each optionally substituted by 1, 2, 3, or 4 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
R^{a3}, R^{b3}, R^{c3}, and R^{d3} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
or R^{c1} and R^{d1} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
or R^{c2} and R^{d2} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
each R^{e1}, R^{e2}, and R^{e3} is independently selected from H, C₁₋₄ alkyl, and CN;
m is 0 or 1,
n is 0, 1, or 2;
p is 0, 1, or 2;
q is 0, 1, or 2, wherein p+q is 0, 1, or 2;
r is 0 or 1;
s is 0 or 1, where r+s is 0 or 1; and
t is 1, 2, or 3;
wherein any aforementioned heteroaryl or heterocycloalkyl group comprises 1, 2, 3, or 4 ring-forming heteroatoms independently selected from O, N, and S;
wherein one or more ring-forming C or N atoms of any aforementioned heterocycloalkyl group is optionally substituted by an oxo (═O) group; and
wherein one or more ring-forming S atoms of any aforementioned heterocycloalkyl group is optionally substituted by one or two oxo (═O) groups.

2. A method of treating or preventing asthma in a patient comprising administering to the patient a therapeutically effective amount of a compound of Formula I: 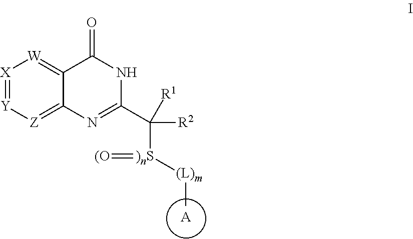
or a pharmaceutically acceptable salt thereof, wherein:
W is CR^{W} or N;
X is CR^{X} or N;
Y is CR^{Y} or N;
Z is CR^{Z} or N;
wherein no more than two of W, X, Y, and Z are simultaneously N;
Ring A is monocyclic or polycyclic C₃₋₁₄ cycloalkyl or Ring A is monocyclic or polycyclic 4-18 membered heterocycloalkyl, wherein Ring A is optionally substituted by 1, 2, 3, or 4 R^{A}, and Ring A is attached to the -(L)ₘ- moiety of Formula I through a non-aromatic ring when Ring A is polycyclic;
L is —(CR⁵R⁶)ₜ—, —(CR⁵R⁶)ₚ—O—(CR⁵R⁶)_{q}—, —(CR⁵R⁶)ₚ—S—(CR⁵R⁶)_{q}—, —(CR⁵R⁶)ₚ—NR³—(CR⁵R⁶)_{q}—, —(CR⁵R⁶)ₚ—CO—(CR⁵R⁶)_{q}—, —(CR⁵R⁶)ᵣ—C(O)O—(CR⁵R⁶)ₛ—, —(CR⁵R⁶)ᵣ—CONR³—(CR⁵R⁶)ₛ—, —(CR⁵R⁶)ₚ—SO—(CR⁵R⁶)_{q}—, —(CR⁵R⁶)ₚ—SO₂—(CR⁵R⁶)_{q}—, —(CR⁵R⁶)ᵣ—SONR³—(CR⁵R⁶)ₛ—, or —NR³CONR⁴—;
R¹ and R² are each, independently, selected from H and methyl;
R³ and R⁴ are each, independently, selected from H and C₁₋₄ alkyl;
R⁵ and R⁶ are each, independently, selected from H, halo, C₁₋₄ alkyl, C₁₋₄ alkoxy, C₁₋₄ haloalkyl, amino, C₁₋₄ alkylamino, and C₂₋₈ dialkylamino;
each R^{A} is independently selected from halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{A} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
R^{W}, R^{X}, R^{Y}, and R^{Z} are each, independently, selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR²C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃-7 cycloalkyl-C₁₋₄ alkyl, 5-10 membered heteroaryl-C₁₋₄ alkyl, and 4-10 membered heterocycloalkyl-C₁₋₄ alkyl of R^{W}, R^{X}, R^{Y}, or R^{Z} are each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR²C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR²S(O)R^{b2}, NR²S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
wherein when W is CR^{W}, X is CR^{X}, Y is CR^{Y}, and Z is CR^{Z}, then at least one of R^{W}, R^{X}, R^{Y}, and R^{Z} is other than H;
each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR²S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{e2}R^{d2};
each R^{a1}, R^{b1}, R^{c1}, R^{d1}, R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a1}, R^{b1}, R^{c1}, R^{d1}, R^{a2}, R^{b2}, R^{c2}, or R^{d2} is optionally substituted with 1, 2, 3, 4, or 5 substituents independently selected from Cy³, Cy³-C₁₋₄ alkyl, halo, C₁₋₄ alkyl, C₁₋₄ haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
each Cy³ is C₆₋₁₀ aryl, C₃₋₇ cycloalkyl, 5-10 membered heteroaryl, or 4-10 membered heterocycloalkyl, each optionally substituted by 1, 2, 3, or 4 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
R^{a3}, R^{b3}, R^{c3}, and R^{d3} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
or R^{c1} and R^{d1} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
or R^{c2} and R^{d2} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
each R^{c1}, R^{e2}, and R^{e3} is independently selected from H, C₁₋₄ alkyl, and CN;
m is 0 or 1,
n is 0, 1, or 2;
p is 0, 1, or 2;
q is 0, 1, or 2, wherein p+q is 0, 1, or 2;
r is 0 or 1;
s is 0 or 1, where r+s is 0 or 1; and
t is 1, 2, or 3;
wherein any aforementioned heteroaryl or heterocycloalkyl group comprises 1, 2, 3, or 4 ring-forming heteroatoms independently selected from O, N, and S;
wherein one or more ring-forming C or N atoms of any aforementioned heterocycloalkyl group is optionally substituted by an oxo (═O) group; and
wherein one or more ring-forming S atoms of any aforementioned heterocycloalkyl group is optionally substituted by one or two oxo (═O) groups.

3. The method of claim 1, wherein W is CR^{W}; X is CR^{X}; Y is CR^{Y}; and Z is CR^{Z}.

4. The method of claim 1, wherein W is N; X is CR^{X}; Y is CR^{Y}; and Z is CR^{Z}.

5. The method of claim 1, wherein W is CR^{W}; X is N; Y is CR^{Y}; and Z is CR^{Z}.

6. The method of claim 1, wherein W is CR^{W}; X is CR^{X}; Y is N; and Z is CR^{Z}.

7. The method of claim 1, wherein W is CR^{W}; X is CR^{X}; Y is CR^{Y}; and Z is N.

8. The method of claim 1, wherein Ring A is monocyclic or polycyclic C₃₋₁₄ cycloalkyl optionally substituted by 1, 2, 3, or 4 R^{A}, wherein Ring A is attached to the -(L)ₘ- moiety of Formula I through a non-aromatic ring when Ring A is polycyclic.

9. The method of claim 1, wherein Ring A is monocyclic C₃₋₇ cycloalkyl optionally substituted by 1, 2, 3, or 4 R^{A}.

10. The method of claim 1, wherein Ring A is cyclobutyl, cyclopentyl, cyclohexyl, or cycloheptyl optionally substituted by 1, 2, 3, or 4 R^{A}.

11. The method of claim 1, wherein Ring A is cyclohexyl optionally substituted by 1, 2, 3, or 4 R^{A}.

12. The method of claim 1, wherein Ring A is monocyclic or polycyclic 4-18 membered heterocycloalkyl optionally substituted by 1, 2, 3, or 4 R^{A}, and wherein Ring A is attached to the -(L)ₘ- moiety of Formula I through a non-aromatic ring when Ring A is polycyclic.

13. The method of claim 1, wherein Ring A is monocyclic 4-7 membered heterocycloalkyl optionally substituted by 1, 2, 3, or 4 R^{A}.

14. The method of claim 1, wherein Ring A is oxetanyl, tetrahydropyranyl, oxepanyl, azetidinyl, pyrrolidinyl, piperidinyl, azepanyl, or tetrahydrothiopyranyl optionally substituted by 1, 2, 3, or 4 R^{A}.

15. The method of claim 1, wherein Ring A is oxetanyl, tetrahydropyranyl, oxepanyl, azetidinyl, pyrrolidinyl, piperidinyl, or azepanyl, optionally substituted by 1, 2, 3, or 4 R^{A}.

16. The method of claim 1, wherein Ring A is piperidinyl optionally substituted by 1, 2, 3, or 4 R^{A}.

17. The method of claim 1, wherein Ring A is piperidin-4-yl optionally substituted by 1, 2, 3, or 4 R^{A}.

18. The method of claim 1, wherein Ring A is tetrahydropyranyl optionally substituted by 1, 2, 3, or 4 R^{A}.

19. The method of claim 1, wherein Ring A is tetrahydropyran-4-yl optionally substituted by 1, 2, 3, or 4 R^{A}.

20. The method of claim 1, wherein L is —(CR⁵R⁶)ₜ—.

21. The method of claim 1, wherein L is —(CR⁵R⁶)ₜ— and t is 1.

22. The method of claim 1, wherein L is —CH₂—.

23. The method of claim 1, wherein m is 0.

24. The method of claim 1, wherein m is 1.

25. The method of claim 1, wherein n is 0.

26. The method of claim 1, wherein R¹ and R² are both H.

27. The method of claim 1, wherein R¹ is methyl and R² is H.

28. The method of claim 1, wherein each R^{A} is independently selected from C₁₋₆ alkyl, halo, C₁₋₆ haloalkyl, OR^{a1}, C(O)R^{b1}, C(O)NR^{c1}R^{d1}, C(O)OR^{a1}, NR^{c1}R^{d1}, S(O)₂R^{b1}, 4-10 membered heterocycloalkyl, 5-10 membered heteroaryl, 4-10 membered heterocycloalkyl-C₁₋₄ alkyl and 5-10 membered heteroaryl-C₁₋₄ alkyl; wherein said C₁₋₆ alkyl, C₁₋₆ haloalkyl, 4-10 membered heterocycloalkyl, 5-10 membered heteroaryl, 4-10 membered heterocycloalkyl-C₁₋₄ alkyl and 5-10 membered heteroaryl-C₁₋₄ alkyl are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

29. The method of claim 1, wherein each R^{A} is independently selected from C₁₋₆ alkyl, OR^{a1}, C(O)R^{b1} NR^{c1}R^{d1} and S(O)₂R^{b1}; wherein said C₁₋₆ alkyl is optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

30. The method of claim 1, wherein each R^{A} is independently selected from halo, C₁₋₆ alkyl, C₁₋₆ haloalkyl, C₆₋₁₀ aryl-C₁₋₄ alkyl, 5-10 membered heteroaryl-C₁₋₄ alkyl, 4-10 membered heterocycloalkyl-C₁₋₄ alkyl, CN, OR^{a1}, NR^{c1}R^{d1}, C(O)NR^{c1}R^{d1}, NR^{c1}C(O)R^{b1}, C(O)R^{b1}, C(O)OR^{a1}, and S(O)₂R^{b1}, wherein said C₁₋₆ alkyl, C₁₋₆ haloalkyl, C₆₋₁₀ aryl-C₁₋₄ alkyl, 5-10 membered heteroaryl-C₁₋₄ alkyl, and 4-10 membered heterocycloalkyl-C₁₋₄ alkyl are each optionally substituted with 1, 2, 3, 4, or 5 substituents independently selected from halo, CN, OR^{a1}, NR^{c1}R^{d1}, C(O)R^{b1}, and NR^{c1}C(O)R^{b1}.

31. The method of claim 1, wherein each R^{A} is independently selected from halo, C₁₋₆ haloalkyl, OR^{a1}, C(O)NR^{c1}R^{d1}, and C(O)OR^{a1}.

32. The method of claim 1, wherein R^{A} is OR^{a1}.

33. The method of claim 1, wherein R^{a1} is H, C₁₋₆ alkyl, or C₁₋₆ haloalkyl.

34. The method of claim 1, wherein each R^{W}, R^{X}, R^{Y}, and R^{Z} is independently selected from H, halo, C₁₋₆ alkyl, C₁₋₆ haloalkyl, 5-10 membered heteroaryl, 4-10 membered heterocycloalkyl, C₆₋₁₀ aryl-C₁₋₄ alkyl, CN, OR^{a2}, C(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, and NR^{c2}S(O)₂NR^{c2}R^{d2}; wherein said C₁₋₆ alkyl, C₁₋₆ haloalkyl, 5-10 membered heteroaryl, 4-10 membered heterocycloalkyl, and C₆₋₁₀ aryl-C₁₋₄ alkyl of R^{W}, R^{X}, R^{Y}, and R^{Z} are each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}.

35. The method of claim 1, wherein each R^{W}, R^{X}, R^{Y}, and R^{Z} is independently selected from H, halo, C₁₋₆ alkyl, C₁₋₆ haloalkyl, 5-10 membered heteroaryl, 4-10 membered heterocycloalkyl, C₆₋₁₀ aryl-C₁₋₄ alkyl, CN, OR^{a2}, C(O)NR^{c2}R^{d2}, NR^{c2}R^{d2}, and NR^{c2}C(O)R^{b2}; wherein said C₁₋₆ alkyl, C₁₋₆ haloalkyl, 5-10 membered heteroaryl, 4-10 membered heterocycloalkyl, and C₆₋₁₀ aryl-C₁₋₄ alkyl of R^{W}, R^{X}, R^{Y}, and R^{Z} are each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}.

36. The method of claim 1, wherein W is CR^{W} and R^{W} is other than H.

37. The method of claim 1, wherein W is CR^{W} and R^{W} is H.

38. The method of claim 1, wherein R^{W} is selected from C₁₋₆ alkyl, C₁₋₆ haloalkyl, halo, and OR^{a2}, wherein said C₁₋₆ alkyl and C₁₋₆ haloalkyl are each optionally substituted with OR^{a2}.

39. The method of claim 1, wherein R^{W} is selected from C₁₋₆ alkyl, C₁₋₆ haloalkyl, CN, halo, and OR^{a2}, wherein said C₁₋₆ alkyl and C₁₋₆ haloalkyl are each optionally substituted with OR^{a2}.

40. The method of claim 1, wherein R^{W} is halo.

41. The method of claim 1, wherein R^{W} is F.

42. The method of claim 1, wherein X is CR^{X} and R^{X} is other than H.

43. The method of claim 1, wherein X is CR^{X} and R^{X} is H.

44. The method of claim 1, wherein R^{X} is selected from C₁₋₆ alkyl, halo, and OR^{a2}.

45. The method of claim 1, wherein Y is CR^{Y} and R^{Y} is other than H.

46. The method of claim 1, wherein Y is CR^{Y} and R^{Y} is H.

47. The method of claim 1, wherein Y is CR^{Y} and R^{Y} is independently selected from C₁₋₆ alkyl, OR^{a2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, and NR^{c2}S(O)₂NR^{c2}R^{d2}.

48. The method of claim 1, wherein Y is CR^{Y} and R^{Y} is independently selected from C₁₋₆ alkyl, C₃₋₇ cycloalkyl-C₁₋₄ alkyl, 5-10 membered heteroaryl, 4-10 membered heterocycloalkyl, halo, CN, OR^{a2}, SR^{a2}, C(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, and NR^{c2}S(O)₂NR^{c2}R^{d2}, wherein said C₁₋₆ alkyl, C₃₋₇ cycloalkyl-C₁₋₄ alkyl, 5-10 membered heteroaryl, and 4-10 membered heterocycloalkyl of R are each optionally substituted with 1, 2, 3, 4, or 5 substituents independently selected from halo, C₁₋₆ alkyl, C₁₋₆ haloalkyl, CN, NO₂, OR^{a2}, NR^{c2}R^{d2}, and S(O)₂R^{b2}.

49. The method of claim 1, wherein Y is CR^{Y} and R^{Y} is independently selected from NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, and NR^{c2}S(O)₂NR^{c2}R^{d2}.

50. The method of claim 1, wherein Y is CR^{Y} and R^{Y} is independently selected from C₁₋₆ alkyl and OR^{a2}.

51. The method of claim 1, wherein R^{a2} is selected from H, C₁₋₆ alkyl, C₁₋₆ haloalkyl, C₆₋₁₀ aryl, C₃₋₇ cycloalkyl, 4-10 membered heterocycloalkyl, C₆₋₁₀ aryl-C₁₋₄ alkyl, C₃₋₇ cycloalkyl-C₁₋₄ alkyl, and 4-10 membered heterocycloalkyl-C₁₋₄ alkyl, wherein said C₁₋₆ alkyl, C₁₋₆ haloalkyl, C₆₋₁₀ aryl, C₃₋₇ cycloalkyl, 4-10 membered heterocycloalkyl, C₆₋₁₀ aryl-C₁₋₄ alkyl, C₃₋₇ cycloalkyl-C₁₋₄ alkyl, and 4-10 membered heterocycloalkyl-C₁₋₄ alkyl are each optionally substituted with 1, 2, 3, 4, or 5 substituents independently selected from C₁₋₄ alkyl, C₁₋₄ haloalkyl, halo, CN, OR^{a3}, C(O)R^{b3}, C(O)OR^{a3} and S(O)₂R^{b3}.

52. The method of claim 1, wherein Y is CR^{Y} and R^{Y} is independently selected from NR^{c2}R^{d2} and NR^{c2}C(O)R^{b2}.

53. The method of claim 1, wherein R^{c2} and R^{d2} are each independently selected from H, C₁₋₆ alkyl, C₁₋₆ haloalkyl, C₆₋₁₀ aryl, C₃₋₇ cycloalkyl, 4-10 membered heterocycloalkyl, C₆₋₁₀ aryl-C₁₋₄ alkyl, C₃₋₇ cycloalkyl-C₁₋₄ alkyl, and 4-10 membered heterocycloalkyl-C₁₋₄ alkyl, wherein said C₁₋₆ alkyl, C₁₋₆ haloalkyl, C₆₋₁₀ aryl, C₃₋₇ cycloalkyl, 4-10 membered heterocycloalkyl, C₆₋₁₀ aryl-C₁₋₄ alkyl, C₃₋₇ cycloalkyl-C₁₋₄ alkyl, and 4-10 membered heterocycloalkyl-C₁₋₄ alkyl are each optionally substituted with 1, 2, 3, 4, or 5 substituents independently selected from C₁₋₄ alkyl, C₁₋₄ haloalkyl, halo, CN, OR^{a3}, C(O)R^{b3}, C(O)OR^{a3} and S(O)₂R^{b3}.

54. The method of claim 1, wherein Z is CR^{Z} and R^{Z} is other than H.

55. The method of claim 1, wherein Z is CR^{Z} and R^{Z} is H.

56. The method of claim 1, wherein Z is CR^{Z} and R^{Z} is C₁₋₆ alkyl.

57. The method of claim 1, wherein Z is CR^{Z} and R^{Z} is C₁₋₆ alkyl, halo, or CN.

58. The method of claim 1, wherein the treatment comprises administering a compound having Formula II: 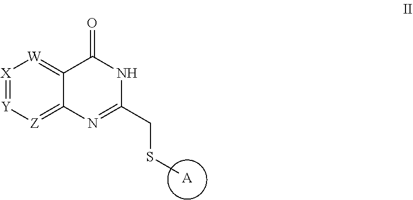
or a pharmaceutically acceptable salt thereof.

59. The method of claim 1, wherein the treatment comprises administering a compound having Formula IIIA, IIIB, IIIC, IIID, or IIIE: 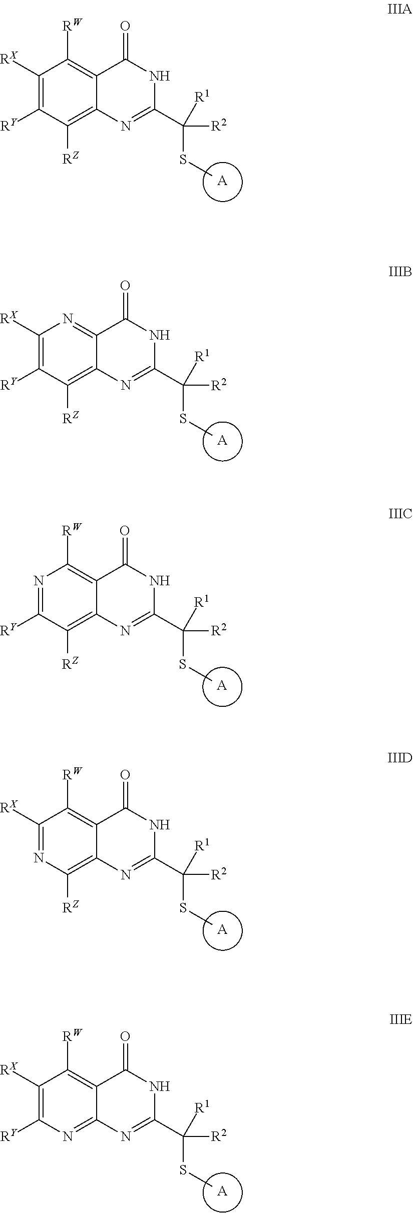
or a pharmaceutically acceptable salt thereof.

60. The method of claim 1, wherein the treatment comprises administering a compound having Formula IVA or IVB: 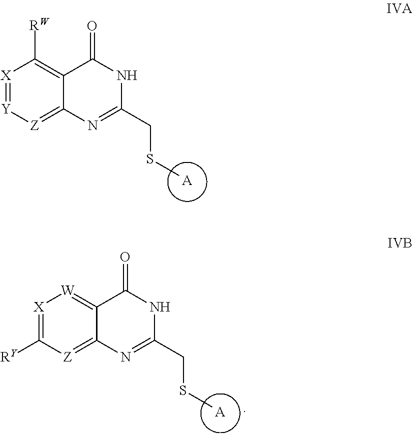
or a pharmaceutically acceptable salt thereof.

61. The method of claim 1, wherein the treatment comprises administering a compound, or a pharmaceutically acceptable salt thereof, selected from:
4-Oxo-2-(((tetrahydro-2H-pyran-4-yl)thio)methyl)-3,4-dihydroquinazoline-7-carbonitrile;
8-Methyl-2-(((tetrahydro-2H-pyran-4-yl)thio)methyl)quinazolin-4(3H)-one;
6-Methyl-2-(((tetrahydro-2H-pyran-4-yl)thio)methyl)quinazolin-4(3H)-one;
6-Methoxy-2-(((tetrahydro-2H-pyran-4-yl)thio)methyl)quinazolin-4(3H)-one;
8-Chloro-2-(((tetrahydro-2H-pyran-4-yl)thio)methyl)quinazolin-4(3H)-one;
8-Methoxy-2-(((tetrahydro-2H-pyran-4-yl)thio)methyl)quinazolin-4(3H)-one;
8-Methyl-2-(1-((tetrahydro-2H-pyran-4-yl)thio)ethyl)quinazolin-4(3H)-one;
5-Fluoro-8-methyl-2-(((tetrahydro-2H-pyran-4-yl)thio)methyl)quinazolin-4(3H)-one;
5-Methyl-2-(((tetrahydro-2H-pyran-4-yl)thio)methyl)quinazolin-4(3H)-one;
8-Benzyl-2-(((tetrahydro-2H-pyran-4-yl)thio)methyl)quinazolin-4(3H)-one;
7-Benzyl-2-(((tetrahydro-2H-pyran-4-yl)thio)methyl)quinazolin-4(3H)-one;
8-Methyl-2-((((tetrahydro-2H-pyran-4-yl)methyl)thio)methyl)quinazolin-4(3H)-one;
8-Methyl-2-((piperidin-4-ylthio)methyl)quinazolin-4(3H)-one trifluoroacetate;
8-Methyl-2-(((1-methylpiperidin-4-yl)thio)methyl)quinazolin-4(3H)-one;
8-Methyl-2-((pyrrolidin-3-ylthio)methyl)quinazolin-4(3H)-one;
8-Methyl-2-(((1-methylpyrrolidin-3-yl)thio)methyl)quinazolin-4(3H)-one;
2-(((1-Acetylpiperidin-4-yl)thio)methyl)-8-methylquinazolin-4(3H)-one;
8-Methyl-2-(((1-(pyridin-2-ylmethyl)piperidin-4-yl)thio)methyl)quinazolin-4(3H)-one;
8-Methyl-2-(((tetrahydro-2H-pyran-4-yl)sulfonyl)methyl)quinazolin-4(3H)-one;
2-((Azepan-4-ylthio)methyl)-8-methylquinazolin-4(3H)-one;
2-(((4-(Dimethylamino)cyclohexyl)thio)methyl)-8-methylquinazolin-4(3H)-one;
2-(((4-Hydroxycyclohexyl)thio)methyl)-8-methylquinazolin-4(3H)-one;
2-((((trans)-4-Hydroxycyclohexyl)thio)methyl)-8-methylquinazolin-4(3H)-one;
2-((((cis)-4-Hydroxycyclohexyl)thio)methyl)-8-methylquinazolin-4(3H)-one;
2-((Azetidin-3-ylthio)methyl)-8-methylquinazolin-4(3H)-one;
2-((((trans)-4-Methoxycyclohexyl)thio)methyl)-8-methylquinazolin-4(3H)-one;
2-((((cis)-4-Methoxycyclohexyl)thio)methyl)-8-methylquinazolin-4(3H)-one;
4-Oxo-2-(((tetrahydro-2H-pyran-4-yl)thio)methyl)-3,4-dihydroquinazoline-8-carbonitrile;
7-Phenoxy-2-(((tetrahydro-2H-pyran-4-yl)thio)methyl)quinazolin-4(3H)-one;
7-Fluoro-2-(((tetrahydro-2H-pyran-4-yl)thio)methyl)quinazolin-4(3H)-one;
7-Methoxy-2-(((tetrahydro-2H-pyran-4-yl)thio)methyl)quinazolin-4(3H)-one;
8-Methyl-2-(((1-methylpiperidin-3-yl)thio)methyl)quinazolin-4(3H)-one;
7-Fluoro-8-methyl-2-(((tetrahydro-2H-pyran-4-yl)thio)methyl)quinazolin-4(3H)-one;
5-Chloro-8-methyl-2-(((tetrahydro-2H-pyran-4-yl)thio)methyl)quinazolin-4(3H)-one;
8-Methyl-2-(((tetrahydro-2H-pyran-4-yl)thio)methyl)-5-(trifluoromethyl)quinazolin-4(3H)-one;
2-(((Tetrahydro-2H-pyran-4-yl)thio)methyl)pyrido[3,2-d]pyrimidin-4(3H)-one;
2-(((Tetrahydro-2H-pyran-4-yl)thio)methyl)pyrido[3,4-d]pyrimidin-4(3H)-one;
2-((((trans)-3-(Benzyloxy)cyclobutyl)thio)methyl)-8-methylquinazolin-4(3H)-one;
8-Methyl-2-((oxetan-3-ylthio)methyl)quinazolin-4(3H)-one;
2-(((Tetrahydro-2H-pyran-4-yl)thio)methyl)pyrido[4,3-d]pyrimidin-4(3H)-one;
8-Methyl-2-(((tetrahydro-2H-pyran-4-yl)thio)methyl)pyrido[3,2-d]pyrimidin-4(3H)-one;
8-Methyl-2-(((tetrahydro-2H-pyran-4-yl)thio)methyl)pyrido[3,4-d]pyrimidin-4(3H)-one;
2-(((Tetrahydro-2H-pyran-4-yl)thio)methyl)pyrido[2,3-d]pyrimidin-4(3H)-one;
6-Chloro-8-methyl-2-(((tetrahydro-2H-pyran-4-yl)thio)methyl)quinazolin-4(3H)-one;
7,8-Difluoro-2-(((tetrahydro-2H-pyran-4-yl)thio)methyl)quinazolin-4(3H)-one;
7-Fluoro-2-((((trans)-4-hydroxycyclohexyl)thio)methyl)quinazolin-4(3H)-one;
2-(((trans-3-Hydroxycyclobutyl)thio)methyl)-8-methylquinazolin-4(3H)-one;
8-Methyl-2-((piperidin-3-ylthio)methyl)quinazolin-4(3H)-one;
2-(((trans-4-Aminocyclohexyl)thio)methyl)-8-methylquinazolin-4(3H)-one;
2-(((cis-4-Aminocyclohexyl)thio)methyl)-8-methylquinazolin-4(3H)-one;
5-Fluoro-8-methyl-2-((piperidin-4-ylthio)methyl)quinazolin-4(3H)-one;
2-(((trans-3-Aminocyclobutyl)thio)methyl)-8-methylquinazolin-4(3H)-one;
2-(((4-Aminocycloheptyl)thio)methyl)-8-methylquinazolin-4(3H)-one;
2-(((trans-4-Aminocycloheptyl)thio)methyl)-8-methylquinazolin-4(3H)-one;
2-(((cis-4-Aminocycloheptyl)thio)methyl)-8-methylquinazolin-4(3H)-one;
5-Fluoro-2-(((4-hydroxycyclohexyl)thio)methyl)-8-methylquinazolin-4(3H)-one;
5-Fluoro-2-(((trans-4-hydroxycyclohexyl)thio)methyl)-8-methylquinazolin-4(3H)-one;
5-Fluoro-2-(((cis-4-hydroxycyclohexyl)thio)methyl)-8-methylquinazolin-4(3H)-one;
2-(((4-Hydroxycyclohexyl)thio)methyl)-8-methyl-5-(trifluoromethyl)quinazolin-4(3H)-one;
2-(((trans-4-Hydroxycyclohexyl)thio)methyl)-8-methyl-5-(trifluoromethyl) quinazolin-4(3H)-one;
2-(((cis-4-Hydroxycyclohexyl)thio)methyl)-8-methyl-5-(trifluoromethyl) quinazolin-4(3H)-one;
2-(((trans-4-(Hydroxymethyl)cyclohexyl)thio)methyl)-8-methylquinazolin-4(3H)-one;
2-(((cis-4-(Hydroxymethyl)cyclohexyl)thio)methyl)-8-methylquinazolin-4(3H)-one;
2-(((4-(Aminomethyl)cyclohexyl)thio)methyl)-8-methylquinazolin-4(3H)-one;
2-(((cis-4-(Aminomethyl)cyclohexyl)thio)methyl)-8-methylquinazolin-4(3H)-one;
2-(((trans-4-(Aminomethyl)cyclohexyl)thio)methyl)-8-methylquinazolin-4(3H)-one;
2-(((4-((Dimethylamino)methyl)cyclohexyl)thio)methyl)-8-methylquinazolin-4(3H)-one;
2-(((cis-4-((Dimethylamino)methyl)cyclohexyl)thio)methyl)-8-methyl quinazolin-4(3H)-one;
2-(((trans-4-((Dimethylamino)methyl)cyclohexyl)thio)methyl)-8-methyl quinazolin-4(3H)-one;
2-(((trans-3-(Hydroxymethyl)cyclohexyl)thio)methyl)-8-methylquinazolin-4(3H)-one;
2-(((cis-3-(Hydroxymethyl)cyclohexyl)thio)methyl)-8-methylquinazolin-4(3H)-one;
2-((((cis)-3-((Dimethylamino)methyl)cyclohexyl)thio)methyl)-8-methyl quinazolin-4(3H)-one;
8-Methyl-2-(((trans-4-((methylamino)methyl)cyclohexyl)thio)methyl) quinazolin-4(3H)-one;
7-Amino-2-(((tetrahydro-2H-pyran-4-yl)thio)methyl)quinazolin-4(3H)-one;
N-(4-Oxo-2-(((tetrahydro-2H-pyran-4-yl)thio)methyl)-3,4-dihydroquinazolin-7-yl)acetamide;
N-(4-Oxo-2-(((tetrahydro-2H-pyran-4-yl)thio)methyl)-3,4-dihydroquinazolin-7-yl)benzamide;
N-Methyl-4-oxo-2-(((tetrahydro-2H-pyran-4-yl)thio)methyl)-3,4-dihydro quinazoline-7-carboxamide;
4-Oxo-N-phenyl-2-(((tetrahydro-2H-pyran-4-yl)thio)methyl)-3,4-dihydro quinazoline-7-carboxamide;
7-(Phenylamino)-2-(((tetrahydro-2H-pyran-4-yl)thio)methyl)quinazolin-4(3H)-one;
7-(Pyridin-3-ylamino)-2-(((tetrahydro-2H-pyran-4-yl)thio)methyl)quinazolin-4(3H)-one;
7-(Pyridin-2-ylamino)-2-(((tetrahydro-2H-pyran-4-yl)thio)methyl)quinazolin-4(3H)-one;
7-((4-Methoxyphenyl)amino)-2-(((tetrahydro-2H-pyran-4-yl)thio)methyl)quinazolin-4(3H)-one;
7-((3-Methoxyphenyl)amino)-2-(((tetrahydro-2H-pyran-4-yl)thio)methyl)quinazolin-4(3H)-one;
7-((2-Methoxyphenyl)amino)-2-(((tetrahydro-2H-pyran-4-yl)thio)methyl)quinazolin-4(3H)-one;
7-(Pyrazin-2-ylamino)-2-(((tetrahydro-2H-pyran-4-yl)thio)methyl)quinazolin-4(3H)-one;
7-(Pyridin-4-ylamino)-2-(((tetrahydro-2H-pyran-4-yl)thio)methyl)quinazolin-4(3H)-one;
7-(Pyrimidin-5-ylamino)-2-(((tetrahydro-2H-pyran-4-yl)thio)methyl)quinazolin-4(3H)-one;
7-((1-Methyl-1H-imidazol-2-yl)amino)-2-(((tetrahydro-2H-pyran-4-yl)thio)methyl)quinazolin-4(3H)-one;
2-(((Tetrahydro-2H-pyran-4-yl)thio)methyl)-7-(thiazol-2-ylamino)quinazolin-4(3H)-one;
7-((2-Methylpyridin-3-yl)amino)-2-(((tetrahydro-2H-pyran-4-yl)thio)methyl)quinazolin-4(3H)-one;
7-((4-Methylpyridin-3-yl)amino)-2-(((tetrahydro-2H-pyran-4-yl)thio)methyl)quinazolin-4(3H)-one;
7-((5-Methylpyridin-3-yl)amino)-2-(((tetrahydro-2H-pyran-4-yl)thio)methyl)quinazolin-4(3H)-one;
7-(4-Amino-1H-pyrazol-1-yl)-2-(((tetrahydro-2H-pyran-4-yl)thio)methyl) quinazolin-4(3H)-one;
7-(Isoxazol-3-ylamino)-2-(((tetrahydro-2H-pyran-4-yl)thio)methyl)quinazolin-4(3H)-one;
8-Methyl-7-(phenylamino)-2-(((tetrahydro-2H-pyran-4-yl)thio)methyl) quinazolin-4(3H)-one;
7-(Benzyloxy)-2-(((tetrahydro-2H-pyran-4-yl)thio)methyl)quinazolin-4(3H)-one;
2-(((4-Hydroxycyclohexyl)thio)methyl)-7-(phenylamino)quinazolin-4(3H)-one;
2-(((trans-4-Hydroxycyclohexyl)thio)methyl)-7-(phenylamino)quinazolin-4(3H)-one;
2-(((cis-4-Hydroxycyclohexyl)thio)methyl)-7-(phenylamino)quinazolin-4(3H)-one;
2-(((cis-4-Hydroxycyclohexyl)thio)methyl)-7-(pyridin-3-ylamino)quinazolin-4(3H)-one;
2-(((trans-4-Hydroxycyclohexyl)thio)methyl)-7-(pyridin-3-ylamino)quinazolin-4(3H)-one;
7-(Cyclopentylamino)-2-(((trans-4-hydroxycyclohexyl)thio)methyl) quinazolin-4(3H)-one;
7-(Cyclopentylamino)-2-(((cis-4-hydroxycyclohexyl)thio)methyl) quinazolin-4(3H)-one;
2-(((trans-4-(Hydroxymethyl)cyclohexyl)thio)methyl)-7-(phenylamino) quinazolin-4(3H)-one;
2-(((cis-4-(Hydroxymethyl)cyclohexyl)thio)methyl)-7-(phenylamino) quinazolin-4(3H)-one;
2-(((cis-4-(Hydroxymethyl)cyclohexyl)thio)methyl)-7-(pyridin-3-ylamino) quinazolin-4(3H)-one;
2-(((trans-4-(Hydroxymethyl)cyclohexyl)thio)methyl)-7-(pyridin-3-ylamino) quinazolin-4(3H)-one;
7-(Cyclohexylamino)-2-(((tetrahydro-2H-pyran-4-yl)thio)methyl)quinazolin-4(3H)-one;
7-(Dimethylamino)-2-(((tetrahydro-2H-pyran-4-yl)thio)methyl)quinazolin-4(3H)-one;
7-(Methylamino)-2-(((tetrahydro-2H-pyran-4-yl)thio)methyl)quinazolin-4(3H)-one;
7-Morpholino-2-(((tetrahydro-2H-pyran-4-yl)thio)methyl)quinazolin-4(3H)-one;
7-(4-Methylpiperazin-1-yl)-2-(((tetrahydro-2H-pyran-4-yl)thio)methyl)quinazolin-4(3H)-one;
7-((1-Methylpiperidin-4-yl)amino)-2-(((tetrahydro-2H-pyran-4-yl)thio)methyl)quinazolin-4(3H)-one;
7-((Tetrahydro-2H-pyran-4-yl)amino)-2-(((tetrahydro-2H-pyran-4-yl)thio)methyl)quinazolin-4(3H)-one;
7-(Cyclopentylamino)-2-(((tetrahydro-2H-pyran-4-yl)thio)methyl)quinazolin-4(3H)-one;
7-(Isopropylamino)-2-(((tetrahydro-2H-pyran-4-yl)thio)methyl)quinazolin-4(3H)-one;
7-((Pyridin-4-ylmethyl)amino)-2-(((tetrahydro-2H-pyran-4-yl)thio)methyl)quinazolin-4(3H)-one;
7-((Pyridin-2-ylmethyl)amino)-2-(((tetrahydro-2H-pyran-4-yl)thio)methyl)quinazolin-4(3H)-one;
7-(Benzylamino)-2-(((tetrahydro-2H-pyran-4-yl)thio)methyl)quinazolin-4(3H)-one;
7-((1-Phenylethyl)amino)-2-(((tetrahydro-2H-pyran-4-yl)thio)methyl)quinazolin-4(3H)-one;
2-(((Tetrahydro-2H-pyran-4-yl)thio)methyl)-7-((tetrahydrofuran-3-yl)amino)quinazolin-4(3H)-one;
7-(Cyclobutylamino)-2-(((tetrahydro-2H-pyran-4-yl)thio)methyl)quinazolin-4(3H)-one;
7-((Pyridin-3-ylmethyl)amino)-2-(((tetrahydro-2H-pyran-4-yl)thio)methyl)quinazolin-4(3H)-one;
7-(Cyclopropylamino)-2-(((tetrahydro-2H-pyran-4-yl)thio)methyl)quinazolin-4(3H)-one;
7-(Cyclohexyl(methyl)amino)-2-(((tetrahydro-2H-pyran-4-yl)thio)methyl)quinazolin-4(3H)-one;
7-[(1-Benzyl-3-piperidyl)amino]-2-(tetrahydropyran-4-ylsulfanylmethyl)-3H-quinazolin-4-one;
7-(3-Piperidylamino)-2-(tetrahydropyran-4-ylsulfanylmethyl)-3H-quinazolin-4-one;
7-((1-Benzylpiperidin-4-yl)amino)-2-(((tetrahydro-2H-pyran-4-yl)thio)methyl) quinazolin-4(3H)-one;
7-(Piperidin-4-ylamino)-2-(((tetrahydro-2H-pyran-4-yl)thio)methyl)quinazolin-4(3H)-one;
7-(Pyrrolidin-3-ylamino)-2-(((tetrahydro-2H-pyran-4-yl)thio)methyl)quinazolin-4(3H)-one;
7-((1-Acetylpiperidin-4-yl)amino)-2-(((tetrahydro-2H-pyran-4-yl)thio)methyl) quinazolin-4(3H)-one;
7-((1-Acetylpiperidin-3-yl)amino)-2-(((tetrahydro-2H-pyran-4-yl)thio) methyl)quinazolin-4(3H)-one;
7-((1-Methylpiperidin-3-yl)amino)-2-(((tetrahydro-2H-pyran-4-yl)thio) methyl)quinazolin-4(3H)-one
7-((1-Acetylpyrrolidin-3-yl)amino)-2-(((tetrahydro-2H-pyran-4-yl)thio) methyl)quinazolin-4(3H)-one;
8-Methyl-7-phenoxy-2-(((tetrahydro-2H-pyran-4-yl)thio)methyl)quinazolin-4(3H)-one;
7-(Cyclohexylamino)-2-(((trans-4-(hydroxymethyl)cyclohexyl)thio)methyl)quinazolin-4(3H)-one;
7-(Cyclohexylamino)-2-(((cis-4-(hydroxymethyl)cyclohexyl)thio)methyl)quinazolin-4(3H)-one;
8-Methyl-2-(((1-((1-methyl-1H-imidazol-2-yl)methyl)piperidin-4-yl)thio) methyl)quinazolin-4(3H)-one;
N-(4-((4-(((8-Methyl-4-oxo-3,4-dihydroquinazolin-2-yl)methyl)thio)piperidin-1-yl)methyl)phenyl)acetamide;
2-(((1-(4-(Dimethylamino)benzyl)piperidin-4-yl)thio)methyl)-8-methylquinazolin-4(3H)-one;
4-((4-(((8-Methyl-4-oxo-3,4-dihydroquinazolin-2-yl)methyl)thio)piperidin-1-yl)methyl)benzonitrile;
2-(((1-((1H-Pyrazol-3-yl)methyl)piperidin-4-yl)thio)methyl)-8-methylquinazolin-4(3H)-one;
8-Methyl-2-(((1-((1-methyl-1H-indazol-3-yl)methyl)piperidin-4-yl)thio)methyl)-quinazolin-4(3H)-one;
2-(((1-((1,3-Dimethyl-1H-pyrazol-4-yl)methyl)piperidin-4-yl)thio)methyl)-8-methylquinazolin-4(3H)-one;
8-Methyl-2-(((1-((6-methylpyridin-2-yl)methyl)piperidin-4-yl)thio)methyl)quinazolin-4(3H)-one;
8-Methyl-2-(((1-((3-methylpyridin-2-yl)methyl)piperidin-4-yl)thio)methyl)quinazolin-4(3H)-one;
8-Methyl-2-(((1-phenethylpiperidin-4-yl)thio)methyl)quinazolin-4(3H)-one;
8-Methyl-2-(((1-((1-methyl-1H-indazol-6-yl)methyl)piperidin-4-yl)thio)methyl)quinazolin-4(3H)-one;
8-Methyl-2-(((1-((3-methyl-1H-pyrazol-4-yl)methyl)piperidin-4-yl)thio)methyl)quinazolin-4(3H)-one;
N-(3-((4-(((8-Methyl-4-oxo-3,4-dihydroquinazolin-2-yl)methyl)thio)piperidin-1-yl)methyl)phenyl)acetamide;
2-(((1-((1H-Pyrrolo[3,2-c]pyridin-3-yl)methyl)piperidin-4-yl)thio)methyl)-8-methylquinazolin-4(3H)-one;
2-(((1-(Imidazo[1,2-a]pyridin-3-ylmethyl)piperidin-4-yl)thio)methyl)-8-methylquinazolin-4(3H)-one;
2-(((1-((1-Benzyl-1H-imidazol-5-yl)methyl)piperidin-4-yl)thio)methyl)-8-methylquinazolin-4(3H)-one;
2-(((1-((1-Benzyl-1H-pyrazol-4-yl)methyl)piperidin-4-yl)thio)methyl)-8-methylquinazolin-4(3H)-one;
2-(2-((4-(((8-Methyl-4-oxo-3,4-dihydroquinazolin-2-yl)methyl)thio)piperidin-1-yl)methyl)phenoxy)acetonitrile;
8-Methyl-2-(((1-((2-oxoindolin-6-yl)methyl)piperidin-4-yl)thio)methyl)quinazolin-4(3H)-one;
2-(((1-((5-Methoxypyridin-2-yl)methyl)piperidin-4-yl)thio)methyl)-8-methylquinazolin-4(3H)-one;
8-Methyl-2-(((1-((4-methyl-3,4-dihydro-2H-benzo[b][1,4]oxazin-7-yl)methyl)piperidin-4-yl)thio)methyl)quinazolin-4(3H)-one;
(S)-2-(((1-(2,3-Dihydroxypropyl)piperidin-4-yl)thio)methyl)-8-methylquinazolin-4(3H)-one;
(R)-2-(((1-(2,3-Dihydroxypropyl)piperidin-4-yl)thio)methyl)-8-methylquinazolin-4(3H)-one;
(S)-8-Methyl-2-(((1-(pyrrolidin-2-ylmethyl)piperidin-4-yl)thio)methyl)quinazolin-4(3H)-one;
2-(((1-(2-Hydroxyethyl)piperidin-4-yl)thio)methyl)-8-methylquinazolin-4(3H)-one;
2-(((1-(2-Aminoethyl)piperidin-4-yl)thio)methyl)-8-methylquinazolin-4(3H)-one;
N-(2-(4-(((8-Methyl-4-oxo-3,4-dihydroquinazolin-2-yl)methyl)thio)piperidin-1-yl)ethyl)picolinamide;
2-(((1-(3-Aminopropyl)piperidin-4-yl)thio)methyl)-8-methylquinazolin-4(3H)-one;
2-(((1-Glycylpiperidin-4-yl)thio)methyl)-8-methylquinazolin-4(3H)-one;
2-(((1-(3-Aminopropanoyl)piperidin-4-yl)thio)methyl)-8-methylquinazolin-4(3H)-one;
2-(((1-(3-(Dimethylamino)propanoyl)piperidin-4-yl)thio)methyl)-8-methylquinazolin-4(3H)-one;
(R)-1-(4-Amino-5-(4-(((8-methyl-4-oxo-3,4-dihydroquinazolin-2-yl) methyl)thio)piperidin-1-yl)-5-oxopentyl)guanidine;
(S)-1-(4-Amino-5-(4-(((8-methyl-4-oxo-3,4-dihydroquinazolin-2-yl)methyl) thio)piperidin-1-yl)-5-oxopentyl)guanidine;
2-(((1-(L-Lysyl)piperidin-4-yl)thio)methyl)-8-methylquinazolin-4(3H)-one;
2-(((1-(D-Lysyl)piperidin-4-yl)thio)methyl)-8-methylquinazolin-4(3H)-one;
8-Methyl-2-(((1-(3-(pyridin-2-yl)propanoyl)piperidin-4-yl)thio)methyl)quinazolin-4(3H)-one;
8-Methyl-2-(((1-(methylsulfonyl)piperidin-4-yl)thio)methyl)quinazolin-4(3H)-one;
8-Methyl-2-(((1-(pyridin-2-ylsulfonyl)piperidin-4-yl)thio)methyl)quinazolin-4(3H)-one;
7-(Cyclopentylamino)-5-fluoro-2-((piperidin-4-ylthio)methyl)quinazolin-4(3H)-one;
7-(Cyclobutylamino)-5-fluoro-2-((piperidin-4-ylthio)methyl)quinazolin-4(3H)-one;
N-(((trans)-4-(((8-Methyl-4-oxo-3,4-dihydroquinazolin-2-yl)methyl)thio)cyclohexyl)methyl)acetamide;
7-(Cyclopentylamino)-2-((piperidin-4-ylthio)methyl)quinazolin-4(3H)-one;
7-(Cyclopentylamino)-2-((((1R,4R)-4-(hydroxymethyl)cyclohexyl)thio)-methyl)quinazolin-4(3H)-one;
2-((((trans)-4-(2-Aminoethyl)cyclohexyl)thio)methyl)-8-methylquinazolin-4(3H)-one;
2-(((3-(Aminomethyl)cyclobutyl)thio)methyl)-8-methylquinazolin-4(3H)-one;
2-((((trans)-3-(2-Aminoethyl)cyclopentyl)thio)methyl)-8-methylquinazolin-4(3H)-one 7-(Cyclopentylamino)-5-fluoro-2-(((tetrahydro-2H-pyran-4-yl)thio)methyl)quinazolin-4(3H)-one;
7-(Cyclopentylamino)-5-fluoro-2-((((1R,4R)-4-hydroxycyclohexyl)thio)methyl)-quinazolin-4(3H)-one;
7-(Cyclopentylamino)-2-(((tetrahydro-2H-pyran-4-yl)thio)methyl)pyrido[2,3-d]pyrimidin-4(3H)-one;
(S)-7-((Tetrahydro-2H-pyran-3-yl)amino)-2-(((tetrahydro-2H-pyran-4-yl)thio)methyl)quinazolin-4(3H)-one;
7-(Cyclopentylamino)-2-(((tetrahydro-2H-pyran-4-yl)thio)methyl)pyrido[3,2-d]pyrimidin-4(3H)-one;
7-(Cyclopentylamino)-2-(((tetrahydro-2H-pyran-4-yl)thio)methyl)pyrido[4,3-d]pyrimidin-4(3H)-one;
2-((Azepan-4-ylthio)methyl)-7-(cyclopentylamino)quinazolin-4(3H)-one;
2-(((3-(Aminomethyl)cyclopentyl)thio)methyl)-8-methylquinazolin-4(3H)-one;
7-((3-Methylisoxazol-5-yl)amino)-2-(((tetrahydro-2H-pyran-4-yl)thio)methyl)quinazolin-4(3H)-one;
(R)-7-((1-(Methylsulfonyl)piperidin-3-yl)amino)-2-(((tetrahydro-2H-pyran-4-yl)thio)methyl)quinazolin-4(3H)-one;
7-(Cyclobutylamino)-2-((((1R,4R)-4-hydroxycyclohexyl)thio)methyl)quinazolin-4(3H)-one;
7-((1-(Methylsulfonyl)azetidin-3-yl)amino)-2-(((tetrahydro-2H-pyran-4-yl)thio)methyl)quinazolin-4(3H)-one;
(R)-7-((1-(Methylsulfonyl)piperidin-3-yl)amino)-2-((piperidin-4-ylthio)methyl)quinazolin-4(3H)-one;
7-(Cyclopentyloxy)-2-(((tetrahydro-2H-pyran-4-yl)thio)methyl)quinazolin-4(3H)-one;
8-Methyl-2-((oxepan-4-ylthio)methyl)quinazolin-4(3H)-one;
7-(Cyclopentylamino)-2-((((1R,4R)-4-hydroxycyclohexyl)thio)methyl)-5-(trifluoromethyl)quinazolin-4(3H)-one;
7-(Cyclobutylamino)-2-((piperidin-4-ylthio)methyl)quinazolin-4(3H)-one;
(R)-7-((1-(Methylsulfonyl)piperidin-3-yl)amino)-2-(((tetrahydro-2H-pyran-4-yl)thio)methyl)pyrido[2,3-d]pyrimidin-4(3H)-one;
7-Isobutyl-2-(((tetrahydro-2H-pyran-4-yl)thio)methyl)quinazolin-4(3H)-one;
7-(Cyclopentylamino)-5-methyl-2-(((tetrahydro-2H-pyran-4-yl)thio)methyl) quinazolin-4(3H)-one;
cis-4-(((8-Methyl-4-oxo-3,4-dihydroquinazolin-2-yl)methyl)thio)cyclohexane-1-carboxamide;
trans-4-(((8-Methyl-4-oxo-3,4-dihydroquinazolin-2-yl)methyl)thio)cyclohexane-1-carboxamide;
5-Chloro-7-(cyclopentylamino)-2-((piperidin-4-ylthio)methyl)quinazolin-4(3H)-one;
7-(Cyclopentylamino)-5-methoxy-2-(((tetrahydro-2H-pyran-4-yl)thio)methyl)quinazolin-4(3H)-one;
Methyl 4-(((7-(cyclopentylamino)-4-oxo-3,4-dihydroquinazolin-2-yl)methyl)thio)piperidine-1-carboxylate;
2-((trans)-4-(((8-Methyl-4-oxo-3,4-dihydroquinazolin-2-yl)methyl)thio)cyclohexyl) acetamide;
7-(Cyclopentylamino)-5-fluoro-2-(((trans-3-fluoropiperidin-4-yl)thio)methyl)quinazolin-4(3H)-one;
7-(Cyclopentylamino)-5-fluoro-2-((((3S,4S)-3-fluoropiperidin-4-yl)thio)methyl)quinazolin-4(3H)-one;
7-(Cyclopentylamino)-5-fluoro-2-((((3R,4R)-3-fluoropiperidin-4-yl)thio)methyl)quinazolin-4(3H)-one;
7-(Cyclopentylamino)-5-fluoro-2-((((cis)-3-fluoropiperidin-4-yl)thio)methyl)quinazolin-4(3H)-one;
7-(Cyclopentylamino)-5-fluoro-2-((((3R,4S)-3-fluoropiperidin-4-yl)thio)methyl)quinazolin-4(3H)-one;
7-(Cyclopentylamino)-5-fluoro-2-((((3S,4R)-3-fluoropiperidin-4-yl)thio)methyl)quinazolin-4(3H)-one;
7-(Cyclopentylamino)-5-fluoro-2-(((1-(2-hydroxyacetyl)piperidin-4-yl)thio)methyl)quinazolin-4(3H)-one;
2-((Cyclohexylthio)methyl)-7-(cyclopentylamino)-5-fluoroquinazolin-4(3H)-one;
cis-4-(((7-(Cyclopentylamino)-5-fluoro-4-oxo-3,4-dihydroquinazolin-2-yl)methyl)thio)cyclohexane-1-carboxylic acid;
trans-4-(((7-(Cyclopentylamino)-5-fluoro-4-oxo-3,4-dihydroquinazolin-2-yl)methyl)thio)cyclohexane-1-carboxylic acid;
trans-4-(((7-(Cyclopentylamino)-5-fluoro-4-oxo-3,4-dihydroquinazolin-2-yl)methyl)thio)cyclohexane-1-carboxamide;
7-(Cyclopropylmethoxy)-2-(((tetrahydro-2H-pyran-4-yl)thio)methyl)quinazolin-4(3H)-one;
4-(((7-(Cyclopentylamino)-5-fluoro-4-oxo-3,4-dihydroquinazolin-2-yl)methyl)thio)-N,N-dimethylpiperidine-1-carboxamide;
2-(((cis-6-(Hydroxymethyl)tetrahydro-2H-pyran-3-yl)thio)methyl)-8-methylquinazolin-4(3H)-one;
7-(Cyclopentylamino)-5-fluoro-2-(((trans-3-(trifluoromethyl)piperidin-4-yl)thio)methyl)quinazolin-4(3H)-one;
7-(Cyclopentylamino)-5-fluoro-2-(((cis-4-fluoropyrrolidin-3-yl)thio)methyl)quinazolin-4(3H)-one;
7-(Cyclopentylamino)-5-(hydroxymethyl)-2-(((tetrahydro-2H-pyran-4-yl)thio)methyl)quinazolin-4(3H)-one;
7-(Cyclopentylamino)-5-(fluoromethyl)-2-(((tetrahydro-2H-pyran-4-yl)thio)methyl)quinazolin-4(3H)-one;
7-(Cyclopentylamino)-6-fluoro-2-((piperidin-4-ylthio)methyl)quinazolin-4(3H)-one;
7-(Cyclopentylamino)-5-fluoro-2-(((trans-2-(trifluoromethyl)piperidin-4-yl)thio)methyl)quinazolin-4(3H)-one;
7-(Cyclopentylamino)-5-fluoro-2-(((cis-2-(trifluoromethyl)piperidin-4-yl)thio)methyl)quinazolin-4(3H)-one;
7-(Cyclopropylmethoxy)-2-((piperidin-4-ylthio)methyl)pyrido[2,3-d]pyrimidin-4(3H)-one;
7-((Cyclobutylmethyl)amino)-6-methoxy-2-((piperidin-4-ylthio)methyl)quinazolin-4(3H)-one;
7-((2,2-Difluorocyclopentyl)amino)-5-fluoro-2-(((tetrahydro-2H-pyran-4-yl)thio)methyl)quinazolin-4(3H)-one;
7-(Cyclopentylamino)-5,6-difluoro-2-((piperidin-4-ylthio)methyl)quinazolin-4(3H)-one;
5-Fluoro-7-((trans-4-morpholinocyclohexyl)amino)-2-(((tetrahydro-2H-pyran-4-yl)thio)methyl)quinazolin-4(3H)-one;
5-Fluoro-7-((cis-4-morpholinocyclohexyl)amino)-2-(((tetrahydro-2H-pyran-4-yl)thio)methyl)quinazolin-4(3H)-one;
7-(Cyclopropylmethoxy)-5-fluoro-2-(((trans-4-hydroxycyclohexyl)thio)methyl)quinazolin-4(3H)-one;
5-Fluoro-7-((tetrahydro-2H-pyran-4-yl)methoxy)-2-(((tetrahydro-2H-pyran-4-yl)thio)methyl)quinazolin-4(3H)-one;
7-(Cyclobutylmethoxy)-5-methyl-2-(((tetrahydro-2H-pyran-4-yl)thio)methyl)quinazolin-4(3H)-one;
5-Fluoro-2-(((tetrahydro-2H-pyran-4-yl)thio)methyl)-7-((tetrahydrofuran-3-yl)methoxy)quinazolin-4(3H)-one;
(R)-5-Fluoro-2-(((tetrahydro-2H-pyran-4-yl)thio)methyl)-7-((tetrahydrofuran-3-yl)methoxy)quinazolin-4(3H)-one;
(S)-5-Fluoro-2-(((tetrahydro-2H-pyran-4-yl)thio)methyl)-7-((tetrahydrofuran-3-yl)methoxy)quinazolin-4(3H)-one;
7-(Cyclopentylamino)-5-fluoro-2-(((trans-6-fluoroazepan-4-yl)thio)methyl)quinazolin-4(3H)-one;
7-(Cyclopentylamino)-5-fluoro-2-(((cis-6-fluoroazepan-4-yl)thio)methyl)quinazolin-4(3H)-one;
2-((((cis)-6-(Aminomethyl)tetrahydro-2H-pyran-3-yl)thio)methyl)-7-(cyclopentylamino)-5-fluoroquinazolin-4(3H)-one;
2-(((trans-4-(Aminomethyl)-4-fluorocyclohexyl)thio)methyl)-7-(cyclopentylamino)-5-fluoroquinazolin-4(3H)-one;
2-(((cis-4-(Aminomethyl)-4-fluorocyclohexyl)thio)methyl)-7-(cyclopentylamino)-5-fluoroquinazolin-4(3H)-one;
6-Fluoro-7-((tetrahydro-2H-pyran-4-yl)amino)-2-(((tetrahydro-2H-pyran-4-yl)thio)methyl)quinazolin-4(3H)-one;
7-(Cyclopentylamino)-5-fluoro-2-(((1-methylpiperidin-4-yl)thio)methyl)quinazolin-4(3H)-one;
7-(Cyclohexylamino)-5-fluoro-2-(((tetrahydro-2H-pyran-4-yl)thio)methyl)quinazolin-4(3H)-one;
7-(Cyclohexylamino)-5-fluoro-2-((piperidin-4-ylthio)methyl)quinazolin-4(3H)-one;
7-(Cyclohexylamino)-5-fluoro-2-((((1r,4r)-4-hydroxycyclohexyl)thio)methyl)quinazolin-4(3H)-one;
(R)-5-Fluoro-7-((1-(methylsulfonyl)piperidin-3-yl)amino)-2-(((tetrahydro-2H-pyran-4-yl)thio)methyl)quinazolin-4(3H)-one;
7-(Cyclobutylamino)-5-fluoro-2-(((tetrahydro-2H-pyran-4-yl)thio)methyl)quinazolin-4(3H)-one;
7-((2-Cyclopentylethyl)amino)-2-(((tetrahydro-2H-pyran-4-yl)thio)methyl)quinazolin-4(3H)-one;
5-Chloro-7-(cyclopentylamino)-2-(((tetrahydro-2H-pyran-4-yl)thio)methyl)quinazolin-4(3H)-one;
7-(Cyclopentylamino)-2-(((1-(2,2,2-trifluoroethyl)piperidin-4-yl)thio)methyl)quinazolin-4(3H)-one;
7-(Cyclopentylamino)-5-fluoro-2-(((1-(oxetan-3-yl)piperidin-4-yl)thio)methyl)quinazolin-4(3H)-one;
7-((2-(Tetrahydro-2H-pyran-4-yl)ethyl)amino)-2-(((tetrahydro-2H-pyran-4-yl)thio)methyl)quinazolin-4(3H)-one;
7-(Cyclopentylamino)-5-methyl-2-((piperidin-4-ylthio)methyl)quinazolin-4(3H)-one;
7-(Cyclopentylamino)-2-(((1-(2,2-difluoroethyl)piperidin-4-yl)thio)methyl)quinazolin-4(3H)-one;
7-(Cyclopentylamino)-2-(((1-(3,3,3-trifluoropropyl)piperidin-4-yl)thio)methyl)quinazolin-4(3H)-one;
2-(((cis-6-(Hydroxymethyl)tetrahydro-2H-pyran-2-yl)thio)methyl)-8-methylquinazolin-4(3H)-one;
7-((Cyclobutylmethyl)amino)-5-fluoro-2-((piperidin-4-ylthio)methyl)quinazolin-4(3H)-one;
7-(((2,2-Difluorocyclopropyl)methyl)amino)-5-fluoro-2-((piperidin-4-ylthio)methyl)quinazolin-4(3H)-one;
7-(Cyclopentylamino)-5-fluoro-2-(((1-(2,2,2-trifluoroethyl)piperidin-4-yl)thio)methyl)quinazolin-4(3H)-one;
7-(Cyclopentylamino)-2-(((1-(2,2-difluoropropyl)piperidin-4-yl)thio)methyl)quinazolin-4(3H)-one;
7-((Cyclopropylmethyl)amino)-5-fluoro-2-((piperidin-4-ylthio)methyl)quinazolin-4(3H)-one;
7-((3,3-Difluorocyclopentyl)amino)-5-fluoro-2-((piperidin-4-ylthio)methyl)quinazolin-4(3H)-one;
2-(((trans-4-Hydroxycyclohexyl)thio)methyl)-7-(((R)-1-(methylsulfonyl)piperidin-3-yl)amino)quinazolin-4(3H)-one;
(R)-2-(((1-Acetylpiperidin-4-yl)thio)methyl)-7-((1-(methylsulfonyl)piperidin-3-yl)amino)quinazolin-4(3H)-one;
5-Fluoro-2-(((trans-4-hydroxycyclohexyl)thio)methyl)-7-(((R)-1-(methylsulfonyl)piperidin-3-yl)amino)quinazolin-4(3H)-one;
7-(Cyclopentylamino)-2-(((1,1-dioxidotetrahydro-2H-thiopyran-4-yl)thio)methyl)-5-fluoroquinazolin-4(3H)-one;
7-((Cyclopropylmethyl)amino)-5-fluoro-2-(((trans-4-hydroxycyclohexyl)thio)methyl)quinazolin-4(3H)-one;
5-Fluoro-2-((piperidin-4-ylthio)methyl)-7-(((tetrahydro-2H-pyran-4-yl)methyl)amino)quinazolin-4(3H)-one;
7-(Cyclopentylamino)-2-(((1-(1,1-dioxidothietan-3-yl)piperidin-4-yl)thio)methyl)-5-fluoroquinazolin-4(3H)-one;
7-((Cyclopropylmethyl)amino)-5-fluoro-2-(((1-(oxetan-3-yl)piperidin-4-yl)thio)methyl)quinazolin-4(3H)-one;
7-(Cyclopropylmethoxy)-2-((piperidin-4-ylthio)methyl)quinazolin-4(3H)-one;
7-(Cyclopentylamino)-5-fluoro-2-(((1-(2-(methylsulfonyl)ethyl)piperidin-4-yl)thio)methyl)quinazolin-4(3H)-one;
5-Fluoro-7-((2-morpholinoethyl)amino)-2-((piperidin-4-ylthio)methyl)quinazolin-4(3H)-one;
7-(Cyclopropylmethoxy)-5-fluoro-2-((piperidin-4-ylthio)methyl)quinazolin-4(3H)-one;
7-(Cyclopentylamino)-5-fluoro-2-(((1-(2-hydroxy-2-methylpropanoyl)piperidin-4-yl)thio)methyl)quinazolin-4(3H)-one;
7-(Cyclobutylmethoxy)-5-fluoro-2-((piperidin-4-ylthio)methyl)quinazolin-4(3H)-one;
7-(Cyclopentylamino)-5-fluoro-2-(((1-(pyridin-2-ylmethyl)piperidin-4-yl)thio)methyl)quinazolin-4(3H)-one;
7-(Cyclopentylmethoxy)-5-fluoro-2-((piperidin-4-ylthio)methyl)quinazolin-4(3H)-one;
2-(4-(((7-(Cyclopentylamino)-5-fluoro-4-oxo-3,4-dihydroquinazolin-2-yl)methyl)thio)piperidin-1-yl)-N-methylacetamide;
7-(((2,2-Difluorocyclopropyl)methyl)amino)-5-methyl-2-((piperidin-4-ylthio)methyl)quinazolin-4(3H)-one;
2-(4-(((7-(Cyclopentylamino)-5-fluoro-4-oxo-3,4-dihydroquinazolin-2-yl)methyl)thio)piperidin-1-yl)acetonitrile;
2-(trans-4-(((7-(Cyclopentylamino)-5-fluoro-4-oxo-3,4-dihydroquinazolin-2-yl)methyl)thio)cyclohexyl)acetamide;
5-Fluoro-7-((2-morpholinoethyl)amino)-2-(((tetrahydro-2H-pyran-4-yl)thio)methyl)quinazolin-4(3H)-one;
5-Fluoro-2-(((tetrahydro-2H-pyran-4-yl)thio)methyl)-7-((1-(2,2,2-trifluoroethyl)piperidin-4-yl)amino)quinazolin-4(3H)-one;
7-((Cyclobutylmethyl)amino)-6-fluoro-2-((piperidin-4-ylthio)methyl)quinazolin-4(3H)-one;
7-(Cyclohexylamino)-6-fluoro-2-(((tetrahydro-2H-pyran-4-yl)thio)methyl)quinazolin-4(3H)-one;
7-(Cyclopropylmethoxy)-5-fluoro-2-(((tetrahydro-2H-pyran-4-yl)thio)methyl)quinazolin-4(3H)-one;
7-((Cyclopropylmethyl)amino)-6-fluoro-2-(((tetrahydro-2H-pyran-4-yl)thio)methyl)quinazolin-4(3H)-one;
7-(Cyclopentylamino)-6-fluoro-2-(((trans-4-hydroxycyclohexyl)thio)methyl)quinazolin-4(3H)-one;
7-(Cyclopentylamino)-5,6-difluoro-2-(((tetrahydro-2H-pyran-4-yl)thio)methyl)quinazolin-4(3H)-one;
7-(Cyclopropylmethoxy)-5-fluoro-2-(((cis-3-fluoropiperidin-4-yl)thio)methyl)quinazolin-4(3H)-one;
7-((Cyclobutylmethyl)amino)-2-(((1,1-dioxidotetrahydro-2H-thiopyran-4-yl)thio)methyl)-6-fluoroquinazolin-4(3H)-one;
7-(Cyclopropylmethoxy)-5-fluoro-2-(((trans-3-fluoropiperidin-4-yl)thio)methyl)quinazolin-4(3H)-one;
5-Fluoro-2-(((tetrahydro-2H-pyran-4-yl)thio)methyl)-7-((1-(3,3,3-trifluoropropyl)piperidin-4-yl)methoxy)quinazolin-4(3H)-one;
7-((1-(2,2-Difluoropropyl)piperidin-4-yl)methoxy)-5-fluoro-2-(((tetrahydro-2H-pyran-4-yl)thio)methyl)quinazolin-4(3H)-one;
7-((1-(2,2-Difluoroethyl)piperidin-4-yl)methoxy)-5-fluoro-2-(((tetrahydro-2H-pyran-4-yl)thio)methyl)quinazolin-4(3H)-one;
5-Fluoro-7-((1-(oxetan-3-yl)piperidin-4-yl)methoxy)-2-(((tetrahydro-2H-pyran-4-yl)thio)methyl)quinazolin-4(3H)-one;
5-Fluoro-7-((1-(oxetan-3-yl)piperidin-4-yl)amino)-2-(((tetrahydro-2H-pyran-4-yl)thio)methyl)quinazolin-4(3H)-one;
7-((Cyclobutylmethyl)amino)-6-fluoro-2-(((cis-3-fluoropiperidin-4-yl)thio)methyl)quinazolin-4(3H)-one;
7-(Cyclobutylmethoxy)-2-(((1,1-dioxidotetrahydro-2H-thiopyran-4-yl)thio)methyl)-5-fluoroquinazolin-4(3H)-one;
5-Fluoro-7-((trans-2-fluorocyclopentyl)amino)-2-(((tetrahydro-2H-pyran-4-yl)thio)methyl)quinazolin-4(3H)-one;
5-Fluoro-7-isobutoxy-2-(((tetrahydro-2H-pyran-4-yl)thio)methyl)quinazolin-4(3H)-one;
7-(Cyclobutylmethoxy)-5-fluoro-2-(((1-(2-hydroxyacetyl)piperidin-4-yl)thio)methyl)quinazolin-4(3H)-one;
7-(Cyclobutylmethoxy)-2-(((2,2-dimethyltetrahydro-2H-pyran-4-yl)thio)methyl)-5-fluoroquinazolin-4(3H)-one;
7-(Cyclobutylmethoxy)-2-((cyclohexylthio)methyl)-5-fluoroquinazolin-4(3H)-one;
2-((Cyclohexylthio)methyl)-7-(cyclopentylamino)-5,6-difluoroquinazolin-4(3H)-one;
trans-4-(((7-(Cyclobutylmethoxy)-5-fluoro-4-oxo-3,4-dihydroquinazolin-2-yl)methyl)thio)cyclohexane-1-carboxamide;
7-((1-(2,2-Difluoroethyl)piperidin-3-yl)methoxy)-5-fluoro-2-(((tetrahydro-2H-pyran-4-yl)thio)methyl)quinazolin-4(3H)-one;
7-(Cyclopentylamino)-5,6-difluoro-2-(((trans-4-hydroxycyclohexyl)thio)methyl)quinazolin-4(3H)-one;
7-(Cyclopentylmethoxy)-5-fluoro-2-(((trans-4-hydroxycyclohexyl)thio)methyl)quinazolin-4(3H)-one;
7-((2,2-Difluorocyclopropyl)methoxy)-5-fluoro-2-(((tetrahydro-2H-pyran-4-yl)thio)methyl)quinazolin-4(3H)-one;
7-(Cyclopentylamino)-2-(((1,1-dioxidotetrahydro-2H-thiopyran-4-yl)thio)methyl)-5,6-difluoroquinazolin-4(3H)-one;
7-((3,3-Difluorocyclobutyl)methoxy)-5-fluoro-2-(((tetrahydro-2H-pyran-4-yl)thio)methyl)quinazolin-4(3H)-one;
5-Fluoro-2-(((trans-4-hydroxycyclohexyl)thio)methyl)-7-((tetrahydro-2H-pyran-3-yl)methoxy)quinazolin-4(3H)-one;
5-Fluoro-7-((tetrahydro-2H-pyran-3-yl)methoxy)-2-(((tetrahydro-2H-pyran-4-yl)thio)methyl)quinazolin-4(3H)-one;
5-Fluoro-2-(((tetrahydro-2H-pyran-4-yl)thio)methyl)-7-((tetrahydrofuran-2-yl)methoxy)quinazolin-4(3H)-one;
(R)-5-Fluoro-2-(((tetrahydro-2H-pyran-4-yl)thio)methyl)-7-((tetrahydrofuran-2-yl)methoxy)quinazolin-4(3H)-one;
(S)-5-Fluoro-2-(((tetrahydro-2H-pyran-4-yl)thio)methyl)-7-((tetrahydrofuran-2-yl)methoxy)quinazolin-4(3H)-one;
5,6-Difluoro-2-(((tetrahydro-2H-pyran-4-yl)thio)methyl)-7-(((tetrahydrofuran-3-yl)methyl)amino)quinazolin-4(3H)-one;
(S)-5,6-Difluoro-2-(((tetrahydro-2H-pyran-4-yl)thio)methyl)-7-(((tetrahydrofuran-3-yl)methyl)amino)quinazolin-4(3H)-one;
(R)-5,6-Difluoro-2-(((tetrahydro-2H-pyran-4-yl)thio)methyl)-7-(((tetrahydrofuran-3-yl)methyl)amino)quinazolin-4(3H)-one;
5-Fluoro-2-((((trans)-4-hydroxycyclohexyl)thio)methyl)-7-((tetrahydrofuran-3-yl)methoxy)quinazolin-4(3H)-one;
5-Fluoro-2-((((trans)-4-hydroxycyclohexyl)thio)methyl)-7-(((R)-tetrahydrofuran-3-yl)methoxy)quinazolin-4(3H)-one;
5-Fluoro-2-((((trans)-4-hydroxycyclohexyl)thio)methyl)-7-(((S)-tetrahydrofuran-3-yl)methoxy)quinazolin-4(3H)-one;
5-Fluoro-7-(((trans)-3-fluoro-1-methylpiperidin-4-yl)methoxy)-2-(((tetrahydro-2H-pyran-4-yl)thio)methyl)quinazolin-4(3H)-one;
5-Fluoro-7-(((3S,4S)-3-fluoro-1-methylpiperidin-4-yl)methoxy)-2-(((tetrahydro-2H-pyran-4-yl)thio)methyl)quinazolin-4(3H)-one;
5-Fluoro-7-(((3R,4R)-3-fluoro-1-methylpiperidin-4-yl)methoxy)-2-(((tetrahydro-2H-pyran-4-yl)thio)methyl)quinazolin-4(3H)-one;
5,6-Difluoro-7-(((cis)-3-methoxycyclobutyl)amino)-2-(((tetrahydro-2H-pyran-4-yl)thio)methyl)quinazolin-4(3H)-one;
N-((cis)-4-(((7-(Cyclopropylmethoxy)-5-fluoro-4-oxo-3,4-dihydroquinazolin-2-yl)methyl)thio)cyclohexyl)acetamide;
N-((trans)-4-(((7-(Cyclopropylmethoxy)-5-fluoro-4-oxo-3,4-dihydroquinazolin-2-yl)methyl)thio)cyclohexyl)acetamide;
7-(((cis)-3-Ethoxycyclobutyl)amino)-5,6-difluoro-2-(((tetrahydro-2H-pyran-4-yl)thio)methyl)quinazolin-4(3H)-one;
5-Fluoro-2-((((cis)-4-hydroxy-4-methylcyclohexyl)thio)methyl)-7-((tetrahydro-2H-pyran-4-yl)methoxy)quinazolin-4(3H)-one;
7-((1-Acetylpiperidin-4-yl)methoxy)-5-fluoro-2-(((tetrahydro-2H-pyran-4-yl)thio)methyl)quinazolin-4(3H)-one;
2-((((trans)-4-(Aminomethyl)-4-fluorocyclohexyl)thio)methyl)-7-(cyclobutylmethoxy)-5-fluoroquinazolin-4(3H)-one;
5-Fluoro-7-(((3S,4S)-3-fluoro-1-methylpiperidin-4-yl)methoxy)-2-((((trans)-4-hydroxycyclohexyl)thio)methyl)quinazolin-4(3H)-one;
5-Fluoro-7-(((3R,4R)-3-fluoro-1-methylpiperidin-4-yl)methoxy)-2-((((trans)-4-hydroxycyclohexyl)thio)methyl)quinazolin-4(3H)-one
7-((Cyclopropylmethyl)amino)-5,6-difluoro-2-(((tetrahydro-2H-pyran-4-yl)thio)methyl)quinazolin-4(3H)-one;
5,6-Difluoro-7-(((tetrahydro-2H-pyran-4-yl)methyl)amino)-2-(((tetrahydro-2H-pyran-4-yl)thio)methyl)quinazolin-4(3H)-one;
7-((Cyclobutylmethyl)amino)-2-(((1,1-dioxidotetrahydro-2H-thiopyran-4-yl)thio)methyl)-5,6-difluoroquinazolin-4(3H)-one;
5-Fluoro-7-(((trans)-2-fluorocyclopentyl)amino)-2-((((trans)-4-hydroxycyclohexyl)thio)methyl)quinazolin-4(3H)-one;
5-Fluoro-7-(((cis)-2-fluorocyclopentyl)amino)-2-((((trans)-4-hydroxycyclohexyl)thio)methyl)quinazolin-4(3H)-one;
5-Fluoro-2-((((trans)-4-hydroxycyclohexyl)thio)methyl)-7-((tetrahydro-2H-pyran-4-yl)methoxy)quinazolin-4(3H)-one;
5-Fluoro-7-(oxetan-3-ylmethoxy)-2-(((tetrahydro-2H-pyran-4-yl)thio)methyl)quinazolin-4(3H)-one;
7-((1,4-Dioxan-2-yl)methoxy)-5-fluoro-2-(((tetrahydro-2H-pyran-4-yl)thio)methyl)quinazolin-4(3H)-one;
7-((2,2-Difluorocyclohexyl)amino)-5-fluoro-2-(((tetrahydro-2H-pyran-4-yl)thio)methyl)quinazolin-4(3H)-one;
5,6-Difluoro-7-(((trans)-4-(4-methylpiperazin-1-yl)cyclohexyl)amino)-2-(((tetrahydro-2H-pyran-4-yl)thio)methyl)quinazolin-4(3H)-one;
5,6-Difluoro-7-(((cis)-4-(4-methylpiperazin-1-yl)cyclohexyl)amino)-2-(((tetrahydro-2H-pyran-4-yl)thio)methyl)quinazolin-4(3H)-one;
(R)-5,6-Difluoro-7-((tetrahydro-2H-pyran-3-yl)amino)-2-(((tetrahydro-2H-pyran-4-yl)thio)methyl)quinazolin-4(3H)-one;
7-(((R)-1-Acetylpyrrolidin-3-yl)amino)-5,6-difluoro-2-((((trans)-4-hydroxycyclohexyl)thio)methyl)quinazolin-4(3H)-one;
7-((2,2-Difluorocyclopentyl)amino)-5-fluoro-2-((((trans)-4-hydroxycyclohexyl)thio)methyl)quinazolin-4(3H)-one;
7-((1,1-Dioxidotetrahydro-2H-thiopyran-4-yl)methoxy)-5-fluoro-2-(((tetrahydro-2H-pyran-4-yl)thio)methyl)quinazolin-4(3H)-one;
5-Fluoro-7-(((trans)-3-fluoropiperidin-4-yl)methoxy)-2-(((tetrahydro-2H-pyran-4-yl)thio)methyl)quinazolin-4(3H)-one;
5-Chloro-7-((tetrahydro-2H-pyran-4-yl)methoxy)-2-(((tetrahydro-2H-pyran-4-yl)thio)methyl)quinazolin-4(3H)-one;
5,6-Difluoro-2-((((trans)-4-hydroxycyclohexyl)thio)methyl)-7-((1-(3,3,3-trifluoropropyl)piperidin-4-yl)amino)quinazolin-4(3H)-one;
7-((5,5-Dimethyltetrahydrofuran-3-yl)methoxy)-5-fluoro-2-((((trans)-4-hydroxycyclohexyl)thio)methyl)quinazolin-4(3H)-one;
5-Fluoro-2-((((trans)-4-methoxycyclohexyl)thio)methyl)-7-((tetrahydro-2H-pyran-4-yl)methoxy)quinazolin-4(3H)-one;
5-Fluoro-2-((((cis)-4-methoxycyclohexyl)thio)methyl)-7-((tetrahydro-2H-pyran-4-yl)methoxy)quinazolin-4(3H)-one;
5-Fluoro-2-(((4-methyltetrahydro-2H-pyran-4-yl)thio)methyl)-7-((tetrahydro-2H-pyran-4-yl)methoxy)quinazolin-4(3H)-one;
5-Fluoro-7-(((cis)-2-hydroxycyclopentyl)methoxy)-2-(((tetrahydro-2H-pyran-4-yl)thio)methyl)quinazolin-4(3H)-one;
(trans)-4-((5,6-Difluoro-4-oxo-2-(((tetrahydro-2H-pyran-4-yl)thio)methyl)-3,4-dihydroquinazolin-7-yl)amino)cyclohexane-1-carbonitrile;
(cis)-4-((5,6-Difluoro-4-oxo-2-(((tetrahydro-2H-pyran-4-yl)thio)methyl)-3,4-dihydroquinazolin-7-yl)amino)cyclohexane-1-carbonitrile;
5,6-Difluoro-7-(((trans)-3-methoxycyclobutyl)amino)-2-(((tetrahydro-2H-pyran-4-yl)thio)methyl)quinazolin-4(3H)-one;
5,6-Difluoro-2-((((trans)-4-hydroxycyclohexyl)thio)methyl)-7-(((cis)-3-methoxycyclobutyl)amino)quinazolin-4(3H)-one;
5-Methyl-7-((tetrahydro-2H-pyran-4-yl)methoxy)-2-(((tetrahydro-2H-pyran-4-yl)thio)methyl)quinazolin-4(3H)-one;
5-Fluoro-2-((((cis)-4-hydroxycyclohexyl)thio)methyl)-7-((tetrahydro-2H-pyran-4-yl)methoxy)quinazolin-4(3H)-one;
2-(((4,4-Difluorocyclohexyl)thio)methyl)-5-fluoro-7-((tetrahydro-2H-pyran-4-yl)methoxy)quinazolin-4(3H)-one;
7-((1-Acetylpyrrolidin-3-yl)methoxy)-5-fluoro-2-(((tetrahydro-2H-pyran-4-yl)thio)methyl)quinazolin-4(3H)-one;
7-(2-Cyclohexylethyl)-5-fluoro-2-(((tetrahydro-2H-pyran-4-yl)thio)methyl)quinazolin-4(3H)-one;
7-(((1-Acetylpiperidin-4-yl)methyl)amino)-5,6-difluoro-2-(((tetrahydro-2H-pyran-4-yl)thio)methyl)quinazolin-4(3H)-one;
5-Fluoro-7-(((tetrahydro-2H-pyran-4-yl)methyl)thio)-2-(((tetrahydro-2H-pyran-4-yl)thio)methyl)quinazolin-4(3H)-one;
5-Fluoro-7-(((cis)-4-fluoropyrrolidin-3-yl)methoxy)-2-(((tetrahydro-2H-pyran-4-yl)thio)methyl)quinazolin-4(3H)-one;
5-Fluoro-7-(((cis)-4-fluoro-1-methylpyrrolidin-3-yl)methoxy)-2-(((tetrahydro-2H-pyran-4-yl)thio)methyl)quinazolin-4(3H)-one;
5-Fluoro-2-((((cis)-4-hydroxy-4-methylcyclohexyl)thio)methyl)-7-((tetrahydrofuran-3-yl)methoxy)quinazolin-4(3H)-one;
5,6-Difluoro-2-((((cis)-4-hydroxy-4-methylcyclohexyl)thio)methyl)-7-(((cis)-3-methoxycyclobutyl)amino)quinazolin-4(3H)-one;
5-Fluoro-7-((tetrahydro-2H-pyran-4-yl)methoxy)-2-((((trans)-4-(trifluoromethoxy)cyclohexyl)thio)methyl)quinazolin-4(3H)-one;
5-Bromo-2-(((tetrahydro-2H-pyran-4-yl)thio)methyl)-7-((tetrahydrofuran-3-yl)methoxy)quinazolin-4(3H)-one;
5,6-Difluoro-2-((((trans)-4-hydroxycyclohexyl)thio)methyl)-7-(((trans)-4-methoxycyclohexyl)amino)quinazolin-4(3H)-one;
N-((trans)-4-(((7-(Cyclopropylmethoxy)-5-fluoro-4-oxo-3,4-dihydroquinazolin-2-yl)methyl)thio)cyclohexyl)propionamide;
5,6-Difluoro-2-((((trans)-4-hydroxycyclohexyl)thio)methyl)-7-(((cis)-4-methoxycyclohexyl)amino)quinazolin-4(3H)-one;
N-(4-(((7-(Cyclopropylmethoxy)-5-fluoro-4-oxo-3,4-dihydroquinazolin-2-yl)methyl)thio)-1-methylcyclohexyl)acetamide;
5,6-Difluoro-2-((((trans)-4-hydroxycyclohexyl)thio)methyl)-7-(((R)-tetrahydro-2H-pyran-3-yl)amino)quinazolin-4(3H)-one;
5-Fluoro-2-((((trans)-3-hydroxycyclobutyl)thio)methyl)-7-((tetrahydro-2H-pyran-4-yl)methoxy)quinazolin-4(3H)-one;
4-Oxo-2-(((tetrahydro-2H-pyran-4-yl)thio)methyl)-7-((tetrahydrofuran-3-yl)methoxy)-3,4-dihydroquinazoline-5-carbonitrile;
5,6-Difluoro-7-(neopentylamino)-2-(((tetrahydro-2H-pyran-4-yl)thio)methyl)quinazolin-4(3H)-one;
5-Fluoro-7-(((cis)-3-hydroxy-3-methylcyclobutyl)methoxy)-2-(((tetrahydro-2H-pyran-4-yl)thio)methyl)quinazolin-4(3H)-one;
5-Fluoro-7-(((trans)-3-hydroxy-3-methylcyclobutyl)methoxy)-2-(((tetrahydro-2H-pyran-4-yl)thio)methyl)quinazolin-4(3H)-one;
N-((cis)-3-(((5-Fluoro-4-oxo-7-((tetrahydro-2H-pyran-4-yl)methoxy)-3,4-dihydroquinazolin-2-yl)methyl)thio)cyclobutyl)acetamide;
5-Fluoro-7-(((cis)-3-fluoro-1-methylpiperidin-4-yl)methoxy)-2-(((tetrahydro-2H-pyran-4-yl)thio)methyl)quinazolin-4(3H)-one;
N-((trans)-4-(((5,6-Difluoro-7-(((cis)-3-methoxycyclobutyl)amino)-4-oxo-3,4-dihydroquinazolin-2-yl)methyl)thio)cyclohexyl)acetamide;
7-((1-(Cyclopropanecarbonyl)piperidin-4-yl)methoxy)-5-fluoro-2-(((tetrahydro-2H-pyran-4-yl)thio)methyl)quinazolin-4(3H)-one;
N-((trans)-4-(((5-Fluoro-4-oxo-7-((tetrahydrofuran-3-yl)methoxy)-3,4-dihydroquinazolin-2-yl)methyl)thio)cyclohexyl)acetamide;
N-((trans)-4-(((7-(Cyclobutylamino)-5,6-difluoro-4-oxo-3,4-dihydroquinazolin-2-yl)methyl)thio)cyclohexyl)acetamide;
N-((trans)-3-(((5-Fluoro-4-oxo-7-((tetrahydro-2H-pyran-4-yl)methoxy)-3,4-dihydroquinazolin-2-yl)methyl)thio)cyclobutyl)acetamide;
7-(1-Cyclopentylethoxy)-5-fluoro-2-(((tetrahydro-2H-pyran-4-yl)thio)methyl)-5,6,7,8-tetrahydroquinazolin-4(3H)-one;
N-((trans)-4-(((7-(Cyclopropylmethoxy)-5-fluoro-4-oxo-3,4-dihydroquinazolin-2-yl)methyl)thio)cyclohexyl)cyclopropanecarboxamide;
7-((1-Acetylpiperidin-4-yl)methoxy)-5-fluoro-2-((((trans)-4-hydroxycyclohexyl)thio)methyl)quinazolin-4(3H)-one;
5-Fluoro-7-((1-isobutyrylpiperidin-4-yl)methoxy)-2-(((tetrahydro-2H-pyran-4-yl)thio)methyl)quinazolin-4(3H)-one;
5-Fluoro-7-((1-propionylpiperidin-4-yl)methoxy)-2-(((tetrahydro-2H-pyran-4-yl)thio)methyl)quinazolin-4(3H)-one;
5-Fluoro-7-(piperidin-4-ylmethoxy)-2-(((tetrahydro-2H-pyran-4-yl)thio)methyl)quinazolin-4(3H)-one;
5,6-Difluoro-7-((1-(tetrahydro-2H-pyran-4-yl)ethyl)amino)-2-(((tetrahydro-2H-pyran-4-yl)thio)methyl)quinazolin-4(3H)-one;
7-((1-Acetylpiperidin-3-yl)methoxy)-5-fluoro-2-(((tetrahydro-2H-pyran-4-yl)thio)methyl)quinazolin-4(3H)-one;
5,6-Difluoro-2-(((tetrahydro-2H-pyran-4-yl)thio)methyl)-7-(((cis)-3-(trifluoromethoxy)cyclobutyl)amino)quinazolin-4(3H)-one;
7-Amino-5,6-difluoro-2-(((tetrahydro-2H-pyran-4-yl)thio)methyl)quinazolin-4(3H)-one;
7-(Cyclopropylmethoxy)-2-((((trans)-4-(dimethylamino)cyclohexyl)thio)methyl)-5-fluoro-7,8-dihydroquinazolin-4(3H)-one;
5-Fluoro-2-((((cis)-3-hydroxycyclobutyl)thio)methyl)-7-((tetrahydro-2H-pyran-4-yl)methoxy)quinazolin-4(3H)-one;
5,6-Difluoro-7-((tetrahydro-2H-pyran-4-yl)methoxy)-2-(((tetrahydro-2H-pyran-4-yl)thio)methyl)quinazolin-4(3H)-one;
5,6-Difluoro-7-((2-methoxy-2-methylpropyl)amino)-2-(((tetrahydro-2H-pyran-4-yl)thio)methyl)quinazolin-4(3H)-one;
5,6-Difluoro-7-((((cis)-3-fluoro-1-methylpiperidin-4-yl)methyl)amino)-2-(((tetrahydro-2H-pyran-4-yl)thio)methyl)quinazolin-4(3H)-one;
7-((1-Acetylpiperidin-4-yl)methoxy)-5-fluoro-2-((((cis)-4-hydroxy-4-methylcyclohexyl)thio)methyl)quinazolin-4(3H)-one;
Methyl 4-(((5-fluoro-4-oxo-2-(((tetrahydro-2H-pyran-4-yl)thio)methyl)-3,4-dihydroquinazolin-7-yl)oxy)methyl)piperidine-1-carboxylate;
5-Fluoro-2-((((trans)-4-hydroxy-4-methylcyclohexyl)thio)methyl)-7-((tetrahydro-2H-pyran-4-yl)methoxy)quinazolin-4(3H)-one;
7-(Cyclopentylamino)-5-fluoro-2-((((trans)-3-fluoro-1-methylpiperidin-4-yl)thio)methyl)quinazolin-4(3H)-one;
7-(Cyclopentylamino)-5-fluoro-2-((((cis)-3-fluoro-1-methylpiperidin-4-yl)thio)methyl)quinazolin-4(3H)-one;
5-Fluoro-7-((4-methylmorpholin-2-yl)methoxy)-2-(((tetrahydro-2H-pyran-4-yl)thio)methyl)quinazolin-4(3H)-one;
5-Fluoro-7-((1-methylpiperidin-4-yl)methoxy)-2-(((tetrahydro-2H-pyran-4-yl)thio)methyl)quinazolin-4(3H)-one;
5-Fluoro-7-(neopentyloxy)-2-(((tetrahydro-2H-pyran-4-yl)thio)methyl)quinazolin-4(3H)-one;
7-((1-Acetylpiperidin-4-yl)methoxy)-5-fluoro-2-((((trans)-4-hydroxy-4-methylcyclohexyl)thio)methyl)quinazolin-4(3H)-one;
5-Fluoro-7-((tetrahydro-2H-pyran-4-yl)methoxy)-2-((((cis)-4-(trifluoromethoxy)cyclohexyl)thio)methyl)quinazolin-4(3H)-one;
7-(((1-Acetylpiperidin-4-yl)methyl)amino)-5,6-difluoro-2-((((trans)-4-hydroxycyclohexyl)thio)methyl)quinazolin-4(3H)-one;
5,6-Difluoro-7-(methylamino)-2-(((tetrahydro-2H-pyran-4-yl)thio)methyl)quinazolin-4(3H)-one;
5-Fluoro-2-(((tetrahydro-2H-pyran-4-yl)thio)methyl)-7-(3,3,3-trifluoro-2,2-dimethylpropoxy)quinazolin-4(3H)-one;
7-((1-Acetylpiperidin-4-yl)methoxy)-5-fluoro-2-((((cis)-4-hydroxycyclohexyl)thio)methyl)quinazolin-4(3H)-one;
7-((1-Acetylpiperidin-4-yl)methoxy)-5-chloro-2-(((tetrahydro-2H-pyran-4-yl)thio)methyl)quinazolin-4(3H)-one;
5-Fluoro-7-((1-(2-methoxyacetyl)piperidin-4-yl)methoxy)-2-(((tetrahydro-2H-pyran-4-yl)thio)methyl)quinazolin-4(3H)-one;
5,6-Difluoro-7-((((trans)-3-fluoro-1-methylpiperidin-4-yl)methyl)amino)-2-(((tetrahydro-2H-pyran-4-yl)thio)methyl)quinazolin-4(3H)-one;
N-((trans)-4-(((5-Fluoro-4-oxo-7-((tetrahydro-2H-pyran-4-yl)methoxy)-3,4-dihydroquinazolin-2-yl)methyl)thio)cyclohexyl)acetamide; and
7-((3,3-Difluoro-1-methylpiperidin-4-yl)methoxy)-5-fluoro-2-(((tetrahydro-2H-pyran-4-yl)thio)methyl)quinazolin-4(3H)-one.

62. The method of claim 1, wherein the inflammatory disease is selected from asthma, atopic dermatitis, psoriasis, rhinitis, systemic sclerosis, keloids, an eosinophilic disorder, pulmonary fibrosis, and a type 2 cytokine pathology.

63. The method of claim 1, wherein the inflammatory disease is atopic dermatitis.

64. The method of claim 1, wherein the inflammatory disease is systemic sclerosis.

65. The method of claim 2, wherein said asthma is steroid-insensitive asthma, steroid-refractory asthma, steroid-resistant asthma, atopic asthma, nonatopic asthma, persistent asthma, severe asthma, or steroid-refractory severe asthma.

66. The method of claim 65, wherein said severe asthma is T2 high endotype, T2 low endotype, or non-T2 endotype.

67. The method of claim 65, wherein said severe asthma is T2 low endotype or non-T2 endotype.

68. A method of:
(a) reducing the level of airway mucus in lung tissue,
(b) reducing blood serum IgE,
(c) reducing immune cell infiltration and activation in bronchoalveolar fluid,
(d) reducing the level of one or more inflammatory cytokines in bronchoalveolar fluid or in lung tissue, or
(e) reducing the level of one or more alarmins in bronchoalveolar fluid or lung tissue, in a patient, said method comprising administering to the patient a therapeutically effective amount of a compound of Formula I: 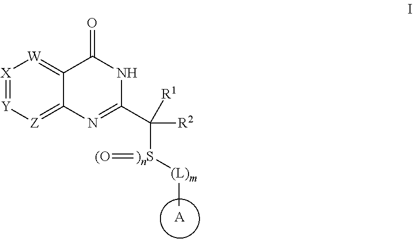
or a pharmaceutically acceptable salt thereof, wherein:
W is CR^{W} or N;
X is CR^{X} or N;
Y is CR^{Y} or N;
Z is CR^{Z} or N;
wherein no more than two of W, X, Y, and Z are simultaneously N;
Ring A is monocyclic or polycyclic C₃₋₁₄ cycloalkyl or Ring A is monocyclic or polycyclic 4-18 membered heterocycloalkyl, wherein Ring A is optionally substituted by 1, 2, 3, or 4 R^{A}, and Ring A is attached to the -(L)ₘ- moiety of Formula I through a non-aromatic ring when Ring A is polycyclic;
L is —(CR⁵R⁶)ₜ—, —(CR⁵R⁶)ₚ—O—(CR⁵R⁶)_{q}—, —(CR⁵R⁶)ₚ—S—(CR⁵R⁶)_{q}—, —(CR⁵R⁶)ₚ—NR³—(CR⁵R⁶)_{q}—, —(CR⁵R⁶)ₚ—CO—(CR⁵R⁶)_{q}—, —(CR⁵R⁶)ᵣ—C(O)O—(CR⁵R⁶)ₛ—, —(CR⁵R⁶)ᵣ—CONR³—(CR⁵R⁶)ₛ—, —(CR⁵R⁶)ₚ—SO—(CR⁵R⁶)_{q}—, —(CR⁵R⁶)ₚ—SO₂—(CR⁵R⁶)_{q}—, —(CR⁵R⁶)ᵣ—SONR³—(CR⁵R⁶)ₛ—, or —NR³CONR⁴—;
R¹ and R² are each, independently, selected from H and methyl;
R³ and R⁴ are each, independently, selected from H and C₁₋₄ alkyl;
R⁵ and R⁶ are each, independently, selected from H, halo, C₁₋₄ alkyl, C₁₋₄ alkoxy, C₁₋₄ haloalkyl, amino, C₁₋₄ alkylamino, and C₂₋₈ dialkylamino;
each R^{A} is independently selected from halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{A} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
R^{W}, R^{X}, R^{Y}, and R^{Z} are each, independently, selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃-7 cycloalkyl-C₁₋₄ alkyl, 5-10 membered heteroaryl-C₁₋₄ alkyl, and 4-10 membered heterocycloalkyl-C₁₋₄ alkyl of R^{W}, R^{X}, R^{Y}, or R^{Z} are each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
wherein when W is CR^{W}, X is CR^{X}, Y is CR^{Y}, and Z is CR^{Z}, then at least one of R^{W}, R^{X}, R^{Y}, and R^{Z} is other than H;
each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{e2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each R^{a1}, R^{b1}, R^{c1}, R^{d1}, R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a1}, R^{b1}, R^{c1}, R^{d1}, R^{a2}, R^{b2}, R^{c2}, or R^{d2} is optionally substituted with 1, 2, 3, 4, or 5 substituents independently selected from Cy³, Cy³-C₁₋₄ alkyl, halo, C₁₋₄ alkyl, C₁₋₄ haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
each Cy³ is C₆₋₁₀ aryl, C₃₋₇ cycloalkyl, 5-10 membered heteroaryl, or 4-10 membered heterocycloalkyl, each optionally substituted by 1, 2, 3, or 4 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3} NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
R^{a3}, R^{b3}, R^{c3}, and R^{d3} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
or R^{c1} and R^{d1} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
or R^{c2} and R^{d2} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
each R^{e1}, R^{e2}, and R^{e3} is independently selected from H, C₁₋₄ alkyl, and CN;
m is 0 or 1,
n is 0, 1, or 2;
p is 0, 1, or 2;
q is 0, 1, or 2, wherein p+q is 0, 1, or 2;
r is 0 or 1;
s is 0 or 1, where r+s is 0 or 1; and
t is 1, 2, or 3;
wherein any aforementioned heteroaryl or heterocycloalkyl group comprises 1, 2, 3, or 4 ring-forming heteroatoms independently selected from O, N, and S;
wherein one or more ring-forming C or N atoms of any aforementioned heterocycloalkyl group is optionally substituted by an oxo (═O) group; and
wherein one or more ring-forming S atoms of any aforementioned heterocycloalkyl group is optionally substituted by one or two oxo (═O) groups.

69-131. (canceled)

132. The method of claim 1 further comprising administering to the patient a second therapeutic agent in combination with the compound of Formula I, or a pharmaceutically acceptable salt thereof.

133. The method of claim 1, wherein the inflammatory disease is an inflammatory bowel disease.
